SEQUENCE LISTING (178 sequences)

SEQ ID NO: 1 - DNA, 47 bp, homo sapiens
tgaggtgggttggcgactaaggcgcaccggtgggcactgcggggaca

SEQ ID NO: 2 - protein, 936 aa, artificial sequence
  CD38ex1_T2-L1 TALEN
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 3 - protein, 942 aa, artificial sequence
  CD38ex1_T2-R1 TALEN
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 4 - DNA, 49 bp, homo sapiens
tgcgagttcagcccggtgtccggggacaaaccctgctgccggctctcta

SEQ ID NO: 5 - protein, 970 aa, artificial sequence
  CD38ex1_T4-L  TALEN
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNNGGKQALETVQALLPVLCQAHGLTPQQVVAIASHDGGKQALETVQRLLPVLCQAHGLTPQQVVAIASNNGGKQALETVQRLLPVLCQAHGLTPQQVVAIASNIGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNIGGKQALETVQRLLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQRLLPVLCQAHGLTPQQVVAIASHDGGKQALETVQRLLPVLCQAHGLTPEQVVAIASNNGGKQALETVQALLPVLCQAHGLTPQQVVAIASNNGGKQALETVQRLLPVLCQAHGLTPEQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 6 - protein, 942 aa, artificial sequence
  CD38ex1_T4-R  TALEN
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPQQVVAIASHDGGKQALETVQRLLPVLCQAHGLTPEQVVAIASNNGGKQALETVQRLLPVLCQAHGLTPEQVVAIASNNGGKQALETVQALLPVLCQAHGLTPQQVVAIASHDGGKQALETVQRLLPVLCQAHGLTPQQVVAIASNGGGKQALETVQRLLPVLCQAHGLTPQQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPQQVVAIASNIGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 7 - DNA, 49 bp, homo sapiens
tgatcctcgtcgtggtgctcgcggtggtcgtcccgaggtggcgccagca

SEQ ID NO: 8 - protein, 936 aa, artificial sequence
  CD38ex1_T5-L TALEN
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNNGGKQALETVQALLPVLCQAHGLTPQQVVAIASNIGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNNGGKQALETVQRLLPVLCQAHGLTPEQVVAIASNGGGKQALETVQRLLPVLCQAHGLTPQQVVAIASNNGGKQALETVQRLLPVLCQAHGLTPEQVVAIASNNGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 9 - protein, 942 aa, artificial sequence
  CD38ex1_T5-R TALEN
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASHDGGKQALETVQRLLPVLCQAHGLTPQQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPQQVVAIASNNGGKQALETVQRLLPVLCQAHGLTPEQVVAIASNNGGKQALETVQRLLPVLCQAHGLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPQQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPQQVVAIASNIGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 10 - protein, 451 aa, artificial sequence
  Daratumumab-VH chain
VQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, artificial sequence
  Daratumumab-VL chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 120 aa, artificial sequence
  MOR202-VH chain
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 13 - protein, 109 aa, artificial sequence
  MOR202-VL chain
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 14 - protein, 10 aa, artificial sequence
  HCDR1-1 - VH chain
GFTFSSYYMN

SEQ ID NO: 15 - protein, 5 aa, artificial sequence
  HCDR1-2 - VH chain
SYYMN

SEQ ID NO: 16 - protein, 17 aa, artificial sequence
  HCDR2 - VH chain
GISGDPSNTYYADSVKG

SEQ ID NO: 17 - protein, 11 aa, artificial sequence
  HCDR3 - VH chain
DLPLVYTGFAY

SEQ ID NO: 18 - protein, 5 aa, artificial sequence
  HCDR4 - VH chain
DYWMQ

SEQ ID NO: 19 - protein, 16 aa, artificial sequence
  HCDR5 - VH chain
TIYPGDGDTGYAQKFK

SEQ ID NO: 20 - protein, 11 aa, artificial sequence
  HCDR6 - VH chain
GDYYGSNSLDY

SEQ ID NO: 21 - protein, 11 aa, artificial sequence
  LCDR1 - VH chain
SGDNLRHYYVY

SEQ ID NO: 22 - protein, 7 aa, artificial sequence
  LCDR2 - VH chain
GDSKRPS

SEQ ID NO: 23 - protein, 9 aa, artificial sequence
  LCDR3 - VH chain
QTYTGGASL

SEQ ID NO: 24 - protein, 11 aa, artificial sequence
  LCDR4 - VH chain
KASQDVSTVVA

SEQ ID NO: 25 - protein, 7 aa, artificial sequence
  LCDR5 - VH chain
SASYRYI

SEQ ID NO: 26 - protein, 8 aa, artificial sequence
  LCDR6 - VH chain
QQHSPPYT

SEQ ID NO: 27 - protein, 21 aa, artificial sequence
  CD8&#945;-Signal peptide (SP)
MALPVTALLLPLALLLHAARP

SEQ ID NO: 28 - protein, 15 aa, artificial sequence
  GS linker
GGGGSGGGGSGGGGS

SEQ ID NO: 29 - protein, 16 aa, homo sapiens
GLAVSTISSFFPPGYQ

SEQ ID NO: 30 - protein, 69 aa, homo sapiens
TTTPAPRPPTPAPTIASQPLSLRPEACRPAAGGAVHTRGLDFACDIYIWAPLAGTCGVLLLSLVITLYC

SEQ ID NO: 31 - protein, 231 aa, homo sapiens
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 24 aa, homo sapiens
IYIWAPLAGTCGVLLLSLVITLYC

SEQ ID NO: 33 - protein, 42 aa, homo sapiens
KRGRKKLLYIFKQPFMRPVQTTQEEDGCSCRFPEEEEGGCEL

SEQ ID NO: 34 - protein, 112 aa, homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 442 aa, artificial sequence
  GMB005-V1 CAR
PLALLLHAARPEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDA

SEQ ID NO: 36 - protein, 471 aa, artificial sequence
  GMB005-V2 CAR
PLALLLHAARPEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDA

SEQ ID NO: 37 - protein, 657 aa, artificial sequence [sequence omitted]

SEQ ID NO: 38 - protein, 119 aa, mus musculus
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSS

SEQ ID NO: 39 - protein, 107 aa, mus musculus
DIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIK

SEQ ID NO: 40 - protein, 120 aa, mus musculus
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 41 - protein, 107 aa, mus musculus
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 42 - protein, 121 aa, mus musculus
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSA

SEQ ID NO: 43 - protein, 107 aa, mus musculus
DIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIK

SEQ ID NO: 44 - protein, 120 aa, mus musculus
QVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSS

SEQ ID NO: 45 - protein, 107 aa, mus musculus
ETTVTQSPASLSMAIGEKVTIRCITSTDIDDDMNWYQQKPGEPPKLLISEGNTLRPGVPSRFSSSGYGTDFVFTIENMLSEDVADYYCLQSDNLPLTFGGGTKLEIK

SEQ ID NO: 46 - protein, 120 aa, mus musculus; identical to SEQ ID NO: 44

SEQ ID NO: 47 - protein, 108 aa, mus musculus
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPYTFGGGTKLEIK

SEQ ID NO: 48 - protein, 432 aa, artificial sequence
  Luc63-V1 CAR
MALPVTALLLPLALLLHAARPEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSSGGGGSGGGGSGGGGSDIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIKGLAVSTISSFFPPGYQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 485 aa, artificial sequence
  Luc63-V2 CAR
MALPVTALLLPLALLLHAARPEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSSGGGGSGGGGSGGGGSDIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 647 aa, artificial sequence
  Luc63-V3 CAR
MALPVTALLLPLALLLHAARPEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSSGGGGSGGGGSGGGGSDIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 433 aa, artificial sequence
  Luc90-V1 CAR
MALPVTALLLPLALLLHAARPQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKGLAVSTISSFFPPGYQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 486 aa, artificial sequence
  Luc90-V2 CAR
MALPVTALLLPLALLLHAARPQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 648 aa, artificial sequence
  Luc90-V3 CAR
MALPVTALLLPLALLLHAARPQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 434 aa, artificial sequence
  Luc34-V1 CAR
MALPVTALLLPLALLLHAARPQVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSAGGGGSGGGGSGGGGSDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKGLAVSTISSFFPPGYQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 487 aa, artificial sequence
  Luc34-V2 CAR
MALPVTALLLPLALLLHAARPQVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSAGGGGSGGGGSGGGGSDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 649 aa, artificial sequence
  Luc34-V3 CAR
MALPVTALLLPLALLLHAARPQVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSAGGGGSGGGGSGGGGSDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 433 aa, artificial sequence
  LucX1-V1 CAR
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGGSETTVTQSPASLSMAIGEKVTIRCITSTDIDDDMNWYQQKPGEPPKLLISEGNTLRPGVPSRFSSSGYGTDFVFTIENMLSEDVADYYCLQSDNLPLTFGGGTKLEIKGLAVSTISSFFPPGYQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 487 aa, artificial sequence; identical to SEQ ID NO: 55
  LucX1-V2 CAR

SEQ ID NO: 59 - protein, 648 aa, artificial sequence
  LucX1-V3 CAR
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGGSETTVTQSPASLSMAIGEKVTIRCITSTDIDDDMNWYQQKPGEPPKLLISEGNTLRPGVPSRFSSSGYGTDFVFTIENMLSEDVADYYCLQSDNLPLTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 434 aa, artificial sequence
  LucX2-V1 CAR
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGGSDIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPYTFGGGTKLEIKGLAVSTISSFFPPGYQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 487 aa, artificial sequence
  LucX2-V2 CAR
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGGSDIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPYTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 649 aa, artificial sequence
  LucX2-V3 CAR
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGGSDIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPYTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - DNA, 49 bp, Homo sapiens
tgacttccagagagcaatatggctggttccccaacatgcctcaccctca

SEQ ID NO: 64 - DNA, 17 bp, artificial sequence
  CS1 T01 left TALE
tgacttccagagagcaa

SEQ ID NO: 65 - DNA, 17 bp, artificial sequence
  CS1 T01 right TALE
aacatgcctcaccctca

SEQ ID NO: 66 - DNA, 49 bp, homosapiens
ttccagagagcaatatggctggttccccaacatgcctcaccctcatcta

SEQ ID NO: 67 - DNA, 17 bp, artificial sequence
  CS1 T02 left TALE
ttccagagagcaatatg

SEQ ID NO: 68 - DNA, 17 bp, artificial sequence
  CS1 T02 right TALE
tgcctcaccctcatcta

SEQ ID NO: 69 - DNA, 49 bp, homo sapiens
ttgactctattgtctggaccttcaacacaacccctcttgtcaccataca

SEQ ID NO: 70 - DNA, 17 bp, artificial sequence
  CS1 T03 left TALE
ttgactctattgtctgg

SEQ ID NO: 71 - DNA, 17 bp, artificial sequence
  CS1 T03 right TALE
cctcttgtcaccataca

SEQ ID NO: 72 - DNA, 49 bp, homo sapiens
tggtcttttcttccagtgggacgtagctgagctgcagctgaatcacaca

SEQ ID NO: 73 - DNA, 17 bp, artificial sequence
  CD70 Target 1-left TALE
tggtcttttcttccagt

SEQ ID NO: 74 - DNA, 17 bp, artificial sequence
  CD70 Target 1-right TALE
tgcagctgaatcacaca

SEQ ID NO: 75 - DNA, 49 bp, homo sapiens
tggtgatctgcctcgtggtgtgcatccagcgcttcgcacaggctcagca

SEQ ID NO: 76 - DNA, 17 bp, artificial sequence
  CD70 Target 2-left TALE
tggtgatctgcctcgtg

SEQ ID NO: 77 - DNA, 17 bp, artificial sequence
  CD70 Target 2-right TALE
ttcgcacaggctcagca

SEQ ID NO: 78 - DNA, 57 bp, homo sapiens
tgcgggctgctttggtcccattggtcgcgggcttggtgatctgcctcgtggtgtgca

SEQ ID NO: 79 - DNA, 17 bp, artificial sequence
  CD70 Target 3-left TALE
tgcgggctgctttggtc

SEQ ID NO: 80 - DNA, 17 bp, artificial sequence
  CD70 Target 3-right TALE
ctgcctcgtggtgtgca

SEQ ID NO: 81 - protein, 122 aa, artificial sequence
  anti-CD70 Ab4-VH chain
QVQLVESGGGVVQPGRSLRLSCAASGFTFSNYGIHWVRQAPGKGLEWVAVIWYDGSNKYYADSVKGRFTISRDNSKNTLYLQMNSLRAEDTAVYYCARDGGYSGYDSGFDYWGQGTLVTVSS

SEQ ID NO: 82 - DNA, 1358 bp, artificial sequence
  anti-CD70 Ab4-VH chain
aggtgcagctggtggagtctgggggaggcgtggtccagcctgggaggtccctgagactctcctgtgcagcgtctggattcaccttcagtaactatggcatacactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatggaggatatagtggctacgattcggggtttgactactggggccagggaaccctggtcaccgtctcctcagctagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 83 - protein, 112 aa, artificial sequence
  anti-CD70 Ab4-VL chain
DIVMTQSPLSLPVTPGEPASISCRSSQSLLNSNGYNYLDWYLQKPGQSPQFLIYLGSYRASGVPDRFSGSGSGTDFTLRISRVEAEDVGVYYCIQTLQTPFTFGPGTKVDIK

SEQ ID NO: 84 - DNA, 1476 bp, artificial sequence
  anti-CD70 Ab4-VL chain
gatattgtgatgactcagtctccactctccctgcccgtcacccctggagagccggcctccatctcctgcaggtctagtcagagcctcctgaatagtaatggatacaactatttggattggtacctgcagaagccagggcagtctccacagttcctgatctatttgggttcttatcgggcctccggggtccctgacaggttcagtggcagtggatcaggcacagattttacactgagaatcagcagagtggaggctgaggatgttggggtttattactgtatacaaactctacaaactccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 85 - protein, 122 aa, artificial sequence
  anti-CD70 Ab8-VH chain
QVQLVESGGGVVQPGRSLRLSCAASGFTFSSYGMHWVRQAPGKGLEWVAVIWYDGSDKYFADSVKGRFTISRDNSKNTLYLQMNSLRAEDTAVYYCARDGIAGARYVYFDYWGQGTLVTVSS

SEQ ID NO: 86 - DNA, 560 bp, artificial sequence
  anti-CD70 Ab8-VH chain
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtgataaatactttgcagactccgtgaagggccgattcaccatctccagagacaattccaagaacacgctgtatctgcaaatgaacagcctgagagccgaggacacggctgtgtattactgtgcgagagatgggatagcaggagctcgctacgtctactttgactactggggccagggaaccctggtcaccgtctcctcagctagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccct

SEQ ID NO: 87 - protein, 107 aa, artificial sequence
  anti-CD70 Ab8-VL chain
DIQMTQSPSSLSASVGDRVTITCRASQGISNYLAWFQQKPGKAPKSLIYAASSLQGGVPSKFSGSGSGTDFTLTISSLQPEDFATYYCQQYYNYPFTFGPGTTVDIK

SEQ ID NO: 88 - DNA, 645 bp, artificial sequence
  anti-CD70 Ab8-VL chain
gacatccagatgacccagtctccatcctccctgtctgcatctgtaggagacagagtcaccatcacttgtcgggcgagtcagggcattagcaattatttagcctggtttcagcagaaaccagggaaagcccctaagtccctgatctatgctgcatccagtttgcaaggtggggtcccatcaaagttcagcggcagtggatctgggacagatttcactctcaccatcagcagcctgcagcctgaagattttgcaacttattactgccaacaatattataattacccattcactttcggccctgggaccacagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 89 - protein, 117 aa, artificial sequence
  anti-CD70 1F6-VH chain
QIQLVQSGPEVKKPGETVKISCKASGYTFTNYGMNWVKQAPGKGLKWMGINTYTGEPTYADAFKGRFAFSLETSASTAYLQINNLKNEDTATYFCARDYGDYGMDYWGQGTSVTVSS

SEQ ID NO: 90 - DNA, 411 bp, artificial sequence
  anti-CD70 1F6-VH chain
atggcttgggtgtggaccttgctattcctgatggcagctgcccaaagtgcccaagcacagatccagttggtgcagtctggacctgaggtgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgccttcaagggacggtttgccttctctttggaaacctctgccagcactgcctatttgcagatcaacaacctcaaaaatgaggacacggctacatatttctgtgcaagagactacggcgactatggtatggactactggggtcaaggaacctcagtcaccgtctcctca

SEQ ID NO: 91 - protein, 112 aa, artificial sequence
  anti-CD70 1F6-VL chain
DIVLTQSPASLAVSLGQRATISCRASKSVSTSGYSFMHWYQQKPGQPPKLLIYLASNLESGVPARFSGSGSGTDFTLNIHPVEEEDAATYYCQHSREVPWTFGGGTKLEIKR

SEQ ID NO: 92 - DNA, 396 bp, artificial sequence
  anti-CD70 1F6-VL chain
atggagacagacacactcctgttatgggtactgctgctctgggttccaggttccactggtgacattgtgctgacacagtctcctgcttccttagctgtatctctggggcagagggccaccatctcatgcagggccagcaaaagtgtcagtacatctggctatagttttatgcactggtatcaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggaggttccgtggacgttcggtggaggcaccaagctggaaatcaaacgg

SEQ ID NO: 93 - protein, 464 aa, artificial sequence
  anti-CD70 Ab4-V1 CAR
MALPVTALLLPLALLLHAARPQVQLVESGGGVVQPGRSLRLSCAASGFTFSNYGIHWVRQAPGKGLEWVAVIWYDGSNKYYADSVKGRFTISRDNSKNTLYLQMNSLRAEDTAVYYCARDGGYSGYDSGFDYWGQGTLVTVSSGGGGSGGGGSGGGGSDIVMTQSPLSLPVTPGEPASISCRSSQSLLNSNGYNYLDWYLQKPGQSPQFLIYLGSYRASGVPDRFSGSGSGTDFTLRISRVEAEDVGVYYCIQTLQTPFTFGPGTKVDIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 493 aa, artificial sequence
  anti-CD70 Ab4-V2 CAR
MALPVTALLLPLALLLHAARPQVQLVESGGGVVQPGRSLRLSCAASGFTFSNYGIHWVRQAPGKGLEWVAVIWYDGSNKYYADSVKGRFTISRDNSKNTLYLQMNSLRAEDTAVYYCARDGGYSGYDSGFDYWGQGTLVTVSSGGGGSGGGGSGGGGSDIVMTQSPLSLPVTPGEPASISCRSSQSLLNSNGYNYLDWYLQKPGQSPQFLIYLGSYRASGVPDRFSGSGSGTDFTLRISRVEAEDVGVYYCIQTLQTPFTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 584 aa, artificial sequence
  anti-CD70 Ab4-V3 CAR
MALPVTALLLPLALLLHAARPQVQLVESGGGVVQPGRSLRLSCAASGFTFSNYGIHWVRQAPGKGLEWVAVIWYDGSNKYYADSVKGRFTISRDNSKNTLYLQMNSLRAEDTAVYYCARDGGYSGYDSGFDYWGQGTLVTVSSGGGGSGGGGSGGGGSDIVMTQSPLSLPVTPGEPASISCRSSQSLLNSNGYNYLDWYLQKPGQSPQFLIYLGSYRASGVPDRFSGSGSGTDFTLRISRVEAEDVGVYYCIQTLQTPFTFGPGTKVDIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQ

SEQ ID NO: 96 - protein, 459 aa, artificial sequence
  anti-CD70 Ab8-V1 CAR
MALPVTALLLPLALLLHAARPQVQLVESGGGVVQPGRSLRLSCAASGFTFSSYGMHWVRQAPGKGLEWVAVIWYDGSDKYFADSVKGRFTISRDNSKNTLYLQMNSLRAEDTAVYYCARDGIAGARYVYFDYWGQGTLVTVSSGGGGSGGGGSGGGGSDIQMTQSPSSLSASVGDRVTITCRASQGISNYLAWFQQKPGKAPKSLIYAASSLQGGVPSKFSGSGSGTDFTLTISSLQPEDFATYYCQQYYNYPFTFGPGTTVDIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 488 aa, artificial sequence
  anti-CD70 Ab8-V2 CAR
MALPVTALLLPLALLLHAARPQVQLVESGGGVVQPGRSLRLSCAASGFTFSSYGMHWVRQAPGKGLEWVAVIWYDGSDKYFADSVKGRFTISRDNSKNTLYLQMNSLRAEDTAVYYCARDGIAGARYVYFDYWGQGTLVTVSSGGGGSGGGGSGGGGSDIQMTQSPSSLSASVGDRVTITCRASQGISNYLAWFQQKPGKAPKSLIYAASSLQGGVPSKFSGSGSGTDFTLTISSLQPEDFATYYCQQYYNYPFTFGPGTTVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 674 aa, artificial sequence
  anti-CD70 Ab8-V3 CAR
MALPVTALLLPLALLLHAARPQVQLVESGGGVVQPGRSLRLSCAASGFTFSSYGMHWVRQAPGKGLEWVAVIWYDGSDKYFADSVKGRFTISRDNSKNTLYLQMNSLRAEDTAVYYCARDGIAGARYVYFDYWGQGTLVTVSSGGGGSGGGGSGGGGSDIQMTQSPSSLSASVGDRVTITCRASQGISNYLAWFQQKPGKAPKSLIYAASSLQGGVPSKFSGSGSGTDFTLTISSLQPEDFATYYCQQYYNYPFTFGPGTTVDIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 459 aa, artificial sequence
  anti-CD70 1F6-V1 CAR
MALPVTALLLPLALLLHAARPQIQLVQSGPEVKKPGETVKISCKASGYTFTNYGMNWVKQAPGKGLKWMGINTYTGEPTYADAFKGRFAFSLETSASTAYLQINNLKNEDTATYFCARDYGDYGMDYWGQGTSVTVSSGGGGSGGGGSGGGGSDIVLTQSPASLAVSLGQRATISCRASKSVSTSGYSFMHWYQQKPGQPPKLLIYLASNLESGVPARFSGSGSGTDFTLNIHPVEEEDAATYYCQHSREVPWTFGGGTKLEIKRGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 488 aa, artificial sequence
  anti-CD70 1F6-V2 CAR
MALPVTALLLPLALLLHAARPQIQLVQSGPEVKKPGETVKISCKASGYTFTNYGMNWVKQAPGKGLKWMGINTYTGEPTYADAFKGRFAFSLETSASTAYLQINNLKNEDTATYFCARDYGDYGMDYWGQGTSVTVSSGGGGSGGGGSGGGGSDIVLTQSPASLAVSLGQRATISCRASKSVSTSGYSFMHWYQQKPGQPPKLLIYLASNLESGVPARFSGSGSGTDFTLNIHPVEEEDAATYYCQHSREVPWTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 674 aa, artificial sequence
  anti-CD70 1F6-V3 CAR
MALPVTALLLPLALLLHAARPQIQLVQSGPEVKKPGETVKISCKASGYTFTNYGMNWVKQAPGKGLKWMGINTYTGEPTYADAFKGRFAFSLETSASTAYLQINNLKNEDTATYFCARDYGDYGMDYWGQGTSVTVSSGGGGSGGGGSGGGGSDIVLTQSPASLAVSLGQRATISCRASKSVSTSGYSFMHWYQQKPGQPPKLLIYLASNLESGVPARFSGSGSGTDFTLNIHPVEEEDAATYYCQHSREVPWTFGGGTKLEIKREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 103 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 104 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 105 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 106 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 107 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 108 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 109 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 110 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 111 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 112 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 113 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 114 - protein, 924 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 115 - protein, 924 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 116 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 117 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 118 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 119 - protein, 925 aa, Artificial Sequence
  TAL protein sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 120 - DNA, 23 bp, Homo sapiens
gagaatcaaaatcggtgaatagg

SEQ ID NO: 121 - DNA, 23 bp, Homo sapiens
ttcaaaacctgtcagtgattggg

SEQ ID NO: 122 - DNA, 23 bp, Homo sapiens
tgtgctagacatgaggtctatgg

SEQ ID NO: 123 - DNA, 23 bp, Homo sapiens
cgtcatgagcagattaaacccgg

SEQ ID NO: 124 - DNA, 23 bp, Homo sapiens
tcagggttctggatatctgtggg

SEQ ID NO: 125 - DNA, 23 bp, Homo sapiens
gtcagggttctggatatctgtgg

SEQ ID NO: 126 - DNA, 23 bp, Homo sapiens
ttcggaacccaatcactgacagg

SEQ ID NO: 127 - DNA, 23 bp, Homo sapiens
taaacccggccactttcaggagg

SEQ ID NO: 128 - DNA, 23 bp, Homo sapiens
aaagtcagatttgttgctccagg

SEQ ID NO: 129 - DNA, 23 bp, Homo sapiens
aacaaatgtgtcacaaagtaagg

SEQ ID NO: 130 - DNA, 23 bp, Homo sapiens
tggatttagagtctctcagctgg

SEQ ID NO: 131 - DNA, 23 bp, Homo sapiens
taggcagacagacttgtcactgg

SEQ ID NO: 132 - DNA, 23 bp, Homo sapiens
agctggtacacggcagggtcagg

SEQ ID NO: 133 - DNA, 23 bp, Homo sapiens
gctggtacacggcagggtcaggg

SEQ ID NO: 134 - DNA, 23 bp, Homo sapiens
tctctcagctggtacacggcagg

SEQ ID NO: 135 - DNA, 23 bp, Homo sapiens
tttcaaaacctgtcagtgattgg

SEQ ID NO: 136 - DNA, 23 bp, Homo sapiens
gattaaacccggccactttcagg

SEQ ID NO: 137 - DNA, 23 bp, Homo sapiens
ctcgaccagcttgacatcacagg

SEQ ID NO: 138 - DNA, 23 bp, Homo sapiens
agagtctctcagctggtacacgg

SEQ ID NO: 139 - DNA, 23 bp, Homo sapiens
ctctcagctggtacacggcaggg

SEQ ID NO: 140 - DNA, 23 bp, Homo sapiens
aagttcctgtgatgtcaagctgg

SEQ ID NO: 141 - DNA, 23 bp, Homo sapiens
atcctcctcctgaaagtggccgg

SEQ ID NO: 142 - DNA, 23 bp, Homo sapiens
tgctcatgacgctgcggctgtgg

SEQ ID NO: 143 - DNA, 23 bp, Homo sapiens
acaaaactgtgctagacatgagg

SEQ ID NO: 144 - DNA, 23 bp, Homo sapiens
atttgtttgagaatcaaaatcgg

SEQ ID NO: 145 - DNA, 23 bp, Homo sapiens
catcacaggaactttctaaaagg

SEQ ID NO: 146 - DNA, 23 bp, Homo sapiens
gtcgagaaaagctttgaaacagg

SEQ ID NO: 147 - DNA, 23 bp, Homo sapiens
ccactttcaggaggaggattcgg

SEQ ID NO: 148 - DNA, 23 bp, Homo sapiens
ctgacaggttttgaaagtttagg

SEQ ID NO: 149 - DNA, 23 bp, Homo sapiens
agctttgaaacaggtaagacagg

SEQ ID NO: 150 - DNA, 23 bp, Homo sapiens
tggaataatgctgttgttgaagg

SEQ ID NO: 151 - DNA, 23 bp, Homo sapiens
agagcaacagtgctgtggcctgg

SEQ ID NO: 152 - DNA, 23 bp, Homo sapiens
ctgtggtccagctgaggtgaggg

SEQ ID NO: 153 - DNA, 23 bp, Homo sapiens
ctgcggctgtggtccagctgagg

SEQ ID NO: 154 - DNA, 23 bp, Homo sapiens
tgtggtccagctgaggtgagggg

SEQ ID NO: 155 - DNA, 23 bp, Homo sapiens
cttcttccccagcccaggtaagg

SEQ ID NO: 156 - DNA, 23 bp, Homo sapiens
acacggcagggtcagggttctgg

SEQ ID NO: 157 - DNA, 23 bp, Homo sapiens
cttcaagagcaacagtgctgtgg

SEQ ID NO: 158 - DNA, 23 bp, Homo sapiens
ctggggaagaaggtgtcttctgg

SEQ ID NO: 159 - DNA, 23 bp, Homo sapiens
tcctcctcctgaaagtggccggg

SEQ ID NO: 160 - DNA, 23 bp, Homo sapiens
ttaatctgctcatgacgctgcgg

SEQ ID NO: 161 - DNA, 23 bp, Homo sapiens
acccggccactttcaggaggagg

SEQ ID NO: 162 - DNA, 23 bp, Homo sapiens
ttcttccccagcccaggtaaggg

SEQ ID NO: 163 - DNA, 23 bp, Homo sapiens
cttacctgggctggggaagaagg

SEQ ID NO: 164 - DNA, 23 bp, Homo sapiens
gacaccttcttccccagcccagg

SEQ ID NO: 165 - DNA, 23 bp, Homo sapiens
gctgtggtccagctgaggtgagg

SEQ ID NO: 166 - DNA, 23 bp, Homo sapiens
ccgaatcctcctcctgaaagtgg

SEQ ID NO: 167 - DNA, 47 bp, Homo sapiens
tatatcctttggcagctcacaggtgagtccggccggattctcttcca

SEQ ID NO: 168 - DNA, 47 bp, Homo sapiens
tggttccccaacatgcctcaccctcatctatatcctttggcagctca

SEQ ID NO: 169 - DNA, 45 bp, Homo sapiens
tccccaacatgcctcaccctcatctatatcctttggcagctcaca

SEQ ID NO: 170 - DNA, 49 bp, Homo sapiens
tggttccccaacatgcctcaccctcatctatatcctttggcagctcaca

SEQ ID NO: 171 - protein, 902 aa, Artificial Sequence
  CS1_T04_L1 TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 172 - protein, 902 aa, Artificial Sequence
  CS1_T04_R1 TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 173 - protein, 902 aa, Artificial Sequence
  CS1_T05_L1 TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 174 - protein, 902 aa, Artificial Sequence
  CS1_T05_R1 TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 175 - protein, 902 aa, Artificial Sequence
  CS1_T06_L1 TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 176 - protein, 902 aa, Artificial Sequence
  CS1_T06_R1 TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 177 - protein, 925 aa, Artificial Sequence
  CS1_T07_L1 TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 178 - protein, 925 aa, Artificial Sequence
  CS1_T07_R1 TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

(19) United States
(12) Patent Application Publication
(10) Pub. No.: US 2023/0158070 A1
(43) Pub. Date: May 25, 2023

(54) COMBINATION COMPRISING ALLOGENEIC IMMUNE CELLS DEFICIENT FOR AN ANTIGEN PRESENT ON BOTH T-CELLS AND PATHOLOGICAL CELLS AND THERAPEUTIC ANTIBODY AGAINST SAID ANTIGEN

(71) Applicant: CELLECTIS, Paris (FR)

(72) Inventor: Philippe DUCHATEAU, DRAVEIL (FR)

(21) Appl. No.: 16/965,834

(22) PCT Filed: Jan. 30, 2019
(86) PCT No.: PCT/EP2019/052229

§ 371 Date: Jul. 29, 2020

(30) Foreign Application Priority Data
Jan. 30, 2018 (DK) PA201870063

(51) Int. Cl. A61K 35/17; C07K 14/725; C07K 16/28

(52) U.S. Cl. CPC A61K 35/17; C07K 14/7051; C07K 16/2803; A61K 2039/505

(57) ABSTRACT

The present invention relates to a therapeutic combination of immune cells, preferably allogeneic non-alloreactive TCR-KO immune T cells, wherein a gene coding an antigen marker X present on both T-cells and pathological cells is inactivated and a corresponding therapeutic antibody specific for said antigen marker X, method for preparing the same and use in immunotherapy.

### CROSS REFERENCE TO RELATED APPLICATION

This application is a national phase application under 35 U.S.C. § 371 of PCT International Application No. PCT/EP2019/052229, filed Jan. 30, 2019, which claims the benefit of Denmark Application No. PA201870063, filed Jan. 30, 2018, each of which is herein incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 430,675 Byte ASCII (Text) file named “2021-01-08_38569-251_SQL_ST25.txt,” created on Jan. 8, 2021.

### FIELD OF THE INVENTION

The present invention relates to a therapeutic combination of immune cells, preferably allogeneic non-alloreactive TCR-KO immune T cells, wherein a gene coding an antigen marker X present on both T-cells and pathological cells is inactivated and a corresponding therapeutic antibody specific for said antigen marker X, method for preparing the same and use in immunotherapy.

The present invention also relates to rare cutting endonucleases (ex: Cas9/CRISPR, meganucleases, Zinc-finger nucleases or TAL nucleases, specific for the gene encoding said antigen marker, their use for making allogeneic therapeutic cells, alone or in combination with a therapeutic antibody specific for said marker antigen. The engineered cells can be used in immune T cell depleted hosts, in the presence of said corresponding therapeutic antibody. The invention opens the way to standard and affordable adoptive immunotherapy strategies using in particular T-cells resistant to treatment with anti-T cells therapeutic antibody, for treating a cancer, an infection and an auto-immune disease.

### BACKGROUND OF THE INVENTION

Adoptive immunotherapy, which involves the transfer of antigen-specific T cells generated ex vivo, is a promising strategy to treat viral infections and cancer. The T cells used for adoptive immunotherapy can be generated either by expansion of antigen-specific T cells or redirection of T cells through genetic engineering (Park, Rosenberg et al. 2011, see, e.g., Brenner et al., Current Opinion in Immunology, 22(2): 251-257 (2010). A Genetic modification consists in expressing chimeric antigen receptors (CARs) in immune T cells. CAR are fusion proteins comprised of an antigen recognition moiety and T cell activation domains (see, e.g., Eshhar et al., Proc. Natl. Acad. Sci. USA, 90(2): 720-724 (1993), and Sadelain et al., Current Opinion in Immunology, 21(2): 215-223 (2009)). Often, the antigen recognition moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs successfully redirect T cell cytotoxicity, however, they failed to provide prolonged expansion and anti-tumor activity in vivo. Signaling domains from co-stimulatory molecules including CD28, OX-40 (CD134), and 4-1BB (CD137) added alone (second generation) or in combination (third generation) to the cytoplasmic domain of CARSs enhance survival and increase proliferation of CAR modified T cells. Other domains seem to be determinant in the capacity of CARs to bind their target such as the hinge or stalk domain.

The current protocol for treatment of patients using adoptive immunotherapy is based on autologous cell transfer. In this approach, T lymphocytes are recovered from patients, genetically modified or selected ex vivo, cultivated and expanded in vitro in order to amplify the number of cells and finally infused into the patient. In addition to lymphocyte infusion, the host may be manipulated in other ways that support the engraftment of the T cells or their participation in an immune response, for example pre-conditioning (with radiation or chemotherapy) and administration of lymphocyte growth factors (such as IL-2). Each patient receives an individually fabricated treatment, using the patient's own lymphocytes (i.e. an autologous therapy). Autologous therapies face consequently substantial technical and logistic hurdles to practical application, their generation requires expensive dedicated facilities and expert personnel, they must be generated in a short time following a patient's diagnosis, and in many cases, pretreatment of the patient has resulted in degraded immune function, such that the patient's lymphocytes may be poorly functional and present in very low numbers. Because of these hurdles, each patient's autologous cell preparation is effectively a new product, resulting in substantial variations in efficacy and safety.

Ideally, one would like to use a standardized therapy in which allogeneic therapeutic cells could be pre-manufactured, characterized in detail, and available for immediate administration to patients. By allogeneic it is meant that the cells are obtained from individuals belonging to the same species but are genetically dissimilar. The use of allogeneic cells may nevertheless encounter many drawbacks. In immune-competent hosts allogeneic cells are rapidly rejected, a process termed host versus graft rejection (HvG), and this substantially limits the efficacy of the transferred cells. In immune-incompetent or compatible hosts, allogeneic cells are able to engraft, but their endogenous T-cell receptors (TCR) specificities may still recognize the host tissue as foreign, resulting in graft versus host disease (GvHD), which can lead to serious tissue damage and death.

In order to provide less alloreactive allogeneic T-cells, preferably allogeneic non-alloreactive TCR-KO immune T cells, the inventors previously disclosed a method to genetically engineer immune cells, in which different effector genes, in particular those encoding T-cell receptors, were inactivated by using specific TAL-nucleases, better known under the trade mark TALEN™ (Cellectis, 8, rue de la Croix Jarry, 75013 PARIS). This method has proven to be highly efficient in primary cells using RNA transfection as part of a platform allowing the mass production of allogeneic T-cells (WO 2013/176915).

Effector cells and pathological cells may express a number of common antigens such as those described in WO2015121454A1.

Thus, CD38 (cluster of differentiation 38), also known as cyclic ADP ribose hydrolase is a glycoprotein found on the surface of many immune cells (white blood cells), in particular on T-cells, including CD4+, CD8+, B lymphocytes and natural killer cells. CD38 also functions in cell adhesion, signal transduction and calcium signaling. Structural information about this protein can be found in the UniProtKB/Swiss-Prot database under reference P28907. In humans, the CD38 protein is encoded by the CD38 gene which located on chromosome 4. CD38 is a multifunctional ectoenzyme that catalyzes the synthesis and hydrolysis of cyclic ADP-ribose (cADPR) from NAD+ to ADP-ribose. These reaction products are deemed essential for the regulation of intracellular Ca2+. Also, loss of CD38 function was associated with impaired immune responses and metabolic disturbances (Malavasi F., et al. (2008). “Evolution and function of the ADP ribosyl cyclase/CD38 gene family in physiology and pathology”. Physiol. Rev. 88(3): 841-86).

CD38 protein is also a marker of HIV infection, leukemias, myelomas, solid tumors, type II diabetes mellitus and bone metabolism, as well as some other genetically determined conditions. In particular, it has been used as a prognostic marker in leukemia (Ibrahim, S. et al. (2001) CD38 expression as an important prognostic factor in B-cell chronic lymphocytic leukemia. Blood 98:181-186).

The cell surface glycoprotein CS1 (also referred in the literature as SLAMF7, CD319 or CRACC—NCBI Reference Sequence: NP_067004.3) is highly and ubiquitously expressed on the surface of non-pathological (normal) lymphocytes and myeloma cells (Hsi E D, et al. Clin Cancer Res 2008 14:2775-84). CS1 is expressed at very low levels in the majority of immune effector cells, including natural killer (NK) cells, some subsets of T cells, and normal B cells, and is almost undetectable on myeloid cells (Hsi E D, et al. Clin Cancer Res 2008 14:2775-84). Notably, CS1 is negligibly expressed in human hematopoietic stem cells (Hsi E D, et al. Clin Cancer Res 2008 14:2775-84), which can be used for stem cell transplantation to treat hematologic malignancies, including MM. The functions of CS1 in MM remain incompletely understood, and it has been documented that CS1 may play a role in myeloma cell adhesion, clonogenic growth, and tumorigenicity (Benson D M Jr, et al. J Clin Oncol 2012 30:2013-5; Tai Y T, et al. Blood 2009 113:4309-18).

Similarly, cluster of Differentiation 70 (CD70, CD27LG or TNFSF7) is a member of the tumor necrosis factor (TNF) superfamily and the ligand for CD27, a TNF superfamily receptor which is expressed on normal T cells and may be over expressed in pathological cells. The transient interaction between CD27 and CD70 provides T cell costimulation complementary to that provided by CD28. CD70 is expressed on hematological cancers such as Non-Hodgkin's Lymphoma and Hodgkin's disease as well as on solid tumors such as Glioblastoma and Renal Cell Carcinoma; with its expression on Renal Cell Carcinoma being nearly uniform (see e.g., Grewal I., et al., Expert Opinion on Therapeutic Targets, 12(3): 341-351 (2008)). CD70 antibodies are currently used to induce or produce a lymphodepletion, or to treat particular diseases (see Cancer Res 2006; 66(4): 2328-37).

Expression, even transient, of such antigens on T cells may be an obstacle during the manufacturing and production of CAR T cells due to potential target-driven T cell differentiation, exhaustion, and fratricide. To palliate this, the inventors previously disclosed genetically engineer T-cells in which, a gene encoding an antigen X expressed on both T cells and pathological cells was inactivated by using specific TAL-nucleases. In these cells, a TCR subunit may be also inactivated so that cell surface expression of endogenous TCR remains undetectable as described in WO2016142532A1 which is incorporated here in its entirety. A CAR which gene is inserted into the genome of these cells can both, inhibit cell surface expression of the TCR and specifically redirects said cells against the antigen X while providing cells with a TCR-like mediated activity.

The inventors now disclosed a combination of said engineered cell with a therapeutic antibody specific for X. The specificity of said therapeutic antibody, for the same antigen X expressed on T cells and pathological cells, targeted by the CAR and which gene is inactivated in effector allogeneic T cells makes the combination especially efficient and fast in reducing the tumoral mass as compared to either T cells or therapeutic antibody alone.

Surprisingly, the combination of drugs of the invention allows, a better circumscription of cancer cells due to the possibility of accessing different compartments in the body where cancer cells migrated and niched, as compared to individuals treated with CART cells or the antibody specific for the corresponding antigen, alone.

Targeting the same antigen marker in cancer cells using two different pathways seem also to allow reducing significantly the amount of undesired cellular immune response (such as cytokine release syndrome).

### SUMMARY OF THE INVENTION

In general, the present invention discloses a combination of immune cells comprising an inactivated antigen marker X, and a therapeutic antibody specific for X, with X being expressed on both immune cell and pathological cells. This combination is provided for the treatment of a disease mediated by pathological cells or tissues expressing X or over expressing X as compared to normal tissue.

In particular embodiments, inactivated means that a gene encoding a protein (X) in immune cells is inactivated such as X is not expressed at the cell surface or is expressed and inactive. In combination with a therapeutic antibody specific for said antigen marker X, engineered immune cells are particularly efficient in reducing the tumor mass, reducing or preventing the escape or relapse of immune cells. Examples of such antigen markers X are found in Table 4 to 14. An illustration of such antigen marker is CD38, CS1 or CD70. By antigen marker is meant the whole protein or an immune-reactive fragment thereof.

The present invention provides a combination comprising an immune cell comprising at least one inactivated gene coding a cell surface antigen X, with X being expressed on both immune cell and pathological cell and a therapeutic antibody specific for said cell surface antigen X.

The present invention provides a combination comprising engineered immune cells, or a population of engineered immune cells, comprising at least one inactivated gene coding a cell surface antigen X, with X being expressed on both immune cells and pathological cells, and a therapeutic antibody specific for said cell surface antigen X, preferably a homogenous population of immune cells, more preferably a homogenous population of immune cells expressing less than 90% X at the cell surface as compared to non engineered immune cells. In preferred embodiments, the immune cells comprise an inactivated alpha and/or beta TCR gene(s) and/or inactivated beta 2 Microglobulin gene and an inactivated antigen marker X gene, and a therapeutic antibody specific for X, with X being expressed on both immune cell and pathological cells, preferably a homogenous population of immune cells, more preferably a homogenous population of immune cells expressing less than 90% alpha beta TCR and less than 90% X at the cell surface as compared to non engineered immune cells.

The combination of the invention comprises an immune cell comprising at least one inactivated gene coding a cell surface antigen, said at least one inactivated gene coding a cell surface antigen comprising a genetic modification affecting cell surface expression of said cell surface antigen and
a) a therapeutic antibody specific for said cell surface antigen.

Preferably, the present invention provides a combination comprising
a) an immune T cell comprising at least one inactivated gene coding a cell surface antigen, said at least one inactivated gene coding a cell surface antigen comprising a genetic modification affecting cell surface expression of said cell surface antigen and
b) a therapeutic antibody specific for said cell surface antigen.

The present invention provides a combination comprising:
a) an immune T cell comprising at least one inactivated gene coding a cell surface antigen, and
b) a therapeutic antibody specific for said cell surface antigen.

In other embodiments the present invention provides a combination comprising
a) an immune T cell comprising at least one inactivated gene coding a cell surface antigen comprising a genetic modification affecting cell surface expression of said cell surface antigen and
b) a therapeutic antibody specific for said cell surface antigen,

provided that said gene is not a gene coding a component of the alpha beta TCR which modification inhibits cell surface expression of the alpha beta TCR, such as a gene coding the constant part of the TCR alpha subunit (TRAC A gene), and said therapeutic antibody is not an antibody selectively binding to an antigen present at the surface of alpha beta TCR positive (αβTCR+) cells.

In another embodiment the present invention provides a combination comprising
a) an immune T cell comprising at least one inactivated gene coding a cell surface antigen comprising a genetic modification affecting cell surface expression of said cell surface antigen and
b) a therapeutic antibody specific for said cell surface antigen, provided that is excluded: if said gene is a gene coding a component of the alpha beta TCR which modification inhibits cell surface expression of the alpha beta TCR, such as a gene coding the constant part of the TCR alpha subunit (TRAC A gene), said therapeutic antibody is an antibody selectively binding to an antigen present at the surface of alpha beta TCR positive (αβTCR+) cells

When generating “off the shelve” engineered immune cells, the invention generally concerns a combination for immunotherapy comprising:
a) an allogeneic immune T cell(s) comprising at least one inactivated gene coding a cell surface antigen X, said at least one inactivated gene coding a cell surface antigen X comprising a genetic modification affecting cell surface expression of said cell surface antigen X and
b) a therapeutic antibody specific for said cell surface antigen X, with X being expressed on both T immune cells and pathological cells.

An allogeneic immune T cell means an alpha beta TCR deficient immune T cell or cells comprising more than 90% TCR-negative T cells, preferably said allogeneic immune T cells comprising a rare cutting endonuclease mediated TCR deficient gene, which product is not expressed at the cell surface.

In preferred embodiments, said allogeneic immune T cells comprise a TALEN-mediated TCR alpha inactivated gene, even more preferably said TALEN-mediated TCR alpha inactivated gene is inactivated by insertion of a polynucleotide into the constant part of the TCR alpha subunit (TRAC A gene), even more preferably, said TALEN®-modified endogenous αβ-TCR negative human primary T cell wherein the constant region of the genomic TCR gene (TRAC gene) comprises a genetic modification generated by a TALEN® and affecting cell surface expression of the endogenous alpha beta TCR, said genomic TRAC gene comprising from 5′ to 3′:

(a) a 5′ region of said human genomic TRAC gene upstream,

(b) a recognition domain for a TALEN®,

(c) a gap or an insertion as compared to the wild type TRAC gene affecting the cell surface expression of the extracellular domain or transmembrane domain of the alpha beta TCR, said insertion comprising an exogenous polynucleotide selected from a noncoding sequence such as, a stop codon, an IRES, a coding sequence such as a sequence coding for a self-cleaving peptide in frame with the TRAC open reading frame, a sequence coding a chimeric antigen receptor (CAR), a sequence coding a TCR, a sequence coding a protein conferring sensitivity to a drug, a sequence coding a protein conferring resistance to a drug, a cytokine, a termination sequence, a combination thereof,

(c′) optionally a second TALEN® recognition domain,

(d) a 3′ region of the genomic TRAC gene.

In more preferred embodiments said allogeneic T cell may be a human primary allogeneic T cell or a homogenous population of human primary allogeneic T cells, preferably a human primary CD8 allogeneic T cell, human primary CD4 allogeneic T cell, a combination thereof.

In even more preferred embodiments, a combination of the invention comprises a TALEN®-modified endogenous αβ-TCR negative human primary allogeneic T cell comprising a recognition domain for a TALEN® comprising any one of the following sequences ttgtcccacagATATC, ttgtcccacagATATCCAG, CCGTGTACCAGCTGAGA, a combination thereof, and a TALEN®-inactivated antigen marker X gene, and a therapeutic antibody specific for X, with X being expressed on both immune cell and pathological cells,

In another embodiment the present invention provides a combination comprising
a) an allogeneic immune T cell comprising at least one inactivated gene coding a cell surface antigen X comprising a genetic modification affecting cell surface expression of said cell surface antigen X and
b) a therapeutic antibody specific for said cell surface antigen X,

provided that said gene is not a gene coding a component of the alpha beta TCR which modification inhibits cell surface expression of the alpha beta TCR, such as a gene coding the constant part of the TCR alpha subunit (TRAC A gene), and said therapeutic antibody is not an antibody selectively binding to an antigen present at the surface of alpha beta TCR positive (αβTCR+) cells.

Affecting cell surface expression of X means decreasing cell surface expression to undetectable level of X as compared to a positive control expressing said antigen X or inactivating the activity of said surface antigen X.

A genetic modification means a mutation, a deletion or an insertion, resulting in an inactivation (silencing) of a gene, and/or inactivation of transcription, translation, and/or inactivation of protein expression, inactivation of protein expression at the cell surface, inactivation of the activity of the protein coded by said gene.

The following embodiments of the invention are provided:
2) The combination according to any one of item 1 and embodiments described above wherein said immune cell is an immune T cell, a hematopoietic stem cell, preferably a TCR negative immune T cell, a TCR negative hematopoietic stem cell.

In particular embodiments said immune T cell is a population of T cells, preferably homogenous population of T cells.

A TCR negative immune cell means a cell comprising a TCR KO gene, or a TCR deficient gene, deficiency resulting in a disruption of cell surface expression of the TCR.

In the present invention alpha beta TCR deficient cells express less than 90%, preferably less than 95% alpha beta TCR at the cell surface as determined by flow cytometry.

Inhibition of cell surface expression of an antigen, means that in 80 to 90%, preferably in more than 99%, even more preferably in more than 99.9% of total cells, cell surface expression of said antigen is undetectable by flow cytometry.

Particular embodiments encompass any one of the followings:
3) The combination according to item 1 or 2 wherein said immune cell is an immune T cell, immune NK T cell, immune CD8 T cell, immune CD4 T cells, or a T cell, preferably an inflammatory T-cell, cytotoxic T-cell, regulatory T-cell or helper T-cell, more preferably Cytotoxic CD4 T− cell, Cytotoxic CD8 T− lymphocyte, even more preferably an immune T cell, and even more preferably a cytotoxic CD8 T lymphocyte.
4) The combination according to anyone of item 1 to 3 wherein said gene encoding an antigen marker X, is selected from any one of the genes in Table 4 to 14, preferably CD38, CS1 and CD70, more preferably, CD38, or CS1.
5) The combination according to anyone of item 1 to 4 wherein said genetic modification comprises a mutation, an insertion or a deletion generated using a rare cutting endonuclease.
6) The combination according to any one of item 1 to 5 wherein said genetic modification is generated using a rare cutting endonuclease selected from a Meganuclease, a transcription activator-like (TAL)-nuclease, a zing-finger nuclease (ZFN), or a RNA/DNA guided endonucleases, preferably a transcription activator-like (TAL)-nuclease.
7) The combination according to any one of item 1 to 6 wherein said genetic modification is generated by a TAL-Nuclease, preferably a mRNA encoding a TAL-Nuclease. 8) The combination according to any one of item 1 to 7 wherein said genetic modification is an insertion and is generated by a TAL-Nuclease.
9) The combination according to any one of item 1 to 8 wherein said immune T cell express a recombinant TCR, a chimeric antigen receptor (CAR), a single chain CAR or a multichain CAR.
10) The combination according to any one of item 1 to 9 wherein said immune T cell express at least one CAR specific for a cell surface marker selected from any cluster of differentiation molecules (e.g. CD16, CD19, CD20, CD22, CD30, CD38, CD40, CD64, CD70, CD78, CD79a CD79b, CD96, CLL1, CD116, CD117, CD71, CD45, CD123 and CD138), CS1, a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), disialoganglioside GD2, o-acethyl GD2, GD3, mesothelin, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RUI, RU2 (AS), intestinal carboxyl esterase, hsp70-2, HSP70, Flt3, WT1, MUC16, PRAME, TSPAN10, CLAUDIN18.2, DLL3, LY6G6D, Liv-1, CHRNA2, ADAM10, M-CSF, prostase, prostase specific antigen (PSA), PAP, NY-ESO-1, LAGA-Ia, p53, prostein, PSMA, surviving and telomerase, prostate-carcinoma tumor antigen-1 (PCTA-1), MAGE, ELF2M, neutrophil elastase, ephrin B2, insulin growth factor (IGF1)-I, IGF-II, IGFI receptor, mesothelin, a major histocompatibility complex (MHC) molecule presenting a tumor-specific peptide epitope, 5T4, RORI, Nkp30, NKG2D, tumor stromal antigens, the extra domain A (EDA) and extra domain B (EDB) of fibronectin and the A1 domain of tenascin-C (TnC A1) and fibroblast associated protein (fap); a lineage-specific or tissue specific antigen such as CD3, CD4, CD8, CD24, CD25, CD33, CD34, CD133, CD138, CTLA-4, B7-1 (CD80), B7-2 (CD86), GM-CSF, CD123, HSP70, FAP, HER2, CD79a, CD79b, CD123, MUC-1, and CS1, a cytokine receptor, endoglin, a major histocompatibility complex (MHC) molecule, BCMA (CD269, TNFRSF 17), or a virus-specific surface antigen such as an HIV specific antigen (such as HIV gp120, NEF, gp41); an EBV-specific antigen, a CMV-specific antigen, a HPV-specific antigen, a Lasse Virus-specific antigen, an Influenza Virus-specific antigen as well as any derivate or variant of these surface markers. an endogenous small antigen presented by HLA class I at the surface of the cells.
11) The combination according to any one of item 1 to 10 wherein said immune T cell express a CAR which target specifically a cell surface marker selected from CD19, CD38, HSP70, CD30, FAP, HER2, CD79a, CD79b, CD123, CD22, CLL-1, MUC-1, GD2, O-acetyl-GD2, and CS1.
The combination according to any one of item 1 to 9 wherein said immune T cell express a CAR which target specifically a cell surface marker selected from CD38, HSP70, CD30, FAP, HER2, CD123, CD22, CS1, CLL-1.
The combination according to any one of item 11 wherein said immune T cell express a CAR targeting specifically CD38.
The combination according to any one of item 11 wherein said immune T cell express a CAR targeting specifically CD123.
The combination according to any one of item 11 wherein said immune T cell express a CAR targeting specifically CD22.
The combination according to any one of item 11 wherein said immune T cell express a CAR targeting specifically CS1.
The combination according to any one of item 11 wherein said immune T cell express a CAR targeting specifically CLL-1.
The combination according to any one of the above item 1 to 10 wherein said immune T cell express a recombinant TCR isolated from a tumor.
12) The combination according to any one of item 1 to 11 wherein said immune T cell express at least one CAR specific for a cell surface marker selected from any cluster of differentiation molecules (e.g. CD16, CD20, CD22, CD30, CD38, CD40, CD64, CD78, CD79a CD79b, CD96, CLL1, CD116, CD117, CD71, CD45, CD123 and CD138), CS1, a tumor-associated surface antigen, such as ErbB2 (HER2/neu), carcinoembryonic antigen (CEA), epithelial cell adhesion molecule (EpCAM), epidermal growth factor receptor (EGFR), disialoganglioside GD2, o-acethyl GD2, GD3, mesothelin, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RUI, RU2 (AS), intestinal carboxyl esterase, hsp70-2, HSP70, M-CSF, prostase, prostase specific antigen (PSA), PAP, NY-ESO-1, LAGA-Ia, p53, prostein, PSMA, surviving and telomerase, prostate-carcinoma tumor antigen-1 (PCTA-1), MAGE, ELF2M, neutrophil elastase, ephrin B2, insulin growth factor (IGF1)-I, IGF-II, IGFI receptor, mesothelin, a major histocompatibility complex (MHC) molecule presenting a tumor-specific peptide epitope, 5T4, RORI, Nkp30, NKG2D, tumor stromal antigens, the extra domain A (EDA) and extra domain B (EDB) of fibronectin and the A1 domain of tenascin-C (TnC A1) and fibroblast associated protein (fap); a lineage-specific or tissue specific antigen such as CD3, CD4, CD8, CD24, CD25, CD34, CD133, CD138, CTLA-4, B7-1 (CD80), B7-2 (CD86), GM-CSF, CD123, FAP, HER2, CD79a, CD79b, CD123, MUC-1, and CS1, cytokine receptors, endoglin, a major histocompatibility complex (MHC) molecule, or a virus-specific surface antigen such as an HIV specific antigen (such as HIV gp120); an EBV-specific antigen, a CMV-specific antigen, a HPV-specific antigen, a Lasse Virus-specific antigen, an Influenza Virus-specific antigen as well as any derivate or variant of these surface markers. an endogenous small antigen presented by HLA class I at the surface of the cells.
13) The combination according to any one of item 1 to 10 wherein said immune T cell express a CAR which target specifically a cell surface marker selected from BCMA, CD33, EGFRVIII, Flt3, WT1, CD70, MUC16, PRAME, TSPAN10, CLAUDIN18.2, DLL3, LY6G6D, Liv-1, CHRNA2, ADAM10.
14) The combination according to any one of item 1 to 13 wherein said CAR comprises an extracellular binding domain, said extracellular binding domain comprising two, three or more than 3 mAb-specific epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab, preferably 2 mAb-specific epitopes specifically recognized by rituximab, or 1 mAb-specific epitopes specifically recognized by QBEND-10 and 3 mAb-specific epitopes specifically recognized by rituximab.
15) The combination according to any one of item 1 to 14 wherein said immune T cell is further engineered by genetically inactivating at least one gene encoding a components of the T-cell receptor (TCR) and/or affecting the expression of molecule of the HLA complex.
   The combination according to any one of item 1 to 14 wherein said immune T cell is further engineered to be less alloreactive as compared to non-engineered cells.
16) The combination according to any one of item 1 to 15 wherein a sequence encoding a CAR is inserted into the constant region of the TCR alpha gene or into the at least one gene coding a cell surface antigen comprising a genetic modification affecting cell surface expression of said cell surface antigen.
17) The combination according to any one of item 1 to 16 wherein said immune T cell is further engineered to be less alloreactive as compared to non-engineered cells, by genetically inactivating the alpha TCR, beta1 TCR, beta2 TCR.
18) The combination according to any one of item 1 to 16 wherein said immune T cell is further engineered by genetically inactivating a gene selected from beta2microglobulin, regulatory factor X-associated ankyrin-containing protein (RFXANK), regulatory factor 5 (RFX5), regulatory factor X-associated protein (RFXAP), and class II transactivator (CIITA), TAP-1, a combination thereof.
The combination according to any one of item 1 to 17 wherein said immune T cell is further engineered to be less alloreactive as compared to non-engineered cells, by genetically inactivating the alphaTCR and beta2microglobulin.
The combination according to any one of item 1 to 15 wherein said immune T cell is further engineered to be less alloreactive as compared to non-engineered cells, by genetically inactivating the alphaTCR and regulatory factor X-associated ankyrin-containing protein (RFXANK) gene.
The combination according to any one of item 1 to 15 wherein said immune T cell is further engineered to be less alloreactive as compared to non-engineered cells, by genetically inactivating the alphaTCR and regulatory factor 5 (RFX5) gene.
The combination according to any one of item 1 to 15 wherein said immune T cell is further engineered to be less alloreactive as compared to non-engineered cells, by genetically inactivating the alphaTCR and regulatory factor X-associated protein (RFXAP) gene.
The combination according to any one of item 1 to 15 wherein said immune T cell is further engineered to be less alloreactive as compared to non-engineered cells, by genetically inactivating the alphaTCR and class II transactivator (CIITA).
The combination according to any one of item 1 to 15 wherein said immune T cell is further engineered to be less alloreactive as compared to non-engineered cells, by genetically inactivating the alphaTCR and TAP-1 gene.
19) The combination according to any one of items 1 to 18 further engineered to resist hypoxia, preferably wherein said CAR comprises an intracellular domain conferring resistance to hypoxia such as at least one HIF 1 alpha domain.
20) The combination according to any one of items 1 to 19 further engineered to resist tumor-inducing inhibition of immune cells.
21) The combination according to any one of items 1 to 20 further engineered to resist tumor-inducing inhibition anti-tumor activity of immune cells mediated by any one of the following molecules, Programmed Death 1 (PD-1), Cytotoxic T-Lymphocyte Antigen 4 (CTLA-4), LAG3 Tim3, BTLA, BY55, TIGIT, B7H5, LAIR1, SIGLEC10, and 2B4.
22) The combination according to anyone of 1 to item 21 comprising immune T cells with a genetic modification of the CD70 gene affecting cell surface expression of CD70 and an anti-CD70 therapeutic antibody, preferably an anti-CD70 therapeutic antibody selected from Vorsetuzumab, Vorsetuzumab mafodotin, SGN CD70A, MDX 1203, ARGX 110, preferably an anti-CD70 therapeutic antibody ARGX-110 or MDX 1203.
23) The combination according to item 22 wherein said genetic modification is generated using a transcription activator-like (TAL)-nuclease specific for CD70, comprising at least 95% homology with SEQ ID NO 102 and SEQ ID NO 103, comprising at least 95% homology with SEQ ID NO 104 and SEQ ID NO 105, comprising at least 95% homology with SEQ ID NO 106 and SEQ ID NO 107 specific for the sequences SEQ ID NO 73 and SEQ ID NO 74; SEQ ID NO 76 and SEQ ID NO 77; SEQ ID NO 79 and SEQ ID N0780, respectively.
24) The combination according to anyone of item 1 to 21 comprising an immune T cell with a genetic modification of the CS-1 gene affecting cell surface expression of CS1 and an anti-CS-1 therapeutic antibody, preferably an anti-CS1 therapeutic antibody selected from Elotuzumab and ABBV838.
25) The combination according to item 24 wherein said genetic modification is generated using a transcription activator-like (TAL)-nuclease specific for CS1, comprising at least 95% homology with SEQ ID NO 108 and SEQ ID NO 109; SEQ ID NO 110 and SEQ ID NO 111; or SEQ ID NO 112 and SEQ ID NO 113 specific for the sequences: SEQ ID NO 64 and SEQ ID NO 65; SEQ ID NO 67 and SEQ ID NO 68; SEQ ID NO 70 and SEQ ID NO 71, respectively.
26) The combination according to anyone of item 1 to 21 comprising an immune T cell with a genetic modification of the CD38 gene affecting cell surface expression of CD38 and an anti-CD38 therapeutic antibody, preferably an anti-CD38 therapeutic antibody selected from Daratumumab HuMax-CD38—Isatuximab HuMax-CD38, MOR202, MOR-03087, EDC8, and GBR 1342.
27) The combination according to item 26 wherein said genetic modification is generated using a transcription activator-like (TAL)-nuclease specific for CD38, comprising at least 95% homology with SEQ ID NO 114 and SEQ ID NO 115; SEQ ID NO 116 and SEQ ID NO 117; or SEQ ID NO 118 and SEQ ID NO 119 specific for a sequence having at least 95% homology with SEQ ID NO 1, SEQ ID NO 4; SEQ ID NO 7, respectively.
28) A pharmaceutical composition comprising a combination according to any one of item 1 or 27 and a pharmaceutically acceptable vehicle.
29) The combination according to any of the item 1 to 27 or the pharmaceutical composition according to item 28 for use in the treatment of a cancer, an infection or an auto-immune disease, preferably a cancer expressing or over expressing said cell surface antigen targeted by said therapeutic antibody, or relapse or refractory forms of said expressing or over expressing cancers.
30) The combination or the pharmaceutical composition for use according to item 29 as a treatment in a lymphodepleted and/or irradiated patient.
31) The combination or the pharmaceutical composition for use according to item 29 or 30 in the treatment or prophylaxis of cancer, wherein said cancer is selected from a hematopoietic or solid cancer, their relapse, refractory or metastatic forms.
32) The combination or the pharmaceutical composition for use according to any one of item 29 to 31 for the treatment of Renal cell Carcinoma, Glioblastoma, glioma such as low grade glioma, Non-Hodgkin's Lymphoma (NHL), Hodgkin's Disease (HD), Waldenstrom's macroglobulinemia, Acute Myeloid Leukemia (AML), Multiple Myeloma (MM), diffuse large-cell lymphoma, follicular lymphoma and Non-Small Cell Lung Cancer.
33) The combination or the pharmaceutical composition for use according to any one of item 29 to 31 in the treatment or prophylaxis of Multiple myeloma (MM), Acute myeloid leukemia (AML), Chronic myeloid leukemia (CML), Acute lymphoblastic leukemia (ALL), Hodgkin lymphoma (HL) (relapsed, refractory), Non-Hodgkin lymphoma (NHL) (relapsed, refractory), Neuroblastoma, Ewing sarcoma, Myelodysplastic syndromes, BPDCN.
34) The combination or the pharmaceutical composition for use according to any one of item 29 to 31 in the treatment or prophylaxis of Gliomas, pancreatic cancer, lung cancer, bladder cancer, colon cancer, breast cancer.
35) The combination or the pharmaceutical composition for use according to any one of item 29 to 31 for the treatment of Non-Hodgkin's Lymphoma (indolent NHLs, follicular NHLs, small lymphocytic lymphoma, lymphoplasmacytic NHL, or marginal zone NHL); Hodgkin's disease (e.g., Reed-Sternberg cells); a cancer of the B-cell lineage, including, e.g., diffuse large B-cell lymphoma, follicular lymphoma, Burkitt's lymphoma, mantle cell lymphoma, Cutaneous T cell lymphoma, B-cell lymphocytic leukemia (e.g., acute lymphocytic leukemia, chronic lymphocytic leukemia); Epstein Barr Virus positive B cell lymphoma; renal cell carcinoma (e.g., clear cell and papillary); nasopharyngeal carcinoma; thymic carcinoma; glioma; glioblastoma; neuroblastoma; astrocytoma; meningioma; Waldenstrom macroglobulinemia; multiple myeloma; colon cancer, stomach cancer, and rectal carcinoma.
36) A combination according to any one of item 1 to 23 comprising an immune T cell with a genetic modification of the CD70 gene affecting cell surface expression of CD70 and an anti-CD70 therapeutic antibody ARGX 110, for use in the treatment of Cutaneous T cell lymphoma.
37) The combination to any one of item 1 to 23 comprising an immune T cell with a genetic modification of the CD70 gene affecting cell surface expression of CD70 and an anti-CD70 therapeutic antibody MDX 1203 for use in the treatment of Renal Cell Carcinoma or Non-hodgkin's Lymphoma.

CD70-expressing cancers that can be treated or prevented using the combination of the invention, for example, different subtypes of Non-Hodgkin's Lymphoma (indolent NHLs, follicular NHLs, small lymphocytic lymphomas, lymphoplasmacytic NHLs, or marginal zone NHLs); Hodgkin's disease (e.g., Reed-Sternberg cells); cancers of the B-cell lineage, including, e.g., diffuse large B-cell lymphomas, follicular lymphomas, Burkitt's lymphoma, mantle cell lymphomas, B-cell lymphocytic leukemias (e.g., acute lymphocytic leukemia, chronic lymphocytic leukemia); Epstein Barr Virus positive B cell lymphomas; renal cell carcinomas (e.g., clear cell and papillary); nasopharyngeal carcinomas; thymic carcinomas; gliomas; glioblastomas; neuroblastomas; astrocytomas; meningiomas; Waldenstrom macroglobulinemia; multiple myelomas; and colon, stomach, and rectal carcinomas. The cancer can be, for example, newly diagnosed, pre-treated or refractory or relapsed. In preferred embodiments, a CD38-expressing cancer has at least about 15,000, at least about 10,000 or at least about 5,000 CD38 molecules/cell.

In preferred embodiments, a CS1-expressing cancer has at least about 15,000, at least about 10,000 or at least about 5,000 CS1 molecules/cell.

In preferred embodiments, a CD70-expressing cancer has at least about 15,000, at least about 10,000 or at least about 5,000 CD70 molecules/cell.
38) The combination according to any one of item 1 to 21, 24 and 25 for the treatment of carcinoma, blastoma, and sarcoma, preferably MM, leukemia, melanoma, relapse or refractory CS-1 expressing MM or a complication related to CS-1 expressing MM.
39) The combination according to any one of item 1 to 21, 24 and 25 for the treatment of any one of the following CS1 expressing cancers: Multiple Myeloma, Acute Myeloid Leukemia, Myelodysplastic Syndrome, Smoldering Multiple Myeloma, monoclonal gammopathy of unknown significance (MGUS), plasma cell leukemia, Non-Hodgkin's Lymphoma.
40) The combination according to any one of item 1 to 21, 24 and 25 for the treatment of any one of the following CS1 expressing cancers: Multiple Myeloma, Non-Hodgkin Lymphoma, Diffuse Large B Cell Lymphoma, Mantle-Cell Lymphoma, Follicular Lymphoma, Indolent B Cell Lymphoma, Primary Mediastinal Lymphoma, Lymphoplasmacytic Lymphoma.
41) The combination according to any one of item 1 to 21, 24 and 25 for the treatment of any one of the following CS1 expressing cancers: NK cell lymphoma, NK or T cell lymphoma, angioimmunoblastic T-cell lymphoma (AITL), or peripheral T cell lymphoma not otherwise specified (PTCL-NOS).
   Document WO2010051391 A1 is incorporated herein in its entirety as prior art allowing the combination of CS1 deficient engineered TCR negative T cells and a therapeutic antibody to be prepared and used for the treatment of NK cell lymphoma, NK or T cell lymphoma, angioimmunoblastic T-cell lymphoma (AITL), or peripheral T cell lymphoma not otherwise specified (PTCL-NOS).
42) The combination according to any one of item 1 to 21, 26 and 27 comprising an immune T cell with a genetic modification of the CD38 gene affecting cell surface expression of CD38 and an anti-CD38 therapeutic antibody HuMax-CD38—Isatuximab for the treatment of Prostate Cancer, Non-small Cell Lung Cancer, Plasma Cell Myeloma, MM, T-cell Type Acute Leukemia, Precursor T-lymphoblastic Lymphoma or Leukaemia, prostate cancer, Non-small Cell Lung Cancer.
43) The combination according to any one of item 1 to 21, 26 and 27 comprising an immune T cell with a genetic modification of the CD38 gene affecting cell surface expression of CD38 and an anti-CD38 therapeutic antibody HuMax-CD38—Daratumumab for the treatment of Microsatellite Unstable Colorectal Cancer, Microsatellite Stable Colorectal Cancer, Mismatch Repair Proficient Colorectal Cancer, Mismatch Repair Deficient Colorectal Cancer, Waldenstrom Macroglobulinemia, Malignant Neoplasms of Male Genital Organs, Prostate Cancer, Hematopoietic Cancer, Acute Myelogenous Leukemia, High-Risk Myelodysplastic Syndrome, Plasma cell myeloma, Monoclonal Gammopathy, Smoldering Multiple Myeloma, Membranoproliferative Glomerulonephritis, Multiple Myeloma.
44) The combination according to any one of item 1 to 21, 26 and 27 comprising an immune T cell with a genetic modification of the CD38 gene affecting cell surface expression of CD38 and Isatuximab, for the treatment of T-cell Type Acute Leukemia-Precursor T, lymphoblastic Lymphoma, Leukaemia.
45) The combination according to any one of item 1 to 21, 26 and 27 comprising an immune T cell with a genetic modification of the CD38 gene affecting cell surface expression of CD38 and Isatuximab, for the treatment of a CD38-expressing or CD 38 over expressing hematologic cancer selected from the group of Chronic Lymphocytic Leukemia (CLL), Chronic Myelogenous Leukemia (CML), Acute Myelogenous Leukemia (AML), and Acute Lymphocytic Leukemia (ALL), multiple myeloma (MM).
46) The combination according to any one of item 1 to 21, 26 and 27 comprising an immune T cell with a genetic modification of the CD38 gene affecting cell surface expression of CD38 and Isatuximab, for the treatment of hematologic cancer selected from the group of leukemia, lymphoma and multiple myeloma (MM).
47) The combination according to any one of item 1 to 21, 26 and 27 comprising an immune T cell with a genetic modification of the CD38 gene affecting cell surface expression of CD38 and Isatuximab, for the treatment of hematologic cancer selected from the group of B-cell chronic lymphocytic leukemia (B-CLL), acute lymphoblastic leukemia, chronic myeloid leukemia, chronic lymphocytic leukemia (CLL), chronic myelogenous leukemia or chronic myeloid leukemia (CML), acute myelogenous leukemia or acute myeloid leukemia (AML), acute lymphocytic leukemia (ALL), hairy cell leukemia (HCL), myelodysplasia syndromes (MDS) or chronic myelogenous leukemia (CML-BP) and any subtypes of chronic myelogenous leukemia (CML-BP).
48) The combination according to any one of item 1 to 21, 26 and 27 for the treatment of Microsatellite Unstable Colorectal Cancer, Microsatellite Stable Colorectal Cancer, Mismatch Repair Proficient Colorectal Cancer, Mismatch Repair Deficient Colorectal Cancer, Waldenstrom Macroglobulinemia, Malignant Neoplasms of Male Genital Organs, Prostate Cancer, Hematopoietic Cancer, Acute Myelogenous Leukemia, High-Risk Myelodysplastic Syndrome, Plasma cell myeloma, Monoclonal Gammopathy, Smoldering Multiple Myeloma, Membranoproliferative Glomerulonephritis, Multiple Myeloma.
49) The combination according to any one of item 1 to 21, 26 and 27 for the treatment of T-cell Type Acute Leukemia-Precursor T, lymphoblastic Lymphoma, Leukaemia.
50) The combination according to any one of item 1 to 21, 26 and 27 for the treatment of a CD38-expressing or CD 38 over expressing hematologic cancer selected from the group of Chronic Lymphocytic Leukemia (CLL), Chronic Myelogenous Leukemia (CML), Acute Myelogenous Leukemia (AML), and Acute Lymphocytic Leukemia (ALL), multiple myeloma (MM).
51) The combination according to any one of item 1 to 21, 26 and 27 for the treatment of hematologic cancer selected from the group of leukemia, lymphoma and multiple myeloma (MM).
52) The combination according to any one of item 1 to 21, 26 and 27 for the treatment of hematologic cancer selected from the group of B-cell chronic lymphocytic leukemia (B-CLL), acute lymphoblastic leukemia, chronic myeloid leukemia, chronic lymphocytic leukemia (CLL), chronic myelogenous leukemia or chronic myeloid leukemia (CML), acute myelogenous leukemia or acute myeloid leukemia (AML), acute lymphocytic leukemia (ALL), hairy cell leukemia (HCL), myelodysplasia syndromes (MDS) or chronic myelogenous leukemia (CML-BP) and any subtypes of chronic myelogenous leukemia (CML-BP), leukemia, lymphoma and multiple myeloma.
Non-limiting examples of conditions associated with CD38 expression include but are not limited to, multiple myeloma (Jackson et al. (1988), Clin. Exp. Immunol. 72: 351-356), B-cell chronic lymphocytic leukemia (B-CLL) Durig et al. (2002), Leukemia 16: 30-5; Morabito et al. (2001), Leukemia Research 25: 927-32; Marinov et al. (1993), Neoplasma 40(6): 355-8; and Jelinek et al. (2001), Br. J. Haematol. 115: 854-61), acute lymphoblastic leukemia (Keyhani et al. (1999), Leukemia Research 24: 153-9; and Marinov et al. (1993), Neoplasma 40(6): 355-8), chronic myeloid leukemia (Marinov et al. (1993), Neoplasma 40(6): 355-8), acute myeloid leukemia (Keyhani et al. (1999), Leukemia Research 24: 153-9), chronic lymphocytic leukemia (CLL), chronic myelogenous leukemia or chronic myeloid leukemia (CML), acute myelogenous leukemia or acute myeloid leukemia (AML), acute lymphocytic leukemia (ALL), hairy cell leukemia (HCL), myelodysplasia syndromes (MDS) or chronic myelogenous leukemia (CML-BP) in blastic and all subtypes of these leukemias which are defined by morphological, histochemical and immunological techniques that are well known by those of skill in the art.
53. A method for treating a patient comprising:
   optionally diagnosing said patient for the presence of pathological cells expressing an antigen marker on the cell surface;
   Preparing a combination of a therapeutic antibody specific for an antigen marker expressed at the cell surface of pathological cells and a population of genetically modified immune cells wherein the gene(s) encoding said antigen marker is inactivated according to any of the item 1 to 27 or a pharmaceutical composition of item 28,
   Administering said genetically modified cells to said patient, before, after and/or concomitantly to said therapeutic antibody.

CS 1 (also known as SLAMF7, CRACC, 19A, APEX-1, FOAP 12, and 19A; GENBANK® Accession No. NM 021181.3, Ref. Boles et al, Immunogenetics, 52:302-307 (2001); Bouchon et al, J. Immunol, 167:5517-5521 (2001); Murphy et al, Biochem. J., 361:431-436 (2002)) is a member of the CD2 subset of the immunoglobulin superfamily. Molecules of the CD2 family are involved in a broad range of immunomodulatory functions, such as co-activation, proliferation differentiation, and adhesion of lymphocytes, as well as immunoglobulin secretion, cytokine production, and NK cell cytotoxicity. Several members of the CD2 family, such as CD2, CD58, and CD 150, play a role or have been proposed to play a role in a number of autoimmune and inflammatory diseases, such as psoriasis, rheumatoid arthritis, and multiple sclerosis. It has been reported that CS 1 plays a role in NK cell-mediated cytotoxicity and lymphocyte adhesion (Bouchon, A. et al, J. Immunol, 5517-5521 (2001); Murphy, J. et al, Biochem. J., 361:431-436 (2002)).

Elotuzumab is a humanized monoclonal IgGI antibody directed against CS-1, a cell surface glycoprotein, which is highly and uniformly expressed in multiple myeloma. Elotuzumab induces significant antibody-dependent cellular cytotoxicity (ADCC) against primary multiple myeloma cells in the presence of peripheral lymphocytes (Tai et al, Blood, 112: 1329-1337 (2008)). Results of three studies that evaluated the safety and efficacy of this drug administered alone (Zonder et al, Blood, 120(3):552-559 (2012)), in combination with bortezomib (Jakubowiak et al, J. Clin. Oncol., 30(16): 1960-1965 (Jun. 1, 2012)), or lenalidomide and low-dose dexamethasone (Lonial et al, J. Clin. Oncol., 30: 1953-1959 (2012); and Richardson et al, Blood (ASH Annual Meeting Abstracts), 1 16:986 (2010) for the treatment of patients with relapsed or refractory multiple myeloma, have been reported. All three combinations showed a manageable safety profile and encouraging activity. For example, a Phase VII study evaluating the safety and efficacy of Elotuzumab in combination lenalidomide and low-dose dexamethasone for the treatment of relapsed or refractory multiple myeloma demonstrated a 33 month PFS as well as a 92% response rate for patients receiving the 10 mg/kg dose (Lonial et al., J. Clin. Oncol., 31 (2013) (Suppl., Abstr. 8542)). Phase III clinical trials of lenalidomide/dexamethasone with or without Elotuzumab in previously untreated multiple myeloma patients is ongoing, while another phase III trial designed to evaluate this same combination in the first line setting is also ongoing.
54. A TAL-protein comprising a sequence having at least 95% homology with SEQ ID NO 114 and SEQ ID NO 115; with SEQ ID NO 116 and SEQ ID NO 117; with SEQ ID NO 118 and SEQ ID NO 119; with SEQ ID NO 102 and SEQ ID NO 103; with SEQ ID NO 104 and SEQ ID NO 105; with SEQ ID NO 106 and SEQ ID NO 107; with SEQ ID NO 108 and SEQ ID NO 109; with SEQ ID NO 110 and SEQ ID NO 111; with SEQ ID NO 112 and SEQ ID NO 113; with SEQ ID NO 171 and SEQ ID NO 172; with SEQ ID NO 173 and SEQ ID NO 174; with SEQ ID NO 175 and SEQ ID NO 176; or with SEQ ID NO 177 and SEQ ID NO 178.

In particular embodiments a TAL-protein as any one of the TAL protein of item 54, is provided for use in the manufacturing of CART cells.

A TAL-protein comprising a sequence having at least 95% homology with SEQ ID NO 114 and SEQ ID NO 115; with SEQ ID NO 116 and SEQ ID NO 117; or with SEQ ID NO 118 and SEQ ID NO 119, is provided for modifying the CD38 gene.

A TAL-protein comprising a sequence having at least 95% homology with SEQ ID NO 102 and SEQ ID NO 103; with SEQ ID NO 104 and SEQ ID NO 105; or with SEQ ID NO 106 and SEQ ID NO 107, is provided for modifying the CD70 gene.

A TAL-protein comprising a sequence having at least 95% homology with SEQ ID NO 108 and SEQ ID NO 109; with SEQ ID NO 110 and SEQ ID NO 111; with SEQ ID NO 112 and SEQ ID NO 113; with SEQ ID NO 171 and SEQ ID NO 172; with SEQ ID NO 173 and SEQ ID NO 174; with SEQ ID NO 175 and SEQ ID NO 176; or with SEQ ID NO 177 and SEQ ID NO 178, is provided for modifying the CS1 gene.

A polynucleotide or a vector encoding any one of the TAL protein above is provided.

A TAL-protein comprising a sequence of SEQ ID NO 114 and SEQ ID NO 115.

A TAL-protein comprising a sequence of SEQ ID NO 116 and SEQ ID NO 117.

A TAL-protein comprising a sequence of SEQ ID NO 118 and SEQ ID NO 119.

A TAL-protein comprising a sequence of SEQ ID NO 102 and SEQ ID NO 103.

A TAL-protein comprising a sequence of SEQ ID NO 104 and SEQ ID NO 105.

A TAL-protein comprising a sequence of SEQ ID NO 106 and SEQ ID NO 107.

A TAL-protein comprising a sequence of SEQ ID NO 108 and SEQ ID NO 109.

A TAL-protein comprising a sequence of SEQ ID NO 110 and SEQ ID NO 111.

A TAL-protein comprising a sequence of SEQ ID NO 112 and SEQ ID NO 113.

A TAL-protein comprising a sequence of SEQ ID NO 171 and SEQ ID NO 172.

A TAL-protein comprising a sequence of SEQ ID NO 173 and SEQ ID NO 174.

A TAL-protein comprising a sequence of SEQ ID NO 175 and SEQ ID NO 176.

A TAL-protein comprising a sequence of SEQ ID NO 177 and SEQ ID NO 178.

A TAL-protein according to item 54 comprising a sequence having at least 95% homology with SEQ ID NO 114 and SEQ ID NO 115; comprising a sequence having at least 95% homology with SEQ ID NO 116 and SEQ ID NO 117; comprising a sequence having at least 95% homology with SEQ ID NO 118 and SEQ ID NO 119; comprising a sequence having at least 95% homology with SEQ ID NO 102 and SEQ ID NO 103; comprising a sequence having at least 95% homology with SEQ ID NO 104 and SEQ ID NO 105; comprising a sequence having at least 95% homology with SEQ ID NO 106 and SEQ ID NO 107; comprising a sequence having at least 95% homology with SEQ ID NO 108 and SEQ ID NO 109; comprising a sequence having at least 95% homology with SEQ ID NO 110 and SEQ ID NO 111; comprising a sequence having at least 95% homology with SEQ ID NO 112 and SEQ ID NO 113; comprising a sequence having at least 95% homology with SEQ ID NO 171 and SEQ ID NO 172; comprising a sequence having at least 95% homology with SEQ ID NO 173 and SEQ ID NO 174; comprising a sequence having at least 95% homology with SEQ ID NO 175 and SEQ ID NO 176; or comprising a sequence having at least 95% homology with SEQ ID NO 177 and SEQ ID NO 178 for use in the manufacturing of CART cells.

A TAL-protein comprising a sequence having at least 95% homology with SEQ ID NO 114 and SEQ ID NO 115; with SEQ ID NO 116 and SEQ ID NO 117; with SEQ ID NO 118 and SEQ ID NO 119; is provided for modifying the CD38 gene.

A TAL-protein comprising a sequence having at least 95% homology with SEQ ID NO 102 and SEQ ID NO 103; with SEQ ID NO 104 and SEQ ID NO 105; with SEQ ID NO 106 and SEQ ID NO 107, is provided for modifying the CD70 gene.

A TAL-protein comprising a sequence having at least 95% homology with SEQ ID NO 108 and SEQ ID NO 109; with SEQ ID NO 110 and SEQ ID NO 111; with SEQ ID NO 112 and SEQ ID NO 113 with SEQ ID NO 171 and SEQ ID NO 172; with SEQ ID NO 173 and SEQ ID NO 174; with SEQ ID NO 175 and SEQ ID NO 176; or with SEQ ID NO 177 and SEQ ID NO 178 is provided for modifying the CS1 gene.

A polynucleotide or a vector encoding a protein comprising any one of the TAL protein above is provided.

A polynucleotide or a vector encoding any one of the TAL protein above is provided.

An engineered immune T cell comprising a CD38 modified gene obtained using a TAL-protein or a polynucleotide encoding a sequence having at least 95% homology with SEQ ID NO 114 and SEQ ID NO 115; with SEQ ID NO 116 and SEQ ID NO 117; or with SEQ ID NO 118 and SEQ ID NO 119 is provided.

An engineered immune T cell comprising a CD70 modified gene obtained using a TAL-protein or a polynucleotide encoding a sequence having with SEQ ID NO 102 and SEQ ID NO 103; with SEQ ID NO 104 and SEQ ID NO 105; or with SEQ ID NO 106 and SEQ ID NO 107, is provided.

An engineered immune T cell comprising a CS1 modified gene obtained using a TAL-protein or a polynucleotide encoding a sequence having with SEQ ID NO 108 and SEQ ID NO 109; with SEQ ID NO 110 and SEQ ID NO 111; with SEQ ID NO 112 and SEQ ID NO 113; with SEQ ID NO 171 and SEQ ID NO 172; with SEQ ID NO 173 and SEQ ID NO 174; with SEQ ID NO 175 and SEQ ID NO 176; or with SEQ ID NO 177 and SEQ ID NO 178 is provided.
55. A polynucleotide encoding a protein comprising any one of the TAL-protein of item 54.
56. A vector coding a protein comprising any one of the TAL protein of SEQ ID NO 102 and SEQ ID NO 103, SEQ ID NO 104 and SEQ ID NO 105, SEQ ID NO 106 and SEQ ID NO 107, SEQ ID NO 108 and SEQ ID NO 109, SEQ ID NO 110 and SEQ ID NO 111, SEQ ID NO 112 and SEQ ID NO 113, SEQ ID NO 114 and SEQ ID NO 115, SEQ ID NO 116 and SEQ ID NO 117, SEQ ID NO 118 and SEQ ID NO 119, SEQ ID NO 171 and SEQ ID NO 172, SEQ ID NO 173 and SEQ ID NO 174, SEQ ID NO 175 and SEQ ID NO 176, or SEQ ID NO 177 and SEQ ID NO 178 for use in the manufacturing of CART cells.
57. A vector coding any one of the TAL protein of SEQ ID NO 102 and SEQ ID NO 103, SEQ ID NO 104 and SEQ ID NO 105, SEQ ID NO 106 and SEQ ID NO 107, SEQ ID NO 108 and SEQ ID NO 109, SEQ ID NO 110 and SEQ ID NO 111, SEQ ID NO 112 and SEQ ID NO 113, SEQ ID NO 114 and SEQ ID NO 115, SEQ ID NO 116 and SEQ ID NO 117, SEQ ID NO 118 and SEQ ID NO 119, SEQ ID NO 171 and SEQ ID NO 172, SEQ ID NO 173 and SEQ ID NO 174, SEQ ID NO 175 and SEQ ID NO 176, or SEQ ID NO 177 and SEQ ID NO 178 for use in the manufacturing of CART cells.
58) A method of preparing a combination of
   1) engineered T-cells, said engineered T cells comprising an inactivated gene coding an antigen marker, with
   2) a therapeutic antibody specific for said antigen marker and expressed at the cell surface of a pathological cells comprising the step of:
      (a) Genetically engineering a gene in a T-cell, which is involved in the expression or presentation of an antigen marker, said antigen marker being present both on the surface of said T-cell and the pathological cell; to inactivate said antigen marker and/or alter its binding to its receptor
      (b) Expressing into said T-cells a transgene encoding a chimeric antigen receptor directed against said antigen marker present at the surface of said pathological cell.
      (c) Combining said T-cells with a therapeutic antibody specific for said antigen marker
59) The method according to item 58, wherein said antigen marker is selected from one listed in Table 4 to 14.
60) The method according to item 58, wherein said antigen marker is CD38 or an immuno-reactive fragment thereof.
61) The method according to item 58, wherein said antigen marker is CD70 or an immuno-reactive fragment thereof.
62) The method according to item 58, wherein said antigen marker is CS1 or an immuno-reactive fragment thereof.
63) The method according to any one of items 58 to 62, wherein said method includes a further step of activating and expanding the T-cells.
64) A method according to any one of items 58 to 63, wherein said method includes a further step of purifying the resulting T-cells by excluding the cells presenting said marker antigen at their surface.
65) A method according to any one of items 58 to 64, wherein said method includes a previous step of procuring the T-cells from a donor.
66) A method according to any one of items 58 to 65, wherein said method includes a previous step of procuring the T-cells from a patient who is affected by the development of said pathological cells.
67) A method according to any one of items 58 to 66, wherein said T-cell is derived from a primary stem cell, iPS or hES cell.
68) A method according to item 67, wherein said T-cell is derived from iPS cell derived from said patient affected by the development of said pathological cells.
69) A method according to any one of items 58 to 68, wherein step a) is performed using a rare-cutting endonuclease.
70) A method according to item 69, wherein step a) is performed using a TAL-nuclease.
71) A method according to item 70, wherein step a) is performed using a RNA-guided endonuclease
72) A method according to item 71, wherein the RNA-guided endonuclease is Cas9.
73) A method according to item 71, wherein RNA-guided endonuclease is split into at least 2 polypeptides, one comprising RuvC and another comprising HNH.
74) A method according to item 70, wherein said endonuclease is expressed from transfected mRNA.
75) A method according to any one of items 1 to 74, wherein said method includes a further step of inactivating a gene encoding a component of the T-cell receptor (TCR).
76) A method according to item 75, wherein said component of the T-cell receptor is TCRα.
77) A method according to any one of items 58 to 15, wherein said method includes a further step of inactivating a gene encoding a component of HLA.
78) A method according to any one of items 58 to 77, wherein said method includes a further step of inactivating a gene encoding β2m.
79) A method according to any one of items 58 to 78, wherein said method includes a further step of inactivating a gene encoding an immune checkpoint protein selected from CTLA4, PPP2CA, PPP2CB, PTPN6, PTPN22, PDCD1, LAG3, HAVCR2, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2 and GUCY1B3.
80) A method according to item 79, wherein said gene locus is involved into the expression of PD1 or CTLA-4 genes.
81) A method according to any one of items 58 to 80, wherein said method includes a further step of inactivating a gene conferring sensitivity of the T-cells to chemotherapy or immunosuppressive drugs.
82) The method according to item 81, wherein said further gene encodes CD52.
83) The method according to item 81, wherein said further gene is hypoxanthine-guanine phosphoribosyltransferase (HPRT).
84) The method according to item 81, wherein said further gene encodes a glucocorticoid receptor (GR).
85) The method according to item 81, wherein said further gene is involved in the DCK regulatory pathway, in particular DCK expression.
86) A method according to any one of items 1 to 85, wherein said T-cells in step a) are inflammatory T-lymphocytes, cytotoxic T-lymphocytes, regulatory T-lymphocytes or helper T-lymphocytes.
87) The method according to item 86, wherein said T-cells are derived from CD4+ T-lymphocytes and/or CD8+ T-lymphocytes.
88) A method according to any one of items 58 to 87, wherein said transformed T-cells are expanded in-vitro.
89) A method according to any one of items 58 to 88, wherein said transformed T-cells are expanded in-vivo.
90) A method according to any one of items 58 to 89, wherein said pathological cells are selected from malignant cells or infected cells.
91) A method according to any one of items 58 to 90, wherein said pathological cells are B-cells.
92) A method according to any one of items 58 to 91, wherein said pathological cells are solid tumor cells.
93) A method according to any one of items 58 to 92, for preparing T-cells to be used as a medicament.
94) A method according to item 93 for preparing T-cells for treating a cancer, an immune disease or an infection in a patient in need thereof.
95) A method according to item 94 for treating lymphoma.
96) A method according to item 94 for treating leukemia.
97) A method according to item 94 for treating chronic lymphocytic leukemia (CLL).
98) An engineered T-cell obtainable according to the method of any one of items 58 to 92.
99) A method for treating a patient comprising:
   (a) Diagnosing said patient for the presence of pathological cells expressing at the cell surface an antigen marker in common with T-cells,
   (b) preparing a population of engineered T-cells wherein cell surface expression of said antigen marker in undetectable,
   (c) administrating said engineered T-cells to said patient diagnosed for said pathological cells, concomitantly to the therapeutic antibody, before of after said therapeutic antibody.

According to the invention, the immune cells are engineered in order to inactivate a gene encoding an antigen marker or several antigens markers, or to inactivate the expression of the gene which product is involved in the presentation of such antigen marker on the cell surface.

Inactivation is preferably performed by a genome modification, more particularly through the expression, preferably transient, in the cell of a specific rare-cutting endonuclease able to target a genetic locus directly or indirectly involved in the production or presentation of said antigen marker at the surface of the cell. Different types of rare-cutting endonucleases can be used, such as Meganucleases, TAL-nucleases, zing-finger nucleases (ZFN), or RNA guided endonucleases like Cas9/CRISPR. The specific rare-cutting endonucleases used here, Meganucleases, TAL-nucleases, zing-finger nucleases (ZFN), and RNA guided endonucleases like Cas9/CRISPR are part of the present invention, their use for engineering cells for immunotherapy.

According to a preferred embodiment, the immune cells defective in at least one antigen marker X are endowed with at least one chimeric antigen receptors (CAR) allowing a specific binding of target cells bearing said targeted antigen marker X and the concomitant use of a therapeutic antibody targeting X.

According to another embodiment, the immune cells can be further engineered to make them allogeneic, especially by deleting genes involved into self-recognition, such as those, for instance, encoding components of T-cell receptors (TCR) or HLA complex.

The present invention encompasses the isolated cell, or cell lines, in particular isolated T-cells, or T cell lines comprising the genetic modifications set forth in the detailed description, examples and figures, as well as any of the proteins, polypeptides or vectors useful to engineer said T-cells.

As a result of the invention, the proteins, polynucleotides, vectors and engineered-cells can be used as therapeutic products, ideally as an “off the shelf” product, in methods for treating or preventing cancer, infections or auto-immune disease.

### BRIEF DESCRIPTION OF THE FIGURES AND TABLES

FIG. 1: CD70 TALE-nucleases molecular in primary T-cells. Lines 1 and 3 correspond to exon1 and exon 2, respectively, from untreated cells. Line 2 corresponds to TALE-nuclease targeting CD70 exon1 treated cells (i.e. T02: SEQ ID NO. 104-105). Line 4 corresponds to TALE-nuclease targeting CD70 exon 2 treated T-cells (T03 SEQ ID NO. 106-107). Arrows are showing the PCR cleaved products.

FIG. 2: Quantification of the percentage of CD70 positive cells (FIG. 2A) and of the median fluorescence intensity (MFI) of CD70 positive cells (FIG. 2B) at different time points post electroporation of untreated or TALE-nucleases treated T-cells.

Tables 4 to 14: Examples of antigen markers, which can be targeted with the engineered-cells of the invention for treating different types of cancer.

Table 5 to 13: Surface antigen markers expressed in T-cells, while being over-expressed in tumor cells from various types of cancer.

Table 5: colon tumor cells;

A combination comprising immune cells, comprising an inactivated antigen marker corresponding to any one of the antigen markers X in table 5 and a therapeutic antibody specific for X, for the treatment of colon cancer.

Table 6: breast tumor cells;

A combination comprising immune cells, comprising an inactivated antigen marker corresponding to any one of the antigen markers X in table 6 and a therapeutic antibody specific for X, for the treatment of breast cancer.

Table 7: digestive track tumor cells;

A combination comprising immune cells, comprising an inactivated antigen marker corresponding to any one of the antigen markers X in table 7 and a therapeutic antibody specific for X, for the treatment of digestive tract cancer.

Table 8: kidney tumor cells;

A combination comprising immune cells, comprising an inactivated antigen marker corresponding to any one of the antigen markers in table 8 and a therapeutic antibody specific for X, for the treatment of kidney cancer.

Table 9: liver tumor cells;

A combination comprising immune cells, comprising an inactivated antigen marker corresponding to any one of the antigen markers X in table 9 and a therapeutic antibody specific for X, for the treatment of liver cancer.

Table 10: lung tumor cells;

A combination comprising immune cells, comprising an inactivated antigen marker corresponding to any one of the antigen markers X in table 10 and a therapeutic antibody specific for X, for the treatment of lung cancer.

Table 11: ovary tumor cells;

A combination comprising immune cells, comprising an inactivated antigen marker corresponding to any one of the antigen markers in table 11 and a therapeutic antibody specific for X, for the treatment of ovary cancer.

Table 12: pancreas tumor cells;

A combination comprising immune cells, comprising an inactivated antigen marker corresponding to any one of the antigen markers X in table 12 and a therapeutic antibody specific for X, for the treatment of pancreas cancer.

Table 13: prostate tumor cells;

A combination comprising immune cells, comprising an inactivated antigen marker corresponding to any one of the antigen markers X in table 13 and a therapeutic antibody specific for X, for the treatment of prostate cancer.

Table 14: Main surface antigen markers expressed in T-cells, while being over-expressed in liquid tumor cells from various types of cancer (ALL, AML, CML, MDS, CLL, CTRL).

A combination comprising immune cells, comprising an inactivated antigen marker corresponding to any one of the antigen markers X in table 14 and a therapeutic antibody specific for X, for the treatment of ALL, AML, CML, MDS, CLL, CTRL.

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In a general aspect, the present invention relates to new combinations of engineered immune cells with undetectable level of cell surface expression of an antigen marker X, with X usually expressed on normal non pathological immune cells, with a therapeutic antibody specific for X, for new adoptive immunotherapy strategies in treating diseases linked with the development of pathological cells expressing an antigen marker X, such as cancer cells, infected cells and cells involved in auto-immune diseases. An immune cell is a cytotoxic T cell which activation by an antigen results in lysis of the target cells.

The main objective of the invention is to target pathological cells that bear specific antigen markers in common with T-cells using two immune reagents, a therapeutic antibody and an engineered immune cell, with the particularity that both reagents target the same antigen which is normally expressed on normal immune cells and on pathological cells.

Thus, in individuals treated with the combination of the invention, an immunodepletion may be obtained but the use of immune cells may not be compromised as engineered immune cells of the present invention are resistant to the therapeutic antibody with which they are combined to and can therefore be used in an individual treated with said therapeutic antibody.

By pathological cell is meant any types of cells present in a patient, which are deemed causing health deterioration.

In general, pathological cells are malignant or infected cells that need to be reduced or eliminated to obtain remission of a patient.

Affecting cell surface expression means decreasing cell surface expression to undetectable level or inactivating the activity of said surface antigen.

In particular embodiments the invention provides a combination according to item 1 wherein said immune cell is an immune T cell, a hematopoietic stem cell, preferably a TCR negative immune T cell, a TCR negative hematopoietic stem cell.

In preferred embodiments said immune T cell is a population of T cells, preferably homogenous.

A TCR negative immune cell means a cell comprising a TCR KO gene, or a TCR deficient gene, deficiency resulting in a disruption of cell surface expression of the TCR. Preferably said TCR is an alpha beta TCR.

Inhibition of cell surface expression means that in 80 to 90%, preferably in more than 99%, even more preferably in more than 99.9% of total cells, cell surface expression of said antigen is undetectable by flow cytometry.

In particular embodiments the invention provides a combination comprising an engineered immune CD8 T cell, immune CD4 T cells, inflammatory T-cell cytotoxic T-cell, regulatory T-cell or helper T-cell. Cytotoxic CD4 T− cell, Cytotoxic CD8 T− lymphocyte.

In particular embodiments the invention provides a combination according to anyone of item 1 to 3 wherein said gene is selected from any one of the genes in Table 4 to 14. A genetic modification means a mutation, an insertion or a deletion generated using a rare cutting endonuclease. Insertion comprises a polynucleotide encoding a gene, such as a CAR, a gene conferring resistance to a drug, sensitivity to a drug, encoding a cytokine.

In particular embodiments the invention provides a combination wherein said genetic modification is generated by a TAL-Nuclease, preferably a mRNA encoding a TAL-Nuclease. In particular embodiments the invention provides a combination according to any one of item 11 wherein said immune T cell express a CAR targeting specifically CD38.

In particular embodiments the invention provides a combination according to any one of item 11 wherein said immune T cell express a CAR targeting specifically CD123.

In particular embodiments the invention provides a combination according to any one of item 11 wherein said immune T cell express a CAR targeting specifically CD22.

In particular embodiments the invention provides a combination according to any one of item 11 wherein said immune T cell express a CAR targeting specifically CS1.

In particular embodiments the invention provides a combination according to any one of item 11 wherein said immune T cell express a CAR targeting specifically CLL-1.

In particular embodiments the invention provides a combination according to any one of the above wherein said immune T cell express a recombinant TCR isolated from a tumor. In particular embodiments the invention provides a combination wherein said immune T cell express a CAR which target specifically a cell surface marker selected from BCMA, CD33, EGFRVIII, Flt3, WT1, CD70, MUC16, PRAME, TSPAN10, CLAUDIN18.2, DLL3, LY6G6D, Liv-1, CHRNA2 and ADAM10.

In particular embodiments the invention provides a combination wherein said CAR comprises an extracellular binding domain, said extracellular binding domain comprising two, or three mAb-specific epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab, preferably 2 mAb-specific epitopes specifically recognized by rituximab, or 1 mAb-specific epitopes specifically recognized by QBEND-10 and 3 mAb-specific epitopes specifically recognized by rituximab.

In most preferred embodiments the invention provides a combination according to any one of those disclosed herein wherein said immune T cell is or was further engineered by genetically inactivating at least one gene encoding a component of the T-cell receptor (TCR) and/or affecting the expression of molecule of the HLA complex.

Thus, the invention provides a combination wherein said immune T cell is engineered to be less alloreactive as compared to non-engineered cells and therefore more specific for the pathological cells intended to be treated or destroyed.

The combination according to any one of those above wherein said immune T cell is further engineered to be less alloreactive as compared to non-engineered cells, by genetically inactivating the alphaTCR and beta2microglobulin genes,

The combination according to any one of item 1 to 15 wherein said immune T cell is further engineered to be less alloreactive as compared to non-engineered cells, by genetically inactivating the alphaTCR and regulatory factor X-associated ankyrin-containing protein (RFXANK) gene,

The combination according to any one of item 1 to 15 wherein said immune T cell is further engineered to be less alloreactive as compared to non-engineered cells, by genetically inactivating the alphaTCR and regulatory factor 5 (RFX5) gene,

The combination according to any one of item 1 to 15 wherein said immune T cell is further engineered to be less alloreactive as compared to non-engineered cells, by genetically inactivating the alphaTCR and regulatory factor X-associated protein (RFXAP) gene,

The combination according to any one of item 1 to 15 wherein said immune T cell is further engineered to be less alloreactive as compared to non-engineered cells, by genetically inactivating the alphaTCR and class II transactivator (CIITA),

The combination according to any one of item 1 to 15 wherein said immune T cell is further engineered to be less alloreactive as compared to non-engineered cells, by genetically inactivating the alphaTCR and TAP-1 gene.

In particular embodiments the invention provides a combination according to any one of the above further engineered to resist hypoxia, preferably wherein said CAR or said cells comprises a domain conferring resistance to hypoxia such as at least one HIF 1 alpha domain. The combination according to the present invention further engineered to resist tumor-inducing inhibition anti-tumor activity of immune cells mediated by any one of the following molecules, Programmed Death 1 (PD-1), Cytotoxic T-Lymphocyte Antigen 4 (CTLA-4), LAG3 Tim3, BTLA, BY55, TIGIT, B7H5, LAIR1, SIGLEC10, and 2B4.

CD70-expressing cancers that can be treated or prevented using the combination of the invention, for example, different subtypes of Non-Hodgkin's Lymphoma (indolent NHLs, follicular NHLs, small lymphocytic lymphomas, lymphoplasmacytic NHLs, or marginal zone NHLs); Hodgkin's disease (e.g., Reed-Sternberg cells); cancers of the B-cell lineage, including, e.g., diffuse large B-cell lymphomas, follicular lymphomas, Burkitt's lymphoma, mantle cell lymphomas, B-cell lymphocytic leukemias (e.g., acute lymphocytic leukemia, chronic lymphocytic leukemia); Epstein Barr Virus positive B cell lymphomas; renal cell carcinomas (e.g., clear cell and papillary); nasopharyngeal carcinomas; thymic carcinomas; gliomas; glioblastomas; neuroblastomas; astrocytomas; meningiomas; Waldenstrom macroglobulinemia; multiple myelomas; and colon, stomach, and rectal carcinomas. The cancer can be, for example, newly diagnosed, pre-treated or refractory or relapsed. In some embodiments, a CD70-expressing cancer has at least about 15,000, at least about 10,000 or at least about 5,000 CD70 molecules/cell.

Document WO2010051391 A1 is incorporated herein in its entirety as prior art allowing the combination of CS1 deficient engineered TCR negative T cells and a therapeutic antibody to be prepared and used for the treatment of NK cell lymphoma, NK or T cell lymphoma, angioimmunoblastic T-cell lymphoma (AITL), or peripheral T cell lymphoma not otherwise specified (PTCL-NOS).

Non-limiting examples of conditions associated with CD38 expression include but are not limited to, multiple myeloma (Jackson et al. (1988), Clin. Exp. Immunol. 72: 351-356), B-cell chronic lymphocytic leukemia (B-CLL) Durig et al. (2002), Leukemia 16: 30-5; Morabito et al. (2001), Leukemia Research 25: 927-32; Marinov et al. (1993), Neoplasma 40(6): 355-8; and Jelinek et al. (2001), Br. J. Haematol. 115: 854-61), acute lymphoblastic leukemia (Keyhani et al. (1999), Leukemia Research 24: 153-9; and Marinov et al. (1993), Neoplasma 40(6): 355-8), chronic myeloid leukemia (Marinov et al. (1993), Neoplasma 40(6): 355-8), acute myeloid leukemia (Keyhani et al. (1999), Leukemia Research 24: 153-9), chronic lymphocytic leukemia (CLL), chronic myelogenous leukemia or chronic myeloid leukemia (CML), acute myelogenous leukemia or acute myeloid leukemia (AML), acute lymphocytic leukemia (ALL), hairy cell leukemia (HCL), myelodysplasia syndromes (MDS) or chronic myelogenous leukemia (CML-BP) in blastic and all subtypes of these leukemias which are defined by morphological, histochemical and immunological techniques that are well known by those of skill in the art. “Neoplasm” or “neoplastic condition” or “cancer” refers to a condition associated with proliferation of cells characterized by a loss of normal controls that results in one re more symptoms including, unregulated growth, lack of differentiation, local tissue invasion, and metastasis. In some embodiments of the invention, the hematologic cancer expressing CD38 is a cancer selected from the group of Chronic Lymphocytic Leukemia (CLL), Chronic Myelogenous Leukemia (CML), Acute Myelogenous Leukemia (AML), and Acute Lymphocytic Leukemia (ALL). Furthermore, it is known in the art that CD38 expression is a prognostic indicator for patients with conditions such as, for example, B-cell chronic lymphocytic leukemia (Durig et al. (2002), Leukemia 16: 30-5; and Morabito et al. (2001), Leukemia Research 25: 927-32) and acute myelogenous leukemia (Keyhani et al. (1999), Leukemia Research 24: 153-9). The combination of the invention comprising CD38 negative engineered cells and anti-CD38 therapeutic antibody is intended to be administered to patients with these particular conditions.

CS 1 (also known as SLAMF7, CRACC, 19A, APEX-1, FOAP 12, and 19A; GENBANK® Accession No. NM 021181.3, Ref. Boles et al, Immunogenetics, 52:302-307 (2001); Bouchon et al, J. Immunol, 167:5517-5521 (2001); Murphy et al, Biochem. J., 361:431-436 (2002)) is a member of the CD2 subset of the immunoglobulin superfamily. Molecules of the CD2 family are involved in a broad range of immunomodulatory functions, such as co-activation, proliferation differentiation, and adhesion of lymphocytes, as well as immunoglobulin secretion, cytokine production, and NK cell cytotoxicity. Several members of the CD2 family, such as CD2, CD58, and CD 150, play a role or have been proposed to play a role in a number of autoimmune and inflammatory diseases, such as psoriasis, rheumatoid arthritis, and multiple 10 sclerosis. It has been reported that CS 1 plays a role in NK cell-mediated cytotoxicity and lymphocyte adhesion (Bouchon, A. et al, J. Immunol, 5517-5521 (2001); Murphy, J. et al, Biochem. J., 361:431-436 (2002)).

The present invention provides a TAL-protein comprising a sequence having at least 95% homology with

(SEQ ID NO 114)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVAIASNNGGKQALETVQRLLPVLCQAHGLTPEQVVAIASNIGGKQALETVQALLPVLCQAHGLTPQQV
VAIASNNGGKQALETVQRLLPVLCQAHGLTPQQVAIASNNGGKQALETVQRLLPVLCQAHGLTPQQVVAIASNG
GGKQALETVQRLLPVLCQAHGLTPQQVVAIASNNGGKQALETVQRLLPVLCQAHGLTPQQVVAIASNNGGKQAL
ETVQRLLPVLCQAHGLTPQQVVAIASNNGGKQALETVQRLLPVLCQAHGLTPQQVVAIASNGGGKQALETVQRL
LPVLCQAHGLTPQQVVAIASNGGGKQALETVQRLLPVLCQAHGLTPQQVVAIASNNGGKQALETVQRLLPVLCQ
AHGLTPQQVVAIASNNGGKQALETVQRLLPVLCQAHGLTPQQVVAIASNGGKQALETVQRLLPVLCQAHGLTPQ
QVAIASNNGGKQALETVQRLLPVLCQAHGLTPEQVVAIASNIGGKQALETVQALLPVLCQAHGLTPQQVAIASN
GGGRPALESIVAQLSRPDPALAALTNDHLVALACLGGRPALDAVKKGLGDPISRSQLVKSELEEKKSELRHKLK
YVPHEYIELIEIARNSTQDRILEMKVMEFFMKVYGYRGKHLGGSRKPDGAIYTVGSPIDYGVIVDTKAYSGGYN
LPIGQADEMQRYVEENQTRNKHINPNEWWKVYPSSVTEFKFLFVSGHFKGNYKAQLTRLNHITNCNGAVLSVEE
LLIGGEMIKAGTLTLEEVRRKFNNGEINFAAD
and
(SEQ ID NO 115)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNGGGKQALETVQRLLPVLCQAHGLTPEQ
VVAIASHDGGKQALETVQRLLPVLCQAHGLTPEQVVAIASHDGGKQALETVQRLLPVLCQAHGLTPEQVAIASH
DGGKQALETVQRLLPVLCQAHGLTPQQVVAIASNGGKQALETVQRLLPVLCQAHGLTPQQVVAIASNNGGKQAL
ETVQRLLPVLCQAHGLTPEQVVAIASHDGGKQALETVQRLLPVLCQAHGLTPEQVVAIASNIGGKQALETVQAL
LPVLCQAHGLTPQQVVAIASNNGGKQALETVQRLLPVLCQAHGLTPQQVVAIASNGGGKQALETVQRLLPVLCQ
AHGLTPQQVVAIASNNGGKQALETVQRLLPVLCQAHGLTPEQVVAIASHDGGKQALETVQRLLPVLCQAHGLTP
EQVVAIASHDGGKQALETVQRLLPVLCQAHGLTPEQVVAIASHDGGKQALETVQRLLPVLCQAHGLTPQQVAIA
SNGGGRPALESIVAQLSRPDPALAALTNDHLVALACLGGRPALDAVKKGLGDPISRSQLVKSELEEKKSELRHK
LKYVPHEYIELIEIARNSTQDRILEMKVMEFFMKVYGYRGKHLGGSRKPDGAIYTVGSPIDYGVIVDTKAYSGG
YNLPIGQADEMQRYVEENQTRNKHINPNEWWKVYPSSVTEFKFLFVSGHFKGNYKAQLTRLNHITNCNGAVLSV
EELLIGGEMIKAGTLTLEEVRRKFNNGEINFAAD;
(SEQ ID NO 116)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPEQVVAIASHDGGKQALETVQRLLPVLCQAHGLTPQQ
VVAIASNNGGKQALETVQRLLPVLCQAHGLTPEQVVAIASNIGGKQALETVQALLPVLCQAHGLTPQQVVAIAS
NNGGKQALETVQRLLPVLCQAHGLTPQQVVAIASNGGGKQALETVQRLLPVLCQAHGLTPQQVVAIASNGGGKQ
ALETVQRLLPVLCQAHGLTPEQVVAIASHDGGKQALETVQRLLPVLCQAHGLTPEQVVAIASNIGGKQALETVQ
ALLPVLCQAHGLTPQQVVAIASNNGGKQALETVQRLLPVLCQAHGLTPEQVVAIASHDGGKQALETVQRLLPVL
CQAHGLTPEQVVAIASHDGGKQALETVQRLLPVLCQAHGLTPEQVVAIASHDGGKQALETVQRLLPVLCQAHGL
TPQQVVAIASNNGGKQALETVQRLLPVLCQAHGLTPQQVVAIASNNGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD
and
(SEQ ID NO 117)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPEQVVAIASNIGGKQALETVQALLPVLCQAHGLTPQQVVAIASNNGGKQALETVQRLLPVLCQAHGLTPEQ
VVAIASNIGGKQALETVQALLPVLCQAHGLTPQQVVAIASNNGGKQALETVQRLLPVLCQAHGLTPEQVVAIAS
NIGGKQALETVQALLPVLCQAHGLTPQQVVAIASNNGGKQALETVQRLLPVLCQAHGLTPEQVVAIASHDGGKQ
ALETVQRLLPVLCQAHGLTPEQVVAIASHDGGKQALETVQRLLPVLCQAHGLTPQQVVAIASNNGGKQALETVQ
RLLPVLCQAHGLTPQQVVAIASNNGGKQALETVQRLLPVLCQAHGLTPEQVVAIASHDGGKQALETVQRLLPVL
CQAHGLTPEQVVAIASNIGGKQALETVQALLPVLCQAHGLTPQQVVAIASNNGGKQALETVQRLLPVLCQAHGL
TPEQVVAIASHDGGKQALETVQRLLPVLCQAHGLTPEQVVAIASNIGGKQALETVQALLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD;
(SEQ ID NO 118)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPEQVVAIASNIGGKQALETVQALLPVLCQAHGLTPQQ
VVAIASNGGGKQALETVQRLLPVLCQAHGLTPEQVVAIASHDGGKQALETVQRLLPVLCQAHGLTPEQVVAIAS
HDGGKQALETVQRLLPVLCQAHGLTPQQVVAIASNGGGKQALETVQRLLPVLCQAHGLTPEQVVAIASHDGGKQ
ALETVQRLLPVLCQAHGLTPQQVVAIASNNGGKQALETVQRLLPVLCQAHGLTPQQVVAIASNGGGKQALETVQ
RLLPVLCQAHGLTPEQVVAIASHDGGKQALETVQRLLPVLCQAHGLTPQQVVAIASNNGGKQALETVQRLLPVL
CQAHGLTPQQVVAIASNGGGKQALETVQRLLPVLCQAHGLTPQQVVAIASNNGGKQALETVQRLLPVLCQAHGL
TPQQVVAIASNNGGKQALETVQRLLPVLCQAHGLTPQQVVAIASNGGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD
and
(SEQ ID NO 119)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPEQVVAIASHDGGKQALETVQRLLPVLCQAHGLTPQQ
VVAIASNGGGKQALETVQRLLPVLCQAHGLTPQQVVAIASNNGGKQALETVQRLLPVLCQAHGLTPQQVVAIAS
NNGGKQALETVQRLLPVLCQAHGLTPEQVVAIASHDGGKQALETVQRLLPVLCQAHGLTPQQVVAIASNNGGKQ
ALETVQRLLPVLCQAHGLTPEQVVAIASHDGGKQALETVQRLLPVLCQAHGLTPEQVVAIASHDGGKQALETVQ
RLLPVLCQAHGLTPEQVVAIASNIGGKQALETVQALLPVLCQAHGLTPEQVVAIASHDGGKQALETVQRLLPVL
CQAHGLTPEQVVAIASHDGGKQALETVQRLLPVLCQAHGLTPQQVVAIASNGGGKQALETVQRLLPVLCQAHGL
TPEQVVAIASHDGGKQALETVQRLLPVLCQAHGLTPQQVVAIASNNGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD;
(SEQ ID NO 102)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPEQVVAIASHDGGKQALETVQRLLPVLCQAHGLTPQQ
VVAIASNNGGKQALETVQRLLPVLCQAHGLTPQQVVAIASNNGGKQALETVQRLLPVLCQAHGLTPQQVVAIAS
NNGGKQALETVQRLLPVLCQAHGLTPEQVVAIASHDGGKQALETVQRLLPVLCQAHGLTPQQVVAIASNGGGKQ
ALETVQRLLPVLCQAHGLTPQQVVAIASNNGGKQALETVQRLLPVLCQAHGLTPEQVVAIASHDGGKQALETVQ
RLLPVLCQAHGLTPQQVVAIASNGGGKQALETVQRLLPVLCQAHGLTPQQVVAIASNGGGKQALETVQRLLPVL
CQAHGLTPQQVVAIASNGGGKQALETVQRLLPVLCQAHGLTPQQVVAIASNNGGKQALETVQRLLPVLCQAHGL
TPQQVVAIASNNGGKQALETVQRLLPVLCQAHGLTPQQVVAIASNGGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD
and
(SEQ ID NO 103)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPEQVVAIASHDGGKQALETVQRLLPVLCQAHGLTPEQ
VVAIASNIGGKQALETVQALLPVLCQAHGLTPEQVVAIASHDGGKQALETVQRLLPVLCQAHGLTPEQVVAIAS
NIGGKQALETVQALLPVLCQAHGLTPEQVVAIASHDGGKQALETVQRLLPVLCQAHGLTPEQVVAIASHDGGKQ
ALETVQRLLPVLCQAHGLTPEQVVAIASNIGGKQALETVQALLPVLCQAHGLTPEQVVAIASHDGGKQALETVQ
RLLPVLCQAHGLTPQQVVAIASNNGGKQALETVQRLLPVLCQAHGLTPEQVVAIASNIGGKQALETVQALLPVL
CQAHGLTPQQVVAIASNNGGKQALETVQRLLPVLCQAHGLTPQQVVAIASNNGGKQALETVQRLLPVLCQAHGL
TPEQVVAIASHDGGKQALETVQRLLPVLCQAHGLTPEQVAIASNIGGKQALETVQALLPVLCQAHGLTPQQVVA
IASNGGGRPALESIVAQLSRPDPALAALTNDHLVALACLGGRPALDAVKKGLGDPISRSQLVKSELEEKKSELR
HKLKYVPHEYIELIEIARNSTQDRILEMKVMEFFMKVYGYRGKHLGGSRKPDGAIYTVGSPIDYGVIVDTKAYS
GGYNLPIGQADEMQRYVEENQTRNKHINPNEWWKVYPSSVTEFKFLFVSGHFKGNYKAQLTRLNHITNCNGAVL
SVEELLIGGEMIKAGTLTLEEVRRKFNNGEINFAAD;
(SEQ ID NO 104)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNNGGKQALETVQRLLPVLCQAHGLTPQQ
VVAIASNGGGKQALETVQRLLPVLCQAHGLTPQQVVAIASNNGGKQALETVQRLLPVLCQAHGLTPEQVVAIAS
NIGGKQALETVQALLPVLCQAHGLTPQQVVAIASNGGGKQALETVQRLLPVLCQAHGLTPEQVVAIASHDGGKQ
ALETVQRLLPVLCQAHGLTPQQVVAIASNGGGKQALETVQRLLPVLCQAHGLTPQQVVAIASNNGGKQALETVQ
RLLPVLCQAHGLTPEQVVAIASHDGGKQALETVQRLLPVLCQAHGLTPEQVVAIASHDGGKQALETVQRLLPVL
CQAHGLTPQQVVAIASNGGGKQALETVQRLLPVLCQAHGLTPEQVVAIASHDGGKQALETVQRLLPVLCQAHGL
TPQQVVAIASNNGGKQALETVQRLLPVLCQAHGLTPQQVVAIASNGGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD
and
(SEQ ID NO 105)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPEQVVAIASHDGGKQALETVQRLLPVLCQAHGLTPQQ
VVAIASNGGGKQALETVQRLLPVLCQAHGLTPQQVVAIASNNGGKQALETVQRLLPVLCQAHGLTPEQVVAIAS
NIGGKQALETVQALLPVLCQAHGLTPQQVVAIASNNGGKQALETVQRLLPVLCQAHGLTPEQVVAIASHDGGKQ
ALETVQRLLPVLCQAHGLTPEQVVAIASHDGGKQALETVQRLLPVLCQAHGLTPQQVVAIASNGGGKQALETVQ
RLLPVLCQAHGLTPQQVVAIASNNGGKQALETVQRLLPVLCQAHGLTPQQVVAIASNGGGKQALETVQRLLPVL
CQAHGLTPQQVVAIASNNGGKQALETVQRLLPVLCQAHGLTPEQVVAIASHDGGKQALETVQRLLPVLCQAHGL
TPQQVVAIASNNGGKQALETVQRLLPVLCQAHGLTPEQVVAIASNIGGKQALETVQALLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD;
(SEQ ID NO 106)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNNGGKQALETVQRLLPVLCQAHGLTPQQ
VVAIASNGGGKQALETVQRLLPVLCQAHGLTPEQVVAIASHDGGKQALETVQRLLPVLCQAHGLTPQQVVAIAS
NGGGKQALETVQRLLPVLCQAHGLTPQQVVAIASNGGGKQALETVQRLLPVLCQAHGLTPQQVVAIASNGGGKQ
ALETVQRLLPVLCQAHGLTPQQVVAIASNGGGKQALETVQRLLPVLCQAHGLTPEQVVAIASHDGGKQALETVQ
RLLPVLCQAHGLTPQQVVAIASNGGGKQALETVQRLLPVLCQAHGLTPQQVVAIASNGGGKQALETVQRLLPVL
CQAHGLTPEQVVAIASHDGGKQALETVQRLLPVLCQAHGLTPEQVVAIASHDGGKQALETVQRLLPVLCQAHGL
TPEQVVAIASNIGGKQALETVQALLPVLCQAHGLTPQQVVAIASNNGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD
and
(SEQ ID NO 107)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNGGGKQALETVQRLLPVLCQAHGLTPQQ
VVAIASNNGGKQALETVQRLLPVLCQAHGLTPQQVVAIASNGGGKQALETVQRLLPVLCQAHGLTPQQVVAIAS
NNGGKQALETVQRLLPVLCQAHGLTPEQVVAIASNIGGKQALETVQALLPVLCQAHGLTPQQVVAIASNGGGKQ
ALETVQRLLPVLCQAHGLTPQQVVAIASNGGGKQALETVQRLLPVLCQAHGLTPEQVVAIASHDGGKQALETVQ
RLLPVLCQAHGLTPEQVVAIASNIGGKQALETVQALLPVLCQAHGLTPQQVVAIASNNGGKQALETVQRLLPVL
CQAHGLTPEQVVAIASHDGGKQALETVQRLLPVLCQAHGLTPQQVVAIASNGGGKQALETVQRLLPVLCQAHGL
TPQQVVAIASNNGGKQALETVQRLLPVLCQAHGLTPEQVVAIASHDGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD;
(SEQ ID NO 108)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPEQVVAIASNIGGKQALETVQALLPVLCQAHGLTPEQ
VVAIASHDGGKQALETVQRLLPVLCQAHGLTPQQVVAIASNGGGKQALETVQRLLPVLCQAHGLTPQQVVAIAS
NGGGKQALETVQRLLPVLCQAHGLTPEQVVAIASHDGGKQALETVQRLLPVLCQAHGLTPEQVVAIASHDGGKQ
ALETVQRLLPVLCQAHGLTPEQVVAIASNIGGKQALETVQALLPVLCQAHGLTPQQVVAIASNNGGKQALETVQ
RLLPVLCQAHGLTPEQVVAIASNIGGKQALETVQALLPVLCQAHGLTPQQVVAIASNNGGKQALETVQRLLPVL
CQAHGLTPEQVVAIASNIGGKQALETVQALLPVLCQAHGLTPQQVVAIASNNGGKQALETVQRLLPVLCQAHGL
TPEQVVAIASHDGGKQALETVQRLLPVLCQAHGLTPEQVVAIASNIGGKQALETVQALLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD
and
(SEQ ID NO 109)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPEQVVAIASNIGGKQALETVQALLPVLCQAHGLTPQQ
VVAIASNNGGKQALETVQRLLPVLCQAHGLTPQQVVAIASNNGGKQALETVQRLLPVLCQAHGLTPQQVVAIAS
NNGGKQALETVQRLLPVLCQAHGLTPQQVVAIASNGGGKQALETVQRLLPVLCQAHGLTPQQVVAIASNNGGKQ
ALETVQRLLPVLCQAHGLTPEQVVAIASNIGGKQALETVQALLPVLCQAHGLTPQQVVAIASNNGGKQALETVQ
RLLPVLCQAHGLTPQQVVAIASNNGGKQALETVQRLLPVLCQAHGLTPEQVVAIASHDGGKQALETVQRLLPVL
CQAHGLTPEQVVAIASNIGGKQALETVQALLPVLCQAHGLTPQQVVAIASNGGGKQALETVQRLLPVLCQAHGL
TPQQVVAIASNNGGKQALETVQRLLPVLCQAHGLTPQQVVAIASNGGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD;
(SEQ ID NO 110)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVAIASNGGGKQALETVQRLLPVLCQAHGLTPEQVVAIASHDGGKQALETVQRLLPVLCQAHGLTPEQV
VAIASHDGGKQALETVQRLLPVLCQAHGLTPEQVVAIASNIGGKQALETVQALLPVLCQAHGLTPQQVVAIASN
NGGKQALETVQRLLPVLCQAHGLTPEQVVAIASNIGGKQALETVQALLPVLCQAHGLTPQQVAIASNNGGKQAL
ETVQRLLPVLCQAHGLTPEQVVAIASNIGGKQALETVQALLPVLCQAHGLTPQQVAIASNNGGKQALETVQRLL
PVLCQAHGLTPEQVVAIASHDGGKQALETVQRLLPVLCQAHGLTPEQVVAIASNIGGKQALETVQALLPVLCQA
HGLTPEQVVAIASNIGGKQALETVQALLPVLCQAHGLTPQQVVAIASNGGGKQALETVQRLLPVLCQAHGLTPE
QVVAIASNIGGKQALETVQALLPVLCQAHGLTPQQVVAIASNGGGKQALETVQRLLPVLCQAHGLTPQQVVAIA
SNGGGRPALESIVAQLSRPDPALAALTNDHLVALACLGGRPALDAVKKGLGDPISRSQLVKSELEEKKSELRHK
LKYVPHEYIELIEIARNSTQDRILEMKVMEFFMKVYGYRGKHLGGSRKPDGAIYTVGSPIDYGVIVDTKAYSGG
YNLPIGQADEMQRYVEENQTRNKHINPNEWWKVYPSSVTEFKFLFVSGHFKGNYKAQLTRLNHITNCNGAVLSV
EELLIGGEMIKAGTLTLEEVRRKFNNGEINFAAD
and
(SEQ ID NO 111)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPEQVVAIASNIGGKQALETVQALLPVLCQAHGLTPQQVVAIASNNGGKQALETVQRLLPVLCQAHGLTPEQ
VVAIASNIGGKQALETVQALLPVLCQAHGLTPQQVVAIASNGGGKQALETVQRLLPVLCQAHGLTPQQVVAIAS
NNGGKQALETVQRLLPVLCQAHGLTPEQVVAIASNIGGKQALETVQALLPVLCQAHGLTPQQVVAIASNNGGKQ
ALETVQRLLPVLCQAHGLTPQQVVAIASNNGGKQALETVQRLLPVLCQAHGLTPQQVVAIASNNGGKQALETVQ
RLLPVLCQAHGLTPQQVVAIASNGGGKQALETVQRLLPVLCQAHGLTPQQVVAIASNNGGKQALETVQRLLPVL
CQAHGLTPEQVVAIASNIGGKQALETVQALLPVLCQAHGLTPQQVVAIASNNGGKQALETVQRLLPVLCQAHGL
TPQQVVAIASNNGGKQALETVQRLLPVLCQAHGLTPEQVVAIASHDGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD;
(SEQ ID NO 112)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNGGGKQALETVQRLLPVLCQAHGLTPQQVVAIASNNGGKQALETVQRLLPVLCQAHGLTPEQ
VVAIASNIGGKQALETVQALLPVLCQAHGLTPEQVVAIASHDGGKQALETVQRLLPVLCQAHGLTPQQVVAIAS
NGGGKQALETVQRLLPVLCQAHGLTPEQVVAIASHDGGKQALETVQRLLPVLCQAHGLTPQQVVAIASNGGGKQ
ALETVQRLLPVLCQAHGLTPEQVVAIASNIGGKQALETVQALLPVLCQAHGLTPQQVVAIASNGGGKQALETVQ
RLLPVLCQAHGLTPQQVVAIASNGGGKQALETVQRLLPVLCQAHGLTPQQVVAIASNNGGKQALETVQRLLPVL
CQAHGLTPQQVVAIASNGGGKQALETVQRLLPVLCQAHGLTPEQVVAIASHDGGKQALETVQRLLPVLCQAHGL
TPQQVVAIASNGGGKQALETVQRLLPVLCQAHGLTPQQVVAIASNNGGKQALETVQRLLPVLCQAHGLTPQQVA
IASNGGGRPALESIVAQLSRPDPALAALTNDHLVALACLGGRPALDAVKKGLGDPISRSQLVKSELEEKKSELR
HKLKYVPHEYIELIEIARNSTQDRILEMKVMEFFMKVYGYRGKHLGGSRKPDGAIYTVGSPIDYGVIVDTKAYS
GGYNLPIGQADEMQRYVEENQTRNKHINPNEWWKVYPSSVTEFKFLFVSGHFKGNYKAQLTRLNHITNCNGAVL
SVEELLIGGEMIKAGTLTLEEVRRKFNNGEINFAAD
and
(SEQ ID NO 113)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNGGGKQALETVQRLLPVLCQAHGLTPEQ
VVAIASNIGGKQALETVQALLPVLCQAHGLTPQQVVAIASNGGGKQALETVQRLLPVLCQAHGLTPQQVVAIAS
NNGGKQALETVQRLLPVLCQAHGLTPQQVVAIASNNGGKQALETVQRLLPVLCQAHGLTPQQVVAIASNGGGKQ
ALETVQRLLPVLCQAHGLTPQQVVAIASNNGGKQALETVQRLLPVLCQAHGLTPEQVVAIASNIGGKQALETVQ
ALLPVLCQAHGLTPEQVVAIASHDGGKQALETVQRLLPVLCQAHGLTPEQVVAIASNIGGKQALETVQALLPVL
CQAHGLTPEQVVAIASNIGGKQALETVQALLPVLCQAHGLTPQQVVAIASNNGGKQALETVQRLLPVLCQAHGL
TPEQVVAIASNIGGKQALETVQALLPVLCQAHGLTPQQVVAIASNNGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD;
(SEQ ID NO 171)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPEQVVAIASNIGGKQALETVQALLPVLCQAHGLTPQQVVAIASNGGGKQALETVQRLLPVLCQAHGLTPEQ
VVAIASNIGGKQALETVQALLPVLCQAHGLTPQQVVAIASNGGGKQALETVQRLLPVLCQAHGLTPEQVVAIAS
HDGGKQALETVQRLLPVLCQAHGLTPEQVVAIASHDGGKQALETVQRLLPVLCQAHGLTPQQVVAIASNGGGKQ
ALETVQRLLPVLCQAHGLTPQQVAIASNGGGKQALETVQRLLPVLCQAHGLTPQQVVAIASNGGGKQALETVQR
LLPVLCQAHGLTPQQVVAIASNNGGKQALETVQRLLPVLCQAHGLTPQQVAIASNNGGKQALETVQRLLPVLCQ
AHGLTPEQVVAIASHDGGKQALETVQRLLPVLCQAHGLTPEQVVAIASNIGGKQALETVQALLPVLCQAHGLTP
QQVAIASNNGGKQALETVQRLLPVLCQAHGLTPEQVVAIASHDGGKQALETVQRLLPVLCQAHGLTPQQVAIAS
NGGGRPALESIVAQLSRPDPSGSGSGGDPISRSQLVKSELEEKKSELRHKLKYVPHEYIELIEIARNSTQDRIL
EMKVMEFFMKVYGYRGKHLGGSRKPDGAIYTVGSPIDYGVIVDTKAYSGGYNLPIGQADEMQRYVEENQTRNKH
INPNEWWKVYPSSVTEFKFLFVSGHFKGNYKAQLTRLNHITNCNGAVLSVEELLIGGEMIKAGTLTLEEVRRKF
NNGEINFAAD
and
(SEQ ID NO 172)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNNGGKQALETVQRLLPVLCQAHGLTPEQ
VVAIASNIGGKQALETVQALLPVLCQAHGLTPEQVVAIASNIGGKQALETVQALLPVLCQAHGLTPQQVVAIAS
NNGGKQALETVQRLLPVLCQAHGLTPEQVVAIASNIGGKQALETVQALLPVLCQAHGLTPQQVVAIASNNGGKQ
ALETVQRLLPVLCQAHGLTPEQVVAIASNIGGKQALETVQALLPVLCQAHGLTPEQVVAIASNIGGKQALETVQ
ALLPVLCQAHGLTPQQVVAIASNGGGKQALETVQRLLPVLCQAHGLTPEQVVAIASHDGGKQALETVQRLLPVL
CQAHGLTPEQVVAIASHDGGKQALETVQRLLPVLCQAHGLTPQQVVAIASNNGGKQALETVQRLLPVLCQAHGL
TPQQVVAIASNNGGKQALETVQRLLPVLCQAHGLTPEQVVAIASHDGGKQALETVQRLLPVLCQAHGLTPQQVV
AIASNGGGRPALESIVAQLSRPDPSGSGSGGDPISRSQLVKSELEEKKSELRHKLKYVPHEYIELIEIARNSTQ
DRILEMKVMEFFMKVYGYRGKHLGGSRKPDGAIYTVGSPIDYGVIVDTKAYSGGYNLPIGQADEMQRYVEENQT
RNKHINPNEWWKVYPSSVTEFKFLFVSGHFKGNYKAQLTRLNHITNCNGAVLSVEELLIGGEMIKAGTLTLEEV
RRKFNNGEINFAAD;
(SEQ ID NO; 173)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNNGGKQALETVQRLLPVLCQAHGLTPQQ
VVAIASNGGGKQALETVQRLLPVLCQAHGLTPQQVVAIASNGGGKQALETVQRLLPVLCQAHGLTPEQVVAIAS
HDGGKQALETVQRLLPVLCQAHGLTPEQVVAIASHDGGKQALETVQRLLPVLCQAHGLTPEQVVAIASHDGGKQ
ALETVQRLLPVLCQAHGLTPEQVVAIASHDGGKQALETVQRLLPVLCQAHGLTPEQVAIASNIGGKQALETVQA
LLPVLCQAHGLTPEQVVAIASNIGGKQALETVQALLPVLCQAHGLTPEQVVAIASHDGGKQALETVQRLLPVLC
QAHGLTPEQVVAIASNIGGKQALETVQALLPVLCQAHGLTPQQVVAIASNGGGKQALETVQRLLPVLCQAHGLT
PQQVVAIASNNGGKQALETVQRLLPVLCQAHGLTPEQVVAIASHDGGKQALETVQRLLPVLCQAHGLTPQQVVA
IASNGGGRPALESIVAQLSRPDPSGSGSGGDPISRSQLVKSELEEKKSELRHKLKYVPHEYIELIEIARNSTQD
RILEMKVMEFFMKVYGYRGKHLGGSRKPDGAIYTVGSPIDYGVIVDTKAYSGGYNLPIGQADEMQRYVEENQTR
NKHINPNEWWKVYPSSVTEFKFLFVSGHFKGNYKAQLTRLNHITNCNGAVLSVEELLIGGEMIKAGTLTLEEVR
RKFNNGEINFAAD
and
(SEQ ID NO 174)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPEQVVAIASNIGGKQALETVQALLPVLCQAHGLTPQQ
VVAIASNNGGKQALETVQRLLPVLCQAHGLTPEQVVAIASHDGGKQALETVQRLLPVLCQAHGLTPQQVVAIAS
NGGGKQALETVQRLLPVLCQAHGLTPQQVVAIASNNGGKQALETVQRLLPVLCQAHGLTPEQVVAIASHDGGKQ
ALETVQRLLPVLCQAHGLTPEQVVAIASHDGGKQALETVQRLLPVLCQAHGLTPEQVVAIASNIGGKQALETVQ
ALLPVLCQAHGLTPEQVVAIASNIGGKQALETVQALLPVLCQAHGLTPEQVVAIASNIGGKQALETVQALLPVL
CQAHGLTPQQVVAIASNNGGKQALETVQRLLPVLCQAHGLTPQQVVAIASNNGGKQALETVQRLLPVLCQAHGL
TPEQVVAIASNIGGKQALETVQALLPVLCQAHGLTPQQVVAIASNGGGKQALETVQRLLPVLCQAHGLTPQQVV
AIASNGGGRPALESIVAQLSRPDPSGSGSGGDPISRSQLVKSELEEKKSELRHKLKYVPHEYIELIEIARNSTQ
DRILEMKVMEFFMKVYGYRGKHLGGSRKPDGAIYTVGSPIDYGVIVDTKAYSGGYNLPIGQADEMQRYVEENQT
RNKHINPNEWWKVYPSSVTEFKFLFVSGHFKGNYKAQLTRLNHITNCNGAVLSVEELLIGGEMIKAGTLTLEEV
RRKFNNGEINFAAD;
(SEQ ID NO 175)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPEQVVAIASHDGGKQALETVQRLLPVLCQAHGLTPEQVVAIASHDGGKQALETVQRLLPVLCQAHGLTPEQ
VVAIASHDGGKQALETVQRLLPVLCQAHGLTPEQVVAIASHDGGKQALETVQRLLPVLCQAHGLTPEQVVAIAS
NIGGKQALETVQALLPVLCQAHGLTPEQVVAIASNIGGKQALETVQALLPVLCQAHGLTPEQVVAIASHDGGKQ
ALETVQRLLPVLCQAHGLTPEQVVAIASNIGGKQALETVQALLPVLCQAHGLTPQQVVAIASNGGGKQALETVQ
RLLPVLCQAHGLTPQQVVAIASNNGGKQALETVQRLLPVLCQAHGLTPEQVVAIASHDGGKQALETVQRLLPVL
CQAHGLTPEQVVAIASHDGGKQALETVQRLLPVLCQAHGLTPQQVVAIASNGGGKQALETVQRLLPVLCQAHGL
TPEQVVAIASHDGGKQALETVQRLLPVLCQAHGLTPEQVVAIASNIGGKQALETVQALLPVLCQAHGLTPQQVA
IASNGGGRPALESIVAQLSRPDPSGSGSGGDPISRSQLVKSELEEKKSELRHKLKYVPHEYIELIEIARNSTQD
RILEMKVMEFFMKVYGYRGKHLGGSRKPDGAIYTVGSPIDYGVIVDTKAYSGGYNLPIGQADEMQRYVEENQTR
NKHINPNEWWKVYPSSVTEFKFLFVSGHFKGNYKAQLTRLNHITNCNGAVLSVEELLIGGEMIKAGTLTLEEVR
RKFNNGEINFAAD
and
(SEQ ID NO 176)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNGGGKQALETVQRLLPVLCQAHGLTPQQ
VVAIASNNGGKQALETVQRLLPVLCQAHGLTPEQVVAIASNIGGKQALETVQALLPVLCQAHGLTPQQVVAIAS
NNGGKQALETVQRLLPVLCQAHGLTPEQVVAIASHDGGKQALETVQRLLPVLCQAHGLTPQQVVAIASNGGGKQ
ALETVQRLLPVLCQAHGLTPQQVVAIASNNGGKQALETVQRLLPVLCQAHGLTPEQVVAIASHDGGKQALETVQ
RLLPVLCQAHGLTPEQVVAIASHDGGKQALETVQRLLPVLCQAHGLTPEQVVAIASNIGGKQALETVQALLPVL
CQAHGLTPEQVVAIASNIGGKQALETVQALLPVLCQAHGLTPEQVVAIASNIGGKQALETVQALLPVLCQAHGL
TPQQVVAIASNNGGKQALETVQRLLPVLCQAHGLTPQQVVAIASNNGGKQALETVQRLLPVLCQAHGLTPQQVV
AIASNGGGRPALESIVAQLSRPDPSGSGSGGDPISRSQLVKSELEEKKSELRHKLKYVPHEYIELIEIARNSTQ
DRILEMKVMEFFMKVYGYRGKHLGGSRKPDGAIYTVGSPIDYGVIVDTKAYSGGYNLPIGQADEMQRYVEENQT
RNKHINPNEWWKVYPSSVTEFKFLFVSGHFKGNYKAQLTRLNHITNCNGAVLSVEELLIGGEMIKAGTLTLEEV
RRKFNNGEINFAAD;
(SEQ ID NO 177)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNNGGKQALETVQRLLPVLCQAHGLTPQQ
VVAIASNGGGKQALETVQRLLPVLCQAHGLTPQQVVAIASNGGGKQALETVQRLLPVLCQAHGLTPEQVVAIAS
HDGGKQALETVQRLLPVLCQAHGLTPEQVVAIASHDGGKQALETVQRLLPVLCQAHGLTPEQVVAIASHDGGKQ
ALETVQRLLPVLCQAHGLTPEQVVAIASHDGGKQALETVQRLLPVLCQAHGLTPEQVVAIASNIGGKQALETVQ
ALLPVLCQAHGLTPEQVVAIASNIGGKQALETVQALLPVLCQAHGLTPEQVVAIASHDGGKQALETVQRLLPVL
CQAHGLTPEQVVAIASNIGGKQALETVQALLPVLCQAHGLTPQQVVAIASNGGGKQALETVQRLLPVLCQAHGL
TPQQVVAIASNNGGKQALETVQRLLPVLCQAHGLTPEQVVAIASHDGGKQALETVQRLLPVLCQAHGLTPQQVA
IASNGGGRPALESIVAQLSRPDPALAALTNDHLVALACLGGRPALDAVKKGLGDPISRSQLVKSELEEKKSELR
HKLKYVPHEYIELIEIARNSTQDRILEMKVMEFFMKVYGYRGKHLGGSRKPDGAIYTVGSPIDYGVIVDTKAYS
GGYNLPIGQADEMQRYVEENQTRNKHINPNEWWKVYPSSVTEFKFLFVSGHFKGNYKAQLTRLNHITNCNGAVL
SVEELLIGGEMIKAGTLTLEEVRRKFNNGEINFAAD
and
(SEQ ID NO 178)
MGDPKKKRKVIDIADLRTLGYSQQQQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKIAKRGGVTAVEAVHAWRNALTGAPL
NLTPQQVVAIASNNGGKQALETVQRLLPVLCQAHGLTPQQVVAIASNGGGKQALETVQRLLPVLCQAHGLTPQQ
VVAIASNNGGKQALETVQRLLPVLCQAHGLTPEQVVAIASNIGGKQALETVQALLPVLCQAHGLTPQQVVAIAS
NNGGKQALETVQRLLPVLCQAHGLTPEQVVAIASHDGGKQALETVQRLLPVLCQAHGLTPQQVVAIASNGGGKQ
ALETVQRLLPVLCQAHGLTPQQVVAIASNNGGKQALETVQRLLPVLCQAHGLTPEQVVAIASHDGGKQALETVQ
RLLPVLCQAHGLTPEQVVAIASHDGGKQALETVQRLLPVLCQAHGLTPEQVVAIASNIGGKQALETVQALLPVL
CQAHGLTPEQVVAIASNIGGKQALETVQALLPVLCQAHGLTPEQVVAIASNIGGKQALETVQALLPVLCQAHGL
TPQQVVAIASNNGGKQALETVQRLLPVLCQAHGLTPQQVVAIASNNGGKQALETVQRLLPVLCQAHGLTPQQVV
AIASNGGGRPALESIVAQLSRPDPALAALTNDHLVALACLGGRPALDAVKKGLGDPISRSQLVKSELEEKKSEL
RHKLKYVPHEYIELIEIARNSTQDRILEMKVMEFFMKVYGYRGKHLGGSRKPDGAIYTVGSPIDYGVIVDTKAY
SGGYNLPIGQADEMQRYVEENQTRNKHINPNEWWKVYPSSVTEFKFLFVSGHFKGNYKAQLTRLNHITNCNGAV
LSVEELLIGGEMIKAGTLTLEEVRRKFNNGEINFAAD.

The invention provides a TAL-protein comprising a sequence having at least 95% homology with SEQ ID NO 114 and SEQ ID NO 115; with SEQ ID NO 116 and SEQ ID NO 117; with SEQ ID NO 118 and SEQ ID NO 119; with SEQ ID NO 102 and SEQ ID NO 103; with SEQ ID NO 104 and SEQ ID NO 105; with SEQ ID NO 106 and SEQ ID NO 107; with SEQ ID NO 108 and SEQ ID NO 109; with SEQ ID NO 110 and SEQ ID NO 111; with SEQ ID NO 112 and SEQ ID NO 113; with SEQ ID NO 171 and SEQ ID NO 172; with SEQ ID NO 173 and SEQ ID NO 174; with SEQ ID NO 175 and SEQ ID NO 176; or with SEQ ID NO 177 and SEQ ID NO 178 for use in the manufacturing of CART cells.

The invention provides a TAL-protein comprising a sequence having at least 95% homology with SEQ ID NO 114 and SEQ ID NO 115; with SEQ ID NO 116 and SEQ ID NO 117; or with SEQ ID NO 118 and SEQ ID NO 119; is provided for modifying the CD38 gene,

The invention provides a TAL-protein comprising a sequence having at least 95% homology with SEQ ID NO 102 and SEQ ID NO 103; with SEQ ID NO 104 and SEQ ID NO 105; or with SEQ ID NO 106 and SEQ ID NO 107, is provided for modifying the CD70 gene.

The invention provides a TAL-protein comprising a sequence having at least 95% homology with SEQ ID NO 108 and SEQ ID NO 109; with SEQ ID NO 110 and SEQ ID NO 111; with SEQ ID NO 112 and SEQ ID NO 113; with SEQ ID NO 171 and SEQ ID NO 172; with SEQ ID NO 173 and SEQ ID NO 174; with SEQ ID NO 175 and SEQ ID NO 176; or with SEQ ID NO 177 and SEQ ID NO 178 is provided for modifying the CS1 gene.

The invention provides a polynucleotide or a vector encoding any one of the TAL protein above.

The invention provides a TAL-protein comprising a sequence of SEQ ID NO 114 and SEQ ID NO 115.

The invention provides a TAL-protein comprising a sequence of SEQ ID NO 116 and SEQ ID NO 117.

The invention provides a TAL-protein comprising a sequence of SEQ ID NO 118 and SEQ ID NO 119.

The invention provides a TAL-protein comprising a sequence of SEQ ID NO 102 and SEQ ID NO 103.

The invention provides a TAL-protein comprising a sequence of SEQ ID NO 104 and SEQ ID NO 105.

The invention provides a TAL-protein comprising a sequence of SEQ ID NO 106 and SEQ ID NO 107.

The invention provides a TAL-protein comprising a sequence of SEQ ID NO 108 and SEQ ID NO 109.

The invention provides a TAL-protein comprising a sequence of SEQ ID NO 110 and SEQ ID NO 111.

The invention provides a TAL-protein comprising a sequence of SEQ ID NO 112 and SEQ ID NO 113.

The invention provides a TAL-protein comprising a sequence of SEQ ID NO 171 and SEQ ID NO 172.

The invention provides a TAL-protein comprising a sequence of SEQ ID NO 173 and SEQ ID NO 174.

The invention provides a TAL-protein comprising a sequence of SEQ ID NO 175 and SEQ ID NO 176.

The invention provides a TAL-protein comprising a sequence of SEQ ID NO 177 and SEQ ID NO 178.

The invention provides a TAL-protein according to item 54 comprising a sequence having at least 95% homology with SEQ ID NO 114 and SEQ ID NO 115; comprising a sequence having at least 95% homology with SEQ ID NO 116 and SEQ ID NO 117; comprising a sequence having at least 95% homology with SEQ ID NO 118 and SEQ ID NO 119; comprising a sequence having at least 95% homology with SEQ ID NO 102 and SEQ ID NO 103; comprising a sequence having at least 95% homology with SEQ ID NO 104 and SEQ ID NO 105; comprising a sequence having at least 95% homology with SEQ ID NO 106 and SEQ ID NO 107; comprising a sequence having at least 95% homology with SEQ ID NO 108 and SEQ ID NO 109; comprising a sequence having at least 95% homology with SEQ ID NO 110 and SEQ ID NO 111; comprising a sequence having at least 95% homology with SEQ ID NO 112 and SEQ ID NO 113; comprising a sequence having at least 95% homology with SEQ ID NO 171 and SEQ ID NO 172; comprising a sequence having at least 95% homology with SEQ ID NO 173 and SEQ ID NO 174; comprising a sequence having at least 95% homology with SEQ ID NO 175 and SEQ ID NO 176; or comprising a sequence having at least 95% homology with SEQ ID NO 177 and SEQ ID NO 178 for use in the manufacturing of CART cells.

The invention provides a TAL-protein comprising a sequence having at least 95% homology with SEQ ID NO 114 and SEQ ID NO 115; with SEQ ID NO 116 and SEQ ID NO 117; or with SEQ ID NO 118 and SEQ ID NO 119; is provided for modifying the CD38 gene.

The invention provides a TAL-protein comprising a sequence having at least 95% homology with SEQ ID NO 102 and SEQ ID NO 103; with SEQ ID NO 104 and SEQ ID NO 105; or with SEQ ID NO 106 and SEQ ID NO 107, is provided for modifying the CD70 gene.

A TAL-protein comprising a sequence having at least 95% homology with SEQ ID NO 108 and SEQ ID NO 109; with SEQ ID NO 110 and SEQ ID NO 111; with SEQ ID NO 112 and SEQ ID NO 113; with SEQ ID NO 171 and SEQ ID NO 172; with SEQ ID NO 173 and SEQ ID NO 174; with SEQ ID NO 175 and SEQ ID NO 176; or with SEQ ID NO 177 and SEQ ID NO 178 is provided for modifying the CS1 gene.

The invention provides a polynucleotide or a vector encoding a protein comprising any one of the TAL protein above is provided.

A polynucleotide or a vector encoding any one of the TAL protein above is provided.

An engineered immune T cell comprising a CD38 modified gene obtained using a TAL-protein or a polynucleotide encoding a sequence having at least 95% homology with SEQ ID NO 114 and SEQ ID NO 115; with SEQ ID NO 116 and SEQ ID NO 117; or with SEQ ID NO 118 and SEQ ID NO 119 is provided.

The invention provides an engineered immune T cell comprising a CD70 modified gene obtained using a TAL-protein or a polynucleotide encoding a sequence having with SEQ ID NO 102 and SEQ ID NO 103; with SEQ ID NO 104 and SEQ ID NO 105; or with SEQ ID NO 106 and SEQ ID NO 107, is provided.

An engineered immune T cell comprising a CS1 modified gene obtained using a TAL-protein or a polynucleotide encoding a sequence having with SEQ ID NO 108 and SEQ ID NO 109; with SEQ ID NO 110 and SEQ ID NO 111; with SEQ ID NO 112 and SEQ ID NO 113; with SEQ ID NO 171 and SEQ ID NO 172; with SEQ ID NO 173 and SEQ ID NO 174; with SEQ ID NO 175 and SEQ ID NO 176; or with SEQ ID NO 177 and SEQ ID NO 178 is provided.

The T-cells according to the invention may be endowed with a chimeric antigen receptor directed the antigen marker that is commonly expressed by the pathological cells and T-cells. The expression “known to be present” means that the antigen marker is reported to be found on the surface of the T-cells grown in natural conditions in-vivo, especially in the blood, but not necessarily when they are cultured in-vitro. In any event, the method of the invention results into the absence of the antigen marker on the surface of the engineered T-cell, thereby preventing the chimeric antigen receptor from reacting with the engineered T-cell surface. In this respect, the method for preparing the T cells of the invention may include a further step of purifying the resulting T-cells by excluding the cells presenting said marker antigen on their surface.

Therapeutic Antibodies

Therapeutic antibodies used in the present study may be any therapeutic antibody specific for an antigen expressed on immune cells used for immunotherapy such as T cells of precursors, and on pathological cells preferably any one of the antigens described in tables 4 to 14 of the present invention.

As non-limiting examples of anti-CD70 therapeutic antibodies that may be used with and/or combined to engineered cells with a CD70 KO or inactivated gene, the following therapeutic antibody may be combined with cells in which the CD70 gene was inactivated:

Anti CD70 Therapeutic mAbs
Vorsetuzumab (or SGN-70 or h1F6) as disclosed in WO200473656, preferably (h1F6 clone) as disclosed in WO2006113909 which is a humanized h1F6.
ARGX 110 (or Anti-CD70 SIMPLE Antibody) (clone 41D12 or ARGX110), as disclosed in WO2012123586 which is incorporated here in its entirety.
SGN CD70A, SGN-CD70A is an antibody-drug conjugate that combines an anti-CD70 monoclonal antibody with a synthetic DNA cross-linking molecule, pyrrolobenzodiazepine (PBD) dimer.
Vorsetuzumab mafodotin (SGN-75),
MDX 1203 (BMS-936561) as disclosed in WO2008074004 or in WO2007038637 or in disclosed in WO2009126934.

In a preferred embodiment, the combination of the invention comprises CD70 KO immune cells and ARGX 110 and is used for the treatment of Renal Cell Carcinoma or for Non-hodgkin's Lymphoma.

As examples of anti-CD38 therapeutic antibodies that may be used and combined to CD38 KO engineered cells, the following may be combined with cells in which the CD38 gene was inactivated:

Anti CD38 Therapeutic mAbs
Daratumumab (DARZALEX; Humanised anti-CD38 monoclonal antibody; HuMax-CD38; HuMax®-CD38—Genmab; JNJ-54767414). The combination of the invention may be especially efficient for the treatment of MM.
Isatuximab (Anti-CD38 monoclonal antibody—Sanofi; hu38SB19; SAR-650984) as described in U.S. Pat. No. 8,153,765, incorporated here in its entirety.
MOR202 (or MOR-03087) from MorphoSys as described in WO1999/62526 (Mayo Foundation); WO200206347 (Crucell Holland); US2002164788 (Jonathan Ellis) which is incorporated by reference in its entirety; WO2005/103083 (MorphoSys AG), U.S. Ser. No. 10/588,568, which is incorporated by reference in its entirety, WO2006/125640 (MorphoSys AG), U.S. Ser. No. 11/920,830, which is incorporated by reference in its entirety, and (MorphoSys AG), U.S. Ser. No. 12/089,806, which is incorporated by reference in its entirety; WO2006099875 (Genmab), U.S. Ser. No. 11/886,932, which is incorporated by reference in its entirety; and WO08/047242 (Sanofi-Aventis), U.S. Ser. No. 12/441,466, which is incorporated by reference in its entirety.
The combination of the invention of CD38 KO immune cells and therapeutic anti-CD38 MOR202 antibody may be used for the treatment of MM.
Other Ab may be used such as EDC8 (from Wisconsin Alumni Research Foundation) or GBR 1342 from Glenmark Pharmaceuticals S.A. (bispec for CD3 and CD38), in particular for the treatment of MM.

As example of anti-CS1 antibody that may be used and combined to engineered cells in which the CS1 gene was inactivated the following are preferred:

Anti CS1 Therapeutic mAbs
Elotuzumab (BMS-901608; Empliciti; HuLuc63; PDL063) from PDL BioPharma (developed by AbbVie; Bristol-Myers Squibb), for use in the treatment of MM ABBV838 for use in the treatment of MM.

Elotuzumab is a humanized monoclonal IgGI antibody directed against CS-1, a cell surface glycoprotein, which is highly and uniformly expressed in multiple myeloma.

Elotuzumab induces significant antibody-dependent cellular cytotoxicity (ADCC) against primary multiple myeloma cells in the presence of peripheral lymphocytes (Tai et al, Blood, 112: 1329-1337 (2008)). Results of three studies that evaluated the safety and efficacy of this drug administered alone (Zonder et al, Blood, 120(3):552-559 (2012)), in combination with bortezomib (Jakubowiak et al, J. Clin. Oncol., 30(16): 1960-1965 (Jun. 1, 2012)), or lenalidomide and low-dose dexamethasone (Lonial et al, J. Clin. Oncol., 30: 1953-1959 (2012); and Richardson et al, Blood (ASH Annual Meeting Abstracts), 1 16:986 (2010) for the treatment of patients with relapsed or refractory multiple myeloma, have been reported. All three combinations showed a manageable safety profile and encouraging activity. For example, a Phase VII study evaluating the safety and efficacy of Elotuzumab in combination lenalidomide and low-dose dexamethasone for the treatment of relapsed or refractory multiple myeloma demonstrated a 33 month PFS as well as a 92% response rate for patients receiving the 10 mg/kg dose (Lonial et al., J. Clin. Oncol., 31 (2013) (Suppl., Abstr. 8542)). Phase III clinical trials of lenalidomide/dexamethasone with or without Elotuzumab in previously untreated multiple myeloma patients is ongoing, while another phase III trial designed to evaluate this same combination in the first line setting is also ongoing. Accordingly, the invention provides a combination of CS-1 deficient engineered cells and a therapeutic antibody specific for CS1 namely in combination with bortezomib or lenalidomide and low-dose dexamethasone for the treatment of patients with relapsed or refractory multiple myeloma.

Combination—Step of Combining

Combining said engineered T cells with a therapeutic antibody specific for said antigen marker, may mean that cells and antibody are incubated together so that said therapeutic antibody binds to cells in which the antigen is still expressed. The therapeutic antibody is therefore bound to engineered cells that express the antigen marker. Incubation with appropriate reagent allows an antibody mediated purification in vitro or in vivo of engineered cells that do not express the marker antigen. An appropriate reagent may be beads, preferably magnetic beads coated with a protein that binds to antibodies, such as G or A proteins or protein of the complement. Purification may be performed in vivo or in vitro. A therapeutic antibody is an antibody with therapeutic activity, preferably approved by health authorities. Therapeutic activity means an activity that improves health condition in a patient suffering a disease.

All these examples of therapeutically effective antibody are administered in individual in need thereof. A skilled person will administered it in combination with engineered cells accordingly to the present invention, each at the efficient doses to improve the health condition.

In certain embodiments of the present invention, engineered cells are administered to a patient in conjunction with (e.g., before, simultaneously or following) the therapeutic antibody targeting the cell surface antigen marker which expression is inactivated in said cells.

The active dose(s) of antibody and of engineered immune cells and route of injection are known of the skilled person. The present invention provide a combination adapted to the patients so that the first injection of the combination allows 1) immunodepleting the patient in T cells, 2) grafting said patient with engineered cells 3) destroying pathological cells and/or avoiding relapse refractory forms of said pathological cells.

As shown in Table 4, this invention is applicable to an important number of antigen marker candidates reported to be expressed by tumor cells, but also by T-cells. Some of them, like CD38, have been used as specific markers in diagnostic methods for a while, especially with respect to Leukemia pathological cells, but not in therapy. Indeed, although these markers were identified in the art as quite specific markers, they could not be used as targets for immunotherapy because antibodies directed against these markers would have destroyed or interfered with patients' T-cells.

According to a preferred embodiment of the invention, the gene mutation or inactivation of step a) of the above method is performed using a rare-cutting endonuclease.

By inactivating a gene it is intended that the gene of interest is not expressed in a functional protein form. In particular embodiments, the genetic modification of the method relies on the expression, in provided cells to engineer, of a rare-cutting endonuclease such that same catalyzes cleavage in one targeted gene thereby inactivating said targeted gene. The nucleic acid strand breaks caused by the endonuclease are commonly repaired through the distinct mechanisms of homologous recombination or non-homologous end joining (NHEJ). However, NHEJ is an imperfect repair process that often results in changes to the DNA sequence at the site of the cleavage. Mechanisms involve rejoining of what remains of the two DNA ends through direct re-ligation (Critchlow and Jackson 1998) or via the so-called microhomology-mediated end joining (Betts, Brenchley et al. 2003; Ma, Kim et al. 2003). Repair via non-homologous end joining (NHEJ) often results in small insertions or deletions and can be used for the creation of specific gene knockouts. Said modification may be a substitution, deletion, or addition of at least one nucleotide. Cells in which a cleavage-induced mutagenesis event, i.e. a mutagenesis event consecutive to an NHEJ event, has occurred can be identified and/or selected by well-known method in the art.

The term “rare-cutting endonuclease” refers to a wild type or variant enzyme capable of catalyzing the hydrolysis (cleavage) of bonds between nucleic acids within a DNA or RNA molecule, preferably a DNA molecule. Particularly, said nuclease can be an endonuclease, more preferably a rare-cutting endonuclease which is highly specific, recognizing nucleic acid target sites ranging from 10 to 45 base pairs (bp) in length, usually ranging from 10 to 35 base pairs in length, more usually from 12 to 20 base pairs. The endonuclease according to the present invention recognizes at specific polynucleotide sequences, further referred to as “target sequence” and cleaves nucleic acid inside these target sequences or into sequences adjacent thereto, depending on the molecular structure of said endonuclease. The rare-cutting endonuclease can recognize and generate a single- or double-strand break at specific polynucleotides sequences.

In a particular embodiment, said rare-cutting endonuclease according to the present invention is a RNA-guided endonuclease such as the Cas9/CRISPR complex. RNA guided endonucleases constitute a new generation of genome engineering tool where an endonuclease associates with a RNA molecule. In this system, the RNA molecule nucleotide sequence determines the target specificity and activates the endonuclease (Gasiunas, Barrangou et al. 2012; Jinek, Chylinski et al. 2012; Cong, Ran et al. 2013; Mali, Yang et al. 2013).

Cas 9

Cas9, also named Csn1 (COG3513) is a large protein that participates in both crRNA biogenesis and in the destruction of invading DNA. Cas9 has been described in different bacterial species such as S. thermophiles, Listeria innocua (Gasiunas, Barrangou et al. 2012; Jinek, Chylinski et al. 2012) and S. pyogenes (Deltcheva, Chylinski et al. 2011). The large Cas9 protein (>1200 amino acids) contains two predicted nuclease domains, namely HNH (McrA-like) nuclease domain that is located in the middle of the protein and a splitted RuvC-like nuclease domain (RNase H fold) (Makarova, Grishin et al. (2006).

By “Cas9” is meant an engineered endonuclease or a homologue of Cas9 which is capable of processing target nucleic acid sequence. In particular embodiment, Cas9 can induce a cleavage in the nucleic acid target sequence which can correspond to either a double-stranded break or a single-stranded break. Cas9 variant can be a Cas9 endonuclease that does not naturally exist in nature and that is obtained by protein engineering or by random mutagenesis. Cas9 variants according to the invention can for example be obtained by mutations i.e. deletions from, or insertions or substitutions of at least one residue in the amino acid sequence of a S. pyogenes Cas9 endonuclease (COG3513). In the frame aspects of the present invention, such Cas9 variants remain functional, i.e. they retain the capacity of processing a target nucleic acid sequence. Cas9 variant can also be homologues of S. pyogenes Cas9 which can comprise deletions from, or insertions or substitutions of, at least one residue within the amino acid sequence of S. pyogenes Cas9. Any combination of deletion, insertion, and substitution may also be made to arrive at the final construct, provided that the final construct possesses the desired activity, in particular the capacity of binding a guide RNA or nucleic acid target sequence.

RuvC/RNaseH motif includes proteins that show wide spectra of nucleolytic functions, acting both on RNA and DNA (RNaseH, RuvC, DNA transposases and retroviral integrases and PIWI domain of Argonaut proteins). In the present invention the RuvC catalytic domain of the Cas9 protein can be characterized by the sequence motif: D-[I/L]-G-X-X-S-X-G-W-A, wherein X represents any one of the natural 20 amino acids and [I/L] represents isoleucine or leucine. In other terms, the present invention relates to Cas9 variant which comprises at least D-[I/L]-G-X-X-S-X-G-W-A sequence, wherein X represents any one of the natural 20 amino acids and [I/L] represents isoleucine or leucine.

HNH motif is characteristic of many nucleases that act on double-stranded DNA including colicins, restriction enzymes and homing endonucleases. The domain HNH (SMART ID: SM00507, SCOP nomenclature: HNH family) is associated with a range of DNA binding proteins, performing a variety of binding and cutting functions. The ones with known function are involved in a range of cellular processes including bacterial toxicity, homing functions in groups I and II introns and inteins, recombination, developmentally controlled DNA rearrangement, phage packaging, and restriction endonuclease activity (Dalgaard, Klar et al. 1997). These proteins are found in viruses, archaebacteria, eubacteria, and eukaryotes. Interestingly, as with the LAGLI-DADG and the GIY-YIG motifs, the HNH motif is often associated with endonuclease domains of self-propagating elements like inteins, Group I, and Group II introns (Dalgaard, Klar et al. 1997). The HNH domain can be characterized by the presence of a conserved Asp/His residue flanked by conserved His (amino-terminal) and His/Asp/Glu (carboxy-terminal) residues at some distance. A substantial number of these proteins can also have a CX2C motif on either side of the central Asp/His residue. Structurally, the HNH motif appears as a central hairpin of twisted p-strands, which are flanked on each side by an a helix (Kleanthous, Kuhlmann et al. 1999). In the present invention, the HNH motif can be characterized by the sequence motif: Y-X-X-D-H-X-X-P-X-S-X-X-X-D-X-S, wherein X represents any one of the natural 20 amino acids. The present invention relates to a Cas9 variant which comprises at least Y-X-X-D-H-X-X-P-X-S-X-X-X-D-X-S sequence wherein X represents any one of the natural 20 amino acids.

This invention can be of particular interest to easily do targeted multiplex gene modifications and to create an inducible nuclease system by introduction of the guide RNA to the Cas9 cells. For the purpose of the present invention, the inventors have established that Cas9 protein can be divided into two separate split Cas9 RuvC and HNH domains which can process target nucleic acid sequence together or separately with the guide RNA.

Also the RuvC and HNH domains from different RNA guided endonucleases or Cas homologues may be assembled to improve nuclease efficiency or specificity. The domains from different species can be either split into two proteins or fused to each other to form a variant Cas protein. The Cas9 split system is deemed particularly suitable for an inducible method of genome targeting and to avoid the potential toxic effect of the Cas9 overexpression within the cell. Indeed, a first split Cas9 domain can be introduced into the cell, preferably by stably transforming said cell with a transgene encoding said split domain. Then, the complementary split part of Cas9 can be introduced into the cell, such that the two split parts reassemble into the cell to reconstitute a functional Cas9 protein at the desired time.

The reduction of the size of the split Cas9 compared to wild type Cas9 ease the vectorization and the delivery into the cell, for example, by using cell penetrating peptides. Re-arranging domains from different Cas proteins, allows to modulate the specificity and nuclease activity, for instance, by targeting PAM motifs that are slightly different from S. pyogenes Cas9

Split Cas9 System

The previous characterization of the RuvC and HNH domains has prompted the inventors to engineer Cas9 protein to create split Cas9 protein. Surprisingly, the inventors showed that these two split Cas9 could process together or separately the nucleic acid target. This observation allows developing a new Cas9 system using split Cas9 protein. Each split Cas9 domains can be prepared and used separately. Thus, this split system displays several advantages for vectorization and delivery of the RNA guided endonuclease in T-cells, allowing delivering a shorter and/or inactive protein, and is particularly suitable to induce genome engineering in T-cells at the desired time and thus limiting the potential toxicity of an integrated Cas9 nuclease.

By “Split Cas9” is meant here a reduced or truncated form of a Cas9 protein or Cas9 variant, which comprises either a RuvC or HNH domain, but not both of these domains. Such “Split Cas9” can be used independently with guide RNA or in a complementary fashion, like for instance, one Split Cas9 providing a RuvC domain and another providing the HNH domain. Different split RNA guided endonucleases may be used together having either RuvC and/or NHN domains.

Each Cas9 split domain can be derived from the same or from different Cas9 homologues. Many homologues of Cas9 have been identified in genome databases.

Said Cas9 split domains (RuvC and HNH domains) can be simultaneously or sequentially introduced into the cell such that said split Cas9 domain(s) process the target nucleic acid sequence in the cell. Said Cas9 split domains and guide RNA can be introduced into the cell by using cell penetrating peptides or other transfection methods as described elsewhere.

In another aspect of the invention, only one split Cas9 domain, referred to as compact Cas9 is introduced into said cell. Indeed, surprisingly the inventors showed that the split Cas9 domain comprising the RuvC motif as described above is capable of cleaving a target nucleic acid sequence independently of split domain comprising the HNH motif. Thus, they could establish that the guideRNA does not need the presence of the HNH domain to bind to the target nucleic acid sequence and is sufficiently stable to be bound by the RuvC split domain. In a preferred embodiment, said split Cas9 domain alone is capable of nicking said target nucleic acid sequence.

Each split domain can be fused to at least one active domain in the N-terminal and/or C-terminal end, said active domain can be selected from the group consisting of: nuclease (e.g. endonuclease or exonuclease), polymerase, kinase, phosphatase, methylase, demethylase, acetylase, desacetylase, topoisomerase, integrase, transposase, ligase, helicase, recombinase, transcriptional activator (e.g. VP64, VP16), transcriptional inhibitor (e. g; KRAB), DNA end processing enzyme (e.g. Trex2, Tdt), reporter molecule (e.g. fluorescent proteins, lacZ, luciferase).

HNH domain is responsible for nicking of one strand of the target double-stranded DNA and the RuvC-like RNaseH fold domain is involved in nicking of the other strand (comprising the PAM motif) of the double-stranded nucleic acid target (Jinek, Chylinski et al. 2012). However, in wild-type Cas9, these two domains result in blunt cleavage of the invasive DNA within the same target sequence (proto-spacer) in the immediate vicinity of the PAM (Jinek, Chylinski et al. 2012). Cas 9 can be a nickase and induces a nick event within different target sequences.

As non-limiting example, Cas9 or split Cas9 can comprise mutation(s) in the catalytic residues of either the HNH or RuvC-like domains, to induce a nick event within different target sequences. As non-limiting example, the catalytic residues of the Cas9 protein are those corresponding to amino acids D10, D31, H840, H868, N882 and N891 or aligned positions using CLUSTALW method on homologues of Cas Family members. Any of these residues can be replaced by any other amino acids, preferably by alanine residue. Mutation in the catalytic residues means either substitution by another amino acids, or deletion or addition of amino acids that induce the inactivation of at least one of the catalytic domain of cas9. (cf. In a particular embodiment, Cas9 or split Cas9 may comprise one or several of the above mutations. In another particular embodiment, split Cas9 comprises only one of the two RuvC and HNH catalytic domains. In the present invention, Cas9 from different species, Cas9 homologues, Cas9 engineered and functional variant thereof can be used. The invention envisions the use of any RNA guided endonuclease or split RNA guided endonucleases variants to perform nucleic acid cleavage in a genetic sequence of interest.

Preferably, the Cas9 variants according to the invention have an amino acid sequence sharing at least 70%, preferably at least 80%, more preferably at least 90%, and even more preferably 95% identity with Cas9 of S. pyogenes (COG3513).

Meganucleases

Rare-cutting endonuclease can also be a homing endonuclease, also known under the name of meganuclease. Such homing endonucleases are well-known to the art (Stoddard 2005). Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Preferred homing endonuclease according to the present invention can be an I-CreI variant. A “variant” endonuclease, i.e. an endonuclease that does not naturally exist in nature and that is obtained by genetic engineering or by random mutagenesis can bind DNA sequences different from that recognized by wild-type endonucleases (see international application WO2006/097854).

Said rare-cutting endonuclease can be a modular DNA binding nuclease. By modular DNA binding nuclease is meant any fusion proteins comprising at least one catalytic domain of an endonuclease and at least one DNA binding domain or protein specifying a nucleic acid target sequence. The DNA binding domain is generally a RNA or DNA-binding domain formed by an independently folded polypeptide or protein domain that contains at least one motif that recognizes double- or single-stranded polynucleotides. Many such polypeptides have been described in the art having the ability to bind specific nucleic acid sequences. Such binding domains often comprise, as non-limiting examples, helix-turn helix domains, leucine zipper domains, winged helix domains, helix-loop-helix domains, HMG-box domains, Immunoglobin domains, B3 domain or engineered zinc finger domain.

The present invention provides a rare-cutting endonuclease for inactivating a gene which product is expressed or overexpressed on pathological cells and normal T cell and which product is expressed on the surface of normal T-cells selected from any one of those discloses in table 4 to table 14.

In preferred embodiment the rare-cutting endonuclease of the invention is specific for the CD38 gene or for the CD70 gene or for the CS1 gene.

The rare-cutting endonuclease as above is provided and selected from a Meganuclease, a transcription activator-like (TAL)-nuclease, a zing-finger nuclease (ZFN), or a RNA/DNA guided endonucleases.

The rare-cutting endonuclease as above is provided and selected from a Meganuclease, a transcription activator-like (TAL)-nuclease, a zing-finger nuclease (ZFN), or a RNA/DNA guided endonucleases.

The rare-cutting endonuclease provided here is a transcription activator-like effector (TALE)-nuclease specific for CD38, CD70 or CS-1 gene.

The rare-cutting endonuclease of the invention may be for example specific for one of the following SEQ ID NO. 1, SEQ ID NO. 4, SEQ ID NO. 7, SEQ ID NO. 63, SEQ ID NO. 66, SEQ ID NO. 69, SEQ ID NO. 72, SEQ ID NO. 75, SEQ ID NO. 78, SEQ ID NO. 167, SEQ ID NO. 168, SEQ ID NO. 169 or SEQ ID NO. 170.

The present invention encompasses TAL-nucleases having at least 95% homology with any one of the TAL-nucleases comprising SEQ ID NO 114 and SEQ ID NO 115; SEQ ID NO 116 and SEQ ID NO 117; SEQ ID NO 118 and SEQ ID NO 119; SEQ ID NO 102 and SEQ ID NO 103; SEQ ID NO 104 and SEQ ID NO 105; SEQ ID NO 106 and SEQ ID NO 107; SEQ ID NO 108 and SEQ ID NO 109; SEQ ID NO 110 and SEQ ID NO 111; SEQ ID NO 112 and SEQ ID NO 113; SEQ ID NO 171 and SEQ ID NO 172; SEQ ID NO 173 and SEQ ID NO 174; SEQ ID NO 175 and SEQ ID NO 176; or SEQ ID NO 177 and SEQ ID NO 178.

Zinc-Finger Nucleases

Initially developed to cleave DNA in vitro, “Zinc Finger Nucleases” (ZFNs) are a fusion between the cleavage domain of the type IIS restriction enzyme, FokI, and a DNA recognition domain containing 3 or more C2H2 zinc finger motifs. The heterodimerization at a particular position in the DNA of two individual ZFNs in precise orientation and spacing leads to a double-strand break (DSB) in the DNA. The use of such chimeric endonucleases have been extensively reported in the art as reviewed by Urnov et al. (Genome editing with engineered zinc finger nucleases (2010) Nature reviews Genetics 11:636-646).

Standard ZFNs fuse the cleavage domain to the C-terminus of each zinc finger domain. In order to allow the two cleavage domains to dimerize and cleave DNA, the two individual ZFNs bind opposite strands of DNA with their C-termini a certain distance apart. The most commonly used linker sequences between the zinc finger domain and the cleavage domain requires the 5′ edge of each binding site to be separated by 5 to 7 bp.

The most straightforward method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Numerous selection methods have been used to generate zinc-finger arrays capable of targeting desired sequences. Initial selection efforts utilized phage display to select proteins that bound a given DNA target from a large pool of partially randomized zinc-finger arrays. More recent efforts have utilized yeast one-hybrid systems, bacterial one-hybrid and two-hybrid systems, and mammalian cells.

TAL-Nucleases

“TALE-nuclease” or “MBBBD-nuclease” refers to engineered proteins resulting from the fusion of a DNA binding domain typically derived from Transcription Activator Like Effector proteins (TALE) or Modular Base-per-Base Binding domain (MBBBD), with a catalytic domain having endonuclease activity. Such catalytic domain usually comes from enzymes, such as for instance I-Tevl, ColE7, NucA and Fok-I. TALE-nuclease can be formed under monomeric or dimeric forms depending of the selected catalytic domain (WO2012138927). Such engineered TALE-nucleases are commercially available under the trade name TALEN™ (Cellectis, 8 rue de la Croix Jarry, 75013 Paris, France).

According to a preferred embodiment of the invention, the DNA binding domain is derived from a Transcription Activator like Effector (TALE), wherein sequence specificity is driven by a series of 33-35 amino acids repeats originating from Xanthomonas or Ralstonia bacterial proteins AvrBs3, PthXo1, AvrHah1, PthA, Tal1c as non-limiting examples.

These repeats differ essentially by two amino acids positions that specify an interaction with a base pair (Boch, Scholze et al. 2009; Moscou and Bogdanove 2009). Each base pair in the DNA target is contacted by a single repeat, with the specificity resulting from the two variant amino acids of the repeat (the so-called repeat variable dipeptide, RVD). TALE binding domains may further comprise an N-terminal translocation domain responsible for the requirement of a first thymine base (TO) of the targeted sequence and a C-terminal domain that containing a nuclear localization signals (NLS). A TALE nucleic acid binding domain generally corresponds to an engineered core TALE scaffold comprising a plurality of TALE repeat sequences, each repeat comprising a RVD specific to each nucleotides base of a TALE recognition site. In the present invention, each TALE repeat sequence of said core scaffold is made of 30 to 42 amino acids, more preferably 33 or 34 wherein two critical amino acids (the so-called repeat variable dipeptide, RVD) located at positions 12 and 13 mediates the recognition of one nucleotide of said TALE binding site sequence; equivalent two critical amino acids can be located at positions other than 12 and 13 specially in TALE repeat sequence taller than 33 or 34 amino acids long. Preferably, RVDs associated with recognition of the different nucleotides are HD for recognizing C, NG for recognizing T, NI for recognizing A, NN for recognizing G or A. In another embodiment, critical amino acids 12 and 13 can be mutated towards other amino acid residues in order to modulate their specificity towards nucleotides A, T, C and G and in particular to enhance this specificity. A TALE nucleic acid binding domain usually comprises between 8 and 30 TALE repeat sequences. More preferably, said core scaffold of the present invention comprises between 8 and 20 TALE repeat sequences; again more preferably 15 TALE repeat sequences. It can also comprise an additional single truncated TALE repeat sequence made of 20 amino acids located at the C-terminus of said set of TALE repeat sequences, i.e. an additional C-terminal half-TALE repeat sequence.

Other engineered DNA binding domains can be used as alternative sequences to form so-called modular base-per-base specific nucleic acid binding domains (MBBBD) as described in WO 2014/018601. Said MBBBD can be engineered, for instance, from newly identified proteins, namely EAV36_BURRH, E5AW43_BURRH, E5AW45_BURRH and E5AW46_BURRH proteins from the recently sequenced genome of the endosymbiont fungi Burkholderia rhizoxinica (Lackner, Moebius et al. 2011). These nucleic acid binding polypeptides comprise modules of about 31 to 33 amino acids that are base specific. These modules display less than 40% sequence identity with Xanthomonas TALE common repeats and present more polypeptides sequence variability. The different domains from the above proteins (modules, N and C-terminals) from Burkholderia and Xanthomonas are useful to engineer new proteins or scaffolds having binding properties to specific nucleic acid sequences and may be combined to form chimeric TALE-MBBBD proteins.

In a preferred embodiment, a TAL nuclease of the invention comprises a sequence selected from any one the following pair of sequences: SEQ ID NO 102 and SEQ ID NO 103, SEQ ID NO 104 and SEQ ID NO 105, SEQ ID NO 106 and SEQ ID NO 107, SEQ ID NO 108 and SEQ ID NO 109, SEQ ID NO 110 and SEQ ID NO 111, SEQ ID NO 112 and SEQ ID NO 113, SEQ ID NO 114 and SEQ ID NO 115, SEQ ID NO 116 and SEQ ID NO 117, SEQ ID NO 118 and SEQ ID NO 119, SEQ ID N0171 and SEQ ID NO 172, SEQ ID NO 173 and SEQ ID NO 174, SEQ ID NO 175 and SEQ ID NO 176, SEQ ID NO 177 and SEQ ID NO 178; or a sequence having least 95% homology with any one of said pair of sequences.

Delivery Methods

The inventors have considered any means known in the art to allow delivery inside cells or subcellular compartments of said cells the polynucleotides expressing the endonucleases, their possible co-effectors (e.g. guide RNA, . . . ) as well as the chimeric antigen receptors. These means include viral transduction, electroporation and also liposomal delivery means, polymeric carriers, chemical carriers, lipoplexes, polyplexes, dendrimers, nanoparticles, emulsion, natural endocytosis or phagocytose pathway as non-limiting examples.

As a preferred embodiment of the invention, polynucleotides encoding the endonucleases of the present invention are transfected under mRNA form in order to obtain transient expression and avoid chromosomal integration of foreign DNA, for example by electroporation. The inventors have determined different optimal conditions for mRNA electroporation in T-cell displayed in Table 1. The inventor used the cytoPulse technology which allows, by the use of pulsed electric fields, to transiently permeabilize living cells for delivery of material into the cells (U.S. Pat. No. 6,010,613 and WO 2004/083379). Pulse duration, intensity as well as the interval between pulses can be modified in order to reach the best conditions for high transfection efficiency with minimal mortality. Basically, the first high electric field pulses allow pore formation, while subsequent lower electric field pulses allow to moving the polynucleotide into the cell. In one aspect of the present invention, the inventor describe the steps that led to achievement of >95% transfection efficiency of mRNA in T cells, and the use of the electroporation protocol to transiently express different kind of proteins in T cells. In particular the invention relates to a method of transforming T cell comprising contacting said T cell with RNA and applying to T cell an agile pulse sequence consisting of:
(a) one electrical pulse with a voltage range from 2250 to 3000 V per centimeter, a pulse width of 0.1 ms and a pulse interval of 0.2 to 10 ms between the electrical pulses of step (a) and (b);
(b) one electrical pulse with a voltage range from 2250 to 3000 V with a pulse width of 100 ms and a pulse interval of 100 ms between the electrical pulse of step (b) and the first electrical pulse of step (c); and
(c) 4 electrical pulses with a voltage of 325 V with a pulse width of 0.2 ms and a pulse interval of 2 ms between each of 4 electrical pulses.

In particular embodiment, the method of transforming T cell comprising contacting said T cell with RNA and applying to T cell an agile pulse sequence consisting of:
(a) one electrical pulse with a voltage of 2250, 2300, 2350, 2400, 2450, 2500, 2550, 2400, 2450, 2500, 2600, 2700, 2800, 2900 or 3000V per centimeter, a pulse width of 0.1 ms and a pulse interval of 0.2, 0.5, 1, 2, 3, 4, 5, 6, 7, 8, 9 or 10 ms between the electrical pulses of step (a) and (b);
(b) one electrical pulse with a voltage range from 2250, of 2250, 2300, 2350, 2400, 2450, 2500, 2550, 2400, 2450, 2500, 2600, 2700, 2800, 2900 or 3000V with a pulse width of 100 ms and a pulse interval of 100 ms between the electrical pulse of step (b) and the first electrical pulse of step (c); and
(c) 4 electrical pulses with a voltage of 325 V with a pulse width of 0.2 ms and a pulse interval of 2 ms between each of 4 electrical pulses.

Any values included in the value range described above are disclosed in the present application. Electroporation medium can be any suitable medium known in the art. Preferably, the electroporation medium has conductivity in a range spanning 0.01 to 1.0 milliSiemens.

TABLE 1
Different cytopulse programs used to determine the minimal voltage
required for electroporation in PBMC derived T-cells.

| | Group 1 | | | | Group 2 | | | | Group 3 | | | |
|---|---|---|---|---|---|---|---|---|---|---|---|---|
| Cytopulse | | | duration | Interval | | | duration | Interval | | | duration | Interval |
| program | Pulses | V | (ms) | (ms) | Pulses | V | (ms) | (ms) | Pulses | V | (ms) | (ms) |
| 1 | 1 | 600 | 0.1 | 0.2 | 1 | 600 | 0.1 | 100 | 4 | 130 | 0.2 | 2 |
| 2 | 1 | 900 | 0.1 | 0.2 | 1 | 900 | 0.1 | 100 | 4 | 130 | 0.2 | 2 |
| 3 | 1 | 1200 | 0.1 | 0.2 | 1 | 1200 | 0.1 | 100 | 4 | 130 | 0.2 | 2 |
| 4 | 1 | 1200 | 0.1 | 10 | 1 | 900 | 0.1 | 100 | 4 | 130 | 0.2 | 2 |
| 5 | 1 | 900 | 0.1 | 20 | 1 | 600 | 0.1 | 100 | 4 | 130 | 0.2 | 2 |

Viral Transduction

According to the present invention, the use of retroviral vectors and more preferably of lentiviral vectors is particularly suited for expressing the chimeric antigen receptors into the T-cells. In a preferred embodiment the use of adeno associated viral vectors and more preferably of AAV6, or AAV9 vectors is particularly suited for expressing a gene including chimeric antigen receptors into the T-cells. Methods for viral transduction are well known in the art (Walther et al. (2000) Viral Vectors for Gene Transfer. Drugs. 60(2):249-271). Integrative viral vectors allow the stable integration of the polynucleotides in the T-cells genome and to expressing the chimeric antigen receptors over a longer period of time.

Non Alloreactive T Cells:

Although the method of the invention could be carried out in-vivo as part of a gene therapy, for instance, by using viral vectors targeting T-cells in blood circulation, which would include genetic sequences expressing a specific rare-cutting endonuclease along with other genetic sequences expressing a CAR, the method of the invention is more generally intended to be practiced ex-vivo on cultured T-cells obtainable from patients or donors. The engineered T-cells engineered ex-vivo can be either re-implanted into a patient from where they originate, as part of an autologous treatment, or to be used as part of an allogeneic treatment. In this later case, it is preferable to further engineer the cells to make them non-alloreactive to ensure their proper engraftment. Accordingly, the method of the invention may include additional steps of procuring the T-cells from a donor and to inactivate genes thereof involved in MHC recognition and or being targets of immunosuppressive drugs such as described for instance in WO 2013/176915.

T cell receptors (TCR) are cell surface receptors that participate in the activation of T cells in response to the presentation of antigen. The TCR is generally made from two chains, alpha and beta, which assemble to form a heterodimer and associates with the CD3-transducing subunits to form the T-cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the alpha and beta chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of GVHD. It has been shown that normal surface expression of the TCR depends on the coordinated synthesis and assembly of all seven components of the complex (Ashwell and Klusner 1990). The inactivation of TCRalpha or TCRbeta can result in the elimination of the TCR from the surface of T cells preventing recognition of alloantigen and thus GVHD.

Thus, still according to the invention, engraftment of the allogeneic T-cells may be improved by inactivating at least one gene encoding a TCR component. TCR is rendered not functional in the cells by inactivating TCR alpha gene and/or TCR beta gene(s), preferably by inserting a sequence into the endogenous TCR gene (genomic TCR gene), by using a viral vector preferably an adeno associated virus.

With respect to the use of Cas9/CRISPR system, the inventors have determined appropriate target sequences within the 3 exons encoding TCR, allowing a significant reduction of toxicity in living cells, while retaining cleavage efficiency. The preferred target sequences are noted in Table 2 (+ for lower ratio of TCR negative cells, ++ for intermediate ratio, +++ for higher ratio).

TABLE2
appropriate target sequences for the
guide RNA using Cas9 in T-cells

| Exon | | | | SEQ | effi- |
|---|---|---|---|---|---|
| TCR | Position | Strand | Target genomic sequence | ID | ciency |
| Ex1 | 78 | −1 | GAGAATCAAAATCGGTGAATAGG | 120 | +++ |
| Ex3 | 26 | 1 | TTCAAAACCTGTCAGTGATTGGG | 121 | +++ |
| Ex1 | 153 | 1 | TGTGCTAGACATGAGGTCTATGG | 122 | +++ |
| Ex3 | 74 | −1 | CGTCATGAGCAGATTAAACCCGG | 123 | +++ |
| Ex1 | 4 | −1 | TCAGGGTTCTGGATATCTGTGGG | 124 | +++ |
| Ex1 | 5 | −1 | GTCAGGGTTCTGGATATCTGTGG | 125 | +++ |
| Ex3 | 33 | −1 | TTCGGAACCCAATCACTGACAGG | 126 | +++ |
| Ex3 | 60 | −1 | TAAACCCGGCCACTTTCAGGAGG | 127 | +++ |
| Ex1 | 200 | −1 | AAAGTCAGATTTGTTGCTCCAGG | 128 | ++ |
| Ex1 | 102 | 1 | AACAAATGTGTCACAAAGTAAGG | 129 | ++ |
| Ex1 | 39 | −1 | TGGATTTAGAGTCTCTCAGCTGG | 130 | ++ |
| Ex1 | 59 | −1 | TAGGCAGACAGACTTGTCACTGG | 131 | ++ |
| Ex1 | 22 | −1 | AGCTGGTACACGGCAGGGTCAGG | 132 | ++ |
| Ex1 | 21 | −1 | GCTGGTACACGGCAGGGTCAGGG | 133 | ++ |
| Ex1 | 28 | −1 | TCTCTCAGCTGGTACACGGCAGG | 134 | ++ |
| Ex3 | 25 | 1 | TTTCAAAACCTGTCAGTGATTGG | 135 | ++ |
| Ex3 | 63 | −1 | GATTAAACCCGGCCACTTTCAGG | 136 | ++ |
| Ex2 | 17 | −1 | CTCGACCAGCTTGACATCACAGG | 137 | ++ |
| Ex1 | 32 | −1 | AGAGTCTCTCAGCTGGTACACGG | 138 | ++ |
| Ex1 | 27 | −1 | CTCTCAGCTGGTACACGGCAGGG | 139 | ++ |
| Ex2 | 12 | 1 | AAGTTCCTGTGATGTCAAGCTGG | 140 | ++ |
| Ex3 | 55 | 1 | ATCCTCCTCCTGAAAGTGGCCGG | 141 | ++ |
| Ex3 | 86 | 1 | TGCTCATGACGCTGCGGCTGTGG | 142 | ++ |
| Ex1 | 146 | 1 | ACAAAACTGTGCTAGACATGAGG | 143 | + |
| Ex1 | 86 | −1 | ATTTGTTTGAGAATCAAAATCGG | 144 | + |
| Ex2 | 3 | −1 | CATCACAGGAACTTTCTAAAAGG | 145 | + |
| Ex2 | 34 | 1 | GTCGAGAAAAGCTTTGAAACAGG | 146 | + |
| Ex3 | 51 | −1 | CCACTTTCAGGAGGAGGATTCGG | 147 | + |
| Ex3 | 18 | −1 | CTGACAGGTTTTGAAAGTTTAGG | 148 | + |
| Ex2 | 43 | 1 | AGCTTTGAAACAGGTAAGACAGG | 149 | + |
| Ex1 | 236 | −1 | TGGAATAATGCTGTTGTTGAAGG | 150 | + |
| Ex1 | 182 | 1 | AGAGCAACAGTGCTGTGGCCTGG | 151 | + |
| Ex3 | 103 | 1 | CTGTGGTCCAGCTGAGGTGAGGG | 152 | + |
| Ex3 | 97 | 1 | CTGCGGCTGTGGTCCAGCTGAGG | 153 | + |
| Ex3 | 104 | 1 | TGTGGTCCAGCTGAGGTGAGGGG | 154 | + |
| Ex1 | 267 | 1 | CTTCTTCCCCAGCCCAGGTAAGG | 155 | + |
| Ex1 | 15 | −1 | ACACGGCAGGGTCAGGGTTCTGG | 156 | + |
| Ex1 | 177 | 1 | CTTCAAGAGCAACAGTGCTGTGG | 157 | + |
| Ex1 | 256 | −1 | CTGGGGAAGAAGGTGTCTTCTGG | 158 | + |
| Ex3 | 56 | 1 | TCCTCCTCCTGAAAGTGGCCGGG | 149 | + |
| Ex3 | 80 | 1 | TTAATCTGCTCATGACGCTGCGG | 160 | + |
| Ex3 | 57 | −1 | ACCCGGCCACTTTCAGGAGGAGG | 161 | + |
| Ex1 | 268 | 1 | TTCTTCCCCAGCCCAGGTAAGGG | 162 | + |
| Ex1 | 266 | −1 | CTTACCTGGGCTGGGGAAGAAGG | 163 | + |
| Ex1 | 262 | 1 | GACACCTTCTTCCCCAGCCCAGG | 164 | + |
| Ex3 | 102 | 1 | GCTGTGGTCCAGCTGAGGTGAGG | 165 | + |
| Ex3 | 51 | 1 | CCGAATCCTCCTCCTGAAAGTGG | 166 | + |

MHC antigens are also proteins that played a major role in transplantation reactions. Rejection is mediated by T cells reacting to the histocompatibility antigens on the surface of implanted tissues, and the largest group of these antigens is the major histocompatibility antigens (MHC). These proteins are expressed on the surface of all higher vertebrates and are called HLA antigens (for human leukocyte antigens) in human cells. Like TCR, the MHC proteins serve a vital role in T cell stimulation. Antigen presenting cells (often dendritic cells) display peptides that are the degradation products of foreign proteins on the cell surface on the MHC. In the presence of a co-stimulatory signal, the T cell becomes activated, and will act on a target cell that also displays that same peptide/MHC complex. For example, a stimulated T helper cell will target a macrophage displaying an antigen in conjunction with its MHC, or a cytotoxic T cell (CTL) will act on a virally infected cell displaying foreign viral peptides.

Thus, in order to provide less alloreactive T-cells, the method of the invention can further comprise the step of inactivating or mutating one HLA gene.

The class I HLA gene cluster in humans comprises three major loci, B, C and A, as well as several minor loci. The class II HLA cluster also comprises three major loci, DP, DQ and DR, and both the class I and class II gene clusters are polymorphic, in that there are several different alleles of both the class I and II genes within the population. There are also several accessory proteins that play a role in HLA functioning as well. The TapI and Tap2 subunits are parts of the TAP transporter complex that is essential in loading peptide antigens on to the class I HLA complexes, and the LMP2 and LMP7 proteosome subunits play roles in the proteolytic degradation of antigens into peptides for display on the HLA. Reduction in LMP7 has been shown to reduce the amount of MHC class I at the cell surface, perhaps through a lack of stabilization (Fehling et al. (1999) Science 265:1234-1237). In addition to TAP and LMP, there is the tapasin gene, whose product forms a bridge between the TAP complex and the HLA class I chains and enhances peptide loading. Reduction in tapasin results in cells with impaired MHC class I assembly, reduced cell surface expression of the MHC class I and impaired immune responses (Grandea et al. (2000) Immunity 13:213-222 and Garbi et al. (2000) Nat. Immunol. 1:234-238). Any of the above genes may be inactivated as part of the present invention as disclosed, for instance in WO 2012/012667.

The present invention provides an engineered T cell comprising any one of the genetic modification to a sequence as shown in SEQ ID NO. 1, SEQ ID NO. 4, or SEQ ID NO. 7 of the CD38 gene, of SEQ ID NO. 63, SEQ ID NO. 66, SEQ ID NO. 69, SEQ ID NO. 167, SEQ ID NO. 168, SEQ ID NO. 169 or SEQ ID NO. 170 of the CS1 gene or of SEQ ID NO. 72, SEQ ID NO. 75 or SEQ ID NO. 78 of the CD70 gene.

The present invention provides a combination comprising an engineered T cell comprising any one of the genetic modification to a sequence as shown in SEQ ID NO. 1, SEQ ID NO. 4, SEQ ID NO. 7 of the CD38 gene and a therapeutic antibody specific for the antigen marker encoded by the CD38 gene.

The present invention provides a combination comprising an engineered T cell comprising any one of the genetic modification to a sequence as shown in SEQ ID NO. 63, SEQ ID NO. 66, SEQ ID NO. 69, SEQ ID NO. 167, SEQ ID NO. 168, SEQ ID NO. 169 or SEQ ID NO. 170 of the CS1 gene and a therapeutic antibody specific for the antigen marker encoded by the CS1 gene.

The present invention provides a combination comprising an engineered T cell comprising any one of the genetic modification to a sequence as shown in SEQ ID NO. 72, SEQ ID NO. 75 or SEQ ID NO. 78 of the CD70 gene and a therapeutic antibody specific for the antigen marker encoded by the CD70 gene.

The engineered T cell according to the above comprises a genetic modification to a sequence of SEQ ID NO. 1 by a TAL protein comprising a sequence SEQ ID NO 114 and SEQ ID NO 115, a genetic modification to a sequence of SEQ ID NO. 4 by a TALEN comprising a SEQ ID No 116 and SEQ ID NO 117, or a genetic modification to a sequence of SEQ ID NO. 7 by a TAL protein comprising a sequence SEQ ID No 118 and SEQ ID NO 119; and/or a genetic modification to a sequence of SEQ ID NO. 63 by a TAL protein of sequence SEQ ID NO 108 and SEQ ID NO 109, a genetic modification to a sequence of SEQ ID NO. 66 by a TAL protein of sequence SEQ ID NO 110 and SEQ ID NO 111, a genetic modification to a sequence of SEQ ID NO. 69 by a TAL protein of sequence SEQ ID NO 112 and SEQ ID NO 113, a genetic modification to a sequence of SEQ ID NO. 167 by a TAL protein of sequence SEQ ID NO 171 and SEQ ID NO 172, a genetic modification to a sequence of SEQ ID NO. 168 by a TAL protein of sequence SEQ ID NO 173 and SEQ ID NO 174, or a genetic modification to a sequence of SEQ ID NO. 169 by a TAL protein of sequence SEQ ID NO 171 and SEQ ID NO 172; and/or a genetic modification to a sequence of SEQ ID NO. 72 by a TAL protein of sequence SEQ ID NO 102 and SEQ ID NO 103, a genetic modification to a sequence of SEQ ID NO. 75 by a TAL protein of sequence SEQ ID NO 104 and SEQ ID NO 105, a genetic modification to a sequence of SEQ ID NO. 78 by a TAL protein of sequence SEQ ID NO 106 and SEQ ID NO 107.

The engineered T cell according to the above comprising an insertion of a polynucleotide sequence encoding a gene, preferably a CAR, preferably at the locus described above, into the TRAC gene and at least one a genetic inactivation to a sequence of SEQ ID NO. 1 by a TAL protein comprising a sequence SEQ ID NO 114 and SEQ ID NO 115; a genetic modification to a sequence of SEQ ID NO. 4 by a TALEN comprising a SEQ ID NO 116 and SEQ ID NO 117, a genetic modification to a sequence of SEQ ID NO. 7 by a TAL protein comprising a sequence SEQ ID NO 118 and SEQ ID NO 119, a genetic modification to a sequence of SEQ ID NO. 63 by a TAL protein of sequence SEQ ID NO 108 and SEQ ID NO 109, a genetic modification to a sequence of SEQ ID NO. 66 by a TAL protein of sequence SEQ ID NO 110 and SEQ ID NO 111, a genetic modification to a sequence of SEQ ID NO. 69 by a TAL protein of sequence SEQ ID NO 112 and SEQ ID NO 113, of the CS1 gene, a genetic modification to a sequence of SEQ ID NO. 72 by a TAL protein of sequence SEQ ID NO 102 and SEQ ID NO 103, a genetic modification to a sequence of SEQ ID NO. 75 by a TAL protein of sequence SEQ ID NO 104 and SEQ ID NO 105, a genetic modification to a sequence of SEQ ID NO. 78 of the CD70 gene by a TAL protein of sequence SEQ ID NO 106 and SEQ ID NO 107.

The TAL-nuclease of the invention as any one of the above is provided for use as a medicament, alone or in combination with an therapeutic antibody specific for the same gene.

The combination of the invention as any one of the above and below is provided for the treatment of cancer, infections or auto-immune disease.

Method of Engineering Drug-Resistant T-Cells:

To improve cancer therapy and selective engraftment of allogeneic T-cells, drug resistance can be conferred to the engineered T-cells to protect them from the toxic side effects of chemotherapy or immunosuppressive agents. Indeed, most patients were treated with chemotherapy and immune depleting agents as a standard of care, prior to receiving T-cell immunotherapy. One could take advantage of these treatments to help the selection of the engineered T-cells, either by adding chemotherapy drugs in culture media for expansion of the cells ex-vivo prior to treatment, or by obtaining a selective expansion of the engineered T-cells in-vivo in patients under chemotherapy or immunosuppressive treatments.

Also the drug resistance of T-cells also permits their enrichment in or ex vivo, as T-cells which express the drug resistance gene, will survive and multiply relative to drug sensitive cells. In particular, the present invention relates to a method of engineering allogeneic and drug resistance T-cells resistant for immunotherapy comprising:
(a) Providing a T-cell; or an engineered T cell
(b) Selecting at least one drug;
(c) Modifying T-cell to confer drug resistance to said T-cell;
(d) Expanding said engineered T-cell in the presence of said drug, and optionally the preceding steps may be combined with the steps of the methods as previously described.

Drug resistance can be conferred to a T-cell by inactivating one or more gene(s) responsible for the cell's sensitivity to the drug (drug sensitizing gene(s)), such as the hypoxanthine-guanine phosphoribosyl transferase (HPRT) gene (Genbank: M26434.1). In particular HPRT can be inactivated in engineered T-cells to confer resistance to a cytostatic metabolite, the 6-thioguanine (6TG) which is converted by HPRT to cytotoxic thioguanine nucleotide and which is currently used to treat patients with cancer, in particular leukemias (Hacke, Treger et al. 2013). Another example if the inactivation of the CD3 normally expressed at the surface of the T-cell can confer resistance to anti-CD3 antibodies such as teplizumab.

Drug resistance can also be conferred to a T-cell by expressing a drug resistance gene. Said drug resistance gene refers to a nucleic acid sequence that encodes “resistance” to an agent, such as a chemotherapeutic agent (e.g. methotrexate). In other words, the expression of the drug resistance gene in a cell permits proliferation of the cells in the presence of the agent to a greater extent than the proliferation of a corresponding cell without the drug resistance gene. A drug resistance gene of the invention can encode resistance to antimetabolite, methotrexate, vinblastine, cisplatin, alkylating agents, anthracyclines, cytotoxic antibiotics, anti-immunophilins, their analogs or derivatives, and the like.

Variant alleles of several genes such as dihydrofolate reductase (DHFR), inosine monophosphate dehydrogenase 2 (IMPDH2), calcineurin or methylguanine transferase (MGMT) have been identified to confer drug resistance to a cell. Said drug resistance gene can be expressed in the cell either by introducing a transgene encoding said gene into the cell or by integrating said drug resistance gene into the genome of the cell by homologous recombination. Several other drug resistance genes have been identified that can potentially be used to confer drug resistance to targeted cells (Takebe, Zhao et al. 2001; Sugimoto, Tsukahara et al. 2003; Zielske, Reese et al. 2003; Nivens, Felder et al. 2004; Bardenheuer, Lehmberg et al. 2005; Kushman, Kabler et al. 2007).

DHFR is an enzyme involved in regulating the amount of tetrahydrofolate in the cell and is essential to DNA synthesis. Folate analogs such as methotrexate (MTX) inhibit DHFR and are thus used as anti-neoplastic agents in clinic. Different mutant forms of DHFR which have increased resistance to inhibition by anti-folates used in therapy have been described. In a particular embodiment, the drug resistance gene according to the present invention can be a nucleic acid sequence encoding a mutant form of human wild type DHFR (GenBank: AAH71996.1) which comprises at least one mutation conferring resistance to an anti-folate treatment, such as methotrexate. In particular embodiment, mutant form of DHFR comprises at least one mutated amino acid at position G15, L22, F31 or F34, preferably at positions L22 or F31 ((Schweitzer, Dicker et al. 1990); International application WO 94/24277; U.S. Pat. No. 6,642,043).

As used herein, “antifolate agent” or “folate analogs” refers to a molecule directed to interfere with the folate metabolic pathway at some level. Examples of antifolate agents include, e.g., methotrexate (MTX); aminopterin; trimetrexate (Neutrexin™); edatrexate; N10-propargyl-5,8-dideazafolic acid (CB3717); ZD1694 (Tumodex), 5,8-dideazaisofolic acid (IAHQ); 5,10-dideazatetrahydrofolic acid (DDATHF); 5-deazafolic acid; PT523 (N alpha-(4-amino-4-deoxypteroyl)-N delta-hemiphthaloyl-L-ornithine); 10-ethyl-10-deazaaminopterin (DDATHF, lomatrexol); piritrexim; 10-EDAM; ZD1694; GW1843; Pemetrexate and PDX (10-propargyl-10-deazaaminopterin).

Another example of drug resistance gene can also be a mutant or modified form of ionisine-5′-monophosphate dehydrogenase II (IMPDH2), a rate-limiting enzyme in the de novo synthesis of guanosine nucleotides. The mutant or modified form of IMPDH2 is a IMPDH inhibitor resistance gene. IMPDH inhibitors can be mycophenolic acid (MPA) or its prodrug mycophenolate mofetil (MMF). The mutant IMPDH2 can comprises at least one, preferably two mutations in the MAP binding site of the wild type human IMPDH2 (NP_000875.2) that lead to a significantly increased resistance to IMPDH inhibitor. The mutations are preferably at positions T333 and/or S351 (Yam, Jensen et al. 2006; Sangiolo, Lesnikova et al. 2007: Jonnalagadda, Brown et al. 2013). In a particular embodiment, the threonine residue at position 333 is replaced with an isoleucine residue and the serine residue at position 351 is replaced with a tyrosine residue.

Another drug resistance gene is the mutant form of calcineurin. Calcineurin (PP2B) is an ubiquitously expressed serine/threonine protein phosphatase that is involved in many biological processes and which is central to T-cell activation. Calcineurin is a heterodimer composed of a catalytic subunit (CnA; three isoforms) and a regulatory subunit (CnB; two isoforms). After engagement of the T-cell receptor, calcineurin dephosphorylates the transcription factor NFAT, allowing it to translocate to the nucleus and active key target gene such as IL2. FK506 in complex with FKBP12, or cyclosporine A (CsA) in complex with CyPA block NFAT access to calcineurin's active site, preventing its dephosphorylation and thereby inhibiting T-cell activation (Brewin, Mancao et al. 2009). The drug resistance gene of the present invention can be a nucleic acid sequence encoding a mutant form of calcineurin resistant to calcineurin inhibitor such as FK506 and/or CsA. In a particular embodiment, said mutant form can comprise at least one mutated amino acid of the wild type calcineurin heterodimer a at positions: V314, Y341, M347, T351, W352, L354, K360, preferably double mutations at positions T351 and L354 or V314 and Y341. Correspondence of amino acid positions described herein is frequently expressed in terms of the positions of the amino acids of the form of wild-type human calcineurin heterodimer (GenBank: ACX34092.1).

In another particular embodiment, said mutant form can comprise at least one mutated amino acid of the wild type calcineurin heterodimer b at positions: V120, N123, L124 or K125, preferably double mutations at positions L124 and K125. Correspondence of amino acid positions described herein is frequently expressed in terms of the positions of the amino acids of the form of wild-type human calcineurin heterodimer b polypeptide (GenBank: ACX34095.1).

Another drug resistance gene is 0(6)-methylguanine methyltransferase (MGMT) encoding human alkyl guanine transferase (hAGT). AGT is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG, but retain their ability to repair DNA damage (Maze, Kurpad et al. 1999). In a particular embodiment, AGT mutant form can comprise a mutated amino acid of the wild type AGT position P140 (UniProtKB: P16455).

Another drug resistance gene can be multidrug resistance protein 1 (MDR1) gene. This gene encodes a membrane glycoprotein, known as P-glycoprotein (P-GP) involved in the transport of metabolic byproducts across the cell membrane. The P-Gp protein displays broad specificity towards several structurally unrelated chemotherapy agents. Thus, drug resistance can be conferred to cells by the expression of nucleic acid sequence that encodes MDR-1 (NP_000918).

Drug resistance gene can also be cytotoxic antibiotics, such as ble gene or mcrA gene. Ectopic expression of ble gene or mcrA in an immune cell gives a selective advantage when exposed to the chemotherapeutic agent, respectively the bleomycine or the mitomycin C.

The T-cells can also be made resistant to immunosuppressive agents. An immunosuppressive agent is an agent that suppresses immune function by one of several mechanisms of action. In other words, an immunosuppressive agent is a role played by a compound which is exhibited by a capability to diminish the extent and/or voracity of an immune response. As non-limiting example, an immunosuppressive agent can be a calcineurin inhibitor, a target of rapamycin, an interleukin-2 α-chain blocker, an inhibitor of inosine monophosphate dehydrogenase, an inhibitor of dihydrofolic acid reductase, a corticosteroid or an immunosuppressive antimetabolite. Classical cytotoxic immunosuppressants act by inhibiting DNA synthesis. Others may act through activation of T-cells or by inhibiting the activation of helper cells. The method according to the invention allows conferring immunosuppressive resistance to T cells for immunotherapy by inactivating the target of the immunosuppressive agent in T cells. As non-limiting examples, targets for immunosuppressive agent can be a receptor for an immunosuppressive agent such as: CD52, glucocorticoid receptor (GR), a FKBP family gene member and a cyclophilin family gene member.

In immunocompetent hosts, allogeneic cells are normally rapidly rejected by the host immune system. It has been demonstrated that, allogeneic leukocytes present in non-irradiated blood products will persist for no more than 5 to 6 days. Thus, to prevent rejection of allogeneic cells, the host's immune system must be effectively suppressed. Glucocorticoidsteroids are widely used therapeutically for immunosuppression. This class of steroid hormones binds to the glucocorticoid receptor (GR) present in the cytosol of T cells resulting in the translocation into the nucleus and the binding of specific DNA motifs that regulate the expression of a number of genes involved in the immunologic process. Treatment of T cells with glucocorticoid steroids results in reduced levels of cytokine production leading to T cell anergy and interfering in T cell activation. Alemtuzumab, also known as CAMPATH1-H, is a humanized monoclonal antibody targeting CD52, a 12 amino acid glycosylphosphatidyl-inositol-(GPI) linked glycoprotein (Waldmann and Hale, 2005). CD52 is expressed at high levels on T and B lymphocytes and lower levels on monocytes while being absent on granulocytes and bone marrow precursors. Treatment with Alemtuzumab, a humanized monoclonal antibody directed against CD52, has been shown to induce a rapid depletion of circulating lymphocytes and monocytes. It is frequently used in the treatment of T cell lymphomas and in certain cases as part of a conditioning regimen for transplantation. However, in the case of adoptive immunotherapy the use of immunosuppressive drugs will also have a detrimental effect on the introduced therapeutic T cells. Therefore, to effectively use an adoptive immunotherapy approach in these conditions, the introduced cells would need to be resistant to the immunosuppressive treatment.

As a preferred embodiment of the above steps, said gene of step (b), specific for an immunosuppressive treatment, is CD52, and the immunosuppressive treatment of step (d) comprises a humanized antibody targeting CD52 antigen. As another embodiment, said gene of step (b), specific for an immunosuppressive treatment, is a glucocorticoid receptor (GR) and the immunosuppressive treatment of step d) comprises a corticosteroid such as dexamethasone. As another embodiment, said target gene of step (b), specific for an immunosuppressive treatment, is a FKBP family gene member or a variant thereof and the immunosuppressive treatment of step (d) comprises FK506 also known as Tacrolimus or fujimycin. As another embodiment, said FKBP family gene member is FKBP12 or a variant thereof. As another embodiment, said gene of step (b), specific for an immunosuppressive treatment, is a cyclophilin family gene member or a variant thereof and the immunosuppressive treatment of step (d) comprises cyclosporine.

In a particular embodiment of the invention, the genetic modification step of the method relies on the inactivation of an antigen marker X expressed on both T cell and pathological cells and of at least two genes selected from the group consisting of CD52, dCK, GR, TCR alpha, TCR beta, a combination thereof. The inventor succeeded in engineering at least 5 genes in a primary cell as disclosed in PA 201670503.

In another embodiment, the genetic modification step of the method relies on the inactivation of more than two genes. The genetic modification is preferably operated ex-vivo using at least two RNA guides targeting the different genes.

In a particular embodiment of the invention, the genetic modification step of the method relies on the inactivation of two genes selected from the group consisting of dCK and GR, dCK and TCR alpha, dCK and TCR beta, GR and TCR alpha, GR and TCR beta, TCR alpha and TCR beta. In another embodiment, the genetic modification step of the method relies on the inactivation of more than two genes. The genetic modification is preferably operated ex-vivo using at least two RNA guides targeting the different genes.

Engineering Highly Active T Cells for Immunotherapy

According to the present invention, the cells can be selected from the group consisting of T-cells, preferably inflammatory T-lymphocytes, cytotoxic T-lymphocytes, regulatory T-lymphocytes or helper T-lymphocytes. In another embodiment, said cell can be from the group consisting of CD4+ T-lymphocytes and CD8+T-lymphocytes. They can be extracted from blood or derived from stem cells. The cells may also be stem cells preferably cells can be adult stem cells, embryonic stem cells. In another embodiments cells may be non-human stem cells, cord blood stem cells, progenitor cells, bone marrow stem cells, induced pluripotent stem cells, totipotent stem cells or hematopoietic stem cells. Representative human cells are CD34+ cells, or CD8+ cells. Prior to expansion and genetic modification of the cells of the invention, a source of cells can be obtained from a subject through a variety of non-limiting methods. T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used. In another embodiment, said cell can be derived from a healthy donor, from a patient diagnosed with cancer or from a patient diagnosed with an infection. In another embodiment, said cell is part of a mixed population of cells which present different phenotypic characteristics. In the scope of the present invention is also encompassed a cell line obtained from a transformed T-cell according to the method previously described.

The cells used here may have been already engineered, that is to say inactivated by deleting a TCR subunit, preferably a TCR alpha subunit, made resistant to PNA by inactivating the dCK gene, made resistant to alentuzumab by deleting the D52 gene as described in WO2015075195A1, or in Valton J, Guyot V, Marechal A, et al. A Multidrug-resistant Engineered CAR T Cell for Allogeneic Combination Immunotherapy. Molecular Therapy. 2015; 23(9):1507-1518. doi:10.1038/mt.2015.104.

As a further aspect of the invention, the T-cells according to the invention may be further engineered, preferably genetically engineered, to enhance their activity and/or activation, especially by modulating the expression of proteins involved in overall T-cell regulation, referred to as “immune-checkpoints”.

Immune Check Points

It will be understood by those of ordinary skill in the art, that the term “immune checkpoints” means a group of molecules expressed by T cells. These molecules effectively serve as “brakes” to down-modulate or inhibit an immune response. Immune checkpoint molecules include, but are not limited to Programmed Death 1 (PD-1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as HAVCR2, GenBank accession number: JX049979.1), BTLA (also known as CD272, accession number: NM_181780.3), BY55 (also known as CD160, GenBank accession number: CR541888.1), TIGIT (also known as IVSTM3, accession number: NM_173799), LAIR1 (also known as CD305, GenBank accession number: CR542051.1, {Meyaard, 1997 #122}), SIGLEC10 (GeneBank accession number: AY358337.1), 2B4 (also known as CD244, accession number: NM_001166664.1), PPP2CA, PPP2CB, PTPN6, PTPN22, CD96, CRTAM, SIGLEC7 {Nicoll, 1999 #123}, SIGLEC9 {Zhang, 2000 #124; Ikehara, 2004 #125}, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF {Quigley, 2010 #121}, GUCY1A2, GUCY1A3, GUCY1B2, GUCY1B3 which directly inhibit immune cells. For example, CTLA-4 is a cell-surface protein expressed on certain CD4 and CD8 T cells; when engaged by its ligands (B7-1 and B7-2) on antigen presenting cells, T-cell activation and effector function are inhibited. Thus the present invention relates to a method of engineering T-cells, especially for immunotherapy, comprising genetically modifying T-cells by inactivating at least one protein involved in the immune checkpoint, in particular PD1 and/or CTLA-4 or any immune-checkpoint proteins referred to in Table 3.

TABLE 3
List of genes encoding immune checkpoint proteins.

| | | Genes that can be inactivated |
|---|---|---|
| Pathway | | In the pathway |
| Co-inhibitory | CTLA4 (CD152) | CTLA4, PPP2CA, PPP2CB, PTPN6, |
| receptors | | PTPN22 |
| | PDCD1 (PD-1, CD279) | PDCD1 |
| | CD223 (lag3) | LAG3 |
| | HAVCR2 (tim3) | HAVCR2 |
| | BTLA(cd272) | BTLA |
| | CD160(by55) | CD160 |
| | IgSF family | TIGIT |
| | | CD96 |
| | | CRTAM |
| | LAIR1(cd305) | LAIR1 |
| | SIGLECs | SIGLEC7 |
| | | SIGLEC9 |
| | CD244(2b4) | CD244 |
| Death receptors | TRAIL | TNFRSF10B, TNFRSF10A, CASP8, |
| | | CASP10, CASP3, CASP6, CASP7 |
| | FAS | FADD, FAS |
| Cytokine signalling | TGF-beta signaling | TGFBRII, TGFBRI, SMAD2, SMAD3, |
| | | SMAD4, SMAD10, SKI, SKIL, TGIF1 |
| | IL10 signalling | IL10RA, IL10RB, HMOX2 |
| | IL6 signalling | IL6R, IL6ST |
| Arginine/tryptophan | | EIF2AK4 |
| starvation | | |
| Prevention of TCR | | CSK, PAG1 |
| signalling | | SIT1 |
| Induced Treg | induced Treg | FOXP3 |
| Transcription | transcription factors | PRDM1 (=blimp1, heterozygotes mice |
| factors | controlling exhaustion | control chronic viral infection better |
| controlling | | than wt or conditional KO) |
| exhaustion | | BATF |
| Hypoxia mediated | iNOS induced guanylated | GUCY1A2, GUCY1A3, GUCY1B2, |
| tolerance | cyclase | GUCY1B3 |

Engineered cells encompasses an engineered immune cell comprising at least one inactivated gene X and expressing a chimeric antigen receptor (CAR) or a component of a recombinant T-cell receptor; wherein said cell has been genetically modified to reduce or inactivate the expression of at least one additional endogenous polynucleotide sequence selected from:
a) polynucleotide sequence(s), which expression is(are) involved into reduction of glycolysis and calcium signaling in response to a low glucose condition, such as SERCA3 to increase calcium signaling, miR101 and mir26A to increase glycolysis, BCAT to mobilize glycolytic reserves; and/or
b) polynucleotide sequence(s), which expression up regulate(s) immune checkpoint proteins (e.g.TIM3, CEACAM, LAG3, TIGIT), such as IL27RA, STAT1, STAT3; and/or
c) polynucleotide sequence(s), which expression mediate(s) interaction with HLA-G, such as ILT2 or ILT4; and/or
d) polynucleotide sequence(s), which expression is(are) involved into the down regulation of T-cell proliferation such as SEMA7A, SHARPIN to reduce Treg proliferation, STAT1 to lower apoptosis, PEA15 to increase IL-2 secretion and RICTOR to favor CD8 memory differentiation; and/or
e) polynucleotide sequence(s), which expression is(are) involved into the down regulation of T-cell activation, such as mir21; and/or
f) polynucleotide sequence(s), which expression is(are) involved in signaling pathways responding to cytokines, such as JAK2 and AURKA
g) polynucleotide sequence(s), which expression is(are) involved in T-cell exhaustion, such as DNMT3, miRNA31, MT1A, MT2A, PTGER2.

Engineered T-Cells Expressing Chimeric Antigen Receptors Against Pathological Cells

The chimeric antigen receptors introduced into the T-cells according to the invention can adopt different design such as single-chain or multi-chain CARs. These different designs allow various strategies for improving specificity and binding efficiency towards the targeted pathological cells. Some of these strategies are illustrated in the figures of the present application. Single-chain CARs are the most classical version in the art. Multi-chain CAR architectures were developed by the applicant as allowing modulation of the activity of T-cells in terms of specificity and intensity. The multiple subunits can shelter additional co-stimulation domains or keep such domains at a distance, as well as other types of receptors, whereas classical single chain architecture can sometimes be regarded as too much sensitive and less permissive to multispecific interactions.

Single-Chain CAR

Adoptive immunotherapy, which involves the transfer of autologous antigen-specific T cells generated ex vivo, is a promising strategy to treat viral infections and cancer. The T cells used for adoptive immunotherapy can be generated either by expansion of antigen-specific T cells or redirection of T cells through genetic engineering (Park, Rosenberg et al. 2011). Transfer of viral antigen specific T cells is a well-established procedure used for the treatment of transplant associated viral infections and rare viral-related malignancies. Similarly, isolation and transfer of tumor specific T cells has been shown to be successful in treating melanoma.

Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T cell cytotoxicity. However, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors (Jena, Dotti et al. 2010).

In addition to the CAR targeting the antigen marker, which is common to the pathological cells and the T-cells, such as CD38, it is envisioned to express further CARs directed towards other antigen markers not necessarily expressed by the T-cells, so as to enhancing T-cells specificity.

Examples of chimeric antigen receptor that can be further expressed by the T-cells to create multi-specific cells, are antigen receptors directed against multiple myeloma or lymphoblastic leukemia antigen markers, such as TNFRSF17 (UNIPROT Q02223), SLAMF7 (UNIPROT Q9NQ25), GPRC5D (UNIPROT Q9NZD1), FKBP11 (UNIPROT Q9NYL4), KAMP3, ITGA8 (UNIPROT P53708), and FCRL5 (UNIPROT Q68SN8).

As further examples, the antigen of the target can be from any cluster of differentiation molecules (e.g. CD16, CD64, CD78, CD96, CLL1, CD116, CD117, CD71, CD45, CD71, CD123 and CD138), a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-I, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C (TnC A1) and fibroblast associated protein (fap); a lineage-specific or tissue specific antigen such as CD3, CD4, CD8, CD24, CD25, CD33, CD34, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF 17), or a virus-specific surface antigen such as an HIV-specific antigen (such as HIV gp120); an EBV-specific antigen, a CMV-specific antigen, a HPV-specific antigen, a Lasse Virus-specific antigen, an Influenza Virus-specific antigen as well as any derivate or variant of these surface markers. Antigens are not necessarily surface marker antigens but can be also endogenous small antigens presented by HLA class I at the surface of the cells.

Multi-Subunit CAR

Chimeric antigen receptors from the prior art introduced in T-cells have been formed of single chain polypeptides that necessitate serial appending of signaling domains. However, by moving signaling domains from their natural juxtamembrane position may interfere with their function. To overcome this drawback, the applicant recently designed a multi-chain CAR derived from FcεRI to allow normal juxtamembrane position of all relevant signaling domains. In this new architecture, the high affinity IgE binding domain of FcεRI alpha chain is replaced by an extracellular ligand-binding domain such as scFv to redirect T-cell specificity against cell targets and the N and/or C-termini tails of FcεRI beta chain are used to place costimulatory signals in normal juxtamembrane positions.

Accordingly, the CAR expressed by the engineered T-cell according to the invention can be a multi-chain chimeric antigen receptor (CAR) particularly adapted to the production and expansion of engineered T-cells of the present invention. Such multi-chain CARs comprise at least two of the following components:
a) one polypeptide comprising the transmembrane domain of FcεRI alpha chain and an extracellular ligand-binding domain,
b) one polypeptide comprising a part of N- and C-terminal cytoplasmic tail and the transmembrane domain of FcεRI beta chain and/or
c) at least two polypeptides comprising each a part of intracytoplasmic tail and the transmembrane domain of FcεRI gamma chain, whereby different polypeptides multimerize together spontaneously to form dimeric, trimeric or tetrameric CAR.

According to such architectures, ligands binding domains and signaling domains are born on separate polypeptides. The different polypeptides are anchored into the membrane in a close proximity allowing interactions with each other. In such architectures, the signaling and co-stimulatory domains can be in juxtamembrane positions (i.e. adjacent to the cell membrane on the internal side of it), which is deemed to allow improved function of co-stimulatory domains. The multi-subunit architecture also offers more flexibility and possibilities of designing CARs with more control on T-cell activation. For instance, it is possible to include several extracellular antigen recognition domains having different specificity to obtain a multi-specific CAR architecture. It is also possible to control the relative ratio between the different subunits into the multi-chain CAR. This type of architecture has been recently described by the applicant in PCT/US2013/058005.

The assembly of the different chains as part of a single multi-chain CAR is made possible, for instance, by using the different alpha, beta and gamma chains of the high affinity receptor for IgE (FcεRI) (Metzger, Alcaraz et al. 1986) to which are fused the signaling and co-stimulatory domains. The gamma chain comprises a transmembrane region and cytoplasmic tail containing one immunoreceptor tyrosine-based activation motif (ITAM) (Cambier 1995).

The multi-chain CAR can comprise several extracellular ligand-binding domains, to simultaneously bind different elements in target thereby augmenting immune cell activation and function. In one embodiment, the extracellular ligand-binding domains can be placed in tandem on the same transmembrane polypeptide, and optionally can be separated by a linker. In another embodiment, said different extracellular ligand-binding domains can be placed on different transmembrane polypeptides composing the multi-chain CAR. In another embodiment, the present invention relates to a population of multi-chain CARs comprising each one different extracellular ligand binding domains. In a particular, the present invention relates to a method of engineering immune cells comprising providing an immune cell and expressing at the surface of said cell a population of multi-chain CAR each one comprising different extracellular ligand binding domains. In another particular embodiment, the present invention relates to a method of engineering an immune cell comprising providing an immune cell and introducing into said cell polynucleotides encoding polypeptides composing a population of multi-chain CAR each one comprising different extracellular ligand binding domains. In a particular embodiment the method of engineering an immune cell comprises expressing at the surface of the cell at least a part of FcεRI beta and/or gamma chain fused to a signal-transducing domain and several part of FcεRI alpha chains fused to different extracellular ligand binding domains. In a more particular embodiment, said method comprises introducing into said cell at least one polynucleotide which encodes a part of FcεRI beta and/or gamma chain fused to a signal-transducing domain and several FcεRI alpha chains fused to different extracellular ligand binding domains. By population of multi-chain CARs, it is meant at least two, three, four, five, six or more multi-chain CARs each one comprising different extracellular ligand binding domains. The different extracellular ligand binding domains according to the present invention can preferably simultaneously bind different elements in target thereby augmenting immune cell activation and function.

The present invention also relates to an isolated immune cell which comprises a population of multi-chain CARs each one comprising different extracellular ligand binding domains.

The signal transducing domain or intracellular signaling domain of the multi-chain CAR of the invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the multi-chain CAR is expressed. For example, the effector function of a T cell can be a cytolytic activity or helper activity including the secretion of cytokines.

In the present application, the term “signal transducing domain” refers to the portion of a protein which transduces the effector signal function signal and directs the cell to perform a specialized function.

Preferred examples of signal transducing domain for use in single or multi-chain CAR can be the cytoplasmic sequences of the Fc receptor or T cell receptor and co-receptors that act in concert to initiate signal transduction following antigen receptor engagement, as well as any derivate or variant of these sequences and any synthetic sequence that as the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d. In a preferred embodiment, the signaling transducing domain of the multi-chain CAR can comprise the CD3zeta signaling domain, or the intracytoplasmic domain of the FcεRI beta or gamma chains.

In particular embodiment the signal transduction domain of the multi-chain CAR of the present invention comprises a co-stimulatory signal molecule. A co-stimulatory molecule is a cell surface molecule other than an antigen receptor or their ligands that is required for an efficient immune response.

Ligand binding-domains can be any antigen receptor previously used, and referred to, with respect to single-chain CAR referred to in the literature, in particular scFv from monoclonal antibodies. Bispecific or multi-specific CARs as described in WO 2014/4011988 are incorporated by reference.

The combination of the invention comprises an engineered T cell according to the above endowed with at least one chimeric antigen receptor (CAR) or exogenous TCR.

The combination of the invention comprises an engineered allogeneic T cell according to the above, engineered to be less alloreactive, by engineering or deleting genes encoding components of the HLA complex, either to inactivated cell surface expression, by deleting genes encoding components of the HLA complex, or modifying the sequence to match MHC molecules to those of the patient.

The combination of the invention comprises an engineered allogeneic T cell according to the above, engineered to be less alloreactive, by engineering or deleting genes encoding components of the HLA complex, selected from beta2microglobulin, regulatory factor X-associated ankyrin-containing protein (RFXANK), regulatory factor 5 (RFX5), regulatory factor X-associated protein (RFXAP), and class II transactivator (CIITA), TAP-1, a combination thereof.

The engineered T cell of the combination of the invention according to the above wherein said CAR is specific for: a cluster of differentiation molecule, a tumor-associated surface antigen, a lineage-specific or tissue specific antigen or a virus-specific surface antigen is provided. The engineered T cell according to the above may comprise a CAR wherein said CAR is targeting (and specific for) any of the following antigen surface marker: CD16, CD64, CD78, CD96, CLL1, CD116, CD117, CD71, CD45, CD71, CD123 and CD138; or ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-I, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C (TnC A1) and fibroblast associated protein (fap); or CD3, CD4, CD8, CD24, CD25, CD33, CD34, CD38, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF17); or HIV-specific antigen (such as HIV gp120); an EBV-specific antigen, a CMV-specific antigen, a HPV-specific antigen, a Lasse Virus-specific antigen, an Influenza Virus-specific antigen, as the therapeutic antibody combined. The CAR endowed in the engineered cells in the combination of the invention may be specific for (e.g. CD16, CD19, CD20, CD22, CD30, CD38, CD40, CD64, CD70, CD78, CD79a CD79b, CD96, CLL1, CD116, CD117, CD71, CD45, CD123 and CD138), CS1, a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), disialoganglioside GD2, o-acethyl GD2, GD3, mesothelin, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RUI, RU2 (AS), intestinal carboxyl esterase, hsp70-2, HSP70, Flt3, WT1, MUC16, PRAME, TSPAN10, CLAUDIN18.2, DLL3, LY6G6D, Liv-1, CHRNA2, ADAM10, M-CSF, prostase, prostase specific antigen (PSA), PAP, NY-ESO-1, LAGA-Ia, p53, prostein, PSMA, surviving and telomerase, prostate-carcinoma tumor antigen-1 (PCTA-1), MAGE, ELF2M, neutrophil elastase, ephrin B2, insulin growth factor (IGF1)-I, IGF-II, IGFI receptor, mesothelin, a major histocompatibility complex (MHC) molecule presenting a tumor-specific peptide epitope, 5T4, RORI, Nkp30, NKG2D, tumor stromal antigens, the extra domain A (EDA) and extra domain B (EDB) of fibronectin and the A1 domain of tenascin-C (TnC A1) and fibroblast associated protein (fap); a lineage-specific or tissue specific antigen such as CD3, CD4, CD8, CD24, CD25, CD33, CD34, CD133, CD138, CTLA-4, B7-1 (CD80), B7-2 (CD86), GM-CSF, CD123, HSP70, FAP, HER2, CD79a, CD79b, CD123, MUC-1, and CS1, a cytokine receptor, endoglin, a major histocompatibility complex (MHC) molecule, BCMA (CD269, TNFRSF 17), or a virus-specific surface antigen such as an HIV specific antigen (such as HIV gp120, NEF, gp41); an EBV-specific antigen, a CMV-specific antigen, a HPV-specific antigen, a Lasse Virus-specific antigen, an Influenza Virus-specific antigen as well as any derivate or variant of these surface markers. an endogenous small antigen presented by HLA class I at the surface of the cells.

The combination of the invention may comprise an immune T cell expressing a CAR which targets specifically a cell surface marker selected from CD19, CD38, HSP70, CD30, FAP, HER2, CD79a, CD79b, CD123, CD22, CLL-1, MUC-1, GD2, O-acetyl-GD2, GD3, ROR 1, and CS1.

The combination of the invention may comprise an immune T cell expressing a CAR which targets specifically a cell surface marker selected from any cluster of differentiation molecules (e.g. CD16, CD20, CD22, CD30, CD38, CD40, CD64, CD78, CD79a CD79b, CD96, CLL1, CD116, CD117, CD71, CD45, CD123 and CD138), CS1, a tumor-associated surface antigen, such as ErbB2 (HER2/neu), carcinoembryonic antigen (CEA), epithelial cell adhesion molecule (EpCAM), epidermal growth factor receptor (EGFR), disialoganglioside GD2, o-acethyl GD2, GD3, mesothelin, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RUI, RU2 (AS), intestinal carboxyl esterase, hsp70-2, HSP70, M-CSF, prostase, prostase specific antigen (PSA), PAP, NY-ESO-1, LAGA-Ia, p53, prostein, PSMA, surviving and telomerase, prostate-carcinoma tumor antigen-1 (PCTA-1), MAGE, ELF2M, neutrophil elastase, ephrin B2, insulin growth factor (IGF1)-I, IGF-II, IGFI receptor, mesothelin, a major histocompatibility complex (MHC) molecule presenting a tumor-specific peptide epitope, 5T4, RORI, Nkp30, NKG2D, tumor stromal antigens, the extra domain A (EDA) and extra domain B (EDB) of fibronectin and the A1 domain of tenascin-C (TnC A1) and fibroblast associated protein (fap); a lineage-specific or tissue specific antigen such as CD3, CD4, CD8, CD24, CD25, CD34, CD133, CD138, CTLA-4, B7-1 (CD80), B7-2 (CD86), GM-CSF, CD123, FAP, HER2, CD79a, CD79b, CD123, MUC-1, and CS1, cytokine receptors, endoglin, a major histocompatibility complex (MHC) molecule, or a virus-specific surface antigen such as an HIV specific antigen (such as HIV gp120); an EBV-specific antigen, a CMV-specific antigen, a HPV-specific antigen, a Lasse Virus-specific antigen, an Influenza Virus-specific antigen as well as any derivate or variant of these surface markers. an endogenous small antigen presented by HLA class I at the surface of the cells.

The combination of the invention may comprise an immune T cell expressing a CAR which targets specifically a cell surface marker selected from BCMA, CD33, EGFRVIII, Flt3, WT1, CD70, MUC16, PRAME, TSPAN10, CLAUDIN18.2, DLL3, LY6G6D, Liv-1, CHRNA2, ADAM10.

Activation and Expansion of T Cells

The method according to the invention generally includes a further step of activating and/or expanding the T-cells. This can be done prior to or after genetic modification of the T cells, using the methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. According to these methods, the T cells of the invention can be expanded by contact with a surface having attached thereto an agent that stimulates a CD3 TCR complex associated signal and a ligand that stimulates a co-stimulatory molecule on the surface of the T cells.

In particular, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. For example, the agents providing each signal may be in solution or coupled to a surface. As those of ordinary skill in the art can readily appreciate, the ratio of particles to cells may depend on particle size relative to the target cell. In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. Cell surface proteins may be ligated by allowing paramagnetic beads to which anti-CD3 and anti-CD28 are attached (3×28 beads) to contact the T cells. In one embodiment the cells (for example, 4 to 10 T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. The mixture may be cultured for several hours (about 3 hours) to about 14 days or any hourly integer value in between. In another embodiment, the mixture may be cultured for 21 days. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, -10, -2, 1L-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics

In another particular embodiment, said cells can be expanded by co-culturing with tissue or cells. Said cells can also be expanded in vivo, for example in the subject's blood after administrating said cell into the subject.

Therapeutic Applications

The combination of the invention as described above are intended to be used as a medicament for treating, among others, cancer, infections or immune diseases in a patient in need thereof.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

The combination of the above comprises T cells according to one of the previous methods may be pooled, frozen, and administrated to one or several patients. When they are made non-alloreactive, they are available as an “off the shelf” therapeutic product, which means that they can be universally infused to patients in need thereof in combination with a therapeutic antibody directed against the antigen deleted in cells with which Ab are combined of the invention.

Said treatments are primarily intended to patients diagnosed with cancer, viral infection, autoimmune disorders or Graft versus Host Disease (GvHD). Cancers are preferably leukemias and lymphomas, which have liquid tumors, but may also concern solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

The present invention provides in Tables 4 to 14 with examples of antigen markers, which can be targeted with the combination of the invention for treating different types of cancer as illustrated in table 5 to 14.

Preferred antigen markers used for the immunotherapy of the present invention are more particularly those of table 5 to 14.

Pathological Conditions

The following pathological conditions may be treated efficiently with the combination of the invention: Multiple myeloma (MM), Acute myeloid leukemia (AML), Chronic myeloid leukemia (CML), Acute lymphoblastic leukemia (ALL), Hodgkin lymphoma (HL) (relapsed, refractory), Non-Hodgkin lymphoma (NHL) (relapsed, refractory), Neuroblastoma, Ewing sarcoma, Myelodysplastic syndromes, BPDCN.

The combination or the pharmaceutical composition of the invention is also especially efficient in the treatment or prophylaxis of Gliomas, pancreatic cancer, lung cancer, bladder cancer, colon cancer, breast cancer.

The combination or the pharmaceutical composition of the invention is adapted for the treatment of Non-Hodgkin's Lymphoma (indolent NHLs, follicular NHLs, small lymphocytic lymphoma, lymphoplasmacytic NHL, or marginal zone NHL); Hodgkin's disease (e.g., Reed-Sternberg cells); a cancer of the B-cell lineage, including, e.g., diffuse large B-cell lymphoma, follicular lymphoma, Burkitt's lymphoma, mantle cell lymphoma, Cutaneous T cell lymphoma, B-cell lymphocytic leukemia (e.g., acute lymphocytic leukemia, chronic lymphocytic leukemia); Epstein Barr Virus positive B cell lymphoma; renal cell carcinoma (e.g., clear cell and papillary); nasopharyngeal carcinoma; thymic carcinoma; glioma; glioblastoma; neuroblastoma; astrocytoma; meningioma; Waldenstrom macroglobulinemia; multiple myeloma; colon cancer, stomach cancer, and rectal carcinoma.

A combination comprising an immune T cell with a genetic modification of the CD70 gene affecting cell surface expression of CD70 and an anti-CD70 therapeutic antibody ARGX 110, is used in the treatment of Cutaneous T cell lymphoma.

A combination comprising an immune T cell with a genetic modification of the CD70 gene affecting cell surface expression of CD70 and an anti-CD70 therapeutic antibody MDX 1203 is intended for its use in the treatment of Renal Cell Carcinoma or Non-hodgkin's Lymphoma.

The combination directed to CS1 is used for the treatment of carcinoma, blastoma, and sarcoma, preferably MM, leukemia, melanoma, relapse or refractory CS-1 expressing MM or a complication related to CS-1 expressing MM.

The combination directed to CS1 is used for the treatment of any one of the following CS1 expressing cancers: Multiple Myeloma, Acute Myeloid Leukemia, Myelodysplastic Syndrome, Smoldering Multiple Myeloma, monoclonal gammopathy of unknown significance (MGUS), plasma cell leukemia, Non-Hodgkin's Lymphoma.

The combination directed to CS1 expressing cancers is used for the treatment of Multiple Myeloma, Non-Hodgkin Lymphoma, Diffuse Large B Cell Lymphoma, Mantle-Cell Lymphoma, Follicular Lymphoma, Indolent B Cell Lymphoma, Primary Mediastinal Lymphoma, Lymphoplasmacytic Lymphoma.

The combination directed to CS1 may be used for the treatment of any one of the following CS1 expressing cancers: NK cell lymphoma, NK or T cell lymphoma, angioimmunoblastic T-cell lymphoma (AITL), or peripheral T cell lymphoma not otherwise specified (PTCL-NOS).

The combination directed to CD38 comprising an immune T cell with a genetic modification of the CD38 gene affecting cell surface expression of CD38 and an anti-CD38 therapeutic antibody HuMax-CD38—Isatuximab is for example for the treatment of Prostate Cancer, Non-small Cell Lung Cancer, Plasma Cell Myeloma, MM, T-cell Type Acute Leukemia, Precursor T-Iymphoblastic Lymphoma or Leukaemia, prostate cancer, Non-small Cell Lung Cancer.

The combination comprising an immune T cell with a genetic modification of the CD38 gene affecting cell surface expression of CD38 and an anti-CD38 therapeutic antibody HuMax-CD38—Daratumumab may be used for the treatment of Microsatellite Unstable Colorectal Cancer, Microsatellite Stable Colorectal Cancer, Mismatch Repair Proficient Colorectal Cancer, Mismatch Repair Deficient Colorectal Cancer, Waldenstrom Macroglobulinemia, Malignant Neoplasms of Male Genital Organs, Prostate Cancer, Hematopoietic Cancer, Acute Myelogenous Leukemia, High-Risk Myelodysplastic Syndrome, Plasma cell myeloma, Monoclonal Gammopathy, Smoldering Multiple Myeloma, Membranoproliferative Glomerulonephritis, Multiple Myeloma.

The combination comprising an immune T cell with a genetic modification of the CD38 gene affecting cell surface expression of CD38 and Isatuximab, for the treatment of T-cell Type Acute Leukemia-Precursor T, lymphoblastic Lymphoma, Leukaemia is provided.

The combination comprising an immune T cell with a genetic modification of the CD38 gene affecting cell surface expression of CD38 and Isatuximab, for the treatment of a CD38-expressing or CD 38 over expressing hematologic cancer selected from the group of Chronic Lymphocytic Leukemia (CLL), Chronic Myelogenous Leukemia (CML), Acute Myelogenous Leukemia (AML), and Acute Lymphocytic Leukemia (ALL), multiple myeloma (MM).

The combination comprising an immune T cell with a genetic modification of the CD38 gene affecting cell surface expression of CD38 and Isatuximab, for the treatment of hematologic cancer selected from the group of leukemia, lymphoma and multiple myeloma (MM).

The combination comprising an immune T cell with a genetic modification of the CD38 gene affecting cell surface expression of CD38 and Isatuximab, (directed to CD38) for the treatment of hematologic cancer selected from the group of B-cell chronic lymphocytic leukemia (B-CLL), acute lymphoblastic leukemia, chronic myeloid leukemia, chronic lymphocytic leukemia (CLL), chronic myelogenous leukemia or chronic myeloid leukemia (CML), acute myelogenous leukemia or acute myeloid leukemia (AML), acute lymphocytic leukemia (ALL), hairy cell leukemia (HCL), myelodysplasia syndromes (MDS) or chronic myelogenous leukemia (CML-BP) and any subtypes of chronic myelogenous leukemia (CML-BP).

The combination directed to CD38 for the treatment of Microsatellite Unstable Colorectal Cancer, Microsatellite Stable Colorectal Cancer, Mismatch Repair Proficient Colorectal Cancer, Mismatch Repair Deficient Colorectal Cancer, Waldenstrom Macroglobulinemia, Malignant Neoplasms of Male Genital Organs, Prostate Cancer, Hematopoietic Cancer, Acute Myelogenous Leukemia, High-Risk Myelodysplastic Syndrome, Plasma cell myeloma, Monoclonal Gammopathy, Smoldering Multiple Myeloma, Membranoproliferative Glomerulonephritis, Multiple Myeloma.

The combination as above directed to CD38 for the treatment of T-cell Type Acute Leukemia-Precursor T, lymphoblastic Lymphoma, Leukaemia.

The combination directed to CD38 for the treatment of a CD38-expressing or CD 38 over expressing hematologic cancer selected from the group of Chronic Lymphocytic Leukemia (CLL), Chronic Myelogenous Leukemia (CML), Acute Myelogenous Leukemia (AML), and Acute Lymphocytic Leukemia (ALL), multiple myeloma (MM).

The combination directed to CD38 for the treatment of hematologic cancer selected from the group of leukemia, lymphoma and multiple myeloma (MM).

The combination directed to CD38 for the treatment of hematologic cancer selected from the group of B-cell chronic lymphocytic leukemia (B-CLL), acute lymphoblastic leukemia, chronic myeloid leukemia, chronic lymphocytic leukemia (CLL), chronic myelogenous leukemia or chronic myeloid leukemia (CML), acute myelogenous leukemia or acute myeloid leukemia (AML), acute lymphocytic leukemia (ALL), hairy cell leukemia (HCL), myelodysplasia syndromes (MDS) or chronic myelogenous leukemia (CML-BP) and any subtypes of chronic myelogenous leukemia (CML-BP), leukemia, lymphoma and multiple myeloma.

The present combination of the invention, when armed with specific CARs directed against patient's own immune cells, especially T-cells, allow the inhibition or regulation of said cells, which is a key step for treating auto-immune disease, such as rheumatoid polyarthritis, systemic lupus erythematosus, Sjogren's syndrome, scleroderma, fibromyalgia, myositis, ankylosing spondylitis, insulin dependent diabetes of type I, Hashimoto's thyroiditis, Addison's disease, Crohn's disease, Celiac's disease, amyotrophic lateral sclerosis (ALS) and multiple sclerosis (MS). Accordingly the present invention encompass a method for treating an immune disease by directing engineered T-cells and therapeutic antibody as previously described against patient's own T-cells.

The above combination can take place in combination with one or more therapies selected from the group of antibodies therapy, chemotherapy, cytokines therapy, dendritic cell therapy, gene therapy, hormone therapy, laser light therapy and radiation therapy.

The combination of the invention may comprise allogeneic cells made resistant to chemotherapy drugs and immunosuppressive drugs that are used as standards of care, especially methotrexate and the combination of fludarabine and Cyclophosphamide, are particularly suited for treating various forms of cancer. Indeed, the present invention preferably relies on cells or population of cells, comprising a dCK inactivated gene. As with the therapeutic antibody of the combination disclosed here as an invention, it is expected that the chemotherapy and/or immunosuppressive treatment should help the selection and expansion of the engineered T-cells in-vivo.

In certain embodiments of the present invention, cells are administered to a patient in conjunction with (e.g., before, simultaneously or following) a therapeutic antibody and any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1 1; Henderson et al., Immun. 73:316-321, 1991; Bierer et al., Citrr. Opin. mm n. 5:763-773, 93). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery. Said modified cells obtained by any one of the methods described here can be used in a particular aspect of the invention for treating patients in need thereof against Host versus Graft (HvG) rejection and Graft versus Host Disease (GvHD); therefore in the scope of the present invention is a method of treating patients in need thereof against Host versus Graft (HvG) rejection and Graft versus Host Disease (GvHD) comprising treating said patient by administering to said patient an effective amount of modified cells comprising inactivated TCR alpha and/or TCR beta genes.

According to one embodiment, said combination of the invention can undergo robust in vivo T cell expansion upon administration to a patient, and can persist in the body fluids for an extended amount of time, preferably for a week, more preferably for 2 weeks, even more preferably for at least one month. Although the T-cells of the combination of the invention according to the invention are expected to persist during these periods, their life span into the patient's body are intended not to exceed a year, preferably 6 months, more preferably 2 months, and even more preferably one month.

The administration of the cells or population of cells and therapeutic antibody according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 105 to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration of the combination of the invention is within the judgment of managing physician and depends on the clinical condition of the patient. While individual needs vary, determination of optimal ranges of effective amounts of a given cell type and of a given antibody of the combination, for a particular disease or conditions within the skill of the art. An effective amount means an amount which provides a therapeutic or prophylactic benefit with administered. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

### Examples

The cells or population of cells may be obtained from any source, such as a blood bank or a donor.

Identification of Surface Antigen Marker Expressed on the Surface of T-Cells, while being Overexpressed in Solid Tumors Involved into Different Types of Cancer (Tables 5 to 13)

We used BioGPS microarray data from a panel of normal tissues (Human U133A/GNF1H Gene Atlas) cancer microarray data that also can be downloaded from BioGPS (Human Primary Tumors (U95)) uniprot data that contains the subcellular localization.

We drew the distribution of values coming from normal tissues and determined a threshold value of 5 for the relative expression.

We browsed all the genes assayed with microarrays (44.000 probes representing about 13 000 genes) and checked their localization in the membrane (protein not referred to as being a membrane protein were discarded). Expression in CD8+ T-cells was checked from the BioGPS database. The genes were listed according to the type of cancer where the corresponding expression was the highest (Tables 5 to 13).

Identification of Surface Antigen Marker Expressed on the Surface of T-Cells, while being Overexpressed in Different Liquid Blood Tumors (Table 14)

For that study, no RNA-seq data were available and thus we used microarray data that were obtained from a large study from the MILE consortium (Microarray Innovations in Leukemia), involving 11 laboratories (http://www.ngrl.org.uk/wessex/downloads/tm08/TM08-S4-1_KenMills.pdf—Haferlach et al. 2010, http://www.ncbi.nlm.nih.gov/pubmed/20406941). This raw data include results for ALL (acute lymphoblastic leukemia), AML (acute myelogenous leukemia), CLL (chronic lymphoblastic leukemia) and CML (chronic myelogenous leukemia) and MDS (myelodysplastic syndrome). We also used uniprot data for subcellular localization as usual.

We first drew the overall distribution of values from all genes on all studied tissues. Then, to have an idea of the level necessary for expression, we took a list of genes which are expressed in some liquid tumors and for which therapeutic antibodies are available (CD52, CD 20, CD33, CD19, CD25, CD44, CD47, CD96, CD116, CD117, CD135, TIM-3). For each gene, we looked at the value obtained in the tumor in which it is expressed. Then, we computed the average for each tumor and gene pair for which the gene seems to give a cell membrane protein (cell membrane localization+description of at least one transmembrane domain in the protein). We discarded genes for which the expression in all the tissues was below this threshold of 0.15. We listed and ranked in Table 14, those genes which relative expression in T-cells was above 0.2. Thus, Table 4 provides putative antigen marker candidates for targeting liquid tumor cells as per the invention, in particular for treating ALL, AML, CLL, CML and MDS.

Therapeutic antibody specific for an the antigen identified, when available, and already under clinic phase below, were used.

Several administration protocols were tested first in an animal model of CD38 expressing pathological tissues.

Under these experimental conditions, the dose of Ab and cells necessary to obtained optimized reduction of pathological cells was estimated.

In human subjects, the amount of Ab and cells was determined based on clinical data (Drent E, Groen R W J, Noort W A, et al. Pre-clinical evaluation of CD38 chimeric antigen receptor engineered T cells for the treatment of multiple myeloma. Haematologica. 2016; 101(5):616-625. doi:10.3324/haematol.2015.137620).

Additionally, cells were engineered to be less alloreactive and/or be used in lymphodepleted individuals (TCR-negative and MHC-negative).

Example of Method to Engineer T-Cells According to the Invention for Immunotherapy

For a better understanding of the invention, it is provided below an example of the steps to follow to produce T-cells directed against leukemia CD38 positive cells:
1. Providing T-cells from a cell culture or from a blood sample from one individual patient or from blood bank and activating said T cells using anti-CD3/C28 activator beads (Dynabeads®). The beads provide both the primary and co-stimulatory signals that are required for activation and expansion of T cells.
2. Transducing said cells with a viral vector comprising a transgene encoding a Chimeric antigen receptor consisting of the fusion of CD3zeta activation domain, 4-1BB co-stimulation domain, a transmembrane domain and a hinge from CD28 fused to a sequence encoding the variable chain of an anti-CD38 antibody. For security improvement of the transformed T-cell, a suicide gene sensitive to rituximab and QBEN 10 was be introduced as described in WO 2013/153391.
3. (Optionally) Engineering non alloreactive and/or resistant T cells:

Inactivation of the TCR alpha in said cells was performed as previously described to eliminate the TCR from the surface of the cell and prevent recognition of host tissue as foreign by TCR of allogenic and thus to avoid GvHD by following the protocols set forth in WO 2013/176915. Thus, cells were also engineered to inactivate the TCR alpha subunit and alter the alpha beta TCR expression at the cell surface.

It is also possible to inactive one gene encoding target for an immunosuppressive agent or a chemotherapy drug to render said cells resistant to immunosuppressive or chemotherapy treatment to prevent graft rejection without affecting transplanted T cells. In this example, target of immunosuppressive agents is dCK and immunosuppressive agent is a PNA or a combination of fludarabine and cytarabine as described in WO 2013/176915.

Gene Inactivation is performed by electroporation of T cells with mRNA encoding specific TAL-endonuclease (TALEN™—Cellectis, 8 rue de la Croix Jarry, France). Inactivated T cells are sorted using magnetic beads. For example, T cells still expressing the targeted gene (e.g. CD38) can be removed by fixation on a solid surface, and inactivated cells are not exposed of the stress of being passed through a column. This gentle method increases the concentration of properly engineered T-cells.

Expansion in vitro of engineered T-cells prior to administration to a patient or in vivo following administration to a patient through stimulation of CD3 complex. Before administration step, patients can be subjected to an additional immunosuppressive treatment such as CAMPATH1-H, a humanized monoclonal anti-CD52 antibody, in combination with the therapeutic Ab of the combination disclosed here (eg anti-CD70 Ab).

Optionally exposed said cells with bispecific antibodies ex vivo prior to administration to a patient or in vivo following administration to a patient to bring the engineered cells into proximity to a target antigen.

Functional Analysis of the Engineered T-Cells Electroporated with a Monocistronic mRNA Encoding for an Anti-CD38 Single Chain Chimeric Antigen Receptor (CAR CD38):

To verify that genome engineering did not affect the ability of the engineered T-cells to present anti-tumor activity, especially when provided with a chimeric antigen receptor (CAR CD38), The engineered anti-CD38 CAR T-cells were incubated for 4 hours with Daudi cells expressing CD38 on their surface. The cell surface upregulation of CD107a, a marker of cytotoxic granule release by T lymphocytes (called degranulation) was measured by flow cytometry analysis (Betts, Brenchley et al. 2003).

24 hours post electroporation, cells were stained with a fixable viability dye eFluor-780 and a PE-conjugated goat anti mouse IgG F(ab′)2 fragment specific to assess the cell surface expression of the CAR on the live cells. The vast majority of the live T-cells genetically disrupted for CD38, express the anti-CD38 CAR on their surface. T cells were co-cultured with Daudi (CD38+) cells for 6 hours and analyzed by flow cytometry to detect the expression of the degranulation marker CD107a at their surface (Betts, Brenchley et al. 2003).

The results showed that CD38 disrupted T-cells kept the same ability to degranulate in response to PMA/ionomycin (positive control) or CD38+ Daudi cells. CD107 upregulation is dependent on the presence of a CD38+. These data suggest that the genome engineering of the present T-cells had no negative impact on the ability of T cells to mount a controlled anti-tumor response.

Combination with Therapeutic Anti-CD38 Ab.

The following anti-CD38 Ab were combined with CD38-negative, anti-CD38 CAR allogeneic (TCR KO) T engineered cells: Daratumumab (DARZALEX), Isatuximab or MOR-03087).

The combination of the invention of CD38 KO immune cells and therapeutic anti-CD38 MOR202 or DARZALEX (daratumumab) antibody were used for the treatment of MM.

The resulting data show that daratumumab at the dose of 8 mg/kg of body weight, administered as an intravenous infusion for 8 weeks weekly or bi weekly in combination with anti-CD38 CAR T cells administered the last week, and a second time, significantly reduce tumor mass volume in all patients, with no relapse observed at 24 weeks as compared to individual treated with Ab alone or engineered cells alone.

Examples of TAL Proteins Inactivating the CD70 Gene

A mRNA encoding a TALE-nuclease cleaving the following CD70 genomic sequence were synthesized from plasmid carrying the coding sequences (SEQ ID NO. 104-105, or SEQ ID NO. 106-107) downstream from a T7 promoter, using mMESSAGE mMACHINE™ T7 ULTRA Transcription Kit according to manufacturer's protocol (Ambion™). T lymphocytes isolated from peripheral blood were activated using anti-CD3/CD28 activator beads (Life technologies) and 5 million cells were then transfected by electroporation with 10 μg of each of 2 mRNAs encoding both half TALE-nuclease (or non coding RNA as controls) using a CytoLVT-P instrument. A sample of engineered T-cells were harvested on the day of electroporation and at one, two, five and height days post electroporation, the remaining T-cells were grown in culture media (REF). Fourteen days post electroporation, all T-cells were harvested. For each time point, the harvested T cells were labeled with fluorochrome-conjugated anti-CD70 monoclonal antibody (ref. 130-104-357, Miltenyi Biotec and according to manufacturer's protocol) and analyzed by flow cytometry for the presence of CD70 at their cell surface. At day 5 post electroporation, genomic DNA was also extracted on harvested T-cells and Polymerase Chain Reactions (PCR) were performed to amplify the targeted CD70 loci (Exon 1 for SEQ ID NO. 104-105 or Exon2 for SEQ ID NO. 106-107), these PCR were then submitted to the T7 Endonuclease 1 assay [New England Biolabs, see Vouillot et al. (2015) Comparison of T7E1 and Surveyor Mismatch Cleavage Assays to Detect Mutations Triggered by Engineered Nucleases. G3. 5(3): 407-415.] allowing the quantification of nucleases activity.

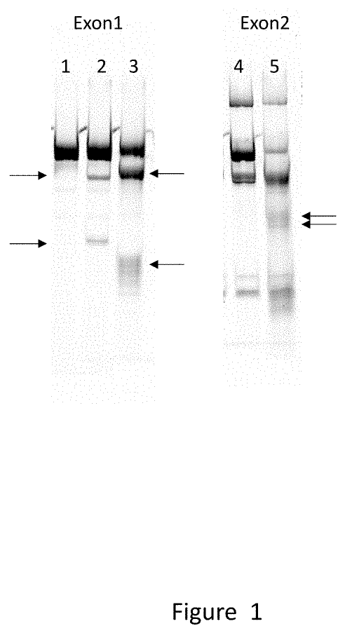

The results show that all TALE-nucleases specific for the CD70 gene showed cleavage activity using T7 Endonuclease 1 assay at day 5 post electroporation (FIG. 1). The TALE-nuclease activities of T02 (SEQ ID NO. 104-105) and T03 (SEQ ID NO. 106-107) were quantified at 54% and 20%, respectively as compared to control.

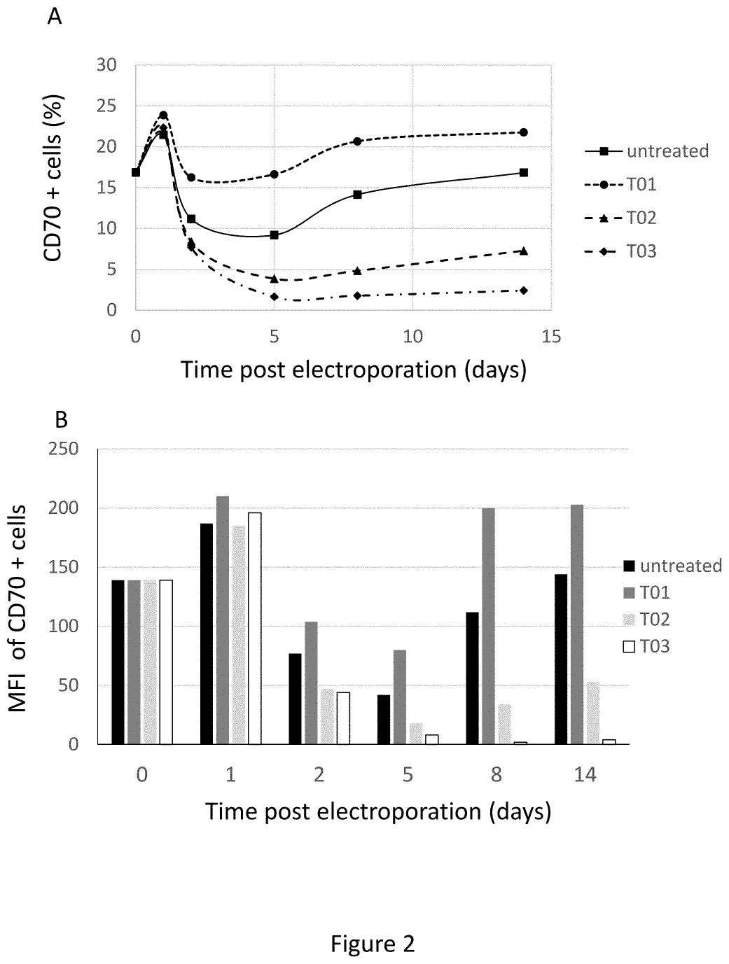

In addition, TALE-nucleases T02 and T03 were also able to decrease either the percentage of CD70 positive cells and/or the intensity of the labelling measured by the Median Fluorescence Intensity as compared to mock engineered cells (controls) (FIG. 2). This is demonstrating that these TALE-nucleases could efficiently inactivate the CD70 gene and the expression of CD70 at the surface of primary T-cells.

In one example (T01), CD70 at cell surface was inactivated (inactive) although partly still expressed at the cell surface. This is presenting another advantage as compared to CD70 deficient cells, probably by disrupting the signaling properties of CD70.

Cells obtained then, were combined with an anti-CD70 therapeutic antibody (CD70 Ab).

The anti-CD70 Ab was the following: Vorsetuzumab (or SGN-70 or h1F6) as disclosed in WO200473656, preferably (h1F6 clone) as disclosed in WO2006113909 which is a humanized h1F6, ARGX 110, SGN CD70A, SGN-CD70A, Vorsetuzumab mafodotin (SGN-75), and MDX 1203 (BMS-936561). The dose chosen was half of the dose giving 50 activity.

In another combination for the study above, the Ab was ARGX 2, at a dose of 5, and 10 mg/kg 3 weeks every 3 weeks with 5×105 and 5×106 cells at the same time, or at the end of the 3 weeks (Clin Cancer Res Oct. 4, 2017 DOI: 10.1158/1078-0432.CCR-17-0613).

Anti-CD70 Ab was used to selectively destroy anti-CD70 expressing cells in individuals suffering a cancer. The cells expressed a CAR allowing the cancer to be targeted and could resist the treatment with anti-CD70 Ab. The amount of engineered cells was also divided by 2 as compared to the effective dose 50 or used at the effective dose 50.

The results surprisingly showed that the combination of the invention was 7 to 10 times more efficient in reducing the tumor mass than either one of the component (engineered cells alone or anti-CD70 Ab alone) of the combination (engineered cells alone or antibody alone). Moreover, individuals receiving both components of the combination had significantly slower relapse and less refractory events than individuals treated with one component of the combination of this invention. The lymphodepletion induced by the CD70 Ab in these individuals and the in vivo selection of more active CD70 negative immune cells used for therapy (by in vivo depletion of any CD70 positive cells) account for these results.

In one arm, cells were engineered to alter cell surface expression of alpha beta TCR as previously described and combined with a CD70 cell surface depletion.

TABLE 4
Cluster of differentiation (CD) antigen markers of various cancers found to be expressed on the surface of T-cells

| Antigen | Other Names | Structure | main Distribution | Function |
|---|---|---|---|---|
| CD1a | T6 | IgSF, MHC-like | cortical thymocytes, Langerhans cells, DC | antigen presentation, with beta2m |
| CD1b | T6 | IgSF, MHC-like | cortical thymocytes, Langerhans cells, DC | antigen presentation, with beta2m |
| CD1c | T6 | IgSF, MHC-like | cortical thymocytes, Langerhans cells, DC, B | antigen presentation, with beta2m |
| | | | subset | |
| CD1d | | IgSF, MHC-like | intestinal epith, B subset, monolow, DC | antigen presentation, with beta2m |
| CD3 gamma, | T3 | IgSF | T, thymocyte subset | with TCR, TCR surface expression/signal |
| CD3 delta | | | | transduction |
| CD3 epsilon | T3 | IgSF | T, thymocyte subset | with TCR, TCR surface expression/signal |
| | | | | transduction |
| CD4 | T4 | IgSF | thymocyte subset, T subset, mono, mac | MHC class II coreceptor, HIV receptor, T cell |
| | | | | differentiation/activation |
| CD5 | T1, Tp67 | Scavenger R SF | thymocytes, T, B subset, B-CLL | CD72 receptor, TCR or BCR signaling, T-B |
| | | | | interaction |
| CD7 | | IgSF | hematopoietic progenitors, thymocytes, T, | T costimulation |
| | | | NK | |
| CD8a | T8, Leu-2 | IgSF | thymocyte subset, T subset, NK | MHC class I coreceptor, receptor for some |
| | | | | mutated HIV-1, T cell differentiation/activation |
| CD8b | | IgSF | thymocyte subset, T subset | |
| CD9 | p24, MRP-1 | TM4SF | pre-B, eosinophils, basophils, platelets, Tact | cellular adhesion and migration |
| CD10 | CALLA, NEP, | type II TM | B precursors, T precursors, neutrophils | zinc-binding metalloproteinase, B cell |
| | gp100 | | | development |
| CD11a | LFA-1, integrin | Integrin family | lymph, gran, mono, mac | CD11a/CD18 receptor for ICAM-1, -2, -3, |
| | alphaL | | | intercellular adhesion, T costimulation |
| CD11b | Mac-1, integrin | Integrin family | myeloid cells, NK | binds CD54, ECM, iC3b |
| | alphaM | | | |
| CD11c | p150, 95, CR4, | Integrin family | DC, myeloid cells, NK, B, T subset | binds CD54, fibrinogen and iC3b |
| | integrin alphaX | | | |
| CD13 | Aminopeptidase | type II TM | myeloid cells | zinc-binding metalloproteinase, antigen |
| | N, APN | | | processing, receptor for corona virus strains |
| CD14 | LPS-R | GPI-linked | mono, mac, Langerhans cells, granlow | receptor for LPS/LBP, LPS recognition |
| CD15 | Lewis-x, Lex | CHO | neutrophils, eosinophils, mono | adhesion |
| CD16a | FcgammaRIIIA | IgSF | neutrophils, mac, NK | component of low affinity Fc receptor, |
| | | | | phagocytosis and ADCC |
| CD16b | FcgammaRIIIB | IgSF | neutrophils | component of low affinity Fc receptor, |
| | | | | phagocytosis and ADCC |
| CD20 | B1, Bp35 | TM4SF | B, T subset | B cell activation |
| CD21 | C3DR, CR2, | CCRSF | B, FDC, T subset | complement C3d and EBV receptor, complex |
| | EBV-R | | | with CD19 and CD81, BCR coreceptor |
| CD22 | BL-CAM, | IgSF, | B | adhesion, B-mono, B-T interactions |
| | Siglec-2 | sialoadhesins | | |
| CD23 | FcepsilonRII | C-type lectin | B, activated mac, eosinophils, FDC, platelets | CD19-CD21-CD81 receptor, IgE low affinity |
| | | | | receptor, signal transduction |
| CD24 | BA-1 | GPI-linked | thymocytes, erythrocytes, peripheral lymph, | binds P-selectin |
| | | | myeloid | |
| CD25 | Tac, p55 | type I TM | Tact, Bact, lymph progenitors | IL-2Ralpha, with IL-2Rbeta and gamma to form |
| | | | | high affinity complex |
| CD31 | PECAM-1 | IgSF | mono, platelets, gran, endoth, lymph subset | CD38 receptor, adhesion |
| CD33 | p67, Siglec-3 | IgSF, | myeloid progenitors, mono, gran, DC, mast | adhesion |
| | | sialoadhesins | cells, Tact | |
| CD37 | | TM4SF | B, Tlow, granlow | signal transduction |
| CD38 | T10 | | variable levels on majority of hematopoietic | ecto-ADP-ribosyl cyclase, cell activation |
| | | | cells, high expression on plasma cells, B and | |
| | | | Tact | |
| CD40 | | TNFRSF | B, mono, mac, FDC, endoth, T subset | CD154 receptor, B differentiation/costimulation, |
| | | | | isotype-switching, rescues B cells from apoptosis |
| CD43 | Leukosialin, | Sialomucin, type | leukocytes, except resting B, plateletslow | inhibition of T cell interaction, CD54R, adhesion |
| | sialophorin | I TM | | |
| CD44 | H-CAM, Pgp-1 | hyaladherin | hematopoietic and non-hematopoietic cells, | binds hyaluronic acid, adhesion |
| | | family | except platelets, hepatocytes, testis | |
| CD45 | LCA, T200, | | hematopoietic cells, multiple isoforms from | tyrosine phosphatase, enhanced TCR & BCR |
| | B220 | | alternative splicing | signals |
| CD45RA | | | B, T subset(naive), mono | exon A isoforms of CD45 |
| CD45RB | | | T subset, B, mono, mac, gran | exon B isoforms of CD45 |
| CD45RO | | | Tact, memory T, B subset, mono, mac, gran | isoform of CD45 lacking A, B, C exons |
| CD46 | MCP | CCRSF | nucleated cells | membrane cofactor protein, binds C3b & C4b |
| | | | | allowing degradation by Factor I, measles virus |
| | | | | receptor |
| CD47 | IAP | IgSF | hematopoietic cells, epith, endoth, fibroblasts, | leukocyte adhesion, migration, activation |
| | | | other tissues | |
| CD48 | Blast-1 | IgSF | broad, all leukocytes | cell adhesion |
| CD52 | CAMPATH-1 | | thymocytes, T, B (not plasma cells), mono, | |
| | | | mac | |
| CD53 | | TM4SF | leukocytes, DC, osteoblasts, osteoclasts | signal transduction |
| CD55 | DAF | GPI-linked | hematopoietic, endoth | binds C3b, complement regulation |
| CD56 | NCAM | IgSF | NK, T subset, neurons, some large granular | adhesion |
| | | | lymphocyte leukemias, myeloid leukemias | |
| CD57 | HNK-1, Leu-7 | | NK subset, T subset | |
| CD58 | LFA-3 | IgSF | hematopoietic, non-hematopoietic cells | CD2 receptor, adhesion |
| CD59 | Protectin, MAC- | GPI-linked | hematopoietic, non-hematopoietic cells | binds complement C8 and C9, blocks assembly |
| | inhibitor | | | of membrane attack complex |
| CD60a | GD3 | CHO | T subset, platelets, thymic epith, astrocytes | costimulation |
| CD63 | LIMP, LAMP-3 | TM4SF | activated platelets, mono, mac | lysosomal membrane protein, moves to cell |
| | | | | surface after activation |
| CD68 | Macrosialin, | Sialomucin | intracellularly in mono, mac, neutrophils, basophils, large lymph, mast cells, | |
| | gp110 | | DC, myeloid progenitors, liver | |
| CD69 | AIM | C-type lectin | Tact, B, NK and gran, thymocytes, platelets, | signal transduction |
| | | | Langerhans cells | |
| CD70 | Ki-24 | TNFSF | Bact and Tact | CD27 ligand, T and B cell costimulation |
| CD74 | Ii, invariant | B, mac, mono, Langerhans cells, DC, Tact | | MHC class II traffic and function |
| | chain | | | |
| CD79a | Iga | IgSF | B | component of BCR, BCR surface expression and |
| | | | | signal transduction |
| CD79b | Igb | IgSF | B | component of BCR, BCR surface expression and |
| | | | | signal transduction |
| CD81 | TAPA-1 | TM4SF | T, B, NK, thymocytes, DC, endoth, fibroblast, | complex with CD19 & CD21, signaling, T |
| | | | neuroblastomas, melanomas | costimulation |
| CD82 | R2 | TM4SF | leukocytes | signal transduction |
| CD83 | HB15 | IgSF | Bact and Tact, DC, Langerhans cells | |
| CDw84 | | | mono, platelets, B, T subset, mac subset | |
| CD86 | B70, B7-2 | IgSF | mono, DC, Bact and Tact | binds to CD28, CD152, T costimulation |
| CD87 | UPA-R | GPI-linked | gran, mono, NK, Tact, endoth, fibroblasts | urokinase plasminogen activator receptor, |
| | | | | inflammatory cell invasion, metastasis |
| CD90 | Thy-1 | IgSF, GPI-linked | CD34+ hematopoietic subset, neurons | hematopoietic stem cell and neuron |
| | | | | differentiation |
| CD94 | KP43 | C-type lectin | NK, T subset | complex with NKG2, inhibits NK function |
| CD95 | Apo-1, Fas | TNFRSF | lymph (high upon activation), mono, | FasL (CD178) receptor, apoptosis |
| | | | neutrophils | |
| CD96 | TACTILE | IgSF | NK, Tact | adhesion of activated T and NK |
| CD97 | | TM7SF | Bact and Tact, mono, gran | |
| CD98 | 4F2 | | T, B, NK, gran, all human cell lines | cellular activation |
| CD99 | MIC2, E2 | | leukocytes | T cell activation, adhesion |
| CD100 | | | hematopoietic cells except immature bone | cell adhesion, cellular activation |
| | | | marrow cells, RBC and platelets | |
| CD103 | HML-1, alpha6, | Integrin family | intraepithelial lymph, lymph subset, activated | with integrin beta7, binds E-cadherin, lymph |
| | integrin alphaE | | lymph | homing/retention |
| CD107a | LAMP-1 | | activated platelets, T, endoth, metastatic tumors | a lysosomal membrane protein |
| CD107b | LAMP-2 | | activated platelets, T, endoth, metastatic tumors | a lysosomal membrane protein |
| CD109 | | | Tact and platelets, CD34+ subset, endoth | |
| CD123 | IL-3R | CRSF | lymph subset, basophils, hematopoietic | IL-3Ralpha, with CDw131 |
| | | | progenitors, mac, DC, megakaryocytes | |
| CD146 | MUC18, S-endo | IgSF | endoth, melanomas, FDC, Tact | adhesion |
| CD154 | CD40L, gp39, | TNFSF | Tact | CD40 ligand, B and DC costimulation |
| | TRAP | | | |
| CD158a | p58.1 | IgSF, KIR family | NK subset, T subset | inhibition of NK cell cytolytic activity, MHC |
| | | | | class-I specific NK receptor |
| CD158b | p58.2 | IgSF, KIR family | NK subset, T subset | inhibition of NK cell cytolytic activity, MHC |
| | | | | class-I specific NK receptor |
| CD163 | 130 kD | Scavenger | mono, mac | |
| | | receptor SF | | |
| CD164 | MGC-24 | epith, mono, | hematopoietic progenitor cell-stromal cell interaction | |
| | | lymphlow, bone | | |
| | | marrow stromal | | |
| | | cells, CD34+ | | |
| | | erythroid | | |
| | | progenitors | | |
| CD168 | RHAMM | | mono, T subset, thymocyte subset, | adhesion, tumor migration, metastasis |
| | | | intracellularly in breast cancer cells | |
| CD171 | L1 | IgSF | CNS, PNS, glial cells, mono, T subset, B, DC, | kidney morphogenesis, lymph node architecture, |
| | | | several human tumor cells | T costimulation, neurohistogenesis, homotypic |
| | | | | interaction, binds CD9, CD24, CD56, CD142, |
| | | | | CD166, integrins |
| CD177 | NB1 | | neutrophil subset | |
| CD178 | FasL, CD95L | TNFSF | Tact, testis | CD95 ligand, apoptosis, immune privilege, |
| | | | | soluble form in serum |
| CD180 | RP-105 | LRRF, TLR | B subset, mono, DC | B cell activation, LPS signaling, with MD-1 |
| | | family | | |
| CD182 | CXCR2, IL-8RB | GPCR1 family | neutrophils, basophils, NK, T subset, mono | binding of IL-8 induces chemotaxis of neutrophils |
| CD185 | CXCR5, BLR1 | GPCR1 family | mature B and Burkitt Lymphoma cells | with chemokine BLC, possible regulatory |
| | | | | function in Burkitt Lymphomagenesis and/or |
| | | | | B differentiation, activation of mature B |
| CD191 | CCR1, MIP- | GPCR1 family | T, mono, stem cell subset | binds C-C type chemokines and transduces |
| | 1alphaR, | | | signal by increasing intracellular calcium ion |
| | RANTES-R | | | levels |
| CD193 | CCR3, CKR3 | GPCR1 family | eosinophils, lower expression in neutrophils | binds eotaxin, eotaxin-3, MCP-3, MCP-4, |
| | | | and mono, T subset | RANTES & MIP-1 delta, alternative coreceptor |
| | | | | with CD4 for HIV-1 infectiongg |
| CD196 | CCR6, LARC | GPCR1 family | T subset, B, DC subset | binds MIP-3alpha/LARC |
| | receptor, DRY6 | | | |
| CD197 | CCR7 | | T subset, DC Subset | 6Ckine and MIP-2beta receptor |
| CD200 | OX-2 | | thymocytes, endoth, B, Tact | inhibition of immune response |
| CD209 | DC-SIGN | | DC subset | ICAM-3 receptor, HIV-1 binding protein |
| CD227 | MUC1, EMA | Mucin family, | epith, stem cell subset, FDC, mono, B subset, | adhesion, signaling, binds CD169, CD54, & |
| | | type I TM | some myelomas | selectins |
| CD231 | TALLA-1, A15 | TM4SF | T leukemias, neuroblastomas, brain neurons | marker for T cell acute lymphoblastic leukemia |
| CD246 | ALK, Ki-1 | | anaplastic T cell leukemias, small intestine, | brain development, implicated in ALK |
| | | | testis, brain, not on normal lymph | lymphomas |
| CD254 | TRANCE, | TNFSF | lymph node & BM stroma Tact | binds OPG and RANK, osteoclast differentiation, |
| | RANKL, OPGL | | | enhances DC to stimulate naïve-T proliferation |
| CD263 | TRAIL-R3, | | peripheral blood lymphocytes | receptor for TRAIL but lacks death domain |
| | DcR1, LIT | | | |
| CD272 | BTLA | IgSF | Tact, B, remains on Th1 | HVEM receptor, inhibitory response |
| CD273 | B7DC, PD-L2, | IgSF | DC subset, mono, mac | PD-1 receptor, costimulation or suppression of T |
| | PDCD1L2 | | | proliferation |
| CD276 | B7-H3 | B7 Family, ASV | in vitro cultured DC and mono, Tact, mammary | costimulation, T activation |
| | | | tissue | |
| CD277 | BT3.1, | B7/BT family, | T, B, NK, mono, DC, endoth, CD34+ cells, | T activation |
| | butyrophilin | ASV | tumor cell lines | |
| | SF3 A1, BTF5 | | | |
| CD279 | PD1, SLEB2 | | Tact and Bact | B7-H1 & B7-DC receptor, autoimmune disease |
| | | | | and peripheral tolerance |
| CD298 | Na+/K+− | | broad | transport sodium & potassium ions across |
| | ATPase beta3 | | | membrane |
| | subunit | | | |
| CD300a | CMRF35H, | IgSF, ASV | NK, mono, neutrophils, T and B subset and | unknown |
| | IRC1, IRp60 | | lymphocytic cell lines, AML | |
| CD300c | CMRF35A, LIR | IgSF | mono, neutrophils, monocytic cell lines, B & T | unknown |
| | | | subsets | |
| CD304 | BDCA4, | semaphorin | neurons, CD4+/CD25+ Treg, DC, endothelial | interacts with VEGF165 & semaphorins, co- |
| | neuropilin 1 | family | and tumor cells | receptor with plexin, axonal guidance, |
| | | | | angiogenesis, cell survival, migration |
| CD305 | LAIR1 | IgSF, ASV | NK, B, T, mono | inhibitory receptor on NK and T cells |
| CD314 | NKG2D, KLR | Type II lectin-like | NK, CD8+ activated, NK1.1+ T, some myeloid | binds MHC class I, MICA, MICB, Rae1 & |
| | | receptor | cells | ULBP4, activates cytolysis and cytokine |
| | | | | production, costimulation |
| CD317 | BST2, HM1.24 | Type II | B, T, NK, mono, DC, fibroblast cell line, | pre-B cell growth, overexpressed in multiple |
| | | | myeloma | myeloma |
| CD319 | CS1, CRACC, | SLAM receptor | B Cells, Dendritic Cells, NK, NKT | multiple myeloma |
| | SLAMF7 | family | | |

TABLE 5
antigen markers expressed on the surface of both colon tumor cells and T-cells

| | | | Relative |
|---|---|---|---|
| | | | Expression |
| | | Relative | in colon |
| | | expression | cancer |
| Antigen | Protein Name | in T-Cell | cells |
| EPCAM | Epithelial cell adhesion molecule | 2.97 | 13.99 |
| IFITM1 | Interferon-induced transmembrane protein 1 | 10.55 | 13.06 |
| CLDN4 | Claudin-4 | 2.87 | 11.62 |
| CDH17 | Cadherin-17 | 1.85 | 11.52 |
| CEACAM1 | Carcinoembryonic antigen-related cell adhesion molecule 1 | 3.33 | 10.84 |
| SLC26A3 | Chloride anion exchanger | 2.57 | 10.59 |
| ATP1A1 | Sodium/potassium-transporting ATPase subunit alpha-1 | 9.28 | 10.51 |
| SI | Isomaltase | 2.86 | 10.46 |
| ABCB1 | Multidrug resistance protein 1 | 6.09 | 10.24 |
| KCNQ1 | Potassium voltage-gated channel subfamily KQT member 1 | 3.36 | 9.99 |
| FCGRT | IgG receptor FcRn large subunit p51 | 4.8 | 9.98 |
| EPHB3 | Ephrin type-B receptor 3 | 5.23 | 9.74 |
| DSG2 | Desmoglein-2 | 3.04 | 8.5 |
| EPHB4 | Ephrin type-B receptor 4 | 6.5 | 8.44 |
| GUCY2C | Heat-stable enterotoxin receptor | 2.23 | 8.05 |
| EPHA2 | Ephrin type-A receptor 2 | 2.8 | 7.95 |
| LY6G6D | Lymphocyte antigen 6 complex locus protein G6f | 2.02 | 7.91 |
| CD97 | CD97 antigen subunit beta | 7.7 | 7.87 |
| SIGMAR1 | Sigma non-opioid intracellular receptor 1 | 4.58 | 7.85 |
| EREG | Epiregulin | 2.93 | 6.9 |
| FAIM2 | Protein lifeguard 2 | 2.94 | 6.82 |
| PIGR | Secretory component | 4.2 | 6.8 |
| SLC7A6 | Y + L amino acid transporter 2 | 8.06 | 6.55 |
| SCNN1D | Amiloride-sensitive sodium channel subunit delta | 1.77 | 5.74 |
| GPR35 | G-protein coupled receptor 35 | 1.98 | 5.5 |
| ABCG2 | ATP-binding cassette sub-family G member 2 | 1.79 | 5.35 |
| LPAR4 | Lysophosphatidic acid receptor 4 | 2.93 | 5.05 |
| GPR161 | G-protein coupled receptor 161 | 2.71 | 4.96 |
| CD1C | T-cell surface glycoprotein CD1c | 2.73 | 4.89 |
| SGCA | Alpha-sarcoglycan | 2.32 | 4.84 |
| CD22 | B-cell receptor CD22 | 4.12 | 4.75 |
| CD22 | B-cell receptor CD22 | 3.58 | 4.75 |
| CD22 | B-cell receptor CD22 | 2.73 | 4.75 |
| CD22 | B-cell receptor CD22 | 2.14 | 4.75 |
| SLC22A18 | Solute carrier family 22 member 18 | 2.32 | 4.62 |
| HTR7 | 5-hydroxytryptamine receptor 7 | 3.02 | 4.46 |
| LCT | Phlorizin hydrolase | 2.32 | 4.24 |
| CD33 | Myeloid cell surface antigen CD33 | 3.42 | 4.14 |
| PVR | Poliovirus receptor | 5.07 | 4.07 |
| PLXDC1 | Plexin domain-containing protein 1 | 5.85 | 3.99 |
| P2RY2 | P2Y purinoceptor 2 | 2.15 | 3.97 |
| CHRNB2 | Neuronal acetylcholine receptor subunit beta-2 | 6.31 | 3.88 |
| PTGDR | Prostaglandin D2 receptor | 4.08 | 3.65 |
| NCR1 | Natural cytotoxicity triggering receptor 1 | 2.63 | 3.33 |
| GYPA | Glycophorin-A | 3.18 | 3.31 |
| TNFRSF8 | Tumor necrosis factor receptor superfamily member 8 | 2 | 2.75 |
| KEL | Kell blood group glycoprotein | 1.93 | 2.48 |
| EDA | Ectodysplasin-A, secreted form | 2.7 | 2.42 |
| ACE | Angiotensin-converting enzyme, soluble form | 2.39 | 2.19 |
| DRD2 | D(2) dopamine receptor | 2.49 | 1.97 |
| CXCR3 | C—X—C chemokine receptor type 3 | 4.19 | 1.66 |
| MC2R | Adrenocorticotropic hormone receptor | 1.94 | 1.43 |

TABLE 6
antigen markers expressed on the surface of both breast tumor cells and T-cells

| | | | Relative |
|---|---|---|---|
| | | | Expression |
| | | Relative | in colon |
| | | expression | cancer |
| Antigen | Protein Name | in T-Cell | cells |
| ABCA8 | ATP-binding cassette sub-family A member 8 | 3.15 | 7.73 |
| ABCC10 | Multidrug resistance-associated protein 7 | 6.48 | 5.29 |
| ABCC6 | Multidrug resistance-associated protein 6 | 2.67 | 2.17 |
| ACCN2 | Acid-sensing ion channel 1 | 3.62 | 2.49 |
| ADAM12 | Disintegrin and metalloproteinase domain-containing | 4.96 | 7.72 |
| | protein 12 | | |
| ADCYAP1R1 | Pituitary adenylate cyclase-activating polypeptide type I | 2.17 | 2.88 |
| | receptor | | |
| ADRA1A | Alpha-1A adrenergic receptor | 3.31 | 4.85 |
| ADRA1B | Alpha-1B adrenergic receptor | 1.49 | 1.6 |
| ADRA1D | Alpha-1D adrenergic receptor | 2.39 | 3.38 |
| ADRA2A | Alpha-2A adrenergic receptor | 2.64 | 1.79 |
| ADRB3 | Beta-3 adrenergic receptor | 2.36 | 2.16 |
| AGER | Advanced glycosylation end product-specific receptor | 2.85 | 2.38 |
| AGTR2 | Type-2 angiotensin II receptor | 3.08 | 3.7 |
| ALK | ALK tyrosine kinase receptor | 4.97 | 4.27 |
| ANO3 | Anoctamin-3 | 2.39 | 3.69 |
| ANPEP | Aminopeptidase N | 3.26 | 10.78 |
| APLNR | Apelin receptor | 2.47 | 2.06 |
| AQP2 | Aquaporin-2 | 2.12 | 1.43 |
| ATP10A | Probable phospholipid-transporting ATPase VA | 3.96 | 6.02 |
| ATP2B2 | Plasma membrane calcium-transporting ATPase 4 | 2.75 | 4.81 |
| ATP2B3 | Plasma membrane calcium-transporting ATPase 3 | 3.7 | 4.14 |
| ATP4A | Potassium-transporting ATPase alpha chain 1 | 1.56 | 11.49 |
| ATP4B | Potassium-transporting ATPase subunit beta | 2.49 | 13.56 |
| ATP6V0A2 | V-type proton ATPase 116 kDa subunit a isoform 2 | 2.51 | 2.57 |
| ATRN | Attractin | 4.09 | 9.44 |
| AVPR1A | Vasopressin V1a receptor | 2.52 | 4.03 |
| AVPR1B | Vasopressin V1b receptor | 2.97 | 3.32 |
| AVPR2 | Vasopressin V2 receptor | 2.68 | 2.93 |
| BAH | Brain-specific angiogenesis inhibitor 1 | 2.73 | 0.33 |
| BAI2 | Brain-specific angiogenesis inhibitor 2 | 2.34 | 4.14 |
| BAI3 | Brain-specific angiogenesis inhibitor 3 | 2.73 | 4.76 |
| BDKRB1 | B1 bradykinin receptor | 2.07 | 3.28 |
| BRS3 | Bombesin receptor subtype-3 | 2.74 | 4.12 |
| BTF3 | Butyrophilin subfamily 3 member A2 | 11.29 | 13.02 |
| C18orf1 | Low-density lipoprotein receptor class A domain-containing | 3.18 | 8.45 |
| | protein 4 | | |
| C3AR1 | C3a anaphylatoxin chemotactic receptor | 3.04 | 5.15 |
| C6orf105 | Androgen-dependent TFPI-regulating protein | 2.34 | 3.84 |
| CASR | Extracellular calcium-sensing receptor | 2.52 | 5 |
| CCBP2 | Atypical chemokine receptor 2 | 1.72 | 3.29 |
| CCKAR | Cholecystokinin receptor type A | 2.46 | 3 |
| CCKBR | Gastrin/cholecystokinin type B receptor | 2.25 | 5.66 |
| CCR2 | C—C chemokine receptor type 2 | 5.94 | 3.56 |
| CCR3 | C—C chemokine receptor type 3 | 1.89 | 4.17 |
| CCR6 | C—C chemokine receptor-like 2 | 3.33 | 5.23 |
| CCR8 | C—C chemokine receptor type 8 | 2.28 | 3.93 |
| CCR9 | C—C chemokine receptor type 9 | 1.68 | 1.98 |
| CD1A | T-cell surface glycoprotein CD1a | 1.98 | 4.88 |
| CD1B | T-cell surface glycoprotein CD1b | 2.35 | 4.94 |
| CD1D | Antigen-presenting glycoprotein CD1d | 2.82 | 4.96 |
| CD300C | CMRF35-like molecule 6 | 2.04 | 5.04 |
| CD4 | T-cell surface glycoprotein CD4 | 2.84 | 6.17 |
| CD40LG | CD40 ligand, soluble form | 2.1 | 3.49 |
| CD5 | T-cell surface glycoprotein CD5 | 3.14 | 1.01 |
| CD63 | CD63 antigen | 8.6 | 13.18 |
| CD84 | SLAM family member 5 | 4.7 | 3.17 |
| CDH15 | Cadherin-15 | 2.07 | 3.55 |
| CDH19 | Protocadherin-16 | 2.82 | 8.4 |
| CDH22 | Cadherin-22 | 3 | 4.9 |
| CDH8 | Cadherin-8 | 3.63 | 5.87 |
| CDON | Cell adhesion molecule-related/down-regulated by | 2.35 | 3.61 |
| | oncogenes | | |
| CHRNA4 | Neuronal acetylcholine receptor subunit alpha-4 | 2.14 | 3.33 |
| CHRNA5 | Neuronal acetylcholine receptor subunit alpha-5 | 2.2 | 4.88 |
| CHRNA6 | Neuronal acetylcholine receptor subunit alpha-6 | 2.26 | 4.93 |
| CHRNB3 | Neuronal acetylcholine receptor subunit beta-3 | 1.85 | 3.91 |
| CHRNE | Acetylcholine receptor subunit epsilon | 2.56 | 2.83 |
| CLDN3 | Claudin-3 | 2.91 | 13.56 |
| CLDN7 | Claudin-7 | 1.89 | 12.87 |
| CLDN8 | Claudin-8 | 2.46 | 10.67 |
| CLDN9 | Claudin-9 | 1.74 | 1.69 |
| CLEC4M | C-type lectin domain family 4 member M | 2.7 | 3.32 |
| CMKLR1 | Chemokine-like receptor 1 | 2.62 | 5 |
| CNNM2 | Metal transporter CNNM2 | 2.47 | 5.32 |
| CNR2 | Cannabinoid receptor 2 | 2.38 | 3.66 |
| CRHR1 | Corticotropin-releasing factor receptor 1 | 2.15 | 10.71 |
| CRHR2 | Corticotropin-releasing factor receptor 2 | 2.32 | 6.44 |
| CSF1 | Processed macrophage colony-stimulating factor 1 | 5.63 | 7.61 |
| CSF1R | Macrophage colony-stimulating factor 1 receptor | 2.2 | 4.02 |
| CSF3R | Granulocyte colony-stimulating factor receptor | 1.85 | 2.8 |
| CX3CL1 | Processed fractalkine | 2.35 | 9.31 |
| CXCR5 | C—X—C chemokine receptor type 5 | 2.07 | 6.06 |
| DAGLA | Sn1-specific diacylglycerol lipase alpha | 2.6 | 2.11 |
| DRD1 | D(1A) dopamine receptor | 2.67 | 5.71 |
| DRD3 | D(3) dopamine receptor | 2.72 | 4.99 |
| DRD4 | D(4) dopamine receptor | 1.49 | 0.89 |
| DRD5 | D(1B) dopamine receptor | 2.26 | 4.91 |
| DSC2 | Desmocollin-2 | 2.26 | 11.12 |
| DSCAM | Down syndrome cell adhesion molecule | 2.54 | 3.76 |
| DSG1 | Desmoglein-1 | 2.62 | 7.71 |
| EMR2 | EGF-like module-containing mucin-like hormone receptor- | 2.25 | 3.38 |
| | like 2 | | |
| EPHA5 | Ephrin type-A receptor 5 | 2.42 | 7.48 |
| EPHA7 | Ephrin type-A receptor 7 | 2.61 | 4.87 |
| ERBB3 | Receptor tyrosine-protein kinase erbB-3 | 2.39 | 12.76 |
| F2RL2 | Proteinase-activated receptor 3 | 3.2 | 5.16 |
| FAM168B | Myelin-associated neurite-outgrowth inhibitor | 8.34 | 11.16 |
| FAP | Seprase | 1.87 | 10.15 |
| FAS | Tumor necrosis factor receptor superfamily member 6 | 5.68 | 7.24 |
| FASLG | FasL intracellular domain | 2.23 | 2.66 |
| FCAR | Immunoglobulin alpha Fc receptor | 2.8 | 3.85 |
| FCER1A | High affinity immunoglobulin epsilon receptor subunit alpha | 2.54 | 4.59 |
| FCGR2A | Low affinity immunoglobulin gamma Fc region receptor II-a | 2.77 | 8.81 |
| FCGR2B | Low affinity immunoglobulin gamma Fc region receptor II-b | 2.46 | 5.35 |
| FGFR2 | Fibroblast growth factor receptor 2 | 4.01 | 9.83 |
| FGFR4 | Fibroblast growth factor receptor 4 | 2.56 | 7.42 |
| FLT3LG | Fms-related tyrosine kinase 3 ligand | 7.86 | 4.37 |
| FPR1 | fMet-Leu-Phe receptor | 3.38 | 5.92 |
| FPR3 | N-formyl peptide receptor 3 | 1.91 | 2.61 |
| FSHR | Follicle-stimulating hormone receptor | 1.89 | 3.78 |
| FZD5 | Frizzled-5 | 2.82 | 5.2 |
| FZD5 | Frizzled-5 | 1.81 | 5.2 |
| FZD9 | Frizzled-9 | 2.66 | 3.16 |
| GABRA1 | Gamma-aminobutyric acid receptor subunit alpha-1 | 2.2 | 6.26 |
| GABRA5 | Gamma-aminobutyric acid receptor subunit alpha-5 | 2.49 | 3.24 |
| GABRA6 | Gamma-aminobutyric acid receptor subunit alpha-6 | 2.54 | 2.98 |
| GABRB1 | Gamma-aminobutyric acid receptor subunit beta-1 | 1.89 | 2.37 |
| GABRB2 | Gamma-aminobutyric acid receptor subunit beta-2 | 2.26 | 3.89 |
| GABRG3 | Gamma-aminobutyric acid receptor subunit gamma-3 | 2.23 | 2.85 |
| GABRP | Gamma-aminobutyric acid receptor subunit pi | 2.93 | 12.34 |
| GABRR1 | Gamma-aminobutyric acid receptor subunit rho-1 | 2.35 | 3.47 |
| GABRR2 | Gamma-aminobutyric acid receptor subunit rho-2 | 4.16 | 5.43 |
| GALR2 | Galanin receptor type 2 | 1.85 | 0.46 |
| GALR3 | Galanin receptor type 3 | 0.68 | 0.48 |
| GCGR | Glucagon receptor | 1.38 | 3.4 |
| GHRHR | Growth hormone-releasing hormone receptor | 1.61 | 3.49 |
| GJA5 | Gap junction alpha-5 protein | 1.72 | 2.05 |
| GJA8 | Gap junction alpha-8 protein | 2.39 | 6.51 |
| GJC1 | Gap junction delta-3 protein | 1.94 | 3.89 |
| GLP1R | Glucagon-like peptide 1 receptor | 5.72 | 3.41 |
| GLRA1 | Glycine receptor subunit alpha-1 | 2.15 | 3.87 |
| GLRA3 | Glycine receptor subunit alpha-3 | 3.19 | 3.1 |
| GNRHR | Gonadotropin-releasing hormone receptor | 2.72 | 4.1 |
| GPNMB | Transmembrane glycoprotein NMB | 2.14 | 13.94 |
| GPR1 | G-protein coupled receptor 1 | 3.83 | 4.1 |
| GPR135 | Probable G-protein coupled receptor 135 | 4.15 | 1.91 |
| GPR143 | G-protein coupled receptor 143 | 1.93 | 3.65 |
| GPR15 | G-protein coupled receptor 15 | 1.81 | 4.41 |
| GPR17 | Uracil nucleotide/cysteinyl leukotriene receptor | 1.93 | 1.74 |
| GPR171 | Probable G-protein coupled receptor 171 | 7.73 | 6.32 |
| GPR18 | N-arachidonyl glycine receptor | 7.05 | 3.52 |
| GPR182 | G-protein coupled receptor 182 | 1.66 | 1.29 |
| GPR19 | Probable G-protein coupled receptor 19 | 1.89 | 5.26 |
| GPR20 | G-protein coupled receptor 20 | 2.02 | 2.53 |
| GPR3 | G-protein coupled receptor 3 | 3.01 | 5.36 |
| GPR31 | 12-(S)-hydroxy-5,8,10,14-eicosatetraenoic acid receptor | 1.63 | 1.64 |
| GPR37L1 | Prosaposin receptor GPR37L1 | 2.23 | 4 |
| GPR39 | G-protein coupled receptor 39 | 1.81 | 1.36 |
| GPR44 | Prostaglandin D2 receptor 2 | 2 | 2.32 |
| GPR45 | Probable G-protein coupled receptor 45 | 2.78 | 5.31 |
| GPR6 | G-protein coupled receptor 6 | 2.56 | 3.38 |
| GPR65 | Psychosine receptor | 6.59 | 4.5 |
| GPR68 | Ovarian cancer G-protein coupled receptor 1 | 2.12 | 1.09 |
| GPR98 | G-protein coupled receptor 98 | 1.89 | 4.7 |
| GRIA1 | Glutamate receptor 1 | 4.17 | 4.77 |
| GRIA3 | Glutamate receptor 3 | 2.51 | 6.83 |
| GRIK2 | Glutamate receptor ionotropic, kainate 5 | 2.56 | 4.94 |
| GRIK3 | Glutamate receptor ionotropic, kainate 3 | 2.05 | 3.58 |
| GRIN1 | Glutamate receptor ionotropic, NMDA 1 | 4.52 | 1.49 |
| GRIN2B | Glutamate receptor ionotropic, NMDA2B | 2.22 | 3.56 |
| GRIN2C | Glutamate receptor ionotropic, NMDA2C | 2.56 | 3.37 |
| GRM1 | Metabotropic glutamate receptor 1 | 3.21 | 3.69 |
| GRM2 | Metabotropic glutamate receptor 2 | 2.04 | 0.44 |
| GRM3 | Metabotropic glutamate receptor 3 | 2.39 | 3.41 |
| GRM4 | Metabotropic glutamate receptor 4 | 5.2 | 3.78 |
| GRM5 | Metabotropic glutamate receptor 5 | 2.26 | 5.28 |
| GRM7 | Metabotropic glutamate receptor 7 | 2.86 | 3.07 |
| GYPB | Glycophorin-B | 2.43 | 4.02 |
| HBP1 | Glycosylphosphatidylinositol-anchored high density | 7.32 | 9.27 |
| | lipoprotein-binding protein 1 | | |
| HCRTR2 | Orexin receptor type 2 | 2.32 | 2.42 |
| HTR1B | 5-hydroxytryptamine receptor 1B | 2.82 | 3.51 |
| HTR1D | 5-hydroxytryptamine receptor 1D | 2.29 | 2.33 |
| HTR1E | 5-hydroxytryptamine receptor 1E | 1.72 | 2.4 |
| HTR2A | 5-hydroxytryptamine receptor 2A | 2.1 | 3.67 |
| HTR2C | 5-hydroxytryptamine receptor 2C | 2.49 | 5.18 |
| HTR4 | 5-hydroxytryptamine receptor 4 | 3.86 | 4.25 |
| ICAM4 | Intercellular adhesion molecule 4 | 2.51 | 2.16 |
| ICOS | Inducible T-cell costimulator | 3.91 | 3.86 |
| IL6R | Interleukin-6 receptor subunit alpha | 4.24 | 3.08 |
| IL6R | Interleukin-6 receptor subunit alpha | 2.64 | 3.08 |
| IL6ST | Interleukin-6 receptor subunit beta | 9.43 | 12.67 |
| IL9R | Interleukin-9 receptor | 2.71 | 2.86 |
| ITGB3 | Integrin beta-3 | 4.16 | 3.69 |
| KCNA3 | Potassium voltage-gated channel subfamily A member 3 | 2.09 | 4.9 |
| KCND2 | Potassium voltage-gated channel subfamily D member 2 | 2.67 | 4.25 |
| KCNH1 | Potassium voltage-gated channel subfamily H member 1 | 2.31 | 4.48 |
| KCNJ4 | Inward rectifier potassium channel 4 | 2.43 | 3.49 |
| KCNMA1 | Calcium-activated potassium channel subunit alpha-1 | 2.35 | 7.17 |
| KCNS1 | Potassium voltage-gated channel subfamily S member 1 | 5.66 | 6.49 |
| KCNV2 | Potassium voltage-gated channel subfamily V member 2 | 2.38 | 4.06 |
| KIR2DL4 | Killer cell immunoglobulin-like receptor 2DL4 | 1.68 | 3.31 |
| KIR3DL1 | Killer cell immunoglobulin-like receptor 3DL1 | 2.56 | 2.73 |
| KIR3DL3 | Killer cell immunoglobulin-like receptor 3DL3 | 1.7 | 3.06 |
| KLRG1 | Killer cell lectin-like receptor subfamily G member 1 | 8.3 | 5.76 |
| LAMP1 | Lysosome-associated membrane glycoprotein 1 | 10.9 | 13.6 |
| LHCGR | Lutropin-choriogonadotropic hormone receptor | 2.23 | 4.92 |
| LNPEP | Leucyl-cystinyl aminopeptidase, pregnancy serum form | 2.68 | 5.05 |
| LPAR2 | Lysophosphatidic acid receptor 2 | 5.5 | 4.23 |
| LRIG2 | Leucine-rich repeats and immunoglobulin-like domains | 3.35 | 5.48 |
| | protein 2 | | |
| LRRTM2 | Leucine-rich repeat transmembrane neuronal protein 2 | 2.42 | 4.24 |
| LTB4R | Leukotriene B4 receptor 1 | 4.96 | 2.26 |
| MAS1 | Proto-oncogene Mas | 1.91 | 3.11 |
| MC1R | Melanocyte-stimulating hormone receptor | 2.94 | 0.96 |
| MC5R | Melanocortin receptor 5 | 2.28 | 1.63 |
| MEP1B | Meprin A subunit beta | 2.61 | 3.87 |
| MFSD5 | Molybdate-anion transporter | 1.98 | 4.72 |
| MOG | Myelin-oligodendrocyte glycoprotein | 3.08 | 4.74 |
| MTNR1B | Melatonin receptor type 1B | 1.61 | 1.67 |
| MUC1 | Mucin-1 subunit beta | 2.73 | 13.68 |
| MUSK | Muscle. skeletal receptor tyrosine-protein kinase | 2.39 | 4.75 |
| NCAM2 | Neural cell adhesion molecule 2 | 2.12 | 4.49 |
| NCR2 | Natural cytotoxicity triggering receptor 2 | 4.79 | 7.09 |
| NCR3 | Natural cytotoxicity triggering receptor 3 | 4.55 | 2.74 |
| NIPA2 | Magnesium transporter NIPA2 | 6.77 | 3.9 |
| NLGN1 | Neuroligin-1 | 2.62 | 7.71 |
| NLGN4Y | Neuroligin-4, Y-linked | 2.52 | 5.26 |
| NMBR | Neuromedin-B receptor | 1.68 | 2.47 |
| NPHS1 | Nephrin | 2.74 | 4.33 |
| NPY2R | Neuropeptide Y receptor type 2 | 2.68 | 4.43 |
| NPY5R | Neuropeptide Y receptor type 5 | 2.38 | 5.05 |
| NTSR2 | Neurotensin receptor type 2 | 1.72 | 3 |
| OPRD1 | Delta-type opioid receptor | 2.26 | 2.14 |
| OPRL1 | Nociceptin receptor | 2.31 | 1.51 |
| OPRM1 | Mu-type opioid receptor | 3.18 | 4.01 |
| OR10H3 | Olfactory receptor 10H3 | 1.63 | 4.02 |
| OR1E1 | Olfactory receptor 1E1 | 3.04 | 4.77 |
| OR2F1 | Olfactory receptor 2F1 | 2.64 | 5.73 |
| OR2F2 | Olfactory receptor 2F2 | 2.19 | 2.3 |
| OR2H1 | Olfactory receptor 2H1 | 3.39 | 3.82 |
| OR2H2 | Olfactory receptor 2H2 | 3.79 | 6.37 |
| OR2J2 | Olfactory receptor 2J2 | 2.41 | 2.16 |
| OR2J2 | Olfactory receptor 2J2 | 1.93 | 2.16 |
| OR5I1 | Olfactory receptor 5I1 | 1.85 | 2.8 |
| OR7E24 | Olfactory receptor 7E24 | 2.5 | 3.47 |
| P2RX7 | P2X purinoceptor 7 | 2.36 | 2.15 |
| PANX1 | Pannexin-1 | 2.14 | 4.38 |
| PCDHA9 | Protocadherin alpha-9 | 2.82 | 3.56 |
| PCDHB11 | Protocadherin beta-11 | 1.91 | 5.23 |
| PCDHGA8 | Protocadherin gamma-A8 | 3.13 | 4.48 |
| PLA2R1 | Soluble secretory phospholipase A2 receptor | 2.91 | 5.16 |
| PLXNA3 | Plexin-A3 | 2.42 | 3.25 |
| POP1 | Blood vessel epicardial substance | 1.74 | 2.59 |
| PPYR1 | Neuropeptide Y receptor type 4 | 2.2 | 2.75 |
| PTGER1 | Prostaglandin E2 receptor EP1 subtype | 1.96 | 0.94 |
| PTGFR | Prostaglandin F2-alpha receptor | 2.75 | 4.89 |
| PTGIR | Prostacyclin receptor | 2.78 | 2.12 |
| PTPRJ | Receptor-type tyrosine-protein phosphatase eta | 2.63 | 4.6 |
| PTPRR | Receptor-type tyrosine-protein phosphatase R | 2.47 | 9.99 |
| PVRL1 | Poliovirus receptor-related protein 1 | 2.52 | 4.51 |
| PVRL2 | Poliovirus receptor-related protein 2 | 3.84 | 10.05 |
| ROS1 | Proto-oncogene tyrosine-protein kinase ROS | 2.93 | 3.38 |
| S1PR2 | Sphingosine 1-phosphate receptor 2 | 1.74 | 1.17 |
| S1PR4 | Sphingosine 1-phosphate receptor 4 | 4 | 0.21 |
| SCNN1B | Amiloride-sensitive sodium channel subunit beta | 1.89 | 3.16 |
| SCNN1G | Amiloride-sensitive sodium channel subunit gamma | 2.23 | 2.61 |
| SEMA4D | Semaphorin-4D | 10.66 | 1.56 |
| SEMA6A | Semaphorin-6A | 4.55 | 7.81 |
| SEMA6C | Semaphorin-6C | 5.02 | 3.73 |
| SGCB | Beta-sarcoglycan | 2.69 | 3.45 |
| SGCB | Beta-sarcoglycan | 2.04 | 3.45 |
| SLC12A3 | Solute carrier family 12 member 3 | 2.26 | 3.36 |
| SLC14A1 | Urea transporter 1 | 2.97 | 6.21 |
| SLC14A2 | Urea transporter 2 | 2.85 | 4.4 |
| SLC16A1 | Monocarboxylate transporter 1 | 3.46 | 8.84 |
| SLC16A2 | Monocarboxylate transporter 8 | 1.77 | 5.17 |
| SLC16A6 | Monocarboxylate transporter 7 | 2.41 | 11.66 |
| SLC22A1 | Solute carrier family 22 member 1 | 2.95 | 11.61 |
| SLC22A6 | Solute carrier family 22 member 6 | 2.26 | 2.53 |
| SLC5A12 | Sodium-coupled monocarboxylate transporter 2 | 2.98 | 4.45 |
| SLC6A1 | Sodium- and chloride-dependent GABA transporter 1 | 2.45 | 4.3 |
| SLC6A4 | Sodium-dependent serotonin transporter | 2.17 | 2.66 |
| SLC6A6 | Sodium- and chloride-dependent taurine transporter | 2.54 | 4.13 |
| SLC7A7 | Y + L amino acid transporter 1 | 2.22 | 9.78 |
| SLC8A1 | Sodium/calcium exchanger 1 | 2.07 | 2.36 |
| SLC9A1 | Sodium/hydrogen exchanger 1 | 3.15 | 5.54 |
| SLC9A3 | Sodium/hydrogen exchanger 3 | 2.12 | 3.15 |
| SLCO1A2 | Solute carrier organic anion transporter family member 1A2 | 3.87 | 4.98 |
| SLCO2B1 | Solute carrier organic anion transporter family member 2B1 | 4.43 | 8.92 |
| SORT1 | Sortilin | 2.93 | 4.6 |
| SSTR2 | Somatostatin receptor type 2 | 3.08 | 4.47 |
| SSTR3 | Somatostatin receptor type 3 | 2.23 | 1.5 |
| SSTR4 | Somatostatin receptor type 4 | 1.83 | 1.53 |
| SSTR5 | Somatostatin receptor type 5 | 2.57 | 1.47 |
| TACR1 | Substance-P receptor | 2.66 | 3.2 |
| TACR3 | Neuromedin-K receptor | 2.32 | 5.7 |
| TLR6 | Toll-like receptor 6 | 2.2 | 4.58 |
| TMPRSS6 | Transmembrane protease serine 6 | 4.02 | 3.69 |
| TNFSF11 | Tumor necrosis factor ligand superfamily member 11, | 2.57 | 5.18 |
| TNFSF14 | Tumor necrosis factor ligand superfamily member 14, | 3.34 | 2.83 |
| | soluble form | | |
| TPO | Thyroid peroxidase | 1.96 | 1.89 |
| TRAT1 | T-cell receptor-associated transmembrane adapter 1 | 7.51 | 5.29 |
| TRHR | Thyrotropin-releasing hormone receptor | 2 | 4.18 |
| TRPM1 | Transient receptor potential cation channel subfamily M | 2.43 | 5.22 |
| | member 1 | | |
| TSHR | Thyrotropin receptor | 2.9 | 4.87 |
| TSHR | Thyrotropin receptor | 2.12 | 4.87 |
| UNC93A | Protein unc-93 homolog A | 2.64 | 4.94 |
| VIPR2 | Vasoactive intestinal polypeptide receptor 2 | 2.58 | 3.37 |
| ZP2 | Processed zona pellucida sperm-binding protein 2 | 1.94 | 3.55 |

TABLE 7
antigen markers expressed on the surface of both digestive tumor cells and T-cells

| | | | Relative |
|---|---|---|---|
| | | | Expression |
| | | Relative | in colon |
| | | expression | cancer |
| Antigen | Protein Name | in T-Cell | cells |
| ACVR1B | Activin receptor type-1B | 5.16 | 10.48 |
| AMIGO2 | Amphoterin-induced protein 2 | 6.73 | 8.2 |
| ATP1B1 | Sodium/potassium-transporting ATPase subunit beta-1 | 2.64 | 12.31 |
| ATP8B1 | Probable phospholipid-transporting ATPase IC | 8.22 | 2.17 |
| CCR7 | C—C chemokine receptor type 7 | 10.25 | 11.52 |
| CD164 | Sialomucin core protein 24 | 10.27 | 12.12 |
| CD180 | CD180 antigen | 2.5 | 6.47 |
| CD40 | Tumor necrosis factor receptor superfamily member 5 | 5.02 | 6 |
| CD53 | Leukocyte surface antigen CD53 | 10.79 | 11.3 |
| CD79A | B-cell antigen receptor complex-associated protein alpha | 3.74 | 9.17 |
| | chain | | |
| CD79B | B-cell antigen receptor complex-associated protein beta | 3.6 | 6.66 |
| | chain | | |
| CD8B | T-cell surface glycoprotein CD8 beta chain | 8.43 | 2.62 |
| CELSR1 | Cadherin EGF LAG seven-pass G-type receptor 1 | 2.72 | 8.68 |
| CLCN5 | H(+)/Cl(−) exchange transporter 5 | 2.71 | 4.97 |
| CLDN18 | Claudin-18 | 3.05 | 14.51 |
| CLIC1 | Chloride intracellular channel protein 1 | 9.94 | 13.83 |
| COL13A1 | Collagen alpha-1 (XIII) chain | 2.96 | 6.24 |
| DIO3 | Type III iodothyronine deiodinase | 2.04 | 2.9 |
| EDNRA | Endothelin-1 receptor | 2.9 | 8.96 |
| EMR1 | EGF-like module-containing mucin-like hormone receptor- | 1.83 | 7.29 |
| | like 1 | | |
| ENPP1 | Nucleotide pyrophosphatase | 2.57 | 9.66 |
| EPHB1 | Ephrin type-B receptor 1 | 2.02 | 6.33 |
| EPHB1 | Ephrin type-B receptor 1 | 1.81 | 6.33 |
| F2R | Proteinase-activated receptor 1 | 3.04 | 9.78 |
| F2RL1 | Proteinase-activated receptor 2, alternate cleaved 2 | 3.31 | 9.47 |
| FCER2 | Low affinity immunoglobulin epsilon Fc receptor soluble | 2.49 | 8.77 |
| | form | | |
| GABBR1 | Gamma-aminobutyric acid type B receptor subunit 1 | 5.1 | 8.52 |
| GABRA3 | Gamma-aminobutyric acid receptor subunit alpha-3 | 2.12 | 3.84 |
| GPR183 | G-protein coupled receptor 183 | 4.79 | 10.22 |
| GPR37 | Prosaposin receptor GPR37 | 3.1 | 8.23 |
| GPRC5A | Retinoic acid-induced protein 3 | 1.87 | 13.69 |
| GRPR | Gastrin-releasing peptide receptor | 2.04 | 3.35 |
| GYPC | Glycophorin-C | 9.22 | 7.58 |
| IL1R2 | Interleukin-1 receptor type 2, soluble form | 2.82 | 12.83 |
| KIAA0319 | Dyslexia-associated protein KIAA0319 | 2.43 | 5.61 |
| LAMP2 | Lysosome-associated membrane glycoprotein 2 | 4.05 | 11.29 |
| LRP8 | Low-density lipoprotein receptor-related protein 8 | 4.24 | 8.84 |
| LSR | Lipolysis-stimulated lipoprotein receptor | 4.99 | 11.48 |
| MICB | MHC class I polypeptide-related seguence B | 5.27 | 9.89 |
| MMP16 | Matrix metalloproteinase-16 | 3.19 | 6.18 |
| MS4A1 | B-lymphocyte antigen CD20 | 2.15 | 8.02 |
| MYOF | Myoferlin | 2.41 | 11.56 |
| NAT1 | Sodium-coupled neutral amino acid transporter 3 | 3.49 | 12.09 |
| NFASC | Neurofascin | 3.78 | 8.28 |
| NPY1R | Neuropeptide Y receptor type 1 | 2.32 | 6.93 |
| OR2B6 | Olfactory receptor 2B6 | 2.78 | 4.24 |
| P2RY10 | Putative P2Y purinoceptor 10 | 3.39 | 6.62 |
| PCDH1 | Protocadherin-1 | 4.45 | 10.07 |
| PROM1 | Prominin-1 | 2.52 | 11.77 |
| PSEN1 | Presenilin-1 CTF12 | 2.94 | 8.83 |
| PTGER2 | Prostaglandin E2 receptor EP2 subtype | 6.33 | 6.74 |
| PTGER4 | Prostaglandin E2 receptor EP4 subtype | 8.62 | 5.12 |
| PTPRK | Receptor-type tyrosine-protein phosphatase kappa | 2.14 | 10.9 |
| RET | Extracellular cell-membrane anchored RET cadherin 120 | 2.38 | 12.3 |
| | kDa fragment | | |
| SERINC3 | Serine incorporator 3 | 7.93 | 12.01 |
| SIT1 | Sodium- and chloride-dependent transporter XTRP3 | 5.92 | 4.82 |
| SLAMF1 | Signaling lymphocytic activation molecule | 4.4 | 9.03 |
| SLC29A1 | Eguilibrative nucleoside transporter 1 | 2.07 | 6.12 |
| SLC39A6 | Zinc transporter ZIP6 | 6.69 | 15.23 |
| SLC7A5 | Large neutral amino acids transporter small subunit 1 | 3.79 | 10.98 |
| STX4 | Syntaxin-4 | 5.68 | 7.67 |
| TGFBR3 | Transforming growth factor beta receptor type 3 | 7.55 | 7.29 |
| TGOLN2 | Trans-Golgi network integral membrane protein 2 | 9.59 | 11.3 |
| TLR1 | Toll-like receptor 1 | 2.34 | 4.57 |
| TMED10 | Transmembrane emp24 domain-containing protein 10 | 9.34 | 12.24 |
| TMEM97 | Transmembrane protein 97 | 2.75 | 9.02 |
| TNF | Tumor necrosis factor, soluble form | 1.63 | 3.18 |
| TNFRSF17 | Tumor necrosis factor receptor superfamily member 17 | 1.89 | 10.47 |
| TNFRSF1B | Tumor necrosis factor-binding protein 2 | 5.51 | 9.4 |
| VDAC1 | Voltage-dependent anion-selective channel protein 1 | 6.52 | 11.5 |

TABLE 8
antigen markers expressed on the surface of both kidney tumor cells and T-cells

| | | | Relative |
|---|---|---|---|
| | | | Expression |
| | | Relative | in colon |
| | | expression | cancer |
| Antigen | Protein Name | in T-Cell | cells |
| ADORA3 | Adenosine receptor A3 | 1.89 | 4.56 |
| ATP11A | Probable phospholipid-transporting ATPase IH | 3.62 | 8.8 |
| BSG | Basigin | 4.77 | 11.34 |
| BTN3A2 | Butyrophilin subfamily 3 member A2 | 10.86 | 8.19 |
| C10orf72 | V-set and transmembrane domain-containing protein 4 | 2.04 | 6.85 |
| CADM3 | Cell adhesion molecule 3 | 3.57 | 6.39 |
| CD8A | T-cell surface glycoprotein CD8 alpha chain | 10.35 | 6.6 |
| CDH16 | Cadherin-16 | 2.17 | 7.09 |
| CDH4 | Cadherin-4 | 2.15 | 3.6 |
| CDH5 | Cadherin-5 | 2.5 | 9.55 |
| CHL1 | Processed neural cell adhesion molecule L1-like protein | 2.69 | 10.43 |
| CHRNB1 | Acetylcholine receptor subunit beta | 2.12 | 3.6 |
| CLIC4 | Chloride intracellular channel protein 4 | 3.34 | 13.12 |
| CNR1 | Cannabinoid receptor 1 | 2.26 | 5.64 |
| CRIM1 | Processed cysteine-rich motor neuron 1 protein | 3.57 | 12.39 |
| CSPG4 | Chondroitin sulfate proteoglycan 4 | 3.33 | 6.59 |
| CYBB | Cytochrome b-245 heavy chain | 2.86 | 8.07 |
| EDNRB | Endothelin B receptor | 3.04 | 8.97 |
| FLT1 | Vascular endothelial growth factor receptor 1 | 2.75 | 8.5 |
| FZD1 | Frizzled-1 | 2.72 | 7.59 |
| GJC2 | Gap junction gamma-2 protein | 2.09 | 2.94 |
| GLRB | Glycine receptor subunit beta | 2.51 | 7.15 |
| GPER | G-protein coupled estrogen receptor 1 | 2.34 | 8.64 |
| GPM6A | Neuronal membrane glycoprotein M6-a | 2.95 | 6.88 |
| GPR162 | Probable G-protein coupled receptor 162 | 2.75 | 2.81 |
| GPR4 | G-protein coupled receptor 4 | 2.93 | 8.09 |
| GRM8 | Metabotropic glutamate receptor 8 | 3.43 | 8.25 |
| HLA-DPB1 | HLA class II histocompatibility antigen, DP beta 1 chain | 9.93 | 13.99 |
| HTR6 | 5-hydroxytryptamine receptor 6 | 4.83 | 10.07 |
| INSR | Insulin receptor subunit beta | 3.44 | 8.95 |
| ITM2B | Bri23 peptide | 11.16 | 12.19 |
| KCNJ1 | ATP-sensitive inward rectifier potassium channel 1 | 2.5 | 4.17 |
| KDR | Vascular endothelial growth factor receptor 2 | 2.99 | 9.95 |
| KL | Klotho peptide | 2.83 | 7.59 |
| LAIR1 | Leukocyte-associated immunoglobulin-like receptor 1 | 5.64 | 4.25 |
| MFAP3 | Microfibril-associated glycoprotein 3 | 3.7 | 7.3 |
| MFAP3L | Microfibrillar-associated protein 3-like | 3.44 | 8.7 |
| MICA | MHC class I polypeptide-related sequence A | 4.07 | 2.01 |
| NCAM1 | Neural cell adhesion molecule 1 | 2.45 | 7.31 |
| NOTCH3 | Notch 3 intracellular domain | 3.21 | 12.41 |
| NOTCH4 | Notch 4 intracellular domain | 5.89 | 8.84 |
| OLR1 | Oxidized low-density lipoprotein receptor 1, soluble form | 2.84 | 8.41 |
| P2RY14 | P2Y purinoceptor 14 | 2.63 | 4.63 |
| PCDH17 | Protocadherin-17 | 1.7 | 7.36 |
| PDGFRB | Platelet-derived growth factor receptor beta | 2.68 | 10.48 |
| PECAM1 | Platelet endothelial cell adhesion molecule | 7.7 | 10.85 |
| PLXND1 | Plexin-D1 | 5.02 | 11.68 |
| PPAP2B | Lipid phosphate phosphohydrolase 3 | 4.17 | 12.46 |
| PTAFR | Platelet-activating factor receptor | 3.01 | 4.81 |
| PTGER3 | Prostaglandin E2 receptor EP3 subtype | 4.76 | 10.26 |
| PTH1R | Parathyroid hormone/parathyroid hormone-related peptide | 2.35 | 7.31 |
| | receptor | | |
| RAMP3 | Receptor activity-modifying protein 3 | 1.79 | 8.84 |
| ROR2 | Tyrosine-protein kinase transmembrane receptor ROR2 | 3.2 | 5.98 |
| S1PR1 | Sphingosine 1-phosphate receptor 1 | 5.17 | 6.51 |
| SCARB1 | Scavenger receptor class B member 1 | 3.01 | 10.4 |
| SLC13A3 | Solute carrier family 13 member 3 | 3.32 | 7.89 |
| SLC16A4 | Monocarboxylate transporter 5 | 2.88 | 12.54 |
| SLC17A3 | Sodium-dependent phosphate transport protein 4 | 1.58 | 11.55 |
| SLC28A1 | Sodium/nucleoside cotransporter 1 | 4.76 | 6.3 |
| SLC2A5 | Solute carrier family 2, facilitated glucose transporter | 2.74 | 8.5 |
| | member 5 | | |
| SLC39A14 | Zinc transporter ZIP14 | 2.66 | 11.63 |
| SLC6A13 | Sodium- and chloride-dependent GABA transporter 2 | 2.75 | 7.44 |
| SLC7A8 | Large neutral amino acids transporter small subunit 2 | 5.03 | 10.46 |
| SLCO2A1 | Solute carrier organic anion transporter family member 2A1 | 3.46 | 8.06 |
| TBXA2R | Thromboxane A2 receptor | 4.01 | 3.64 |
| TGFBR2 | TGF-beta receptor type-2 | 10.41 | 10.94 |
| THSD7A | Thrombospondin type-1 domain-containing protein 7A | 3.05 | 8 |
| TIE1 | Tyrosine-protein kinase receptor Tie-1 | 2.04 | 4.41 |
| TNFRSF1A | Tumor necrosis factor-binding protein 1 | 6.84 | 10.52 |
| TNFSF12 | Tumor necrosis factor ligand superfamily member 12, | 4.35 | 4.1 |
| | secreted form | | |
| VAMP5 | Vesicle-associated membrane protein 5 | 3.49 | 6.18 |

TABLE 9
antigen markers expressed on the surface of both liver tumor cells and T-cells

| | | | Relative |
|---|---|---|---|
| | | | Expression |
| | | Relative | in colon |
| | | expression | cancer |
| Antigen | Protein Name | in T-Cell | cells |
| ABCB4 | Multidrug resistance protein 3 | 2.02 | 3.7 |
| ADAM10 | Disintegrin and metalloproteinase domain-containing protein 10 | 9.42 | 9.41 |
| ATR | Anthrax toxin receptor 1 | 6.98 | 9.9 |
| BST2 | Bone marrow stromal antigen 2 | 7.38 | 12.45 |
| BTN3A3 | Butyrophilin subfamily 3 member A3 | 9.72 | 7.48 |
| C9 | Complement component C9b | 2.41 | 10.52 |
| CHRND | Acetylcholine receptor subunit delta | 2.43 | 4.05 |
| CLDN14 | Claudin-14 | 2.79 | 2.4 |
| EPOR | Erythropoietin receptor | 4.67 | 10.55 |
| ERBB2 | Receptor tyrosine-protein kinase erbB-2 | 2.36 | 14.12 |
| F2RL3 | Proteinase-activated receptor 4 | 2.17 | 2.61 |
| GJB1 | Gap junction beta-1 protein | 2.96 | 9.4 |
| GPR126 | G-protein coupled receptor 126 | 2.23 | 11.32 |
| IL1R1 | Interleukin-1 receptor type 1, soluble form | 2.88 | 12.57 |
| ITGB1 | Integrin beta-1 | 8.76 | 13.48 |
| NAALADL1 | N-acetylated-alpha-linked acidic dipeptidase-like protein | 3.03 | 1.46 |
| OR7A5 | Olfactory receptor 7A5 | 1.51 | 3.83 |
| SGCD | Delta-sarcoglycan | 3.99 | 7.21 |
| SIGLEC6 | Sialic acid-binding Ig-like lectin 6 | 3.57 | 3.49 |
| SLC38A3 | Sodium-coupled neutral amino acid transporter 3 | 1.89 | 8.91 |
| TFR2 | Transferrin receptor protein 2 | 2.74 | 10.47 |

TABLE 10
antigen markers expressed on the surface of both lung tumor cells and T-cells

| | | | Relative |
|---|---|---|---|
| | | | Expression |
| | | Relative | in colon |
| | | expression | cancer |
| Antigen | Protein Name | in T-Cell | cells |
| ABCB6 | ATP-binding cassette sub-family B member 6, | 2.88 | 9.82 |
| | mitochondrial | | |
| ABCC1 | Multidrug resistance-associated protein 1 | 7.05 | 8.16 |
| ACCN1 | Acid-sensing ion channel 2 | 2.25 | 0.8 |
| ADAM23 | Disintegrin and metalloproteinase domain-containing | 2.51 | 4.73 |
| | protein 23 | | |
| ADORA1 | Adenosine receptor A1 | 4.49 | 8.22 |
| ADORA2B | Adenosine receptor A2b | 1.66 | 7.5 |
| AJAP1 | Adherens junction-associated protein 1 | 1.85 | 6.24 |
| APLP1 | C30 | 2.22 | 6.02 |
| AQP3 | Aquaporin-3 | 8.38 | 13.88 |
| ATP10D | Probable phospholipid-transporting ATPase VD | 2.43 | 7.4 |
| ATP1A3 | Sodium/potassium-transporting ATPase subunit alpha-3 | 3.01 | 3.13 |
| ATP1B2 | Sodium/potassium-transporting ATPase subunit beta-2 | 3.21 | 3.8 |
| ATP1B3 | Sodium/potassium-transporting ATPase subunit beta-3 | 8.6 | 14.26 |
| AXL | Tyrosine-protein kinase receptor UFO | 2.51 | 9.58 |
| BEST1 | Bestrophin-1 | 2.49 | 4.44 |
| BTC | Betacellulin | 2.86 | 4.59 |
| BTN3A1 | Butyrophilin subfamily 3 member A1 | 10.66 | 11.63 |
| CALCR | Calcitonin receptor | 2.95 | 8.62 |
| CALCRL | Calcitonin gene-related peptide type 1 receptor | 2.12 | 7.67 |
| CCR1 | C—C chemokine receptor type 1 | 2.63 | 9.77 |
| CD163 | Soluble CD163 | 2.66 | 8.76 |
| CD300A | CMRF35-like molecule 8 | 7.96 | 4.23 |
| CD300A | CMRF35-like molecule 8 | 2.29 | 4.23 |
| CD68 | Macrosialin | 4.02 | 8.92 |
| CD74 | HLA class II histocompatibility antigen gamma chain | 9.1 | 13.44 |
| CD86 | T-lymphocyte activation antigen CD86 | 2.93 | 5.04 |
| CHRNA3 | Neuronal acetylcholine receptor subunit alpha-3 | 2.54 | 4.62 |
| CHRNA3 | Neuronal acetylcholine receptor subunit alpha-3 | 2 | 4.62 |
| CKAP4 | Cytoskeleton-associated protein 4 | 6.15 | 11.94 |
| CLCA2 | Calcium-activated chloride channel regulator 2, 35 kDa | 2.99 | 9.81 |
| | form | | |
| CLDN5 | Claudin-5 | 3.66 | 7.73 |
| CLSTN1 | CTF1-alpha | 8.26 | 12.51 |
| CNIH3 | Protein cornichon homolog 3 | 2.7 | 6.09 |
| COMT | Catechol O-methyltransferase | 7.78 | 12.13 |
| CSPG5 | Chondroitin sulfate proteoglycan 5 | 2.84 | 5.69 |
| CXCR6 | C—X—C chemokine receptor type 6 | 3.16 | 3.91 |
| CXCR7 | Atypical chemokine receptor 3 | 2.5 | 8.95 |
| DCHS1 | Protocadherin-16 | 4.29 | 2.28 |
| DSC3 | Desmocollin-2 | 2.82 | 8.95 |
| DSG3 | Desmoglein-3 | 2.23 | 10.73 |
| EGFR | Epidermal growth factor receptor | 3.8 | 10.92 |
| FAT2 | Protocadherin Fat 2 | 2.25 | 9.29 |
| FCER1G | High affinity immunoglobulin epsilon receptor subunit | 3.13 | 8.96 |
| | gamma | | |
| FCGR1A | High affinity immunoglobulin gamma Fc receptor I | 2.09 | 9.65 |
| FLT4 | Vascular endothelial growth factor receptor 3 | 3.19 | 3.36 |
| FPR2 | N-formyl peptide receptor 2 | 2.9 | 7.14 |
| FURIN | Furin | 6.42 | 7.5 |
| FZD6 | Frizzled-6 | 2.64 | 10.45 |
| GABBR2 | Gamma-aminobutyric acid type B receptor subunit 2 | 3.79 | 9.19 |
| GABRB3 | Gamma-aminobutyric acid receptor subunit beta-3 | 2.46 | 8.83 |
| GABRD | Gamma-aminobutyric acid receptor subunit delta | 1.72 | 1.67 |
| GABRE | Gamma-aminobutyric acid receptor subunit epsilon | 1.85 | 9.18 |
| GIPR | Gastric inhibitory polypeptide receptor | 3.43 | 5.37 |
| GJA1 | Gap junction alpha-1 protein | 2.84 | 12.65 |
| GJB3 | Gap junction beta-3 protein | 3.72 | 3.79 |
| GJB5 | Gap junction beta-5 protein | 1.77 | 6.69 |
| GLRA2 | Glycine receptor subunit alpha-2 | 2.26 | 6.15 |
| GPR109B | Hydroxycarboxylic acid receptor 3 | 1.77 | 2.91 |
| GPR12 | G-protein coupled receptor 12 | 2 | 1.76 |
| GPR176 | Probable G-protein coupled receptor 176 | 2.05 | 3.86 |
| GPR50 | Melatonin-related receptor | 2.26 | 3.16 |
| GRIK1 | Glutamate receptor ionotropic, kainate 1 | 4.66 | 5.65 |
| GRIN2D | Glutamate receptor ionotropic, NMDA 2D | 2.17 | 2.32 |
| HCRTR1 | Orexin receptor type 1 | 2.34 | 3.56 |
| HLA-DPA1 | HLA class II histocompatibility antigen, DP alpha 1 chain | 8.31 | 12.86 |
| HLA-DQA1 | HLA class II histocompatibility antigen, DQ alpha 1 chain | 2.35 | 11.44 |
| HLA-DQB1 | HLA class II histocompatibility antigen, DQ beta 1 chain | 7.4 | 12.71 |
| HLA-DRA | HLA class II histocompatibility antigen, DR alpha chain | 6.42 | 14.18 |
| HLA-DRB4 | HLA class II histocompatibility antigen, DR beta 4 chain | 2.72 | 11.24 |
| IGSF9B | Protein turtle homolog B | 3.92 | 2.81 |
| IL1RAP | Interleukin-1 receptor accessory protein | 3.99 | 11.4 |
| IL1RL1 | Interleukin-1 receptor-like 1 | 2.55 | 5.15 |
| IL4R | Soluble interleukin-4 receptor subunit alpha | 4.15 | 9.56 |
| IL7R | Interleukin-7 receptor subunit alpha | 11.62 | 11.26 |
| ITGA6 | Integrin alpha-6 light chain | 7.99 | 12.76 |
| JPH3 | Junctophilin-3 | 2.34 | 2.5 |
| KCNS3 | Potassium voltage-gated channel subfamily S member 3 | 2.45 | 8.91 |
| KIT | Mast/stem cell growth factor receptor Kit | 2.85 | 8.67 |
| KITLG | Soluble KIT ligand | 2.58 | 7.27 |
| LILRB3 | Leukocyte immunoglobulin-like receptor subfamily B | 5.65 | 8.03 |
| | member 3 | | |
| LILRB4 | Leukocyte immunoglobulin-like receptor subfamily B | 3.12 | 10.44 |
| | member 4 | | |
| LPAR1 | Lysophosphatidic acid receptor 1 | 4.12 | 5.47 |
| LPHN3 | Latrophilin-3 | 2.85 | 6.43 |
| MMP24 | Processed matrix metalloproteinase-24 | 5.19 | 5.73 |
| MPZ | Myelin protein P0 | 2.56 | 3.63 |
| MUC4 | Mucin-4 beta chain | 3.04 | 10.34 |
| NCKAP1L | Nck-associated protein 1-like | 6.69 | 7.51 |
| NKG7 | Protein NKG7 | 10.92 | 3.66 |
| NOTCH2 | Notch 2 intracellular domain | 6.62 | 6.22 |
| NRCAM | Neuronal cell adhesion molecule | 2.78 | 8.16 |
| NRG2 | Neuregulin-2 | 3.55 | 9.22 |
| NRXN1 | Neurexin-1 | 2.56 | 5.33 |
| NTRK2 | BDNF/NT-3 growth factors receptor | 2.56 | 10.7 |
| NTSR1 | Neurotensin receptor type 1 | 1.74 | 9.74 |
| P2RY1 | P2Y purinoceptor 1 | 2.34 | 7.62 |
| P2RY6 | P2Y purinoceptor 6 | 4.27 | 5.79 |
| PCDH8 | Protocadherin-8 | 2.67 | 9.29 |
| PCDHA3 | Protocadherin alpha-3 | 2.14 | 3.54 |
| PIK3IP1 | Phosphoinositide-3-kinase-interacting protein 1 | 8.68 | 3.47 |
| PLXNA2 | Plexin-A2 | 2.88 | 7.3 |
| PRR4 | Processed poliovirus receptor-related protein 4 | 3.24 | 8.02 |
| PTPRE | Receptor-type tyrosine-protein phosphatase epsilon | 6.03 | 7.92 |
| PTPRO | Receptor-type tyrosine-protein phosphatase U | 10.46 | 9.01 |
| PTPRU | Receptor-type tyrosine-protein phosphatase U | 3.72 | 6.18 |
| RABAC1 | Prenylated Rab acceptor protein 1 | 7.54 | 8.82 |
| SCTR | Secretin receptor | 2.2 | 2.48 |
| SECTM1 | Secreted and transmembrane protein 1 | 2.42 | 6.9 |
| SGCE | Epsilon-sarcoglycan | 2.15 | 9.65 |
| SGCG | Gamma-sarcoglycan | 2.56 | 5.74 |
| SLC16A3 | Monocarboxylate transporter 4 | 5.89 | 12.72 |
| SLC16A7 | Monocarboxylate transporter 2 | 5.39 | 6.97 |
| SLC20A2 | Sodium-dependent phosphate transporter 2 | 2.51 | 12.69 |
| SLC26A4 | Pendrin | 3.57 | 9.39 |
| SLC2A1 | Solute carrier family 2, facilitated glucose transporter | 5.1 | 5.83 |
| | member 1 | | |
| SLC4A7 | Sodium bicarbonate cotransporter 3 | 4.89 | 8.7 |
| SLCO3A1 | Solute carrier organic anion transporter family member 3A1 | 4.87 | 7.91 |
| SYNE2 | Nesprin-2 | 9.43 | 10.43 |
| TACR2 | Substance-K receptor | 2.23 | 6.68 |
| TFRC | Transferrin receptor protein 1, serum form | 7.32 | 14.31 |
| TMEFF1 | Tomoregulin-1 | 3.22 | 5.05 |
| TMPRSS11D | Transmembrane protease serine 11D catalytic chain | 2.35 | 8.32 |

TABLE 11
antigen markers expressed on the surface of both ovary tumor cells and T-cells;

| | | | Relative |
|---|---|---|---|
| | | | Expression |
| | | Relative | in colon |
| | | expression | cancer |
| Antigen | Protein Name | in T-Cell | cells |
| ACVR2B | Activin receptor type-2B | 2.1 | 4.26 |
| ADAM28 | Disintegrin and metalloproteinase domain-containing | 2.83 | 9.22 |
| | protein 28 | | |
| ADRA2C | Alpha-2C adrenergic receptor | 4.6 | 5.13 |
| ATP2B1 | Plasma membrane calcium-transporting ATPase 1 | 5.3 | 11.49 |
| ATP2B4 | Plasma membrane calcium-transporting ATPase 4 | 8.21 | 10.1 |
| ATP7A | Copper-transporting ATPase 1 | 3.91 | 7.31 |
| CD200 | OX-2 membrane glycoprotein | 2.83 | 10.51 |
| CD47 | Leukocyte surface antigen CD47 | 9.88 | 10.42 |
| CDH12 | Cadherin-12 | 2.31 | 5.91 |
| CDH18 | Cadherin-18 | 2.28 | 4.79 |
| CDH2 | Cadherin-2 | 3.72 | 11.97 |
| CDH6 | Cadherin-6 | 2.77 | 8.68 |
| CDIPT | CDP-diacylglycerol--inositol 3-phosphatidyltransferase | 8.88 | 10.73 |
| CELSR2 | Cadherin EGF LAG seven-pass G-type receptor 2 | 2.66 | 8.38 |
| CHRNA1 | Acetylcholine receptor subunit alpha | 2.42 | 5.71 |
| CLSTN3 | Calsyntenin-3 | 3.87 | 4.54 |
| CX3CR1 | CX3C chemokine receptor 1 | 9 | 11.42 |
| DDR1 | Epithelial discoidin domain-containing receptor 1 | 3.83 | 12.36 |
| EPHA1 | Ephrin type-A receptor 1 | 2.02 | 5.96 |
| EPHA4 | Ephrin type-A receptor 4 | 2.39 | 8.56 |
| ERBB4 | ERBB4 intracellular domain | 2.29 | 9.76 |
| FGFR1 | Fibroblast growth factor receptor 1 | 5.42 | 11.4 |
| FGFR3 | Fibroblast growth factor receptor 3 | 2.95 | 11.35 |
| FZD2 | Frizzled-2 | 1.91 | 8.06 |
| FZD7 | Frizzled-7 | 2.55 | 10.24 |
| GJA4 | Gap junction alpha-4 protein | 2.04 | 6.7 |
| GPR125 | Probable G-protein coupled receptor 125 | 2.35 | 7.88 |
| GPR56 | GPR56 C-terminal fragment | 8.6 | 11.27 |
| GPR64 | G-protein coupled receptor 64 | 2.04 | 8.57 |
| GPRC5B | G-protein coupled receptor family C group 5 member B | 1.96 | 10.29 |
| GRIA2 | Glutamate receptor 2 | 1.96 | 11.78 |
| GRIK5 | Glutamate receptor ionotropic, kainate 5 | 5.79 | 3.36 |
| GRIN2A | Glutamate receptor ionotropic, NMDA 2A | 1.68 | 2.96 |
| HEG1 | Protein HEG homolog 1 | 4.8 | 10.1 |
| HRH1 | Histamine H1 receptor | 2.31 | 6.26 |
| HTR3A | 5-hydroxytryptamine receptor 3A | 2.1 | 9.35 |
| IFITM2 | Interferon-induced transmembrane protein 2 | 10.27 | 11.36 |
| IFITM3 | Interferon-induced transmembrane protein 3 | 8.55 | 13.48 |
| KCNH2 | Potassium voltage-gated channel subfamily H member 2 | 2.09 | 5.36 |
| KCNJ12 | ATP-sensitive inward rectifier potassium channel 12 | 2.29 | 2.21 |
| L1CAM | Neural cell adhesion molecule L1 | 2.61 | 8.73 |
| LGR5 | Leucine-rich repeat-containing | 2.45 | 12.12 |
| | G-protein coupled receptor 5 | | |
| LPHN1 | Latrophilin-1 | 4.5 | 5.56 |
| LPHN1 | Latrophilin-1 | 1.63 | 5.56 |
| LPHN2 | Latrophilin-2 | 1.93 | 7.14 |
| MGA | Glucoamylase | 5.15 | 5.65 |
| NEO1 | Neogenin | 1.85 | 10.31 |
| NPTN | Neuroplastin | 8.46 | 13.14 |
| NRG1 | Neuregulin-1 | 2.61 | 6.53 |
| NTRK1 | High affinity nerve growth factor receptor | 2.09 | 2.49 |
| PCDH7 | Protocadherin-7 | 2.89 | 8.52 |
| PCDH9 | Protocadherin-9 | 2.99 | 6.15 |
| PDGFRA | Platelet-derived growth factor receptor alpha | 3.69 | 8.44 |
| PDGFRA | Platelet-derived growth factor receptor alpha | 2.26 | 8.44 |
| PLXNB1 | Plexin-B1 | 2.26 | 6.71 |
| PLXNB2 | Plexin-B2 | 3.1 | 10.68 |
| PODXL | Podocalyxin | 2.73 | 11.41 |
| PRSS8 | Prostasin heavy chain | 2.07 | 10.77 |
| PTH2R | Parathyroid hormone 2 receptor | 1.85 | 8.67 |
| PVRL3 | Poliovirus receptor-related protein 3 | 2.56 | 10.15 |
| SCNN1A | Amiloride-sensitive sodium channel subunit alpha | 5.97 | 10.63 |
| SLC29A2 | Equilibrative nucleoside transporter 2 | 2.93 | 1.89 |
| SSPN | Sarcospan | 3.49 | 9.16 |
| STAR | Heat-stable enterotoxin receptor | 2.36 | 7.13 |
| TGFA | Transforming growth factor alpha | 2.64 | 1.71 |
| TMED1 | Transmembrane emp24 domain-containing protein 1 | 4.79 | 9.3 |
| TMEM59 | Transmembrane protein 59 | 8.83 | 12.74 |
| TNFRSF25 | Tumor necrosis factor receptor superfamily member 25 | 7.53 | 4.27 |
| TYRO3 | Tyrosine-protein kinase receptor TYRO3 | 4.11 | 10.27 |
| UPK2 | Uroplakin-2 | 2.29 | 7.49 |

TABLE 12
antigen markers expressed on the surface of both pancreas tumor cells and T-cells

| | | | Relative |
|---|---|---|---|
| | | | Expression |
| | | Relative | in colon |
| | | expression | cancer |
| Antigen | Protein Name | in T-Cell | cells |
| ADAM 9 | Disintegrin and metalloproteinase domain-containing | 3.49 | 10.99 |
| | protein 9 | | |
| B4GALT1 | Processed beta-1,4-galactosyltransferase 1 | 7.44 | 8.99 |
| BDKRB2 | B2 bradykinin receptor | 2.52 | 4.44 |
| CA9 | Carbonic anhydrase 9 | 3.34 | 11.9 |
| CACNA1C | Voltage-dependent L-type calcium channel subunit alpha-1C | 2.36 | 4.54 |
| CD58 | Lymphocyte function-associated antigen 3 | 6.51 | 8.16 |
| CDH11 | Cadherin-11 | 2.85 | 10.38 |
| CDH3 | Cadherin-3 | 1.96 | 10.91 |
| CFTR | Cystic fibrosis transmembrane conductance regulator | 3.12 | 11.45 |
| CHRNB4 | Neuronal acetylcholine receptor subunit beta-4 | 2.38 | 0.66 |
| CLDN10 | Claudin-10 | 2.36 | 11.5 |
| CXCR4 | C—X—C chemokine receptor type 4 | 11.74 | 10.98 |
| DAG1 | Beta-dystroglycan | 5.65 | 10.98 |
| DDR2 | Discoidin domain-containing receptor 2 | 2.34 | 8 |
| DMPK | Myotonin-protein kinase | 3.7 | 4.21 |
| FAT1 | Protocadherin Fat 1, nuclear form | 3.3 | 12.45 |
| HTR2B | 5-hydroxytryptamine receptor 2B | 2.22 | 7.73 |
| LDLR | Low-density lipoprotein receptor | 2.93 | 12.14 |
| NCKAP1 | Nck-associated protein 1 | 3.34 | 11.99 |
| PMP22 | Peripheral myelin protein 22 | 2.09 | 10.66 |
| PNPLA2 | Patatin-like phospholipase domain-containing protein 2 | 5.46 | 3.45 |
| PNPLA2 | Patatin-like phospholipase domain-containing protein 2 | 2.35 | 3.45 |
| TEK | Angiopoietin-1 receptor | 3.87 | 8.52 |
| TGFBR1 | TGF-beta receptor type-1 | 2.17 | 4.3 |

TABLE 13
antigen markers expressed on the surface of both prostate tumor cells and T-cells

| | | | Relative |
|---|---|---|---|
| | | | Expression |
| | | Relative | in colon |
| | | expression | cancer |
| Antigen | Protein Name | in T-Cell | cells |
| ACCN3 | Acid-sensing ion channel 3 | 2.47 | 2.03 |
| ADRB1 | Beta-1 adrenergic receptor | 2.85 | 5.09 |
| ADRB2 | Beta-2 adrenergic receptor | 5.74 | 9.43 |
| AGTR1 | Type-1 angiotensin II receptor | 2.81 | 11.62 |
| APLP2 | Amyloid-like protein 2 | 7.06 | 13.06 |
| ATP1A2 | Sodium/potassium-transporting ATPase subunit alpha-2 | 3.07 | 7.55 |
| ATP8A1 | Probable phospholipid-transporting ATPase IA | 7.23 | 9.16 |
| CADM1 | Cell adhesion molecule 1 | 4.42 | 12.28 |
| CHRM3 | Muscarinic acetylcholine receptor M3 | 1.85 | 9.23 |
| CHRNA2 | Neuronal acetylcholine receptor subunit alpha-2 | 2.83 | 5.34 |
| CXADR | Coxsackievirus and adenovirus receptor | 3.31 | 12.74 |
| DPP4 | Dipeptidyl peptidase 4 soluble form | 6.42 | 11.22 |
| ECE1 | Endothelin-converting enzyme 1 | 7.14 | 4.7 |
| ENPP4 | Bis(5′-adenosyl)-triphosphatase ENPP4 | 6.57 | 7.49 |
| EPHA3 | Ephrin type-A receptor 3 | 2.84 | 7.85 |
| ERG | Potassium voltage-gated channel subfamily H member 2 | 2.72 | 11.3 |
| FAM38A | Piezo-type mechanosensitive ion channel component 1 | 8.4 | 9.57 |
| FOLH1 | Glutamate carboxypeptidase 2 | 2.96 | 13.18 |
| GABRA2 | Gamma-aminobutyric acid receptor subunit alpha-2 | 3 | 6.42 |
| GHR | Growth hormone-binding protein | 2.52 | 6.84 |
| GPM6B | Neuronal membrane glycoprotein M6-b | 3.22 | 6.56 |
| GPR116 | Probable G-protein coupled receptor 116 | 3.69 | 10.09 |
| HBEGF | Heparin-binding EGF-like growth factor | 2.87 | 8.12 |
| JAM3 | Junctional adhesion molecule C | 4.29 | 7.26 |
| KCND3 | Potassium voltage-gated channel subfamily D member 3 | 3.09 | 9.77 |
| LIFR | Leukemia inhibitory factor receptor | 2.71 | 6.8 |
| LRBA | Lipopolysaccharide-responsive and beige-like anchor | 5.35 | 9.26 |
| | protein | | |
| MME | Neprilysin | 2.62 | 8.05 |
| NOV | Plexin-A1 | 2.43 | 10.41 |
| NRP1 | Neuropilin-1 | 3.17 | 7.85 |
| OPRK1 | Kappa-type opioid receptor | 2.07 | 4.92 |
| PLXNB3 | Plexin-B3 | 2.57 | 3.59 |
| PPAP2A | Lipid phosphate phosphohydrolase 1 | 3.6 | 11.55 |
| SCAMP5 | Secretory carrier-associated membrane protein 5 | 3.03 | 8.43 |
| SLC23A2 | Solute carrier family 23 member 2 | 3.55 | 7.04 |
| SLC2A4 | Solute carrier family 2, facilitated glucose transporter | 2.67 | 5.96 |
| | member 4 | | |
| SLC36A1 | Proton-coupled amino acid transporter 1 | 3.38 | 9.28 |
| SLC4A4 | Electrogenic sodium bicarbonate cotransporter 1 | 3.14 | 11.29 |
| STIM1 | Stromal interaction molecule 1 | 3.68 | 6.51 |
| TMPRSS2 | Transmembrane protease serine 2 catalytic chain | 2.67 | 9.63 |
| TRPV6 | Transient receptor potential cation channel subfamily V | 4.84 | 8.09 |
| | members | | |
| VIPR1 | Vasoactive intestinal polypeptide receptor 1 | 4.41 | 7.73 |
| YIPF3 | Protein YIPF3, 36 kDa form III | 14 | 14.3 |

TABLE 14
antigen markers expressed on the surface of T-cells and overexpressed
in liquid tumor cells (ALL, AML, CML, MDS, CLL, CTRL)

| | | Relative |
|---|---|---|
| | | expression |
| Antigen | Protein Name | on T cell |
| CD63 | CD63 antigen | 0.83 |
| CXCR4 | C—X—C chemokine receptor type 4 | 0.82 |
| IFITM2 | Interferon-induced transmembrane protein 2 | 0.82 |
| ITM2B | Bri23 peptide | 0.81 |
| BTF3 | Butyrophilin subfamily 3 member A2 | 0.8 |
| HLA-DRB1 | HLA class II histocompatibility antigen, DRB1-12 beta chain | 0.79 |
| HLA-DRA | HLA class II histocompatibility antigen, DR alpha chain | 0.78 |
| IFITM3 | Interferon-induced transmembrane protein 3 | 0.78 |
| NKG7 | Protein NKG7 | 0.78 |
| FCER1G | High affinity immunoglobulin epsilon receptor subunit gamma | 0.78 |
| IFITM1 | Interferon-induced transmembrane protein 1 | 0.76 |
| NPTN | Neuroplastin | 0.76 |
| GYPC | Glycophori n-C | 0.76 |
| GPR160 | Probable G-protein coupled receptor 160 | 0.76 |
| HLA-DPB1 | HLA class II histocompatibility antigen, DP beta 1 chain | 0.75 |
| BRI3 | CT-BRI3 | 0.75 |
| SLC38A2 | Sodium-coupled neutral amino acid transporter 2 | 0.74 |
| C5AR1 | C5a anaphylatoxin chemotactic receptor 1 | 0.74 |
| CDIPT | CDP-diacylglycerol-inositol 3-phosphatidyltransferase | 0.73 |
| TNFSF13B | Tumor necrosis factor ligand superfamily member 13b, soluble | 0.73 |
| | form | |
| CSF3R | Granulocyte colony-stimulating factor receptor | 0.73 |
| HLA-DPA1 | HLA class II histocompatibility antigen, DP alpha 1 chain | 0.71 |
| CD164 | Sialomucin core protein 24 | 0.71 |
| CD97 | CD97 antigen subunit beta | 0.7 |
| C3AR1 | C3a anaphylatoxin chemotactic receptor | 0.69 |
| P2RY8 | P2Y purinoceptor 8 | 0.68 |
| BSG | Basigin | 0.68 |
| APLP2 | Amyloid-like protein 2 | 0.67 |
| TFRC | Transferrin receptor protein 1, serum form | 0.67 |
| MGAM | Glucoamylase | 0.67 |
| GYPA | Glycophori n-A | 0.67 |
| TMED10 | Transmembrane emp24 domain-containing protein 10 | 0.66 |
| FCGRT | IgG receptor FcRn large subunit p51 | 0.66 |
| CKAP4 | Cytoskeleton-associated protein 4 | 0.66 |
| DYSF | Dysferlin | 0.66 |
| SPPL2A | Signal peptide peptidase-like 2A | 0.65 |
| LAMP2 | Lysosome-associated membrane glycoprotein 2 | 0.65 |
| SLC7A5 | Large neutral amino acids transporter small subunit 1 | 0.65 |
| TNFRSF1B | Tumor necrosis factor-binding protein 2 | 0.64 |
| TREM1 | Triggering receptor expressed on myeloid cells 1 | 0.64 |
| GPR183 | G-protein coupled receptor 183 | 0.63 |
| SERINC3 | Serine incorporator 3 | 0.63 |
| CD58 | Lymphocyte function-associated antigen 3 | 0.63 |
| GYPB | Glycophorin-B | 0.63 |
| RABAC1 | Prenylated Rab acceptor protein 1 | 0.62 |
| KCNH2 | Potassium voltage-gated channel subfamily H member 2 | 0.62 |
| FPR1 | fMet-Leu-Phe receptor | 0.62 |
| P2RY13 | P2Y purinoceptor 13 | 0.62 |
| CLEC5A | C-type lectin domain family 5 member A | 0.62 |
| SLC7A7 | Y + L amino acid transporter 1 | 0.61 |
| MICB | MHC class I polypeptide-related sequence B | 0.61 |
| CD300LF | CMRF35-like molecule 1 | 0.61 |
| GJB6 | Gap junction beta-6 protein | 0.61 |
| ATP1A1 | Sodium/potassium-transporting ATPase subunit alpha-1 | 0.6 |
| PTGER4 | Prostaglandin E2 receptor EP4 subtype | 0.6 |
| CD8A | T-cell surface glycoprotein CD8 alpha chain | 0.6 |
| PTGER2 | Prostaglandin E2 receptor EP2 subtype | 0.6 |
| GPR97 | Probable G-protein coupled receptor 97 | 0.6 |
| IMP3 | Signal peptide peptidase-like 2A | 0.59 |
| LAMP1 | Lysosome-associated membrane glycoprotein 1 | 0.59 |
| LILRB3 | Leukocyte immunoglobulin-like receptor subfamily B member 3 | 0.59 |
| GPR109B | Hydroxycarboxylic acid receptor 3 | 0.59 |
| SAT2 | Sodium-coupled neutral amino acid transporter 2 | 0.58 |
| GPR65 | Psychosine receptor | 0.58 |
| AMICA1 | Junctional adhesion molecule-like | 0.58 |
| PAG1 | Phosphoprotein associated with glycosphingolipid-enriched | 0.58 |
| | microdomains 1 | |
| ENPP4 | Bis(5′-adenosyl)-triphosphatase ENPP4 | 0.57 |
| SLC40A1 | Solute carrier family 40 member 1 | 0.57 |
| OLR1 | Oxidized low-density lipoprotein receptor 1, soluble form | 0.57 |
| LRRC33 | Negative regulator of reactive oxygen species | 0.56 |
| IL7R | Interleukin-7 receptor subunit alpha | 0.56 |
| LAIR1 | Leukocyte-associated immunoglobulin-like receptor 1 | 0.56 |
| ITM2C | CT-BRI3 | 0.56 |
| GPR84 | G-protein coupled receptor 84 | 0.56 |
| SLC12A7 | Solute carrier family 12 member 7 | 0.55 |
| PTAFR | Platelet-activating factor receptor | 0.55 |
| CD33 | Myeloid cell surface antigen CD33 | 0.55 |
| SLC22A16 | Solute carrier family 22 member 16 | 0.55 |
| CCR7 | C—C chemokine receptor type 7 | 0.54 |
| TLR1 | Toll-like receptor 1 | 0.54 |
| TGOLN2 | Trans-Golgi network integral membrane protein 2 | 0.54 |
| YIPF3 | Protein YIPF3, 36 kDa form III | 0.54 |
| BST2 | Bone marrow stromal antigen 2 | 0.54 |
| MAGT1 | Magnesium transporter protein 1 | 0.54 |
| TMEM173 | Stimulator of interferon genes protein | 0.54 |
| ERMAP | Erythroid membrane-associated protein | 0.54 |
| CEACAM1 | Carcinoembryonic antigen-related cell adhesion molecule 1 | 0.54 |
| NIPA2 | Magnesium transporter NIPA2 | 0.53 |
| PECAM1 | Platelet endothelial cell adhesion molecule | 0.53 |
| CD1D | Antigen-presenting glycoprotein CD1d | 0.53 |
| TMEM59 | Transmembrane protein 59 | 0.53 |
| NCKAP1L | Nck-associated protein 1-like | 0.53 |
| FAS | Tumor necrosis factor receptor superfamily member 6 | 0.53 |
| IL6R | Interleukin-6 receptor subunit alpha | 0.53 |
| TNFRSF1A | Tumor necrosis factor-binding protein 1 | 0.53 |
| KEL | Kell blood group glycoprotein | 0.53 |
| TMEM149 | IGF-like family receptor 1 | 0.52 |
| SLC3A2 | 4F2 cell-surface antigen heavy chain | 0.52 |
| ORAI1 | Calcium release-activated calcium channel protein 1 | 0.52 |
| XKR8 | XK-related protein 8, processed form | 0.52 |
| C9orf46 | Plasminogen receptor (KT) | 0.52 |
| TMEM127 | Transmembrane protein 127 | 0.52 |
| SLC2A1 | Solute carrier family 2, facilitated glucose transporter member 1 | 0.52 |
| FCGR1B | High affinity immunoglobulin gamma Fc receptor IB | 0.52 |
| CXCR2 | C—X—C chemokine receptor type 2 | 0.52 |
| IL4R | Soluble interleukin-4 receptor subunit alpha | 0.51 |
| HSD17B7 | 3-keto-steroid reductase | 0.51 |
| SEMA4D | Semaphorin-4D | 0.51 |
| ZDHHC5 | Palmitoyltransferase ZDHHC5 | 0.51 |
| ADRB2 | Beta-2 adrenergic receptor | 0.51 |
| S1PR4 | Sphingosine 1-phosphate receptor 4 | 0.51 |
| PILRA | Paired immunoglobulin-like type 2 receptor alpha | 0.51 |
| LTB4R | Leukotriene B4 receptor 1 | 0.51 |
| SORT1 | Sortilin | 0.51 |
| SLCO4C1 | Solute carrier organic anion transporter family member 4C1 | 0.51 |
| ANO10 | Anoctamin-10 | 0.51 |
| CLSTN1 | CTF1-alpha | 0.5 |
| RHBDF2 | Inactive rhomboid protein 2 | 0.5 |
| CCR1 | C—C chemokine receptor type 1 | 0.5 |
| EPCAM | Epithelial cell adhesion molecule | 0.5 |
| PNPLA2 | Patatin-like phospholipase domain-containing protein 2 | 0.49 |
| SLC12A6 | Solute carrier family 12 member 6 | 0.49 |
| SLC30A1 | Zinc transporter 1 | 0.49 |
| GPR27 | Probable G-protein coupled receptor 27 | 0.49 |
| EPOR | Erythropoietin receptor | 0.49 |
| CD79A | B-cell antigen receptor complex-associated protein alpha chain | 0.48 |
| HLA-DQB1 | HLA class II histocompatibility antigen, DQ beta 1 chain | 0.48 |
| HBP1 | Glycosylphosphatidylinositol-anchored high density lipoprotein- | 0.48 |
| | binding protein 1 | |
| ABCA7 | ATP-binding cassette sub-family A member 7 | 0.48 |
| RAG1AP1 | Sugar transporter SWEET 1 | 0.48 |
| CD47 | Leukocyte surface antigen CD47 | 0.48 |
| CXCL16 | C—X—C motif chemokine 16 | 0.48 |
| SLC14A1 | Urea transporter 1 | 0.48 |
| TGFBR2 | TGF-beta receptor type-2 | 0.47 |
| LRBA | Lipopolysaccharide-responsive and beige-like anchor protein | 0.47 |
| MFSD5 | Molybdate-anion transporter | 0.47 |
| RELT | Tumor necrosis factor receptor superfamily member 19L | 0.47 |
| ATP2B4 | Plasma membrane calcium-transporting ATPase 4 | 0.47 |
| FURIN | Furin | 0.47 |
| GAPT | Protein GAPT | 0.47 |
| NFAM1 | NFAT activation molecule 1 | 0.47 |
| ATP2B1 | Plasma membrane calcium-transporting ATPase 1 | 0.46 |
| SLC26A11 | Sodium-independent sulfate anion transporter | 0.46 |
| STX4 | Syntaxin-4 | 0.46 |
| NAT1 | Sodium-coupled neutral amino acid transporter 3 | 0.46 |
| STIM1 | Stromal interaction molecule 1 | 0.46 |
| SLC39A4 | Zinc transporter ZIP4 | 0.46 |
| ESYT2 | Extended synaptotagmin-2 | 0.46 |
| TM7SF3 | Transmembrane 7 superfamily member 3 | 0.46 |
| SEMA4A | Semaphorin-4A | 0.46 |
| CYBB | Cytochrome b-245 heavy chain | 0.46 |
| FCAR | Immunoglobulin alpha Fc receptor | 0.46 |
| GABBR1 | Gamma-aminobutyric acid type B receptor subunit 1 | 0.45 |
| CD53 | Leukocyte surface antigen CD53 | 0.45 |
| SIGLEC10 | Sialic acid-binding Ig-like lectin 10 | 0.45 |
| S1PR1 | Sphingosine 1-phosphate receptor 1 | 0.45 |
| BTN3A2 | Butyrophilin subfamily 3 member A2 | 0.45 |
| NOTCH2 | Notch 2 intracellular domain | 0.45 |
| PIK3IP1 | Phosphoinositide-3-kinase-interacting protein 1 | 0.45 |
| FAM168B | Myelin-associated neurite-outgrowth inhibitor | 0.45 |
| LPAR2 | Lysophosphatidic acid receptor 2 | 0.45 |
| ATP1B3 | Sodium/potassium-transporting ATPase subunit beta-3 | 0.45 |
| FLVCR1 | Feline leukemia virus subgroup C receptor-related protein 1 | 0.45 |
| SECTM1 | Secreted and transmembrane protein 1 | 0.45 |
| SLC38A5 | Sodium-coupled neutral amino acid transporter 5 | 0.45 |
| GPR18 | N-arachidonyl glycine receptor | 0.44 |
| LMBR1L | Protein LMBR1L | 0.44 |
| ABCC1 | Multidrug resistance-associated protein 1 | 0.44 |
| SLC22A18 | Solute carrier family 22 member 18 | 0.44 |
| CSF1R | Macrophage colony-stimulating factor 1 receptor | 0.44 |
| EMR1 | EGF-like module-containing mucin-like hormone receptor-like 1 | 0.44 |
| FPR2 | N-formyl peptide receptor 2 | 0.44 |
| KIT | Mast/stem cell growth factor receptor Kit | 0.44 |
| MS4A1 | B-lymphocyte antigen CD20 | 0.43 |
| MICA | MHC class I polypeptide-related sequence A | 0.43 |
| GPR172A | Solute carrier family 52, riboflavin transporter, member 2 | 0.43 |
| F11R | Junctional adhesion molecule A | 0.43 |
| ADAM10 | Disintegrin and metalloproteinase domain-containing protein 10 | 0.43 |
| FAM38A | Piezo-type mechanosensitive ion channel component 1 | 0.43 |
| CD68 | Macrosialin | 0.43 |
| SLC26A6 | Solute carrier family 26 member 6 | 0.43 |
| MCOLN1 | Mucolipin-1 | 0.43 |
| SLCO3A1 | Solute carrier organic anion transporter family member 3A1 | 0.43 |
| PPAP2B | Lipid phosphate phosphohydrolase 3 | 0.43 |
| ICAM4 | Intercellular adhesion molecule 4 | 0.43 |
| CXCR1 | C—X—C chemokine receptor type 1 | 0.43 |
| CD300A | CMRF35-like molecule 8 | 0.43 |
| RELL1 | RELT-like protein 1 | 0.43 |
| TAPBPL | Tapasin-related protein | 0.42 |
| FCGR2C | Low affinity immunoglobulin gamma Fc region receptor II-c | 0.42 |
| SLC16A6 | Monocarboxylate transporter 7 | 0.42 |
| TMED1 | Transmembrane emp24 domain-containing protein 1 | 0.42 |
| CD86 | T-lymphocyte activation antigen CD86 | 0.42 |
| SLC16A3 | Monocarboxylate transporter 4 | 0.42 |
| SLC2A5 | Solute carrier family 2, facilitated glucose transporter member 5 | 0.42 |
| SLC29A1 | Equilibrative nucleoside transporter 1 | 0.42 |
| SLC16A14 | Monocarboxylate transporter 14 | 0.42 |
| P2RY2 | P2Y purinoceptor 2 | 0.42 |
| SUCNR1 | Succinate receptor 1 | 0.42 |
| BTN3A1 | Butyrophilin subfamily 3 member A1 | 0.41 |
| LAT2 | Linker for activation of T-cells family member 2 | 0.41 |
| PLXND1 | Plexin-D1 | 0.41 |
| ECE1 | Endothelin-converting enzyme 1 | 0.41 |
| TGFBR1 | TGF-beta receptor type-1 | 0.41 |
| CCRL2 | C—C chemokine receptor-like 2 | 0.41 |
| TFR2 | Transferrin receptor protein 2 | 0.41 |
| SLC44A1 | Choline transporter-like protein 1 | 0.41 |
| ITGA6 | Integrin alpha-6 light chain | 0.41 |
| PMP22 | Peripheral myelin protein 22 | 0.41 |
| LAX1 | Lymphocyte transmembrane adapter 1 | 0.4 |
| AMIGO2 | Amphoterin-induced protein 2 | 0.4 |
| SLC38A1 | Sodium-coupled neutral amino acid transporter 1 | 0.4 |
| SLC41A1 | Solute carrier family 41 member 1 | 0.4 |
| C2orf89 | Metalloprotease TIKI1 | 0.4 |
| ABCC10 | Multidrug resistance-associated protein 7 | 0.4 |
| CLDN15 | Claudin-15 | 0.4 |
| SLC39A6 | Zinc transporter ZIP6 | 0.4 |
| SLC16A5 | Monocarboxylate transporter 6 | 0.4 |
| TTYH3 | Protein tweety homolog 3 | 0.4 |
| ATP7A | Copper-transporting ATPase 1 | 0.4 |
| COMT | Catechol O-methyltransferase | 0.4 |
| SLC17A5 | Sialin | 0.4 |
| TMIGD2 | Transmembrane and immunoglobulin domain-containing protein 2 | 0.4 |
| CLEC7A | C-type lectin domain family 7 member A | 0.4 |
| SLC31A1 | High affinity copper uptake protein 1 | 0.4 |
| LRRC4 | Leucine-rich repeat-containing protein 4 | 0.4 |
| P2RY10 | Putative P2Y purinoceptor 10 | 0.39 |
| ATP10D | Probable phospholipid-transporting ATPase VD | 0.39 |
| BTN3A3 | Butyrophilin subfamily 3 member A3 | 0.39 |
| LIME1 | Lck-interacting transmembrane adapter 1 | 0.39 |
| TNF | Tumor necrosis factor, soluble form | 0.39 |
| PAQR8 | Membrane progestin receptor beta | 0.39 |
| OXER1 | Oxoeicosanoid receptor 1 | 0.39 |
| TRAT1 | T-cell receptor-associated transmembrane adapter 1 | 0.39 |
| GPBAR1 | G-protein coupled bile acid receptor 1 | 0.39 |
| SLC36A1 | Proton-coupled amino acid transporter 1 | 0.39 |
| PTPRE | Receptor-type tyrosine-protein phosphatase epsilon | 0.39 |
| PROM1 | Prominin-1 | 0.39 |
| CD74 | HLA class II histocompatibility antigen gamma chain | 0.38 |
| CNST | Consortin | 0.38 |
| TMEM49 | Vacuole membrane protein 1 | 0.38 |
| CLIC4 | Chloride intracellular channel protein 4 | 0.38 |
| NAALADL1 | N-acetylated-alpha-linked acidic dipeptidase-like protein | 0.38 |
| ANTXR2 | Anthrax toxin receptor 2 | 0.38 |
| FGFR1 | Fibroblast growth factor receptor 1 | 0.38 |
| IL1RAP | Interleukin-1 receptor accessory protein | 0.38 |
| ATP1B2 | Sodium/potassium-transporting ATPase subunit beta-2 | 0.38 |
| ABCG2 | ATP-binding cassette sub-family G member 2 | 0.38 |
| CLEC12A | C-type lectin domain family 12 member A | 0.38 |
| HLA-DQA1 | HLA class II histocompatibility antigen, DQ alpha 1 chain | 0.37 |
| B4GALT1 | Processed beta-1,4-galactosyltransferase 1 | 0.37 |
| CNNM3 | Metal transporter CNNM3 | 0.37 |
| ATP1B1 | Sodium/potassium-transporting ATPase subunit beta-1 | 0.37 |
| SLC39A1 | Zinc transporter ZIP1 | 0.37 |
| ATRN | Attractin | 0.37 |
| CYSLTR1 | Cysteinyl leukotriene receptor 1 | 0.37 |
| TRPV2 | Transient receptor potential cation channel subfamily V member 2 | 0.37 |
| SLC27A1 | Long-chain fatty acid transport protein 1 | 0.37 |
| GPR171 | Probable G-protein coupled receptor 171 | 0.37 |
| DAGLB | Sn1-specific diacylglycerol lipase beta | 0.37 |
| KCNQ1 | Potassium voltage-gated channel subfamily KQT member 1 | 0.37 |
| FZD6 | Frizzled-6 | 0.37 |
| CSF2RA | Granulocyte-macrophage colony-stimulating factor receptor | 0.37 |
| | subunit alpha | |
| PTH2R | Parathyroid hormone 2 receptor | 0.37 |
| MARCH1 | E3 ubiquitin-protein ligase MARCH1 | 0.36 |
| BACE2 | Beta-secretase 2 | 0.36 |
| CD5 | T-cell surface glycoprotein CD5 | 0.36 |
| TMEM219 | Insulin-like growth factor-binding protein 3 receptor | 0.36 |
| XPR1 | Xenotropic and polytropic retrovirus receptor 1 | 0.36 |
| CD1C | T-cell surface glycoprotein CD1c | 0.36 |
| CNNM2 | Metal transporter CNNM2 | 0.36 |
| TMEM88 | Transmembrane protein 88 | 0.36 |
| ICOS | Inducible T-cell costimulator | 0.36 |
| KLRG1 | Killer cell lectin-like receptor subfamily G member 1 | 0.36 |
| LRP8 | Low-density lipoprotein receptor-related protein 8 | 0.36 |
| F2R | Proteinase-activated receptor 1 | 0.36 |
| HM13 | Minor histocompatibility antigen H13 | 0.36 |
| EMR2 | EGF-like module-containing mucin-like hormone receptor-like 2 | 0.36 |
| TREML1 | Trem-like transcript 1 protein | 0.36 |
| C17orf60 | Allergin-1 | 0.36 |
| GPR146 | Probable G-protein coupled receptor 146 | 0.36 |
| SLAMF6 | SLAM family member 6 | 0.35 |
| SLC7A6 | Y + L amino acid transporter 2 | 0.35 |
| RELL2 | RELT-like protein 2 | 0.35 |
| LGR6 | Leucine-rich repeat-containing G-protein coupled receptor 6 | 0.35 |
| PANX1 | Pannexin-1 | 0.35 |
| C18orf1 | Low-density lipoprotein receptor class A domain-containing | 0.35 |
| | protein 4 | |
| SLMAP | Sarcolemmal membrane-associated protein | 0.35 |
| CCR5 | C—C chemokine receptor type 5 | 0.35 |
| MUC1 | Mucin-1 subunit beta | 0.35 |
| EMR3 | EGF-like module-containing mucin-like hormone receptor-like 3 | 0.35 |
| | subunit beta | |
| COL23A1 | Collagen alpha-1(XXIII) chain | 0.35 |
| OR2W3 | Olfactory receptor 2W3 | 0.35 |
| LNPEP | Leucyl-cystinyl aminopeptidase, pregnancy serum form | 0.34 |
| PRR7 | Proline-rich protein 7 | 0.34 |
| NOTCH1 | Notch 1 intracellular domain | 0.34 |
| RFT1 | Solute carrier family 52, riboflavin transporter, member 1 | 0.34 |
| TNFRSF25 | Tumor necrosis factor receptor superfamily member 25 | 0.34 |
| ANO6 | Anoctamin-6 | 0.34 |
| AQP3 | Aquaporin-3 | 0.34 |
| ADAM9 | Disintegrin and metalloproteinase domain-containing protein 9 | 0.34 |
| INSR | Insulin receptor subunit beta | 0.34 |
| FZD5 | Frizzled-5 | 0.34 |
| ERG | Potassium voltage-gated channel subfamily H member 2 | 0.34 |
| MME | Neprilysin | 0.34 |
| FCGR2B | Low affinity immunoglobulin gamma Fc region receptor II-b | 0.33 |
| LSR | Lipolysis-stimulated lipoprotein receptor | 0.33 |
| DDR1 | Epithelial discoidin domain-containing receptor 1 | 0.33 |
| CNR2 | Cannabinoid receptor 2 | 0.33 |
| ATR | Anthrax toxin receptor 1 | 0.33 |
| P2RY14 | P2Y purinoceptor 14 | 0.33 |
| VEZT | Vezatin | 0.33 |
| ALG10B | Putative Dol-P-Glc:Glc(2)Man(9)GlcNAc(2)-PP-Dol alpha-1,2- | 0.33 |
| | glucosyltransferase | |
| PAQR7 | Membrane progestin receptor alpha | 0.33 |
| FLT3LG | Fms-related tyrosine kinase 3 ligand | 0.33 |
| CD40LG | CD40 ligand, soluble form | 0.33 |
| FCGR2A | Low affinity immunoglobulin gamma Fc region receptor II-a | 0.33 |
| CLDN12 | Claudin-12 | 0.33 |
| GP6 | Platelet glycoprotein VI | 0.33 |
| EPHB4 | Ephrin type-B receptor 4 | 0.33 |
| SEMA4C | Semaphorin-4C | 0.33 |
| CD300C | CMRF35-like molecule 6 | 0.33 |
| PEAR1 | Platelet endothelial aggregation receptor 1 | 0.33 |
| FFAR2 | Free fatty acid receptor 2 | 0.33 |
| SLC2A6 | Solute carrier family 2, facilitated glucose transporter member 6 | 0.32 |
| TMEM150A | Transmembrane protein 150A | 0.32 |
| ANO8 | Anoctamin-8 | 0.32 |
| CD200R1 | Cell surface glycoprotein CD200 receptor 1 | 0.32 |
| FCER1A | High affinity immunoglobulin epsilon receptor subunit alpha | 0.32 |
| BEST1 | Bestrophin-1 | 0.32 |
| CLDN5 | Claudin-5 | 0.32 |
| SLC47A1 | Multidrug and toxin extrusion protein 1 | 0.32 |
| SLC5A10 | Sodium/glucose cotransporter 5 | 0.32 |
| CD40 | Tumor necrosis factor receptor superfamily member 5 | 0.31 |
| ANO9 | Anoctamin-9 | 0.31 |
| CLEC2D | C-type lectin domain family 2 member D | 0.31 |
| VIPR1 | Vasoactive intestinal polypeptide receptor 1 | 0.31 |
| SLC16A7 | Monocarboxylate transporter 2 | 0.31 |
| UTS2R | Urotensin-2 receptor | 0.31 |
| CLSTN3 | Calsyntenin-3 | 0.31 |
| GPR35 | G-protein coupled receptor 35 | 0.31 |
| SYT15 | Synaptotagmin-15 | 0.31 |
| FAM57A | Protein FAM57A | 0.31 |
| CD8B | T-cell surface glycoprotein CD8 beta chain | 0.31 |
| IL17RC | Interleukin-17 receptor C | 0.31 |
| GLDN | Gliomedin | 0.31 |
| FZD2 | Frizzled-2 | 0.31 |
| KCNA3 | Potassium voltage-gated channel subfamily A member 3 | 0.3 |
| MGA | Glucoamylase | 0.3 |
| GPR1 | G-protein coupled receptor 1 | 0.3 |
| IL6ST | Interleukin-6 receptor subunit beta | 0.3 |
| PCDHGB5 | Protocadherin gamma-B5 | 0.3 |
| OR1I1 | Olfactory receptor 1I1 | 0.3 |
| PTH1R | Parathyroid hormone/parathyroid hormone-related peptide | 0.3 |
| | receptor | |
| NLGN2 | Neuroligin-2 | 0.3 |
| MMP24 | Processed matrix metalloproteinase-24 | 0.3 |
| CDH22 | Cadherin-22 | 0.3 |
| TNFRSF8 | Tumor necrosis factor receptor superfamily member 8 | 0.3 |
| CHRNG | Acetylcholine receptor subunit gamma | 0.3 |
| PSEN1 | Presenilin-1 CTF12 | 0.3 |
| GPR114 | Probable G-protein coupled receptor 114 | 0.3 |
| PLXNB2 | Plexin-B2 | 0.3 |
| CHRNA2 | Neuronal acetylcholine receptor subunit alpha-2 | 0.3 |
| GPR34 | Probable G-protein coupled receptor 34 | 0.3 |
| LPAR6 | Lysophosphatidic acid receptor 6 | 0.3 |
| ATP8A1 | Probable phospholipid-transporting ATPase IA | 0.3 |
| FZD1 | Frizzled-1 | 0.3 |
| CCR2 | C—C chemokine receptor type 2 | 0.3 |
| P2RY1 | P2Y purinoceptor 1 | 0.3 |
| SLC16A9 | Monocarboxylate transporter 9 | 0.3 |
| C20orf103 | Lysosome-associated membrane glycoprotein 5 | 0.3 |
| ADORA2B | Adenosine receptor A2b | 0.3 |
| CLEC12B | C-type lectin domain family 12 member B | 0.3 |
| FCRL3 | Fc receptor-like protein 3 | 0.29 |
| CD180 | CD180 antigen | 0.29 |
| TIGIT | T-cell immunoreceptor with Ig and ITIM domains | 0.29 |
| PPAP2A | Lipid phosphate phosphohydrolase 1 | 0.29 |
| ATP11C | Probable phospholipid-transporting ATPase IG | 0.29 |
| TNFRSF17 | Tumor necrosis factor receptor superfamily member 17 | 0.29 |
| TNFSF12 | Tumor necrosis factor ligand superfamily member 12, secreted | 0.29 |
| | form | |
| TBXA2R | Thromboxane A2 receptor | 0.29 |
| OR3A3 | Olfactory receptor 3A3 | 0.29 |
| GPR153 | Probable G-protein coupled receptor 153 | 0.29 |
| ATP11A | Probable phospholipid-transporting ATPase IH | 0.29 |
| LRFN1 | Leucine-rich repeat and fibronectin type III domain-containing | 0.29 |
| | protein 1 | |
| OR51B2 | Olfactory receptor 51B2 | 0.29 |
| KCNS1 | Potassium voltage-gated channel subfamily S member 1 | 0.29 |
| OR12D2 | Olfactory receptor 12D2 | 0.29 |
| GRM4 | Metabotropic glutamate receptor 4 | 0.29 |
| NEO1 | Neogenin | 0.29 |
| DRD5 | D(1B) dopamine receptor | 0.29 |
| PLXDC1 | Plexin domain-containing protein 1 | 0.29 |
| GPR157 | Probable G-protein coupled receptor 157 | 0.29 |
| CD300LB | CMRF35-like molecule 7 | 0.29 |
| MARVELD1 | MARVEL domain-containing protein 1 | 0.29 |
| MFAP3 | Microfibril-associated glycoprotein 3 | 0.29 |
| CHRNB1 | Acetylcholine receptor subunit beta | 0.29 |
| PVRL2 | Poliovirus receptor-related protein 2 | 0.29 |
| F2RL1 | Proteinase-activated receptor 2, alternate cleaved 2 | 0.29 |
| GPR124 | G-protein coupled receptor 124 | 0.29 |
| BACE1 | Beta-secretase 1 | 0.29 |
| C6orf105 | Androgen-dependent TFPI-regulating protein | 0.28 |
| CXCR3 | C—X—C chemokine receptor type 3 | 0.28 |
| IGSF8 | Immunoglobulin superfamily member 8 | 0.28 |
| ATP8B1 | Probable phospholipid-transporting ATPase IC | 0.28 |
| TP53I13 | Tumor protein p53-inducible protein 13 | 0.28 |
| MC1R | Melanocyte-stimulating hormone receptor | 0.28 |
| CD84 | SLAM family member 5 | 0.28 |
| CALHM1 | Calcium homeostasis modulator protein 1 | 0.28 |
| CHRNA6 | Neuronal acetylcholine receptor subunit alpha-6 | 0.28 |
| CDH10 | Cadherin-10 | 0.28 |
| SLC16A1 | Monocarboxylate transporter 1 | 0.28 |
| GPRC5D | G-protein coupled receptor family C group 5 member D | 0.28 |
| AGER | Advanced glycosylation end product-specific receptor | 0.28 |
| FASLG | FasL intracellular domain | 0.28 |
| GPR56 | GPR56 C-terminal fragment | 0.28 |
| SIGLEC1 | Sialoadhesin | 0.28 |
| KIR2DL5A | Killer cell immunoglobulin-like receptor 2DL5A | 0.28 |
| PLB1 | Lysophospholipase | 0.28 |
| CD200 | OX-2 membrane glycoprotein | 0.27 |
| ADAM28 | Disintegrin and metalloproteinase domain-containing protein 28 | 0.27 |
| SIT1 | Sodium- and chloride-dependent transporter XTRP3 | 0.27 |
| SLC23A2 | Solute carrier family 23 member 2 | 0.27 |
| CCR10 | C—C chemokine receptor type 10 | 0.27 |
| PRR4 | Processed poliovirus receptor-related protein 4 | 0.27 |
| GJD2 | Gap junction delta-2 protein | 0.27 |
| SLC2A8 | Solute carrier family 2, facilitated glucose transporter member 8 | 0.27 |
| CD209 | CD209 antigen | 0.27 |
| CD274 | Programmed cell death 1 ligand 1 | 0.27 |
| PROM2 | Prominin-2 | 0.27 |
| ATP6V0A2 | V-type proton ATPase 116 kDa subunit a isoform 2 | 0.27 |
| MPZ | Myelin protein P0 | 0.27 |
| TNFRSF18 | Tumor necrosis factor receptor superfamily member 18 | 0.27 |
| MFSD2A | Major facilitator superfamily domain-containing protein 2A | 0.27 |
| HEG1 | Protein HEG homolog 1 | 0.27 |
| OXTR | Oxytocin receptor | 0.27 |
| CD99L2 | CD99 antigen-like protein 2 | 0.27 |
| LILRB4 | Leukocyte immunoglobulin-like receptor subfamily B member 4 | 0.27 |
| SMAGP | Small cell adhesion glycoprotein | 0.27 |
| OR51I2 | Olfactory receptor 51I2 | 0.27 |
| LY6G6D | Lymphocyte antigen 6 complex locus protein G6f | 0.27 |
| KCNQ4 | Potassium voltage-gated channel subfamily KQT member 4 | 0.27 |
| HRH2 | Histamine H2 receptor | 0.27 |
| SLC39A2 | Zinc transporter ZIP2 | 0.27 |
| CLDN10 | Claudin-10 | 0.27 |
| GPM6B | Neuronal membrane glycoprotein M6-b | 0.27 |
| STEAP4 | Metalloreductase STEAP4 | 0.27 |
| APOLD1 | Apolipoprotein L domain-containing protein 1 | 0.27 |
| S1PR3 | Sphingosine 1-phosphate receptors | 0.27 |
| SGMS2 | Phosphatidylcholine:ceramide cholinephosphotransferase 2 | 0.27 |
| KIR2DS5 | Killer cell immunoglobulin-like receptor 2DS5 | 0.27 |
| STAR | Heat-stable enterotoxin receptor | 0.27 |
| NIPA1 | Magnesium transporter NIPA1 | 0.26 |
| CNNM4 | Metal transporter CNNM4 | 0.26 |
| SLAMF1 | Signaling lymphocytic activation molecule | 0.26 |
| KIAA1919 | Sodium-dependent glucose transporter 1 | 0.26 |
| TLR6 | Toll-like receptor 6 | 0.26 |
| CRB3 | Protein crumbs homolog 3 | 0.26 |
| SLC12A9 | Solute carrier family 12 member 9 | 0.26 |
| GPR68 | Ovarian cancer G-protein coupled receptor 1 | 0.26 |
| OR51J1 | Olfactory receptor 51J1 | 0.26 |
| TREML2 | Trem-like transcript 2 protein | 0.26 |
| GPR176 | Probable G-protein coupled receptor 176 | 0.26 |
| FLVCR2 | Feline leukemia virus subgroup C receptor-related protein 2 | 0.26 |
| LPAR1 | Lysophosphatidic acid receptor 1 | 0.26 |
| PANX2 | Pannexin-2 | 0.26 |
| SLC6A6 | Sodium- and chloride-dependent taurine transporter | 0.26 |
| PROKR2 | Prokineticin receptor 2 | 0.26 |
| CLDN9 | Claudin-9 | 0.26 |
| MYOF | Myoferlin | 0.26 |
| LY6G6F | Lymphocyte antigen 6 complex locus protein G6f | 0.26 |
| ESAM | Endothelial cell-selective adhesion molecule | 0.26 |
| NCR3 | Natural cytotoxicity triggering receptor 3 | 0.25 |
| HLA-DQB2 | HLA class II histocompatibility antigen, DQ beta 2 chain | 0.25 |
| SLC4A5 | Electrogenic sodium bicarbonate cotransporter 4 | 0.25 |
| P2RY4 | P2Y purinoceptor 4 | 0.25 |
| ABCB1 | Multidrug resistance protein 1 | 0.25 |
| SLC9A1 | Sodium/hydrogen exchanger 1 | 0.25 |
| CELSR2 | Cadherin EGF LAG seven-pass G-type receptor 2 | 0.25 |
| SYT8 | Synaptotagmin-8 | 0.25 |
| PCDHA9 | Protocadherin alpha-9 | 0.25 |
| TMEM204 | Transmembrane protein 204 | 0.25 |
| PTPRJ | Receptor-type tyrosine-protein phosphatase eta | 0.25 |
| GRPR | Gastrin-releasing peptide receptor | 0.25 |
| SEMA6B | Semaphorin-6B | 0.25 |
| CLCN5 | H(+)/Cl(−) exchange transporter 5 | 0.25 |
| GLRA2 | Glycine receptor subunit alpha-2 | 0.25 |
| PLVAP | Plasmalemma vesicle-associated protein | 0.25 |
| ACVR1B | Activin receptor type-1 B | 0.25 |
| JAM3 | Junctional adhesion molecule C | 0.25 |
| LDLRAD3 | Low-density lipoprotein receptor class A domain-containing | 0.25 |
| | protein 3 | |
| XG | Glycoprotein Xg | 0.25 |
| SLC2A11 | Solute carrier family 2, facilitated glucose transporter member 11 | 0.24 |
| PCDH9 | Protocadherin-9 | 0.24 |
| VAMP5 | Vesicle-associated membrane protein 5 | 0.24 |
| CDHR2 | Cadherin-related family member 2 | 0.24 |
| DRD2 | D(2) dopamine receptor | 0.24 |
| LRIG2 | Leucine-rich repeats and immunoglobulin-like domains protein 2 | 0.24 |
| RAMP3 | Receptor activity-modifying protein 3 | 0.24 |
| SLC39A14 | Zinc transporter ZIP14 | 0.24 |
| STRA6 | Stimulated by retinoic acid gene 6 protein homolog | 0.24 |
| ADRA2C | Alpha-2C adrenergic receptor | 0.24 |
| CLDN19 | Claudin-19 | 0.24 |
| CX3CR1 | CX3C chemokine receptor 1 | 0.24 |
| CD79B | B-cell antigen receptor complex-associated protein beta chain | 0.24 |
| KIR2DL2 | Killer cell immunoglobulin-like receptor 2DL2 | 0.24 |
| CXCR7 | Atypical chemokine receptor 3 | 0.24 |
| OR5L2 | Olfactory receptor 5L2 | 0.24 |
| LRRC52 | Leucine-rich repeat-containing protein 52 | 0.24 |
| JPH1 | Junctophilin-1 | 0.24 |
| ADORA1 | Adenosine receptor A1 | 0.24 |
| GPRC5C | G-protein coupled receptor family C group 5 member C | 0.24 |
| RET | Extracellular cell-membrane anchored RET cadherin 120 kDa | 0.24 |
| | fragment | |
| PVR | Poliovirus receptor | 0.24 |
| ITGB3 | Integrin beta-3 | 0.24 |
| PTGIR | Prostacyclin receptor | 0.24 |
| LPHN1 | Latrophilin-1 | 0.24 |
| OR10J1 | Olfactory receptor 10J1 | 0.24 |
| MFAP3L | Microfibrillar-associated protein 3-like | 0.24 |
| GPNMB | Transmembrane glycoprotein NMB | 0.24 |
| CELSR3 | Cadherin EGF LAG seven-pass G-type receptor 3 | 0.23 |
| CCR6 | C—C chemokine receptor-like 2 | 0.23 |
| DMPK | Myotonin-protein kinase | 0.23 |
| UPK3B | Uroplakin-3b | 0.23 |
| OR1D2 | Olfactory receptor 1D2 | 0.23 |
| OR7D2 | Olfactory receptor 7D2 | 0.23 |
| ITGB1 | Integrin beta-1 | 0.23 |
| HRH3 | Histamine H3 receptor | 0.23 |
| GRIN2C | Glutamate receptor ionotropic, NMDA 2C | 0.23 |
| KIR3DL1 | Killer cell immunoglobulin-like receptor 3DL1 | 0.23 |
| EPHB2 | Ephrin type-B receptor 2 | 0.23 |
| OR2S2 | Olfactory receptor 2S2 | 0.23 |
| KIR2DL4 | Killer cell immunoglobulin-like receptor 2DL4 | 0.23 |
| CNNM1 | Metal transporter CNNM1 | 0.23 |
| MARVELD2 | MARVEL domain-containing protein 2 | 0.23 |
| CXCR6 | C—X—C chemokine receptor type 6 | 0.23 |
| NOV | Plexin-A1 | 0.23 |
| ABCB6 | ATP-binding cassette sub-family B member 6, mitochondrial | 0.23 |
| PVRL1 | Poliovirus receptor-related protein 1 | 0.23 |
| SLC46A2 | Thymic stromal cotransporter homolog | 0.23 |
| ADORA3 | Adenosine receptor A3 | 0.23 |
| GPR125 | Probable G-protein coupled receptor 125 | 0.23 |
| CD22 | B-cell receptor CD22 | 0.22 |
| FZD3 | Frizzled-3 | 0.22 |
| LPAR5 | Lysophosphatidic acid receptor 5 | 0.22 |
| TMEM8B | Transmembrane protein 8B | 0.22 |
| PLXNA1 | Plexin-A1 | 0.22 |
| NPFFR1 | Neuropeptide FF receptor 1 | 0.22 |
| SEZ6L2 | Seizure 6-like protein 2 | 0.22 |
| LRRTM2 | Leucine-rich repeat transmembrane neuronal protein 2 | 0.22 |
| SLC16A11 | Monocarboxylate transporter 11 | 0.22 |
| GRIK5 | Glutamate receptor ionotropic, kainate 5 | 0.22 |
| SYT6 | Synaptotagmin-6 | 0.22 |
| TMEM102 | Transmembrane protein 102 | 0.22 |
| OR8B8 | Olfactory receptor 8B8 | 0.22 |
| GJB1 | Gap junction beta-1 protein | 0.22 |
| GRM6 | Metabotropic glutamate receptor 6 | 0.22 |
| C20orf54 | Solute carrier family 52, riboflavin transporter, member 3 | 0.22 |
| OR52D1 | Olfactory receptor 52D1 | 0.22 |
| SLC46A1 | Proton-coupled folate transporter | 0.22 |
| DSC2 | Desmocollin-2 | 0.22 |
| FAT1 | Protocadherin Fat 1, nuclear form | 0.22 |
| GCGR | Glucagon receptor | 0.22 |
| POP1 | Blood vessel epicardial substance | 0.22 |
| CXADR | Coxsackievirus and adenovirus receptor | 0.22 |
| ABCC6 | Multidrug resistance-associated protein 6 | 0.22 |
| GJA1 | Gap junction alpha-1 protein | 0.22 |
| CXCR5 | C—X—C chemokine receptor type 5 | 0.21 |
| ABCB4 | Multidrug resistance protein 3 | 0.21 |
| CTLA4 | Cytotoxic T-lymphocyte protein 4 | 0.21 |
| TRPV1 | Transient receptor potential cation channel subfamily V member 1 | 0.21 |
| MRGPRX4 | Mas-related G-protein coupled receptor member X4 | 0.21 |
| SIGLEC6 | Sialic acid-binding Ig-like lectin 6 | 0.21 |
| IL9R | Interleukin-9 receptor | 0.21 |
| CHRNB2 | Neuronal acetylcholine receptor subunit beta-2 | 0.21 |
| PDGFRB | Platelet-derived growth factor receptor beta | 0.21 |
| TMPRSS11D | Transmembrane protease serine 11D catalytic chain | 0.21 |
| CDH24 | Cadherin-24 | 0.21 |
| PRRT2 | Proline-rich transmembrane protein 2 | 0.21 |
| GALR3 | Galanin receptor type 3 | 0.21 |
| OR51I1 | Olfactory receptor 51I1 | 0.21 |
| PTPRU | Receptor-type tyrosine-protein phosphatase U | 0.21 |
| LPAR4 | Lysophosphatidic acid receptor 4 | 0.21 |
| ZNRF3 | E3 ubiquitin-protein ligase ZNRF3 | 0.21 |
| P2RY6 | P2Y purinoceptor 6 | 0.21 |
| AGTR1 | Type-1 angiotensin II receptor | 0.21 |
| GPR182 | G-protein coupled receptor 182 | 0.21 |
| PODXL | Podocalyxin | 0.21 |
| BDKRB1 | B1 bradykinin receptor | 0.21 |
| DCHS1 | Protocadherin-16 | 0.21 |
| GRIN3B | Glutamate receptor ionotropic, NMDA 3B | 0.21 |
| PTGDR | Prostaglandin D2 receptor | 0.21 |
| PVRL4 | Processed poliovirus receptor-related protein 4 | 0.21 |
| GPR77 | C5a anaphylatoxin chemotactic receptor 2 | 0.21 |
| PARM1 | Prostate androgen-regulated mucin-like protein 1 | 0.21 |
| OR10H1 | Olfactory receptor 10H1 | 0.21 |
| OR10D3 | Putative olfactory receptor 10D3 | 0.21 |
| TNFSF14 | Tumor necrosis factor ligand superfamily member 14, soluble | 0.21 |
| | form | |
| FCRL5 | Fc receptor-like protein 5 | 0.2 |
| RNF43 | E3 ubiquitin-protein ligase RNF43 | 0.2 |
| AMIGO1 | Amphoterin-induced protein 1 | 0.2 |
| OR1F1 | Olfactory receptor 1F1 | 0.2 |
| SLCO4A1 | Solute carrier organic anion transporter family member 4A1 | 0.2 |
| TTYH2 | Protein tweety homolog 2 | 0.2 |
| GABRR2 | Gamma-aminobutyric acid receptor subunit rho-2 | 0.2 |
| GJD3 | Gap junction delta-3 protein | 0.2 |
| GRID1 | Glutamate receptor ionotropic, delta-1 | 0.2 |
| CLDN1 | Claudin-1 | 0.2 |
| SLC6A13 | Sodium- and chloride-dependent GABA transporter 2 | 0.2 |
| SLC30A8 | Zinc transporter 8 | 0.2 |
| KIR2DL3 | Killer cell immunoglobulin-like receptor 2DL3 | 0.2 |
| GPR78 | G-protein coupled receptor 78 | 0.2 |
| UPK2 | Uroplakin-2 | 0.2 |
| CLDN14 | Claudin-14 | 0.2 |
| EDA | Ectodysplasin-A, secreted form | 0.2 |
| PTGER1 | Prostaglandin E2 receptor EP1 subtype | 0.2 |
| TRPV5 | Transient receptor potential cation channel subfamily V member 5 | 0.2 |
| PRIMA1 | Proline-rich membrane anchor 1 | 0.2 |
| GJA9 | Gap junction alpha-9 protein | 0.2 |
| SLC7A3 | Cationic amino acid transporter 3 | 0.2 |
| SSTR2 | Somatostatin receptor type 2 | 0.2 |
| CD1A | T-cell surface glycoprotein CD1a | 0.2 |
| SLC7A8 | Large neutral amino acids transporter small subunit 2 | 0.2 |
| CLIC6 | Chloride intracellular channel protein 6 | 0.2 |
| EPHA8 | Ephrin type-A receptor 8 | 0.2 |
| SLC20A2 | Sodium-dependent phosphate transporter 2 | 0.2 |
| SCNN1A | Amiloride-sensitive sodium channel subunit alpha | 0.2 |
| OR51B6 | Olfactory receptor 51B6 | 0.2 |
| OR14J1 | Olfactory receptor 14J1 | 0.2 |
| OR10C1 | Olfactory receptor 10C1 | 0.2 |
| OPRL1 | Nociceptin receptor | 0.2 |
| CCR9 | C—C chemokine receptor type 9 | 0.2 |
| JPH4 | Junctophilin-4 | 0.2 |
| HTR1E | 5-hydroxytryptamine receptor 1E | 0.2 |
| MC3R | Melanocortin receptor 3 | 0.2 |
| CD163L1 | Scavenger receptor cysteine-rich type 1 protein M160 | 0.2 |
| SEZ6 | Seizure protein 6 homolog | 0.2 |
| PRSS8 | Prostasin heavy chain | 0.2 |
| CDH26 | Cadherin-like protein 26 | 0.2 |
| ODZ1 | Teneurin C-terminal-associated peptide | 0.2 |
| FGFR3 | Fibroblast growth factor receptor 3 | 0.2 |

### REFERENCES

Bardenheuer, W., K. Lehmberg, et al. (2005). “Resistance to cytarabine and gemcitabine and in vitro selection of transduced cells after retroviral expression of cytidine deaminase in human hematopoietic progenitor cells.” Leukemia 19(12): 2281-8.
Betts, M. R., J. M. Brenchley, et al. (2003). “Sensitive and viable identification of antigen-specific CD8+ T cells by a flow cytometric assay for degranulation.” J Immunol Methods 281(1-2): 65-78.
Boch, J., H. Scholze, et al. (2009). “Breaking the code of DNA binding specificity of TAL-type III effectors.” Science 326(5959): 1509-12.
Brewin, J., C. Mancao, et al. (2009). “Generation of EBV-specific cytotoxic T cells that are resistant to calcineurin inhibitors for the treatment of posttransplantation lymphoproliferative disease.” Blood 114(23): 4792-803.
Cambier, J. C. (1995) “Antigen and Fc Receptor Signaling: The Awesome Power of the Immunoreceptor Tyrosine-I Based Activation Motif (ITAM)” The Journal of Immunology 155 (7) 3281-3285.
Cong, L., F. A. Ran, et al. (2013). “Multiplex genome engineering using CRISPR/Cas systems.” Science 339(6121): 819-23.
Critchlow, S. E. and S. P. Jackson (1998). “DNA end-joining: from yeast to man.” Trends Biochem Sci 23(10): 394-8.
Dalgaard, J. Z., A. J. Klar, et al. (1997). “Statistical modeling and analysis of the LAGLIDADG family of site-specific endonucleases and identification of an intein that encodes a site-specific endonuclease of the HNH family.” Nucleic Acids Res 25(22): 4626-38.
Deltcheva, E., K. Chylinski, et al. (2011). “CRISPR RNA maturation by trans-encoded small RNA and host factor RNase III.” Nature 471(7340): 602-7.
Garneau, J. E., M. E. Dupuis, et al. (2010). “The CRISPR/Cas bacterial immune system cleaves bacteriophage and plasmid DNA.” Nature 468(7320): 67-71.
Gasiunas, G., R. Barrangou, et al. (2012). “Cas9-crRNA ribonucleoprotein complex mediates specific DNA cleavage for adaptive immunity in bacteria.” Proc Natl Acad Sci USA 109(39): E2579-86.
Hacke, K., J. A. Treger, et al. (2013). “Genetic modification of mouse bone marrow by lentiviral vector-mediated delivery of hypoxanthine-Guanine phosphoribosyltransferase short hairpin RNA confers chemoprotection against 6-thioguanine cytotoxicity.” Transplant Proc 45(5): 2040-4.
Jena, B., G. Dotti, et al. (2010). “Redirecting T-cell specificity by introducing a tumor-specific chimeric antigen receptor.” Blood 116(7): 1035-44.
Jinek, M., K. Chylinski, et al. (2012). “A programmable dual-RNA-guided DNA endonuclease in adaptive bacterial immunity.” Science 337(6096): 816-21.
Jonnalagadda, M., C. E. Brown, et al. (2013). “Engineering human T cells for resistance to methotrexate and mycophenolate mofetil as an in vivo cell selection strategy.” PLoS One 8(6): e65519.
Kushman, M. E., S. L. Kabler, et al. (2007). “Expression of human glutathione S-transferase P1 confers resistance to benzo[a]pyrene or benzo[a]pyrene-7,8-dihydrodiol mutagenesis, macromolecular alkylation and formation of stable N2-Gua-BPDE adducts in stably transfected V79MZ cells co-expressing hCYP1A1.” Carcinogenesis 28(1): 207-14.
Lackner, G., N. Moebius, et al. (2011). “Complete genome sequence of Burkholderia rhizoxinica, an Endosymbiont of Rhizopus microsporus.” J Bacteriol 193(3): 783-4.
Ma, J. L., E. M. Kim, et al. (2003). “Yeast Mre11 and Rad1 proteins define a Ku-independent mechanism to repair double-strand breaks lacking overlapping end sequences.” Mol Cell Biol 23(23): 8820-8.
Mak, A. N., P. Bradley, et al. (2012). “The crystal structure of TAL effector PthXo1 bound to its DNA target.” Science 335(6069): 716-9.
Mali, P., L. Yang, et al. (2013). “RNA-guided human genome engineering via Cas9.” Science 339(6121): 823-6.
Metzger, H. et al. (1986) “The Receptor with High Affinity for Immunoglobulin E” Annual Review of Immunology. 4: 419-470
Moscou, M. J. and A. J. Bogdanove (2009). “A simple cipher governs DNA recognition by TAL effectors.” Science 326(5959): 1501.
Nivens, M. C., T. Felder, et al. (2004). “Engineered resistance to camptothecin and antifolates by retroviral coexpression of tyrosyl DNA phosphodiesterase-I and thymidylate synthase.” Cancer Chemother Pharmacol 53(2): 107-15.
Park, T. S., S. A. Rosenberg, et al. (2011). “Treating cancer with genetically engineered T cells.” Trends Biotechnol 29(11): 550-7.
Sangiolo, D., M. Lesnikova, et al. (2007). “Lentiviral vector conferring resistance to mycophenolate mofetil and sensitivity to ganciclovir for in vivo T-cell selection.” Gene Ther 14(21): 1549-54.
Schweitzer, B. I., A. P. Dicker, et al. (1990). “Dihydrofolate reductase as a therapeutic target.” Faseb J 4(8): 2441-52.
Sugimoto, Y., S. Tsukahara, et al. (2003). “Drug-selected co-expression of P-glycoprotein and gp91 in vivo from an MDR1-bicistronic retrovirus vector Ha-MDR-IRES-gp91.” J Gene Med 5(5): 366-76.
Takebe, N., S. C. Zhao, et al. (2001). “Generation of dual resistance to 4-hydroperoxycyclophosphamide and methotrexate by retroviral transfer of the human aldehyde dehydrogenase class 1 gene and a mutated dihydrofolate reductase gene.” Mol Ther 3(1): 88-96.
Waldmann H. and Hale G. (2005) “CAMPATH: from concept to clinic”. Phil. Trans. R. Soc. B 360: 1707-1711.
Yam, P., M. Jensen, et al. (2006). “Ex vivo selection and expansion of cells based on expression of a mutated inosine monophosphate dehydrogenase 2 after HIV vector transduction: effects on lymphocytes, monocytes, and CD34+ stem cells.” Mol Ther 14(2): 236-44.
Zielske, S. P., J. S. Reese, et al. (2003). “In vivo selection of MGMT (P140K) lentivirus-transduced human NOD/SCID repopulating cells without pretransplant irradiation conditioning.” J Clin Invest 112(10): 1561-70.

1. A composition comprising:
(i) engineered immune cells expressing on their cell surface a chimeric antigen receptor (CAR) specific for a cell surface antigen and also comprising at least one gene encoding the cell surface antigen that has been inactivated by genetic modification, wherein the cell surface antigen is expressed on both immune cells and pathological cells; and
(ii) a therapeutic antibody specific for said cell surface antigen.

2. The composition according to claim 1, wherein said immune cells comprise an immune T cell, or a hematopoietic stem cell.

3. The composition according to claim 2, wherein said immune T cell is an inflammatory T-cell, a cytotoxic T-cell, a regulatory T-cell, or a helper T-cell; or wherein said hematopoietic stem cell is a TCR negative hematopoietic stem cell.

4. The composition according to claim 1, wherein said cell surface antigen is selected from the group consisting of the genes listed in any of Tables 4 to 14.

5. The composition according to claim 3, wherein said cytoxic T cell is a cytotoxic CD4 T-cell or a cytotoxic CD8 T-cell.

6. The composition according to claim 1, wherein said genetic modification is generated using a rare cutting endonuclease selected from a Meganuclease, a transcription activator-like (TAL)-nuclease, a zing-finger nuclease (ZFN), or an RNA/DNA guided endonuclease.

7. The composition according to claim 2, wherein said immune T cell is further engineered by genetically inactivating at least one gene encoding a component of the T-cell receptor (TCR), and/or affecting expression of a component of the HLA complex.

8-13. (canceled)

14. The composition according to claim 1, wherein said CAR comprises an extracellular binding domain, said extracellular binding domain comprising at least two mAb-specific epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab.

15-21. (canceled)

22. The composition according to claim 1, wherein said cell surface antigen is CD70, and wherein the therapeutic antibody is selected from the group consisting of Vorsetuzumab, Vorsetuzumab mafodotin, SGN CD70A, MDX 1203, and ARGX 110.

23. The composition according to claim 22, wherein said genetic modification is generated using a transcription activator-like (TAL)-nuclease specific for CD70, comprising at least 95% homology with SEQ ID NO; 102 and SEQ ID NO: 103, comprising at least 95% homology with SEQ ID NO: 104 and SEQ ID NO: 105, or comprising at least 95% homology with SEQ ID NO; 106 and SEQ ID NO; 107 specific for the sequences SEQ ID NO: 73 and SEQ ID NO: 74; SEQ ID NO: 76 and SEQ ID NO: 77 and SEQ ID NO: 79 and SEQ ID NO: 80, respectively.

24. The composition according to claim 1, wherein said cell surface antigen is CS1, and wherein the therapeutic antibody is selected from the group consisting of Elotuzumab and ABBV838.

25. The composition according to claim 24, wherein said genetic modification is generated using a transcription activator-like (TAL)-nuclease specific for CS1, comprising at least 95% homology with SEQ ID NO: 108 and SEQ ID NO: 109 comprising at least 95% homology with SEQ ID NO; 110 and SEQ ID NO: 111, or comprising at least 95% homology with SEQ ID NO; 112 and SEQ ID NO: 113; each specific for the sequences SEQ ID NO: 64 and SEQ ID NO: 65, SEQ ID NO: 67 and SEQ ID NO: 68, SEQ ID NO: 70 and SEQ ID NO; 71, respectively.

26. The composition according to claim 24, wherein said genetic modification is generated using a transcription activator-like (TAL)-nuclease specific for CS1, comprising at least 95% homology with SEQ ID NO: 171 and SEQ ID NO: 172, comprising at least 95% homology with SEQ ID NO; 173 and SEQ ID NO: 174, comprising at least 95% homology with SEQ ID NO; 175 and SEQ ID NO: 176, or comprising at least 95% homology with SEQ ID NO: 177 and SEQ ID NO: 178.

27. The composition according to claim 1, wherein said surface antigen is CD38, and wherein the therapeutic antibody is selected from the group consisting of Daratumumab, Isatuximab, MOR202, MOR-03087, EDC8, and GBR 1342.

28. The composition according to claim 27, wherein said genetic modification is generated using a transcription activator-like (TAL)-nuclease specific for CD38, comprising at least 95% homology with SEQ ID NO; 114 and SEQ ID NO: 115, comprising at least 95% homology with SEQ ID NO; 116 and SEQ ID NO; 117 or comprising at least 95% homology with SEQ ID NO; 118 and SEQ ID NO: 119; specific for the sequences having at least 95% homology with SEQ ID NO: 1, SEQ ID NO: 4; and SEQ ID NO: 7, respectively.

29-53. (canceled)

54. A method for treating a human patient having cancer, the method comprising:
administering a composition comprising (i) engineered immune cells expressing on their cell surface a chimeric antigen receptor (CAR) specific for a cell surface antigen and also comprising at least one gene encoding the cell surface antigen that has been inactivated by genetic modification, wherein the cell surface antigen is expressed on both immune cells and pathological cells of the patient; and (ii) a therapeutic antibody specific for said cell surface antigen;
wherein said engineered immune cells are administered to said patient, before, after and/or concomitantly to administration of said therapeutic antibody, and wherein the cancer comprises cells expressing or overexpressing said cell surface antigen.

55-59. (canceled)

60. The method according to claim 54, wherein said cell surface antigen is selected from the group consisting of the genes listed in any of Tables 4 to 14.

61. The method according to claim 5, wherein said cell surface antigen is CD38 or an immuno-reactive fragment thereof, and wherein the therapeutic antibody is selected from the group consisting of Daratumumab, Isatuximab, MOR202, MOR-03087, EDC8, and GBR 1342.

62. The method according to claim 5, wherein said cell surface antigen is CD70 or an immuno-reactive fragment thereof, and wherein the therapeutic antibody is selected from the group consisting of Vorsetuzumab, Vorsetuzumab mafodotin, SGN CD70A, MDX 1203, and ARGX 110.

63. The method according to claim 5, wherein said cell surface antigen is CS1 or an immuno-reactive fragment thereof, and wherein the therapeutic antibody is selected from the group consisting of Elotuzumab and ABBV838.

64. The method according to claim 61, wherein said genetic modification is generated using a transcription activator-like (TAL-nuclease specific for CD38, comprising at least 95% homology with SEQ ID NO: 114 and SEQ ID NO: 115, comprising at least 95% homology with SEQ ID NO: 116 and SEQ ID NO: 117, or comprising at least 95% homology with SEQ ID NO: 118 and SEQ ID NO: 119, specific for the sequences having at least 95% homology with SEQ ID NO: 1, SEQ ID NO: 4, and SEQ ID NO: 7, respectively.

65. The method according to claim 62, wherein said genetic modification is generated using a transcription activator-like (TAL)-nuclease specific for CD70, comprising at least 95% homology with SEQ ID NO: 102 and SEQ ID NO: 103, comprising at least 95% homology with SEQ ID NO: 104 and SEQ ID NO: 105, comprising at least 95% homolog with SEQ ID NO: 106 and SEQ ID NO: 107 specific for the sequences SEQ ID NO: 73 and SEQ ID NO: 74: SEQ ID NO: 76 and SEQ ID NO: 77, SEQ ID NO: 79 and SEQ ID NO: 80, respectively.

66. The method according to claim 63, wherein said genetic modification is generated using a transcription activator-like (TAL)-nuclease specific for CS1, comprising at least 95% homology with SEQ ID NO: 108 and SEQ ID NO: 109, comprising at least 95% homology with SEQ ID NO: 110 and SEQ ID NO: 111, or comprising at least 95% homology with SEQ ID NO: 112 and SEQ ID NO: 113 each specific for the sequence SEQ ID NO: 64 and SEQ ID NO: 65, SEQ ID NO: 67 and SEQ ID NO: 68, SEQ ID NO: 70 and SEQ ID NO: 71, respectively.

67. The method according to claim 63, wherein said genetic modification is generated using a transcription activator-like (TAL)-nuclease specific for CS1, comprising at least 95% homology with SEQ ID NO: 171 and SEQ ID NO: 172, comprising at least 95% homology with SEQ ID NO: 173 and SEQ ID NO: 174, comprising at least 95% homology with SEQ ID NO: 175 and SEQ ID NO: 176, or comprising at least 95% homology with SEQ ID NO: 177 and SEQ ID NO: 178.

68-69. (canceled)

70. The method according to claim 54, wherein said genetic modification is performed using a rare cutting endonuclease selected from the group consisting of a Meganuclease, a transcription activator-like (TAL)-nuclease, a zing-finger nuclease (ZFN), or an RNA/DNA guided endonuclease.

71-75. (canceled)

76. The method according to claim 54, wherein said engineered immune cells comprise an inactivation of a gene encoding a component of the T-cell receptor (TCR), or a component of the HLA complex.

77-86. (canceled)

87. The method according to claim 54, wherein said engineered immune cells are immune T-cells, inflammatory T-cells, cytotoxic T-cells, regulatory T-cells, or helper T-cells.

88. The method according to claim 87, wherein said T-cells are cytotoxic CD4 T cells or cytotoxic CD8 T-cells.

89-100. (canceled)

101. A method of treating a human patient having a cancer, said method comprising:
administering a composition comprising (i) engineered immune T-cells expressing on their cell surface a chimeric antigen receptor (CAR) specific for CD70 and also comprising a genetic modification of the CD70 gene resulting in the inactivation of said CD70 gene, and (ii) an ARGX 110 anti-CD70 therapeutic antibody, wherein said cancer is Cutaneous T cell lymphoma; or
administering a composition comprising (i′) engineered immune T-cells expressing on their cell surfaces a CAR specific for CD70 and comprising a genetic modification of the CD70 gene resulting in the inactivation of said CD70 gene, and (ii′) an MDX 1203 anti-CD70 therapeutic antibody, wherein said cancer is Renal Cell Carcinoma or Non-Hodgkin's Lymphoma.

102. A method of treating a human patient having a cancer, said method comprising:
administering a composition comprising (i) engineered immune T-cells expressing on their cell surfaces a chimeric antigen receptor (CAR) specific for CS1 and also comprising a genetic modification of the CS1 gene resulting in the inactivation of said CS1 gene, and (ii) an anti-CS1 therapeutic antibody selected from the group consisting of elotuzumab and ABBV838, wherein said cancer is selected from the group consisting of carcinoma, blastoma, and sarcoma.

103. A method of treating a human patient having a cancer, said method comprising:
administering a composition comprising (i) engineered immune T-cells expressing on their cell surface a chimeric antigen receptor (CAR) specific for CD38 and also comprising a genetic modification of the CD38 gene resulting in the inactivation of said CD38 gene, and (ii) an Isatuximab anti-CD38 therapeutic antibody, wherein said cancer is selected from the group consisting of Prostate Cancer, Non-small Cell Lung Cancer, Plasma Cell Myeloma, MM, T-cell Type Acute Leukemia, Precursor T-lymphoblastic Lymphoma or Leukaemia, prostate cancer, Non-small Cell Lung Cancer, or a CD38-expressing or CD38 over expressing hematologic cancer selected from Chronic Lymphocytic Leukemia (CLL), Chronic Myelogenous Leukemia (CML), Acute Myelogenous Leukemia (AML), and Acute Lymphocytic Leukemia (ALL), multiple myeloma (MM), or an hematologic cancer selected from the group of B-cell chronic lymphocytic leukemia (B-CLL), acute lymphoblastic leukemia, chronic myeloid leukemia, chronic lymphocytic leukemia (CLL), chronic myelogenous leukemia or chronic myeloid leukemia (CML), acute myelogenous leukemia or acute myeloid leukemia (AML), acute lymphocytic leukemia (ALL), hairy cell leukemia (HCL), myelodysplasia syndromes (MDS) or of chronic; or myelogenous leukemia (CML-BP) and any subtypes of chronic myelogenous leukemia (CML-BP); or
administering a composition comprising (i′) engineered immune T-cells expressing on their cell surface a CAR specific for CD38 and comprising a genetic modification of the CD38 gene resulting in the inactivation of said CD38 gene, and (ii′) a Daratumumab anti-CD38 therapeutic antibody, wherein said cancer is selected from the group consisting of Microsatellite Unstable Colorectal Cancer, Microsatellite Stable Colorectal Cancer, Mismatch Repair Proficient Colorectal Cancer, Mismatch Repair Deficient Colorectal Cancer, Waldenström Macroglobulinemia, Malignant Neoplasms of Male Genital Organs, Prostate Cancer, Hematopoietic Cancer, Acute Myelogenous Leukemia, High-Risk Myelodysplastic Syndrome, Plasma cell myeloma, Monoclonal Gammopathy, Smoldering Multiple Myeloma, Membranoproliferative Glomerulonephritis, and Multiple Myeloma.
